(19) United States
(12) Patent Application Publication
(10) Pub. No.: US 2024/0189397 A1
(43) Pub. Date: Jun. 13, 2024

(54) Heparin-Associated Polypeptides and Uses Thereof

(71) Applicant: Juvena Therapeutics, Inc., Redwood City, CA (US)

(72) Inventors: Hanadie Yousef, Redwood City, CA (US); Jeremy O'Connell, Palo Alto, CA (US); Thach Mai, South San Francisco, CA (US); Rami Jaafar, San Francisco, CA (US)

(73) Assignee: Juvena Therapeutics, Inc., Redwood City, CA (US)

(21) Appl. No.: 17/843,676

(22) PCT Filed: Dec. 22, 2020
(86) PCT No.: PCT/US2020/066739

§ 371 Date: Jun. 17, 2022

Related U.S. Application Data
(60) Provisional application No. 62/953,425, filed on Dec. 24, 2019.
(60) Provisional application No. 62/967,393, filed on Jan. 29, 2020.

(51) Int. Cl. A61K 38/30; A61K 38/18; A61K 47/64; A61K 47/68; A61P 21/06

(52) U.S. Cl. CPC A61K 38/30; A61K 38/1825; A61K 38/1841; A61K 38/1858; A61K 38/1875; A61K 47/643; A61K 47/6811; A61P 21/06

(57) ABSTRACT

Described herein are therapeutic compositions comprising heparin-associated polypeptides useful for the treatment of soft-tissue and muscle diseases, disorders, and injuries.

### CROSS-REFERENCE TO RELATED APPLICATIONS

This application is a 371 of international application PCT/US2020/066739 filed on Dec. 22, 2020, which claims the benefit of U.S. Provisional Application No. 62/953,425 filed Dec. 24, 2019, and U.S. Provisional Application No. 62/967,393 filed Jan. 29, 2020, all of which are incorporated by reference in their entireties.

### BACKGROUND

As the average life span increases, increasing emphasis is placed upon “healthy aging.” Individuals would like to live more active lifestyles as they age, and as a result, many aging disorders can have a significant impact on the quality of life of aging individuals. Treatments directed to regenerative ends have utility for treating aging diseases. Additionally, many treatments for aging disorders can be applicable to younger individuals who have suffered illness, injury, or who possess genetic or developmental defects leading to premature tissue loss, wasting, or weakening.

### SUMMARY

As individuals age, tissue progenitor cells lose their regenerative potential. Described herein, in certain aspects, are heparin-associated polypeptides that can restore some or all of this regenerative potential, and are thus useful in the treatment of aging disorders that result in tissue loss or underperformance, and rehabilitation from injury. Described herein are therapeutic compositions comprising heparin-associated polypeptides and methods of treating disorders associated with aging, injury, or illness. The therapeutic compositions may comprise one or more heparin-associated polypeptides that possess mitogenic (i.e., regenerative) and/or fusion promoting activity to a somatic cell, such as a tissue progenitor cell. The therapeutic compositions may have activity towards muscle and soft tissue progenitor cells. These compositions may possess utility in treating sarcopenia, cachexia, muscular dystrophy, acute and chronic muscle wasting diseases, and muscle, ligament, or tendon injury, or any combination of these diseases or conditions.

In one aspect, described herein is a composition comprising: (a) a first therapeutic polypeptide comprising a first polypeptide of Table 2, and (b) a second therapeutic polypeptide comprising a second polypeptide of Table 2. The first polypeptide of Table 2 may be a first polypeptide of Table 1 and/or the second polypeptide of Table 2 is a second polypeptide of Table 1. The first polypeptide of Table 2 may comprise THBS1, THBS2, THBS4, FGF17, BMP7, VTN, POSTN, IGF2, or IL-15, or any combination thereof. The second polypeptide of Table 2 may comprise THBS1, THBS2, THBS4, FGF17, BMP7, VTN, POSTN, IGF2, or IL-15, or any combination thereof.

The first polypeptide of Table 2 may comprise BMP7 and/or a sequence comprising at least about 90% homology HAPs ID NO: 72; and the second polypeptide of Table 2 may comprise FGF17 and/or a sequence comprising at least about 90% homology to amino acids 23-216 of HAPs ID NO: 7.

The first polypeptide of Table 2 may comprise BMP7 and/or a sequence comprising at least about 90% homology to HAPs ID NO: 72; and the second polypeptide of Table 2 may comprise IGF2 and/or a sequence comprising at least about 90% homology to amino acids 25-91 of HAPs ID NO: 11.

The first polypeptide of Table 2 may comprise THBS1 and/or a sequence comprising at least about 90% homology to amino acids 19-1170 of HAPs ID NO: 9; and the second polypeptide of Table 2 may comprise THBS2 and/or a sequence comprising at least about 90% homology to amino acids 19-1172 of HAPs ID NO: 4.

The first polypeptide of Table 2 may comprise THBS1 and/or a sequence comprising at least about 90% homology to amino acids 19-1170 of HAPs ID NO: 9; and the second polypeptide of Table 2 may comprise THBS4 and/or a sequence comprising at least about 90% homology to amino acids 27-961 of HAPs ID NO: 8.

The first polypeptide of Table 2 may comprise THBS1 and/or a sequence comprising at least about 90% homology to amino acids 19-1170 of HAPs ID NO: 9; and the second polypeptide of Table 2 may comprise FGF17 and/or a sequence comprising at least about 90% homology to amino acids 23-216 of HAPs ID NO: 7.

The first polypeptide of Table 2 may comprise THBS1 and/or a sequence comprising at least about 90% homology to amino acids 19-1170 of HAPs ID NO: 9; and the second polypeptide of Table 2 may comprise VTN and/or a sequence comprising at least about 90% homology to amino acids 20-478 of HAPs ID NO: 1.

The first polypeptide of Table 2 may comprise THBS1 and/or a sequence comprising at least about 90% homology to amino acids 19-1170 of HAPs ID NO: 9; and the second polypeptide of Table 2 may comprise POSTN and/or a sequence comprising at least about 90% homology to amino acids 22-836 of HAPs ID NO: 6.

The first polypeptide of Table 2 may comprise THBS1 and/or a sequence comprising at least about 90% homology to amino acids 19-1170 of HAPs ID NO: 9; and the second polypeptide of Table 2 may comprise IGF2 and/or a sequence comprising at least about 90% homology to amino acids 25-91 of HAPs ID NO: 11.

The first polypeptide of Table 2 may comprise THBS1 and/or a sequence comprising at least about 90% homology to amino acids 19-1170 of HAPs ID NO: 9; and the second polypeptide of Table 2 may comprise IL-15 and/or a sequence comprising at least about 90% homology to amino acids 49-162 of HAPs ID NO: 10.

The first polypeptide of Table 2 may comprise THBS2 and/or a sequence comprising at least about 90% homology to amino acids 19-1172 of HAPs ID NO: 4; and the second polypeptide of Table 2 may comprise THBS4 and/or a sequence comprising at least about 90% homology to amino acids 27-961 of HAPs ID NO: 8. It may be, the composition further may comprise IL-15 and/or a polypeptide comprising at least about 90% homology to amino acids 49-162 of HAPs ID NO: 10. The composition may further comprise IGF2 and/or a polypeptide comprising at least about 90% homology to amino acids 25-91 of HAPs ID NO: 11.

The first polypeptide of Table 2 may comprise THBS2 and/or a sequence comprising at least about 90% homology to amino acids 19-1172 of HAPs ID NO: 4; and the second polypeptide of Table 2 may comprise FGF17 and/or a sequence comprising at least about 90% homology to amino acids 23-216 of HAPs ID NO: 7.

The first polypeptide of Table 2 may comprise THBS2 and/or a sequence comprising at least about 90% homology to amino acids 19-1172 of HAPs ID NO: 4; and the second polypeptide of Table 2 may comprise VTN and/or a sequence comprising at least about 90% homology to amino acids 20-478 of HAPs ID NO: 1.

The first polypeptide of Table 2 may comprise THBS2 and/or a sequence comprising at least about 90% homology to amino acids 19-1172 of HAPs ID NO: 4; and the second polypeptide of Table 2 may comprise POSTN and/or a sequence comprising at least about 90% homology to amino acids 22-836 of HAPs ID NO: 6.

The first polypeptide of Table 2 may comprise THBS2 and/or a sequence comprising at least about 90% homology to amino acids 19-1172 of HAPs ID NO: 4; and the second polypeptide of Table 2 may comprise IGF2 and/or a sequence comprising at least about 90% homology to amino acids 25-91 of HAPs ID NO: 11.

The first polypeptide of Table 2 may comprise THBS2 and/or a sequence comprising at least about 90% homology to amino acids 19-1172 of HAPs ID NO: 4; and the second polypeptide of Table 2 may comprise IL-15 and/or a sequence comprising at least about 90% homology to amino acids 49-162 of HAPs ID NO: 10.

The first polypeptide of Table 2 may comprise THBS4 and/or a sequence comprising at least about 90% homology to amino acids 27-961 of HAPs ID NO: 8; and the second polypeptide of Table 2 may comprise FGF17 and/or a sequence comprising at least about 90% homology to amino acids 23-216 of HAPs ID NO: 7.

The first polypeptide of Table 2 may comprise THBS4 and/or a sequence comprising at least about 90% homology to amino acids 27-961 of HAPs ID NO: 8; and the second polypeptide of Table 2 may comprise VTN and/or a sequence comprising at least about 90% homology to amino acids 20-478 of HAPs ID NO: 1.

The first polypeptide of Table 2 may comprise THBS4 and/or a sequence comprising at least about 90% homology to amino acids 27-961 of HAPs ID NO: 8; and the second polypeptide of Table 2 may comprise POSTN and/or a sequence comprising at least about 90% homology to amino acids 22-836 of HAPs ID NO: 6.

The first polypeptide of Table 2 may comprise THBS4 and/or a sequence comprising at least about 90% homology to amino acids 27-961 of HAPs ID NO: 8; and the second polypeptide of Table 2 may comprise IGF2 and/or a sequence comprising at least about 90% homology to amino acids 25-91 of HAPs ID NO: 11.

The first polypeptide of Table 2 may comprise THBS4 and/or a sequence comprising at least about 90% homology to amino acids 27-961 of HAPs ID NO: 8; and the second polypeptide of Table 2 may comprise IL-15 and/or a sequence comprising at least about 90% homology to amino acids 49-162 of HAPs ID NO: 10.

The first polypeptide of Table 2 may comprise FGF17 and/or a sequence comprising at least about 90% homology to amino acids 23-216 of HAPs ID NO: 7; and the second polypeptide of Table 2 may comprise VTN and/or a sequence comprising at least about 90% homology to amino acids 20-478 of HAPs ID NO: 1.

The first polypeptide of Table 2 may comprise FGF17 and/or a sequence comprising at least about 90% homology to amino acids 23-216 of HAPs ID NO: 7; and the second polypeptide of Table 2 may comprise POSTN and/or a sequence comprising at least about 90% homology to amino acids 22-836 of HAPs ID NO: 6.

The first polypeptide of Table 2 may comprise FGF17 and/or a sequence comprising at least about 90% homology to amino acids 23-216 of HAPs ID NO: 7; and the second polypeptide of Table 2 may comprise IGF2 and/or a sequence comprising at least about 90% homology to amino acids 25-91 of HAPs ID NO: 11.

The first polypeptide of Table 2 may comprise FGF17 and/or a sequence comprising at least about 90% homology to amino acids 23-216 of HAPs ID NO: 7; and the second polypeptide of Table 2 may comprise IL-15 and/or a sequence comprising at least about 90% homology to amino acids 49-162 of HAPs ID NO: 10.

The first polypeptide of Table 2 may comprise FGF17 and/or a sequence comprising at least about 90% homology to amino acids 23-216 of HAPs ID NO: 7; and the second polypeptide of Table 2 may comprise BMP-7 and/or a sequence comprising at least about 90% homology to amino acids 293-431 of HAPs ID NO: 70.

The first polypeptide of Table 2 may comprise VTN and/or a sequence comprising at least about 90% homology to amino acids 20-478 of HAPs ID NO: 1; and the second polypeptide of Table 2 may comprise POSTN and/or a sequence comprising at least about 90% homology to amino acids 22-836 of HAPs ID NO: 6.

The first polypeptide of Table 2 may comprise VTN and/or a sequence comprising at least about 90% homology to amino acids 20-478 of HAPs ID NO: 1; and the second polypeptide of Table 2 may comprise IGF2 and/or a sequence comprising at least about 90% homology to amino acids 25-91 of HAPs ID NO: 11.

The first polypeptide of Table 2 may comprise VTN and/or a sequence comprising at least about 90% homology to amino acids 20-478 of HAPs ID NO: 1; and the second polypeptide of Table 2 may comprise IL-15 and/or a sequence comprising at least about 90% homology to amino acids 49-162 of HAPs ID NO: 10.

The first polypeptide of Table 2 may comprise POSTN and/or a sequence comprising at least about 90% homology to amino acids 22-836 of HAPs ID NO: 6; and the second polypeptide of Table 2 may comprise IGF2 and/or a sequence comprising at least about 90% homology to amino acids 25-91 of HAPs ID NO: 11.

The first polypeptide of Table 2 may comprise POSTN and/or a sequence comprising at least about 90% homology to amino acids 22-836 of HAPs ID NO: 6; and the second polypeptide of Table 2 may comprise IL-15 and/or a sequence comprising at least about 90% homology to amino acids 49-162 of HAPs ID NO: 10.

The first polypeptide of Table 2 may comprise IGF2 and/or a sequence comprising at least about 90% homology to amino acids 25-91 of HAPs ID NO: 11; and the second polypeptide of Table 2 may comprise IL-15 and/or a sequence comprising at least about 90% homology to amino acids 49-162 of HAPs ID NO: 10.

The first polypeptide of Table 2 may comprise IGF2 and/or a sequence comprising at least about 90% homology to amino acids 25-91 of HAPs ID NO: 11; and the second polypeptide of Table 2 may comprise BMP-7 and/or a sequence comprising at least about 90% homology to amino acids 293-431 of HAPs ID NO: 70.

Also described is a method of producing a composition suitable for the treatment of an aging disorder, a muscle wasting disorder, a muscle injury, or an injury to a connective tissue, or a combination thereof, the method comprising admixing a pharmaceutically acceptable excipient, carrier, or diluent with the mitogenic and/or fusion promoting polypeptide or plurality of mitogenic and/or fusion promoting polypeptides. Also described is a method of producing a mitogenic and/or fusion promoting polypeptide comprising culturing a cell line comprising a nucleic acid encoding mitogenic and/or fusion promoting polypeptide or plurality of mitogenic and/or fusion promoting polypeptides under conditions sufficient to produce the mitogenic and/or fusion promoting polypeptide.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A shows experimental experiment overview of intramuscular administration of the entire pool of heparin-associated polypeptides from undifferentiated hESC cells (HAPs) promoted muscle regeneration and decreased fibrosis in aged mice (FIG. 1B) and genetically obese mice (FIG. 1C). Squares denote injury inducing intramuscular injection (IM) with Barium Chloride; circles denote administration of treatment or vehicle.

FIG. 2A Quantification of the regenerative index calculated as the number of newly regenerated fibers per mm\hat{ }2 of injury area in aged mice following IM treatment with HAPs. Regenerated fibers were identified as fibers with central nuclei, ****p<0.0001, **p=0.0011 (One-way Anova with multiple hypothesis correction)

FIG. 2B Histogram showing the fibrotic index calculated as the percentage of the fibrotic area decreased in aged mice following IM treatment with HAPs **p=0.009, ***p=0.0003. (One-way Anova with multiple hypothesis correction)

FIG. 3A Quantification of the regenerative index calculated as the number of newly regenerated fibers per mm\hat{ }2 of injury area in genetically obese mice following IM treatment with HAPs. Regenerated fibers were identified as fibers with central nuclei, ****p<0.0001, **p=0.0011 (One-way Anova with multiple hypothesis correction)

FIG. 3B Histogram showing the fibrotic index calculated as the percentage of the fibrotic area decreased in genetically obese mice following IM treatment with HAPs **p=0.009, ***p=0.0003. (One-way Anova with multiple hypothesis correction)

FIGS. 4A-4B illustrate representative results from an in vitro assay useful to validate the regenerative capacity of factors identified by mass spectroscopy. FIG. 4A shows proliferation rate changes expressed as % of nuclei stained with BrdU from cells treated with fusion media (neg. control), defined growth media (pos. control), Optimem, supernatant from differentiated HAPs, supernatant from undifferentiated HAPs, heparin binding proteins eluted from supernatant of undifferentiated HAPs under two different conditions, and supernatant of undifferentiated HAPs that has been depleted of heparin binding proteins. FIG. 4B shows data expressed as % imaged area stained for embryonic myosin heavy chain (eMyHC).

FIGS. 4C-4D show differentially enriched genes and pathways driven by heparin-associated proteins (HAPs) cocktail in aged human muscle cells. (C) RNA expression heatmap of top 50 differentially expressed (DE) genes in aged human muscle cells treated with cocktail of HAPs or vehicle. Cells were treated with indicated factor every 24 h for 96 h. (D) HAPs induce extracellular matrix and cell surface interactions to activate signal cascades promoting proliferative homeostasis. Reactome pathway analysis performed on all upregulated or downregulated DE genes (cutoff=p-value 0.01). Enriched pathways were obtained from GSEA Reactome_2016 gene sets. Enrichment is shown according to the p-value. Positive values are upregulated pathways and negative values are downregulated pathways

FIGS. 5A-5B show quantitation and representative images demonstrating the proliferation effect of IGFBP7 (330 ng/mL), POSTN (330 ng/mL), SPON1 (330 ng/mL), MST1 (330 ng/mL), and RARRES2 (330 ng/mL) (FIG. 5A); and VTN (10 μg/mL), FGF17 (500 ng/mL), IGF2 (2 μg/mL), FGF4 (500 ng/mL), FGF1 (500 ng/mL), and FGF6 (1 μg/mL) (FIG. 5B) in injury activated primary mouse myoblasts grown in vitro. FIG. 5C shows quantitation and representative images demonstrating the increased cellular fusion effect of THBS1 (330 ng/mL), THBS2 (330 ng/mL), and STC2 (875 ng/mL) in injury activated primary mouse myoblasts grown in vitro.

FIGS. 6A-6E show quantitation of immunofluorescent stained cell images demonstrating the proliferation effect of specific heparin-associated polypeptides. FIG. 6A shows the effect of IGFBP5 at 1 μg/mL, FIG. 6B shows the effect of THBS4 at 1 μg/mL, FIG. 6C shows the effect of VTN at 10 μg/mL, FIG. 6D shows the effect of FGF17 at 250 ng/mL, and FIG. 6E shows the effect of IGFBP7 at 500 ng/mL—all demonstrated notable effects in injury activated primary human myoblasts, young (18 years old) and aged (both 68 years old), grown in vitro. FIGS. 6F-6G, show quantitation and representative images demonstrating the increased cellular fusion effect of POSTN (1 μg/mL) FIG. 6F, PDGFRL (5 μg/mL) FIG. 6G in injury activated primary human myoblasts, young (18 years) and old (both 69 years old), grown in vitro.

FIG. 7A provides an exemplary proliferative dose response of mouse myoblasts cultured with hPSC-derived factor Thrombospondin 1 (THBS1) applied at 125 ng/mL, 250 ng/mL, and 500 ng/mL, 1000 ng/ml and 200 ng/ml.

FIG. 7B provides an exemplary proliferative dose response of mouse myoblasts cultured with hPSC-derived factor Platelet derived growth factor-like (PDGFRL) proteins were applied at 625 ng/mL, 1250 ng/mL, 2500 ng/mL, 5000 ng/mL, and 10000 ng/mL which demonstrated a dose-dependent increase in % fusion as expressed in % imaged area stained for embryonic myosin heavy chain (eMyHC).

FIG. 7C provides an exemplary proliferative dose response of mouse myoblasts cultured with hPSC-derived factor Fibroblast growth factor 17 (FGF17) applied at 12.5 ng/ml, 25 ng/ml, 50 ng/ml, 100 ng/ml, and 200 ng/ml which demonstrated a dose-dependent increase in proliferating cells as measured by % EdU positive nuclei.

FIG. 7D shows the shows that the combination of the THBS1 and FGF17 produced potentiation type synergy (CI<0.68, p<7.92E-7

FIG. 8 provides examples of synergistic combinations of heparin-associated polypeptides relative to the vehicle only control (FM) or to treatment with either of the individual heparin-associated polypeptides. Combination Index (CI) values and probability values (p-values) from statistical tests for the synergy models for these and other combinations are reported in Table 10.

FIG. 9A shows a bar graph quantitation of % EdU+ mouse myoblast in response to BMP7, demonstrating BMP7 drives proliferation. Error bars indicate standard deviation and asterisks indicate a p-value of less than 0.05 by Welch's One-Tailed T-test.

FIG. 9B shows a bar graph quantitation of cell count of human myoblast in response to BMP7, demonstrating BMP7 drives proliferation or improves cell survival. Myoblast were cultured 72 h in the presence of BMP7 at indicated dose. Fresh media and BMP7 was added every 24 h. Error bars indicate standard deviation and asterisks indicate a p-value of less than 0.05 by Welch's One-Tailed T-test

FIG. 10A shows a bar graph quantitation of % EdU+ mouse myoblast in response to BMP7, IGF2, or BMP7/IGF2 combined. Error bars indicate standard deviation and asterisks indicate a p-value of less than 0.05 by Welch's One-Tailed T-test, values reported in Table 18.

FIGS. 10B-10C show bar graph quantitation of the fold change from fusion media (vehicle control) in male or female human myoblast cell line in response to BMP7, IGF2, or BMP7/IGF2 combined. Error bars indicate standard deviation and asterisks indicate a p-value of less than 0.05 by Welch's One-Tailed T-test.

FIG. 11A shows a bar graph quantitation of % EdU+ mouse myoblast in response to FGF17, BMP7, or FGF17/BMP7 combined. Error bars indicate standard deviation and asterisks indicate a p-value of less than 0.05 by Welch's One-Tailed T-test.

FIGS. 11B-11C shows bar graph quantitation of the fold change from fusion media (vehicle control) in male or female human myoblast cell line in response to FGF17, BMP7, or BMP7/FGF17 combined. Error bars indicate standard deviation and asterisks indicate a p-value of less than 0.05 by Welch's One-Tailed T-test.

FIG. 12A illustrates a histogram plot of the CD36 receptor on the cell surface of mouse myoblasts.

FIG. 12B illustrates a histogram plot of the ITGA3 receptor on the cell surface of mouse myoblasts.

FIG. 12C illustrates a histogram plot of the ITGA6 receptor on the cell surface of mouse myoblasts.

FIG. 12D illustrates a histogram plot of the ITGB1 receptor on the cell surface of mouse myoblasts.

FIG. 12E shows a bar graph of RNA expression in young and aged human myoblast cell lines. Myoblast were cultured 96 h in fusion media. Fresh media was added every 24 h. Expression are expressed as FPKM.

FIG. 13A shows a bar graph of FGF17 receptor RNASEQ expression in young and aged human myoblast cell lines. Myoblast were cultured 96 h in fusion media. Fresh media was added every 24 h. Expression values are expressed as FPKM.

FIG. 13B shows a bar graph of BMP7 receptors RNASEQ expression in young and aged human myoblast cell lines. Myoblast were cultured 96 h in fusion media. Fresh media was added every 24 h. Expression values are expressed as FPKM.

FIG. 13C shows a bar graph of IGF2 and IGF2 receptor RNASEQ expression in young and aged human myoblast cell lines. Myoblast were cultured 96 h in fusion media. Fresh media was added every 24 h. Expression values are expressed as FPKM.

FIGS. 14A and 14B show expression levels of myogenic markers in myoblasts treated with FGF2, BMP7, THBS1, FGF17, THBS4 or IGF2 for 48 and 72 hours respectively.

FIG. 15A-E shows the Gene Ontology terms for transcripts upregulated in aged human myoblasts compared to vehicle as measured by RNA sequencing treated with a pool of all HAPs FIG. 15A, BMP7 FIG. 15B, FGF17 FIG. 15C, IGF2 FIG. 15D, or THBS1 FIG. 15E.

FIG. 16 shows the results of an experiment analysis using an acute injury model in aged mice of the effects of individual heparin-associated polypeptides with proliferative effects in vitro.

FIG. 16A. Administration of 20 ul of heparin-associated polypeptides (HAPs) FGF17 (500 ng/mL, p <2.23E-4), THBS1 (2 μg/mL, p<5.83E-5), THBS2 (2 μg/mL, p<2.67E-4), and VTN (10 μg/mL, p<1.13E-2) resulted in improved new fiber formation (regenerative index) in aged mice compared to vehicle-treated aged mice to levels similar or better than young mice. FIG. 16B. Administration of 20 ul of heparin-associated polypeptides PPDGFRL (5 μg/mL, p<3.85E-2) and IGFBP7 (1 μg/mL, p<6.63E-3) resulted in improved new fiber formation (regenerative index compared to vehicle-treated aged mice. Regenerative index was calculated as the number of newly regenerated fibers per mm\hat{ }2 of injury area. Stars indicate degree of significance from one-way ANOVA test

FIG. 17A and FIG. 17B shows the results of an experiment for in vivo injury and individual heparin-associated polypeptide administration followed by muscle excision, dissociation, ex vivo culturing of activated myoblasts and quantitation by chemical and immunofluorescent labelling. FIG. 17A provides resulting quantitation that demonstrates the regenerative effect of heparin-associated polypeptide administration (FGF17) of 20 ul at 500 ng/ml improved the regeneration of new myoblasts in aged mice above the vehicle-treated aged mice (p<7.57E-8) to a level similar to those seen in young mice. FIG. 17B provides resulting quantitation that demonstrates the regenerative effect of administration of heparin-associated polypeptides FGF17 (500 ng/ml) and THBS4 (2 μg/mL)—each improved the regeneration of new myoblasts in aged mice above the vehicle-treated aged mice (p<1.57E-2, 4.64E-2 respectively, one-sided test) compared to vehicle treated control.

FIG. 18A shows the experimental overview. Intramuscular injection of 1.2% of BaCl₂ (7 ul/TA) was used to generate chemical injury in the TAs of 78 weeks old mice. Factors were administered via intramuscular injection after 2 h and 48 h of muscle injury.

FIG. 18B shows quantification of the regenerative index calculated as the number of newly regenerated fibers per mm² of injury area demonstrating the effect of individual HAPs at saturating doses compared to combination treatments at subsaturating doses had synergistic efficacy: FGF17 at 500 ng/ml, THBS1 at 2000 ng/ml, THBS2 at 2000 ng/l and VTN at 10000 ng/ml. Regenerated fibers were identified as fibers with central nuclei, significant p-values are indicated with a star.

FIG. 18C shows the number of regenerating fibers after treatment with BMP7 combined with IGF2. FIG. 18D shows the fibrotic index calculated as the percentage of the fibrotic area, demonstrating the effect of individual HAPs at maximal dose (FGF17 at 500 ng/ml and THBS1 at 2000 ng/ml) and demonstrating a combined effect when used in pairwise treatments at a below maximal dose (FGF17 and BMP7 at 12.5 ng/ml each; BMP7 and IGF2 at 25 ng/ml and 60 ng/ml). Significant p-values are indicated with a star. One-way Anova corrected for multiples comparison using Tukey method was used to compare the data.

FIG. 19A shows the experiment overview for intramuscular injection of 10 ug of Cardiotoxin (CTX) to generate injury in the TAs of 21M old mice to test muscle regeneration. BMP7 was administered via intramuscular injection after 1d, 3d and 5d of muscle injury. At day 8, mice were euthanized and TAs were collected.

FIG. 19B Representative images and quantification. H&E staining showing the regenerated fibers and the injury area from Vehicle and BMp7 groups.

FIG. 19C Quantification of the regenerative index calculated as the number of newly regenerated fibers per mm\hat{ }2 of injury area. Regenerated fibers were identified as fibers with central nuclei, **p=0.0059 (unpaired t-test)

FIG. 19D Histogram of the frequency of muscle fiber cross sectional area in histological analysis showing muscle fiber size distribution increased in the BMP7 treatment group. *p=0.0325, *p=0.0350 (2-way Anova multiple comparisons).

FIG. 20A shows the experimental overview for assessing muscle regeneration from intramuscular administration of IGF2 in a cardiotoxin injured old mice model. Intramuscular injection of 10 ug of Cardiotoxin/TA was used to generate focal injury in the TAs of 21M old mice. 20 ul of IGF2 (2 ug/ml) or Vehicle (PBS) were administered via intramuscular injection at day 2, 4 and 6. Injured muscles were collected at 7 dpi.

FIG. 20B Representative images and quantification. H&E staining showing the regenerated fibers and the injury area from Vehicle and IGF2 groups.

FIG. 20C The quantification of the regenerative index calculated as the number of newly regenerated fibers per mm\hat{ }2 of injury area is also shown. Regenerated fibers were identified as fibers with central nuclei. significant p-values are shown using unpaired t-test. ** p=0.0016 (Unpaired t-test).

FIG. 20D Histogram of the frequency of muscle fiber cross sectional area in histological analysis showing muscle fiber size distribution increased in the IGF2 treatment group. *p=0.0324, **p=0.0074. 2-way Anova multiple comparisons

FIG. 21A shows the experimental overview. Dexamethasone (25 mg/kg i.p.) was administered to 12 weeks old mice for 20 days simultaneously with a subcutaneous injection of FGF17 (0.5 mg/kg). Muscle weight was assessed on Day 21. Forelimb grip strength and both limb grip strength were measured on Day 7, 13 and 21

FIG. 21B shows the TAs muscle weight over initial body weight shown as the percentage change from vehicle. **p=0.0062. (Unpaired t-test).

FIG. 21C shows the forelimb force measured on Day 21. The bar plot shows the specific forelimb force calculated as the ratio of forelimb force in N over the weight in g, *p=0.0268. (Unpaired t-test).

FIG. 21D shows the specific forelimb force calculated as the ratio of forelimb force in N over the weight in g. **p=0.001 (Unpaired t-test).

FIG. 21E Both limb force measured on Day 21 calculated as the ratio of both limb force in N over the weight in g *p=0.0117. (Unpaired t-test).

FIG. 22A Experiment overview and groups were systemic administration of BMP7 protected against Dexamethasone induced muscle atrophy. Dexamethasone (25 mg/kg i.p.) was administered to 12 weeks old mice for 14 days simultaneously with a subcutaneous injection of BMP7 (0.03 mg/kg) or vehicle (saline). Mice were euthanized on Day 15.

FIG. 22B TAs and FIG. 22C GCs muscle weight were assessed on Day 15. *** p=0.0001, **p=0.0014 (unpaired t-test)

FIG. 22D Measures of muscle strength or function improved by BMP7 treatment. FIG. 22D forelimb maximum force in mN assessed at Day 13 ** p=0.021. (unpaired t-test)

FIG. 22E Specific forelimb maximum force calculated as the ratio of forelimb force in mN over the weight in g ***p=0.0007. (unpaired t-test)

FIG. 22F distance **p=0.0265, FIG. 22G time to exhaustion **p=0.0051, FIG. 22H max speed **p=0.007, FIG. 22I and work **p=0.0056 were measured on Day 14 using an induced treadmill running model set to progressively increase speed 2 m/min every subsequent 2 min. Unpaired t-test was used to compare data.

FIG. 23A shows experiment overview and groups were systemic administration of BMP7 protected against aging induced muscle dysfunction. Subcutaneous injection of BMP7 (30 ug/kg) or vehicle (PBS) were administered to 21-24M old mice for 14 days. Muscle function was assessed at days 13 and 14.

FIG. 23 B,C Treadmill performance measured at day 14 using an induced treadmill running model set to progressively increase speed 2 m/min every subsequent 2 min. (B) Distance ran shown. *p=0.0371 (C) Time to exhaustion *p=0.0298. Unpaired t-test was used to compare data.

FIG. 23 D-G forelimb Grip strength force was assessed at day 13. (D) forelimb Grip strength force was assessed at day 13. The graph shows forelimb grip strength force, **** p<0.0001. (E) the graph shows specific forelimb maximum force calculated as the ratio of forelimb force in mN over the weight in g ***p=0.0001. (F) Bothlimb Grip strength force was assessed at day 13. The graph shows bothlimb grip strength force, *** p=0.0003. (G) the graph shows specific bothlimb maximum force calculated as the ratio of forelimb force in mN over the weight in g **p=0.0011. Unpaired t-test was used to compare data.

FIG. 23H-K show 4 representative graphs out of 37 readouts measured which showed systemic treatment with BMP7 resulted in no adverse events. No changes were observed in (H) the white blood cell count p=0.6503, (I) Alkaline phosphatase activity p<0.9999, (J) Creatinine concentration p=0.5995 and (K) Amylase activity p=0.5468. Unpaired t-test was used to analyze this data.

FIG. 24A provides representative quantitation of immunofluorescence images demonstrating the proliferation enhancing effects of HAPs (hPSC factors) and specific HAPss at various does on primary human myoblasts derived from a patient with type 1 myotonic dystrophy.

FIG. 24B provides quantitation of immunofluorescence images demonstrating the hypertrophy enhancing effects of HAPs (hPSC factors) and specific heparin-associated polypeptides administration at various does on primary human myoblasts derived from a patient with type 1 myotonic dystrophy.

FIG. 25A-C IGF2 treatment promoted proliferation and fusion in DM1 human myoblast (32 year old caucasian female) cells. FIG. 25A Bar graph and table quantitation of % EdU+ human myoblast and FIG. 25B % area eMyHC in response to IGF2. Significant p-values (EdU: Vehicle˜IGF2: 6.8E-3, % eMyHC Area: Vehicle˜IGF2: 1.9E-4) (*p<0.05 by Students Two-Tailed T-test, n=3-6). FIG. 25C Bar graph of MYH3 and CKM expression fold change in DM1 human myoblast in response to indicated treatment compared to vehicle as measured by qPCR. Myoblasts were cultured 96 h in the presence of factors (IGF2 200 ng/mL, n=3). Fresh media and factors were added every 24 h. Mean∓S.D. Significant p-values (MYH3: Vehicle˜IGF2: 1.13E-03, CKM: Vehicle IGF2: 7.67E-03) FIG. 25D Bar graph of ATP1B1 expression fold change in DM1 human myoblast in response to indicated treatment compared to FM (vehicle) as measured by qPCR. Myoblasts were cultured 48 h in the presence of factors (IGF2 200 ng/mL, n=3) Fresh media and factors were added every 24 h. Mean∓S.D. Significant p-values (Vehicle˜IGF2: 3.11E-05) (*p<0.05 by Students Two-Tailed T-test, n=3)

### DETAILED DESCRIPTION

In one aspect, described herein is a composition comprising a mitogenic and/or fusion promoting polypeptide, wherein the polypeptide is a heparin-associated polypeptide secreted from a stem cell or a transformed cell line, wherein the heparin-associated polypeptide possesses mitogenic and/or fusion promoting activity. The composition may be for use in a method of treating an aging disorder, a muscle wasting disorder, a muscle injury, or an injury to a connective tissue, or a combination thereof. The composition may be for use in a method of increasing proliferation of a muscle cell or connective tissue cell precursor in an individual. The individual may be afflicted with or suspected of being afflicted with an aging disorder, a muscle wasting disorder, a muscle injury, or an injury to a connective tissue, or a combination thereof. The aging disorder may be sarcopenia. The muscle wasting disorder is muscular dystrophy. The muscle wasting disorder may be cachexia, e.g., muscular cachexia. In another aspect, described herein, is a composition comprising a mitogenic and/or fusion promoting polypeptide, wherein the mitogenic and/or fusion promoting polypeptide may comprise FST, CTGF, THBS1, THBS2, THBS3, HGFAC, IGFBP3, IGFBP5, IGFBP7, IGFBP4, SFRP1, STC1, STC2, IGFBP2, SPON1, MST1, POSTN, PTN, RARRES2, DRAXIN, CLC, LTBP1, ADAMTS7, NOV, PDGFRL, AGRN, FGFBP3, ADAMTS19, THBS4, PLAT, GDF15, CHRDL1, FRZB, COL1A1, FN1, LAMA1, LAMAB1, LAMAC1, TNC, VTN, MDK, LEFTYA, MATN2, ANOS1, APOB, CLEC3A, COCH, CSF2, FBLN1, FGF-17, TGFBI, CXCL12, HDGF2, NTS, GDNF, VEGF-165, TIMP1, TCN2, PAMR1, WISP2, HGF, IGF-2, ANG, PDGFD, FSTL1, SFRP2, VASN, FGF-4, TIMP2, NDNF, FGF1, CKB, DAG1, PLAU, PTPRS, FLT1, OLFML3, BTC, IL-13, IL-15, APLN, IL-10, HB-EGF, FGF-6, FGF-13, CTNNB1, GLG1, CCL14, NAMPT, BMP-7, HDGF, FGF-19, GASP-1, BMP-2, TGFb1, FGF-18, ADAMTS1 a protein listed in Table 2, or a protein listed in Table 1, or any combination thereof. The mitogenic and/or fusion promoting polypeptide may comprise one or more of VTN, POSTN, FGF17, THBS2, THBS4, IGF2, IL-15, THBS1, and BMP7. The mitogenic and/or fusion promoting polypeptide may comprise VTN. The mitogenic and/or fusion promoting polypeptide may comprise THBS2. The mitogenic and/or fusion promoting polypeptide may comprise POSTN. The mitogenic and/or fusion promoting polypeptide may comprise FGF17. The mitogenic and/or fusion promoting polypeptide may comprise THBS4. The mitogenic and/or fusion promoting polypeptide may comprise IGF2. The mitogenic and/or fusion promoting polypeptide may comprise IL-15. The mitogenic and/or fusion promoting polypeptide may comprise THBS1. The mitogenic and/or fusion promoting polypeptide may comprise BMP7. The composition may comprise a mixture of a plurality of different mitogenic and/or fusion promoting polypeptides. The plurality of different mitogenic and/or fusion promoting polypeptides may comprise three, four, or five different mitogenic and/or fusion promoting polypeptides. The plurality of polypeptides may comprise one, two, three, four, five, six, seven, eight, nine, ten or more polypeptides of Table 2. The plurality of polypeptides may comprise one, two, three, four, five, six, seven, eight, nine, ten or more polypeptides of Table 1. The plurality of mitogenic and/or fusion polypeptides may comprise BMP7 and IGF2. The plurality of mitogenic and/or fusion polypeptides may comprise BMP7 and and FGF17. The plurality of mitogenic and/or fusion promoting polypeptides may comprise IGF2, THBS2, and THBS4. The plurality of mitogenic and/or fusion promoting polypeptides may comprise IL-15, THBS2, and THBS4. The plurality of mitogenic and/or fusion promoting polypeptides may comprise THBS2 and THBS4. The plurality of mitogenic and/or fusion promoting polypeptides may comprise THBS2, THBS4, and VTN. The plurality of mitogenic and/or fusion promoting polypeptides may comprise THBS2, THBS4, and ANOS1. The plurality of mitogenic and/or fusion promoting polypeptides may comprise THBS2, THBS4, and IL-15. The plurality of mitogenic and/or fusion promoting polypeptides may comprise THBS2, THBS4, and IGF2. The plurality of mitogenic and/or fusion promoting polypeptides may comprise THBS1 and FGF17. The plurality of mitogenic and/or fusion promoting polypeptides may comprise THBS2 and VTN. The plurality of mitogenic and/or fusion promoting polypeptides may comprise THBS1 and VTN. The plurality of mitogenic and/or fusion promoting polypeptides may comprise THBS1 and THBS2. The plurality of mitogenic and/or fusion promoting polypeptides may comprise THBS2 and FGF17. The plurality of mitogenic and/or fusion promoting polypeptides may comprise THBS1 and THBS4. The plurality of mitogenic and/or fusion promoting polypeptides may comprise VTN and FGF17. The plurality of mitogenic and/or fusion promoting polypeptides may comprise THBS4 and VTN. The plurality of mitogenic and/or fusion promoting polypeptides may comprise THBS4 and FGF17. The composition may be for use in a method of treating an aging disorder, a muscle wasting disorder, a muscle injury, or an injury to a connective tissue, or any combination thereof. The composition may be for use in a method of increasing proliferation of a muscle cell and/or connective tissue cell precursor in an individual. The individual may be afflicted with or suspected of being afflicted with an aging disorder, a muscle wasting disorder, a muscle injury, or an injury to a connective tissue, or a combination thereof. The aging disorder may be sarcopenia. The muscle wasting disorder may be muscular dystrophy. The muscle wasting disorder may be cachexia, e.g., muscular cachexia.

In the following description, certain specific details are set forth in order to provide a thorough understanding of various embodiments. However, one skilled in the art will understand that the embodiments provided may be practiced without these details. Unless the context requires otherwise, throughout the specification and claims that follow, the word “comprise” and variations thereof, such as, “may comprise” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein a composition that is “consisting essentially” of the recited components is a composition that only has the recited elements as active ingredients, but can comprise other non-active components that do not appreciably modify the function or activity of the recited components. Any list disclosed herein that is recited as “comprising” can be recited as “consisting essentially,” to exclude non-recited polypeptide or protein components.

As used herein “heparin-associated polypeptide” means any polypeptide that directly binds to heparin with a K_{D} of less than 1 micromolar, or any polypeptide that associates with one or more polypeptides that bind directly to heparin with a K_{D} of less than 1 micromolar. This K_{D} can be measured using a method such as surface plasmon resonance. See e.g., Nguyen et al., “Surface plasmon resonance: a versatile technique for biosensor applications.” Sensors (Basel). 2015 May 5; 15(5):10481-510. Alternatively, a heparin-associated polypeptide is one that is enriched by a factor of at least 5-fold, 10-fold, 100-fold, or 1,000 from a complex mixture of polypeptides (e.g., a cell supernatant) by the use of heparin bound to a bead or other matrix support, or co-purifies with such a polypeptide.

As used herein “heparin-binding polypeptide” (HAP) means any polypeptide that directly binds to heparin with a K_{D} of less than 1 micromolar. Heparin-binding polypeptides can interact with heparin at steady-state under normal growth conditions, but in other instances heparin-binding polypeptides may interact with heparin transiently under normal growth conditions or only under certain conditions as a result of a signaling or environmental stimulus. Heparin binding-polypeptides may interact with heparin as a result of post-translational modifications such as phosphorylation, dephosphorylation, acetylation, deacetylation, lipidation, delipidation, glycosylation, or deglycosylation, or combinations thereof.

As used herein “pluripotent stem cell” or “pluripotent cell” (PSC) means a cell that has the ability to differentiate into several different cell types that are derivatives of all of the three germinal layers (endoderm, mesoderm, and ectoderm). Pluripotent stem cells are capable of forming teratomas. Examples of pluripotent stem cells are embryonic stem cells (ESCs), embryonic germ stem cells (EGCs), embryonic Carcinoma Cells (ECCs), and induced pluripotent stem cells (iPSCs). PSC may be from any organism of interest, including, primate, human (hPSCs); canine; feline; murine; equine; porcine; avian; camel; bovine; ovine, and so on.

As used herein “somatic cell” means any cell of an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include muscle cells and muscle progenitor cells, the latter of which may be able to self-renew and naturally give rise to all or some cell types of the skeletal, cardiac, or smooth muscle but cannot give rise to cells of the ectoderm or endoderm lineages.

As used herein the term “about” refers to an amount that is near the stated amount by 10% or less.

As used herein the terms “individual” “subject,” and “patient” are interchangeable. The individual can be mammal such as a horse, cow, pig, chicken, goat, rabbit, mouse, rat, dog, or cat. The individual may be a human person.

The terms “polypeptide” and “protein” are used interchangeably to refer to a polymer of amino acid residues. Polypeptides, including the provided polypeptide chains and other peptides, e.g., linkers and binding peptides, may include amino acid residues including natural and/or non-natural amino acid residues. The terms also include post-translational modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts that produce the proteins, errors due to PCR amplification, or errors in protein translation.

A recombinant protein may be a protein expressed in a system other than a human, e.g., the protein is expressed from bacteria, yeast, or mammalian cells in culture. In some cases, the protein is expressed from Chinese Hamster Ovary cells (CHO cells). In some cases, the protein is expressed from mouse myeloma cells, e.g., (NS0) cells. In some cases, the protein is expressed from E. coli.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or may comprise a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Exogenous” with respect to a nucleic acid or polynucleotide indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example using genetic recombination. Genetic recombination is the breaking and rejoining of DNA strands to form new molecules of DNA encoding a novel set of genetic information. Often exogenous nucleic acids will include a translatable sequence lacking introns that has been cloned from a cDNA.

As described herein a “mitogenic polypeptide” is one that induces one or more stages of mitosis, including interphase, prophase, metaphase, anaphase, and telophase. A mitogenic polypeptide may be one that induces mitosis in any one or more of a soft-tissue cell, a soft-tissue precursor cell, a muscle cell, a muscle precursor cell, or a tenocyte.

As described herein a “fusion promoting” polypeptide is one that promotes fusion of muscle cells or muscle cell precursors. Fusion of muscle precursors like C2C12 cells is an experimental marker of differentiation and can be monitored by increases in eMyHC expression, cell size, or increased number of nuclei per eMyHC positive cell a by a statistically measurable change of at least 25% magnitude (p<0.05) relative to vehicle treated cells grown in otherwise identical conditions.

Reference to a fusion, fusion polypeptide, or fusion protein may refer to a synthetically and/or recombinantly produced molecule in which two or more amino acid sequences are connected, e.g., by a peptide bond and/or linker. In some cases, the two or more amino acid sequences are linked via a linker comprising one or more amino acids. In other cases, the two or more amino acid sequences are not linked via a linker, e.g., the two sequences are directly connected by a peptide bond. In some cases, at least one of the two or more amino acid sequences may comprise a polypeptide described herein. For example, the polypeptide described herein is a polypeptide comprising VTN, POSTN, PDGFRL, THBS2, THBS4, THBS1, IL-15, or IGF2, or a combination thereof.

Reference to a conjugate, polypeptide conjugate, or protein conjugate may refer to a synthetically and/or recombinantly produced molecule comprising a chemical entity covalently bound to one or more amino acids of an amino acid sequence. In some cases, the conjugation is selective such that the chemical entity is connected to a specific amino acid of the amino acid sequence. The amino acid sequence may comprise a polypeptide described herein. For example, the polypeptide described herein is a polypeptide comprising VTN, POSTN, FGF17, THBS2, THBS4, THBS1, IL-15, IGF2, or BMP7, or a combination thereof.

A polypeptide described herein may be a proteoform of a protein listed in Table 2. As used herein a proteoform may describe a molecular form of a protein product arising from a gene encoding a protein, such as a protein listed in Table 2. In some cases, a proteoform includes proteins that arise from the same gene as a result of genetic variation, alternatively spliced RNA transcripts, post-translational modifications, or polypeptide cleavage event.

### Heparin-Associated Polypeptides

In one aspect, polypeptides described herein that are useful for treating an aging disease or injury comprise one or more polypeptides secreted from an induced pluripotent stem cell, an embryonic stem cell, a tissue progenitor cell, or a transformed cell line that bind to heparin. A plurality of 1, 2, 3, 4, 5, 6, 7, 8, 9, 10 or more HAPs are included in a composition may comprise a pharmaceutically acceptable excipient, carrier, or diluent. In some cases, the composition may comprise one, two, three, four, five, six, seven, eight, nine, ten or more polypeptides of Table 2. In some cases, the composition may comprise one, two, three, four, five, six, seven, eight, nine, ten or more polypeptides of Table 1.

In certain aspects, there are three biochemical features that are common across all potential therapeutic HAPs: 1) they are secreted by human pluripotent stem cells; 2) they can be purified by heparin agarose beads from a complex mixture, and 3) their molecular weight equals or exceeds 3.5 kDa.

In certain aspects, there are certain structure-function relationships that potentially link disparate therapeutic polypeptides into a genus of heparin-associated therapeutic polypeptides. Included among these are the ability to be secreted, which may require: 1) an N-terminal signal sequence (aprox. 15-30 amino acids in length); and/or 2) the presence of one or more post translational modifications added in the Endoplasmic Reticulum or the Golgi apparatus to promote stability, such as glycosylation or disulfide bonds. It is estimated that 2,000 to 3,000 genes encoded by the human genome produce a secreted polypeptide in one or more cell types. In addition to being secretory polypeptides the therapeutic polypeptides may comprise a heparin-binding domain, or, alternatively associate with heparin-binding domain comprising polypeptides. Heparin is a linear polymer of saccharides in 1-4 alpha linkages that form a spiraling chain, commonly associated with its role in binding plasma proteins to reduce clotting (See Capila and Lindhart, “Heparin-protein interactions” Angew Chem Int Ed Engl. 2002 Feb. 1; 41(3):391-412). Currently, predicting heparin-binding from protein sequence alone is a challenge for the field due to the structural heterogeneity of heparin polymers and the large and variable number of shallow binding pockets thought to be important for stabilizing the interaction. Several hundred HAPs have been empirically tested for heparin binding, using a few heparin chain configurations. Based on these studies many binding motifs have been proposed, but none have been proven necessary and sufficient. One common motif appears to be a sequence of repeating basic residues that orient onto a common surface of the secondary structure for interacting with the matching pattern of sulfate groups on heparin chains. Therefore, many heparin-binding therapeutic polypeptides may contain patterns of basic residues (arginine or lysine) clustered in some part of the protein, though agnostic to the exact sequence.

The heparin-associated therapeutic polypeptide is a secreted polypeptide. The heparin-associated therapeutic polypeptide may comprise a secreted polypeptide that may comprise 1, 2, 3, 4, 5, 6, 7, 8, 9, 10 or more disulfide bonds. The heparin-associated therapeutic polypeptide may comprise a secreted polypeptide that may comprise 1, 2, 3, 4, 5, 6, 7, 8, 9, 10 or N-liked or 0-linked glycans. The heparin-associated therapeutic polypeptide may be greater than about 3.5 kilodaltons. The heparin-associated may be greater than about 5, 7.5, 10, 15, or 20 kilodaltons. The heparin-associated therapeutic polypeptide may be one that may comprise a region exhibiting enrichment for basic amino acids arginine or lysine. The region can be about 5, 10, 15, 20, 25, 30, 35, 40, 45, 50, 55, 60, 65, 70, 75, 80, 85, 90, 95, or 100 amino acids in length, and comprise an amount of basic residues that is greater than would be 10%, 20%, 30%, 40%, 50%, 60%, 70%, 80%, 100%, 150%, or 200% greater than expected given random chance. The heparin-associated therapeutic polypeptide does not comprise a basic DNA binding motif, such as those found in bZIP transcription factors. The HAP may comprise heparin binding polypeptide.

The HAPs, described herein, can comprise one or more amino acid modifications that promote stability and/or facilitate production. The polypeptide can comprise one or more covalent modifications that promote stability (e.g., PEGylation). Other modifications of the HAP(s) are contemplated herein. For example, the HAP(s) may be linked to one of a variety of non-proteinaceous polymers, e.g., polyethylene glycol, polypropylene glycol, polyoxyalkylenes, or copolymers of polyethylene glycol, polysialic acid, glycolic acid, or polypropylene glycol. The HAP may comprise be fused or conjugated to another protein to increase stability and or bioavailability. The HAP may comprise be a fusion with an Fc region of an immunoglobulin or with serum albumin.

The HAPs described herein can be encapsulated in nanospheres or nanoparticles to increase stability. The nanospheres or nanoparticles may comprise biodegradable or bioabsorbable. Certain types of nanospheres can be deployed such as polylactic acid (PLA), polyglycolic acid (PGA), or Poly(D,L-lactic-coglycolic-acid) (PLGA) microspheres or nanospheres. The HAP may be included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA).

The heparin-associated-polypeptide may be concatemerized to increase stability and or bioavailability. The HAP(s) may comprise concatemers of the same or of different heparin-associated binding polypeptides. Concatemers can be separated by polypeptide linkers, for example a Gly-Ser linker of any suitable length. The Gly-Ser linker may comprise a G₄S₁ linker. The concatemers may comprise 1, 2, 3, 4, 5 or more of the same HAP as a single polypeptide separated by a Gly-Ser linker. The concatemers may comprise 1, 2, 3, 4, 5 or more different HAPs as a single polypeptide separated by a Gly-Ser linker. The concatemers may comprise 1, 2, 3, 4, 5 or more of the same HAP covalently linked through a non-peptide linkage, such as for example a disulfide bridge. The concatemers may comprise 1, 2, 3, 4, 5 or more different HAPs covalently linked through a non-peptide linkage, such as for example a disulfide bridge. The concatemers may comprise 1, 2, 3, 4, 5 or more of the same HAP non-covalently linked, such as for example, by a streptavidin-biotin interaction or protein-protein interaction. The concatemers may comprise 1, 2, 3, 4, 5 or more different HAPs non-covalently linked such as for example, by a streptavidin-biotin interaction or protein-protein interaction.

Additional modifications to HAP comprise deletions of 1, 2, 3, 4, 5, 6, 7, 8, 9, 10, 15, 20, 25, 30, 35, 40, 45, 50, 55, 60, 65, 70, 75, 80, 85, 90, 95, 100 amino acids from the N-terminal or C-terminal ends of the HAP. The HAP may comprise the deletion of known inhibitory domains or deletion of domains not associated with the heparin-associated-polypeptides functions in inducing proliferation of muscle, connective, or soft-tissue cell precursors.

The HAPs herein can comprise cleavage products of a pro-protein. Cleavage of a pro-protein can result in activation or higher activity of said pro-protein. HAPs may be produced that correspond to a cleaved or active form of the pro-protein. The HAPs may comprise only the active domain of a heparin associated pro-protein (e.g., the minimal portion sufficient to create a biological effect).

The HAP may comprise one or more of the polypeptides listed in Table 1 and/or Table 2. The polypeptide may be at least about 85%, 90%, 95%, 97%, 98%, 99%, or 100% identical to a polypeptide listed in Table 1 and/or Table 2, or an isoform thereof. The polypeptide may be at least about 85%, 90%, 95%, 97%, 98%, 99%, or 100% identical to a sequence selected from POLYPEPTIDE ID NOS: 1-44, 55, 56, and 58-72.

The HAP may comprise one or more of the polypeptides listed in Table 2, Table 1, or a proteoform thereof. The HAP may be at least about 85%, 90%, 95%, 97%, 98%, 99%, or 100% identical to a polypeptide listed in Table 2, Table 1, or a proteoform thereof. The HAP may comprise a polypeptide at least about 85%, 90%, 95%, 97%, 98%, 99%, or 100% identical to FST, CTGF, THBS1, THBS2, THBS3, HGFAC, IGFBP3, IGFBP5, IGFBP7, IGFBP4, SFRP1, STC1, STC2, IGFBP2, SPON1, MST1, POSTN, PTN, RARRES2, DRAXIN, CLC, LTBP1, ADAMTS7, NOV, PDGFRL, AGRN, FGFBP3, ADAMTS19, THBS4, PLAT, GDF15, CHRDL1, FRZB, COL1A1, FN1, LAMA1, LAMAB1, LAMAC1, TNC, VTN, MDK, LEFTYA, MATN2, ANOS1, APOB, CLEC3A, COCH, CSF2, FBLN1, FGF-17, TGFBI, CXCL12, HDGF2, NTS, GDNF, VEGF-165, TIMP1, TCN2, PAMR1, WISP2, HGF, IGF-2, ANG, PDGFD, FSTL1, SFRP2, VASN, FGF-4, TIMP2, NDNF, FGF1, CKB, DAG1, PLAU, PTPRS, FLT1, OLFML3, BTC, IL-13, IL-15, APLN, IL-10, HB-EGF, FGF-6, FGF-13, CTNNB1, GLG1, CCL14, NAMPT, BMP-7, HDGF, FGF-19, GASP-1, BMP-2, TGFb1, FGF-18, ADAMTS1 or any combination thereof. The HAP may comprise a polypeptide at least about 85%, 90%, 95%, 97%, 98%, 99%, or 100% identical to VTN, POSTN, FGF17, THBS2, THBS4, THBS1, IL-15, IGF2, or BMP7, or any combination thereof. The HAP may comprise THBS1. The HAP may comprise THBS2. The HAP may comprise THBS4. The HAP may comprise FGF17. The HAP may comprise VTN. The HAP may comprise POSTN. The HAP may comprise IGF2. The HAP may comprise IL-15. The HAP may comprise BMP7. Described herein is a composition that may comprise comprising any 1, 2, 3, 4, 5, 6, 7, 8, 9, 10 or more polypeptides at least about 85%, 90%, 95%, 97%, 98%, 99%, or 100% identical to a polypeptide Table 2, Table 1, FST, CTGF, THBS1, THBS2, THBS3, HGFAC, IGFBP3, IGFBP5, IGFBP7, IGFBP4, SFRP1, STC1, STC2, IGFBP2, SPON1, MST1, POSTN, PTN, RARRES2, DRAXIN, CLC, LTBP1, ADAMTS7, NOV, PDGFRL, AGRN, FGFBP3, ADAMTS19, THBS4, PLAT, GDF15, CHRDL1, FRZB, COL1A1, FN1, LAMA1, LAMAB1, LAMAC1, TNC, VTN, MDK, LEFTYA, MATN2, ANOS1, APOB, CLEC3A, COCH, CSF2, FBLN1, FGF-17, TGFBI, CXCL12, HDGF2, NTS, GDNF, VEGF-165, TIMP1, TCN2, PAMR1, WISP2, HGF, IGF-2, ANG, PDGFD, FSTL1, SFRP2, VASN, FGF-4, TIMP2, NDNF, FGF1, CKB, DAG1, PLAU, PTPRS, FLT1, OLFML3, BTC, IL-13, IL-15, APLN, IL-10, HB-EGF, FGF-6, FGF-13, CTNNB1, GLG1, CCL14, NAMPT, BMP-7, HDGF, FGF-19, GASP-1, BMP-2, TGFb1, FGF-18, ADAMTS1 a proteoform thereof, or a combination thereof; and a pharmaceutically acceptable excipient, carrier, or diluent. The composition may comprise a plurality of peptides from Table 2; and optionally a pharmaceutically acceptable excipient, carrier, or diluent. In some cases the plurality may comprise 1, 2, 3, 4, 5, 6, 7, 8, 9, 10 or more polypeptides of Table 2. In some cases, one or more of the plurality of polypeptides is at least about 85%, 90%, 95%, 97%, 98%, 99%, or 100% identical to a polypeptide of Table 2. The composition may comprise a plurality of peptides from Table 1; and optionally a pharmaceutically acceptable excipient, carrier, or diluent. In some cases the plurality may comprise 1, 2, 3, 4, 5, 6, 7, 8, 9, 10 or more polypeptides of Table 1. In some cases, one or more of the plurality of polypeptides is at least about 85%, 90%, 95%, 97%, 98%, 99%, or 100% identical to a polypeptide of Table 1. The composition may comprise THBS1. The composition may comprise THBS2. The composition may comprise THBS4. The composition may comprise FGF17. The composition may comprise VTN. The composition may comprise POSTN. The composition may comprise IGF2. The composition may comprise IL-15. The composition may comprise BMP7. The composition may comprise IGF2, THBS2, and THBS4. The composition may comprise IL-15, THBS2, and THBS4. The composition may comprise THBS2 and THBS4. The composition may comprise THBS2, THBS4, and VTN. The composition may comprise THBS2, THBS4, and ANOS1. The composition may comprise THBS2, THBS4, and IL-15. The composition may comprise THBS2, THBS4, and IGF2. The composition may comprise THBS1 and FGF17. The composition may comprise THBS2 and VTN. The composition may comprise THBS1 and VTN. The composition may comprise THBS1 and THBS2. The composition may comprise THBS2 and FGF17. The composition may comprise THBS1 and THBS4. The composition may comprise VTN and FGF17. The composition may comprise THBS4 and VTN. The composition may comprise THBS4 and FGF17. The composition may comprise BMP7 and IGF2. The composition may comprise BMP7 and FGF17. A composition may comprise any 1, 2, 3, 4, 5, 6, 7, 8, 9, 10 or more polypeptides at least about 85%, 90%, 95%, 97%, 98%, 99%, or 100% identical to CTGF, THBS1, THBS2, THBS3, HGFAC, IGFBP3, IGFBP5, IGFBP7, IGFBP4, SFRP1, STC1, STC2, IGFBP2; and a pharmaceutically acceptable excipient, carrier, or diluent. Described herein, is a composition that may comprise 1, 2, 3, 4, 5, 6, 7, 8, 9, 10 or more polypeptides, wherein one or more the polypeptides are at least about 85%, 90%, 95%, 97%, 98%, 99%, or 100% identical to VTN, POSTN, FGF17, THBS2, THBS1, IGF2, IL-15, BMP7, or THBS4; and a pharmaceutically acceptable excipient, carrier, or diluent.

Described herein is a composition that may comprise a polypeptide comprising at least about 85%, 90%, 95%, 97%, 98%, 99%, or 100% homology or identity to HAPs ID NO: 1 or amino acids 20-478 of HAPs ID NO: 1, and a pharmaceutically acceptable excipient, carrier, or diluent. In some cases, the polypeptide does not comprise a signal sequence of HAPs ID NO: 1. Described herein is a composition that may comprise a polypeptide comprising at least about 85%, 90%, 95%, 97%, 98%, 99%, or 100% homology or identity to HAPs ID NO: 2, and a pharmaceutically acceptable excipient, carrier, or diluent. In some cases, the polypeptide does not comprise a signal sequence of HAPs ID NO: 2. Described herein is a composition that may comprise a polypeptide comprising at least about 85%, 90%, 95%, 97%, 98%, 99%, or 100% homology or identity to HAPs ID NO: 3, and a pharmaceutically acceptable excipient, carrier, or diluent. In some cases, the polypeptide does not comprise a signal sequence of HAPs ID NO: 3. Described herein is a composition that may comprise a polypeptide comprising at least about 85%, 90%, 95%, 97%, 98%, 99%, or 100% homology or identity to HAPs ID NO: 4 or amino acids 19-1172 of HAPs ID NO: 4, and a pharmaceutically acceptable excipient, carrier, or diluent. In some cases, the polypeptide does not comprise a signal sequence of HAPs ID NO: 4. Described herein is a composition that may comprise a polypeptide comprising at least about 85%, 90%, 95%, 97%, 98%, 99%, or 100% homology or identity to HAPs ID NO: 5, and a pharmaceutically acceptable excipient, carrier, or diluent. In some cases, the polypeptide does not comprise a signal sequence of HAPs ID NO: 5. Described herein is a composition that may comprise polypeptide comprising at least about 85%, 90%, 95%, 97%, 98%, 99%, or 100% homology or identity to HAPs ID NO: 6 or amino acids 22-836 of HAPs ID NO: 6, and a pharmaceutically acceptable excipient, carrier, or diluent. In some cases, the polypeptide does not comprise a signal sequence of HAPs ID NO: 6. Described herein is a composition that may comprise a polypeptide comprising at least about 85%, 90%, 95%, 97%, 98%, 99%, or 100% homology or identity to HAPs ID NO: 7 or amino acids 23-216 of HAPs ID NO: 7, and a pharmaceutically acceptable excipient, carrier, or diluent. In some cases, the polypeptide does not comprise a signal sequence of HAPs ID NO: 7. Described herein is a composition that may comprise a polypeptide comprising at least about 85%, 90%, 95%, 97%, 98%, 99%, or 100% homology or identity to HAPs ID NO: 8 or amino acids 27-961 of HAPs ID NO: 8, and a pharmaceutically acceptable excipient, carrier, or diluent. In some cases, the polypeptide does not comprise a signal sequence of HAPs ID NO: 8. Described herein is a composition that may comprise a polypeptide comprising at least about 85%, 90%, 95%, 97%, 98%, 99%, or 100% homology or identity to HAPs ID NO: 9 or amino acids 19-1170 of HAPs ID NO: 9, and a pharmaceutically acceptable excipient, carrier, or diluent. In some cases, the polypeptide does not comprise a signal sequence of HAPs ID NO: 9. Described herein is a composition that may comprise a polypeptide comprising at least about 85%, 90%, 95%, 97%, 98%, 99%, or 100% homology or identity to HAPs ID NO: 10 or amino acids 49-162 of HAPs ID NO: 10, and a pharmaceutically acceptable excipient, carrier, or diluent. In some cases, the polypeptide does not comprise a signal sequence of HAPs ID NO: 10. Described herein is a composition that may comprise a polypeptide comprising at least about 85%, 90%, 95%, 97%, 98%, 99%, or 100% homology or identity to HAPs ID NO: 11 or amino acids 25-91 of HAPs ID NO: 11, and a pharmaceutically acceptable excipient, carrier, or diluent. In some cases, the polypeptide does not comprise a signal sequence of HAPs ID NO: 11.

Described herein is a composition that may comprise a polypeptide comprising at least about 85%, 90%, 95%, 97%, 98%, 99%, or 100% homology or identity to HAPs ID NO: 12, and a pharmaceutically acceptable excipient, carrier, or diluent. In some cases, the polypeptide does not comprise a signal sequence of HAPs ID NO: 12. Described herein is a composition that may comprise a polypeptide comprising at least about 85%, 90%, 95%, 97%, 98%, 99%, or 100% homology or identity to HAPs ID NO: 13, and a pharmaceutically acceptable excipient, carrier, or diluent. In some cases, the polypeptide does not comprise a signal sequence of HAPs ID NO: 13. Described herein is a composition that may comprise a polypeptide comprising at least about 85%, 90%, 95%, 97%, 98%, 99%, or 100% homology or identity to HAPs ID NO: 14, and a pharmaceutically acceptable excipient, carrier, or diluent. In some cases, the polypeptide does not comprise a signal sequence of HAPs ID NO: 14. Described herein is a composition that may comprise a polypeptide comprising at least about 85%, 90%, 95%, 97%, 98%, 99%, or 100% homology or identity to HAPs ID NO: 15 and a pharmaceutically acceptable excipient, carrier, or diluent. In some cases, the polypeptide does not comprise a signal sequence of HAPs ID NO: 15. Described herein is a composition that may comprise a polypeptide comprising at least about 85%, 90%, 95%, 97%, 98%, 99%, or 100% homology or identity to HAPs ID NO: 16, and a pharmaceutically acceptable excipient, carrier, or diluent. In some cases, the polypeptide does not comprise a signal sequence of HAPs ID NO: 16. Described herein is a composition that may comprise a polypeptide comprising at least about 85%, 90%, 95%, 97%, 98%, 99%, or 100% homology or identity to HAPs ID NO: 17, and a pharmaceutically acceptable excipient, carrier, or diluent. In some cases, the polypeptide does not comprise a signal sequence of HAPs ID NO: 17. Described herein is a composition that may comprise a polypeptide comprising at least about 85%, 90%, 95%, 97%, 98%, 99%, or 100% homology or identity to HAPs ID NO: 18, and a pharmaceutically acceptable excipient, carrier, or diluent. In some cases, the polypeptide does not comprise a signal sequence of HAPs ID NO: 18. Described herein is a composition that may comprise a polypeptide comprising at least about 85%, 90%, 95%, 97%, 98%, 99%, or 100% homology or identity to HAPs ID NO: 19, and a pharmaceutically acceptable excipient, carrier, or diluent. In some cases, the polypeptide does not comprise a signal sequence of HAPs ID NO: 19.

Described herein is a composition that may comprise a polypeptide comprising at least about 85%, 90%, 95%, 97%, 98%, 99%, or 100% homology or identity to HAPs ID NO: 20, and a pharmaceutically acceptable excipient, carrier, or diluent. In some cases, the polypeptide does not comprise a signal sequence of HAPs ID NO: 20. Described herein is a composition that may comprise a polypeptide comprising at least about 85%, 90%, 95%, 97%, 98%, 99%, or 100% homology or identity to HAPs ID NO: 21, and a pharmaceutically acceptable excipient, carrier, or diluent. In some cases, the polypeptide does not comprise a signal sequence of HAPs ID NO: 21. Described herein is a composition that may comprise a polypeptide comprising at least about 85%, 90%, 95%, 97%, 98%, 99%, or 100% homology or identity to HAPs ID NO: 22, and a pharmaceutically acceptable excipient, carrier, or diluent. In some cases, the polypeptide does not comprise a signal sequence of HAPs ID NO: 22. Described herein is a composition that may comprise a polypeptide comprising at least about 85%, 90%, 95%, 97%, 98%, 99%, or 100% homology or identity to HAPs ID NO: 23, and a pharmaceutically acceptable excipient, carrier, or diluent. In some cases, the polypeptide does not comprise a signal sequence of HAPs ID NO: 23. Described herein is a composition that may comprise a polypeptide comprising at least about 85%, 90%, 95%, 97%, 98%, 99%, or 100% homology or identity to HAPs ID NO: 24, and a pharmaceutically acceptable excipient, carrier, or diluent. In some cases, the polypeptide does not comprise a signal sequence of HAPs ID NO: 24. Described herein is a composition that may comprise a polypeptide comprising at least about 85%, 90%, 95%, 97%, 98%, 99%, or 100% homology or identity to HAPs ID NO: 25, and a pharmaceutically acceptable excipient, carrier, or diluent. In some cases, the polypeptide does not comprise a signal sequence of HAPs ID NO: 25. Described herein is a composition that may comprise a polypeptide comprising at least about 85%, 90%, 95%, 97%, 98%, 99%, or 100% homology or identity to HAPs ID NO: 26, and a pharmaceutically acceptable excipient, carrier, or diluent. In some cases, the polypeptide does not comprise a signal sequence of HAPs ID NO: 26. Described herein is a composition that may comprise a polypeptide comprising at least about 85%, 90%, 95%, 97%, 98%, 99%, or 100% homology or identity to HAPs ID NO: 27, and a pharmaceutically acceptable excipient, carrier, or diluent. In some cases, the polypeptide does not comprise a signal sequence of HAPs ID NO: 27. Described herein is a composition that may comprise a polypeptide comprising at least about 85%, 90%, 95%, 97%, 98%, 99%, or 100% homology or identity to HAPs ID NO: 28, and a pharmaceutically acceptable excipient, carrier, or diluent. In some cases, the polypeptide does not comprise a signal sequence of HAPs ID NO: 28. Described herein is a composition that may comprise a polypeptide comprising at least about 85%, 90%, 95%, 97%, 98%, 99%, or 100% homology or identity to HAPs ID NO: 29, and a pharmaceutically acceptable excipient, carrier, or diluent. In some cases, the polypeptide does not comprise a signal sequence of HAPs ID NO: 29.

Described herein is a composition that may comprise a polypeptide comprising at least about 85%, 90%, 95%, 97%, 98%, 99%, or 100% homology or identity to HAPs ID NO: 30, and a pharmaceutically acceptable excipient, carrier, or diluent. In some cases, the polypeptide does not comprise a signal sequence of HAPs ID NO: 30. Described herein is a composition that may comprise a polypeptide comprising at least about 85%, 90%, 95%, 97%, 98%, 99%, or 100% homology or identity to HAPs ID NO: 31, and a pharmaceutically acceptable excipient, carrier, or diluent. In some cases, the polypeptide does not comprise a signal sequence of HAPs ID NO: 31. Described herein is a composition that may comprise a polypeptide comprising at least about 85%, 90%, 95%, 97%, 98%, 99%, or 100% homology or identity to HAPs ID NO: 32, and a pharmaceutically acceptable excipient, carrier, or diluent. In some cases, the polypeptide does not comprise a signal sequence of HAPs ID NO: 32. Described herein is a composition that may comprise a polypeptide comprising at least about 85%, 90%, 95%, 97%, 98%, 99%, or 100% homology or identity to HAPs ID NO: 33, and a pharmaceutically acceptable excipient, carrier, or diluent. In some cases, the polypeptide does not comprise a signal sequence of HAPs ID NO: 33. Described herein is a composition that may comprise a polypeptide comprising at least about 85%, 90%, 95%, 97%, 98%, 99%, or 100% homology or identity to HAPs ID NO: 34, and a pharmaceutically acceptable excipient, carrier, or diluent. In some cases, the polypeptide does not comprise a signal sequence of HAPs ID NO: 34. Described herein is a composition that may comprise a polypeptide comprising at least about 85%, 90%, 95%, 97%, 98%, 99%, or 100% homology or identity to HAPs ID NO: 35, and a pharmaceutically acceptable excipient, carrier, or diluent. In some cases, the polypeptide does not comprise a signal sequence of HAPs ID NO: 35. Described herein is a composition that may comprise a polypeptide comprising at least about 85%, 90%, 95%, 97%, 98%, 99%, or 100% homology or identity to HAPs ID NO: 36, and a pharmaceutically acceptable excipient, carrier, or diluent. In some cases, the polypeptide does not comprise a signal sequence of HAPs ID NO: 36. Described herein is a composition that may comprise a polypeptide comprising at least about 85%, 90%, 95%. 97%. 98%, 99%, or 100% homology or identity to HAPs ID NO: 37, and a pharmaceutically acceptable excipient, carrier, or diluent. In some cases, the polypeptide does not comprise a signal sequence of HAPs ID NO: 37. Described herein is a composition that may comprise a polypeptide comprising at least about 85%, 90%, 95%, 97%, 98%, 99%, or 100% homology or identity to HAPs ID NO: 38, and a pharmaceutically acceptable excipient, carrier, or diluent. In some cases, the polypeptide does not comprise a signal sequence of HAPs ID NO: 38. Described herein is a composition that may comprise a polypeptide comprising at least about 85%, 90%, 95%, 97%, 98%, 99%, or 100% homology or identity to HAPs ID NO: 39, and a pharmaceutically acceptable excipient, carrier, or diluent. In some cases, the polypeptide does not comprise a signal sequence of HAPs ID NO: 39.

Described herein is a composition that may comprise a polypeptide comprising at least about 85%, 90%, 95%, 97%, 98%, 99%, or 100% homology or identity to HAPs ID NO: 40, and a pharmaceutically acceptable excipient, carrier, or diluent. In some cases, the polypeptide does not comprise a signal sequence of HAPs ID NO: 40. Described herein is a composition that may comprise a polypeptide comprising at least about 85%, 90%, 95%, 97%, 98%, 99%, or 100% homology or identity to HAPs ID NO: 41, and a pharmaceutically acceptable excipient, carrier, or diluent. In some cases, the polypeptide does not comprise a signal sequence of HAPs ID NO: 41. Described herein is a composition that may comprise a polypeptide comprising at least about 85%, 90%, 95%, 97%, 98%, 99%, or 100% homology or identity to HAPs ID NO: 42, and a pharmaceutically acceptable excipient, carrier, or diluent. In some cases, the polypeptide does not comprise a signal sequence of HAPs ID NO: 42. Described herein is a composition that may comprise a polypeptide comprising at least about 85%, 90%, 95%, 97%, 98%, 99%, or 100% homology or identity to HAPs ID NO: 43, and a pharmaceutically acceptable excipient, carrier, or diluent. In some cases, the polypeptide does not comprise a signal sequence of HAPs ID NO: 43. Described herein is a composition that may comprise a polypeptide comprising at least about 85%, 90%, 95%, 97%, 98%, 99%, or 100% homology or identity to HAPs ID NO: 44, and a pharmaceutically acceptable excipient, carrier, or diluent. In some cases, the polypeptide does not comprise a signal sequence of HAPs ID NO: 44. Described herein is a composition that may comprise a polypeptide comprising at least about 85%, 90%, 95%, 97%, 98%, 99%, or 100% homology or identity to HAPs ID NO: 58, and a pharmaceutically acceptable excipient, carrier, or diluent. In some cases, the polypeptide does not comprise a signal sequence of HAPs ID NO: 58. Described herein is a composition that may comprise a polypeptide comprising at least about 85%, 90%, 95%, 97%, 98%, 99%, or 100% homology or identity to HAPs ID NO: 59, and a pharmaceutically acceptable excipient, carrier, or diluent. In some cases, the polypeptide does not comprise a signal sequence of HAPs ID NO: 59. Described herein is a composition that may comprise a polypeptide comprising at least about 85%, 90%, 95%, 97%, 98%, 99%, or 100% homology or identity to HAPs ID NO: 60, and a pharmaceutically acceptable excipient, carrier, or diluent. In some cases, the polypeptide does not comprise a signal sequence of HAPs ID NO: 60. Described herein is a composition that may comprise a polypeptide comprising at least about 85%, 90%, 95%, 97%, 98%, 99%, or 100% homology or identity to HAPs ID NO: 61, and a pharmaceutically acceptable excipient, carrier, or diluent. In some cases, the polypeptide does not comprise a signal sequence of HAPs ID NO: 61. Described herein is a composition that may comprise a polypeptide comprising at least about 85%, 90%, 95%, 97%, 98%, 99%, or 100% homology or identity to HAPs ID NO: 62, and a pharmaceutically acceptable excipient, carrier, or diluent. In some cases, the polypeptide does not comprise a signal sequence of HAPs ID NO: 62. Described herein is a composition that may comprise a polypeptide comprising at least about 85%, 90%, 95%, 97%, 98%, 99%, or 100% homology or identity to HAPs ID NO: 63, and a pharmaceutically acceptable excipient, carrier, or diluent. In some cases, the polypeptide does not comprise a signal sequence of HAPs ID NO: 63. Described herein is a composition that may comprise a polypeptide comprising at least about 85%, 90%, 95%, 97%, 98%, 99%, or 100% homology or identity to HAPs ID NO: 64, and a pharmaceutically acceptable excipient, carrier, or diluent. In some cases, the polypeptide does not comprise a signal sequence of HAPs ID NO: 64. Described herein is a composition that may comprise a polypeptide comprising at least about 85%, 90%, 95%, 97%, 98%, 99%, or 100% homology or identity to HAPs ID NO: 65, and a pharmaceutically acceptable excipient, carrier, or diluent. In some cases, the polypeptide does not comprise a signal sequence of HAPs ID NO: 65. Described herein is a composition that may comprise a polypeptide comprising at least about 85%, 90%, 95%, 97%, 98%, 99%, or 100% homology or identity to HAPs ID NO: 66, and a pharmaceutically acceptable excipient, carrier, or diluent. In some cases, the polypeptide does not comprise a signal sequence of HAPs ID NO: 66. Described herein is a composition that may comprise a polypeptide comprising at least about 85%, 90%, 95%, 97%, 98%, 99%, or 100% homology or identity to HAPs ID NO: 67, and a pharmaceutically acceptable excipient, carrier, or diluent. In some cases, the polypeptide does not comprise a signal sequence of HAPs ID NO: 67. Described herein is a composition that may comprise a polypeptide comprising at least about 85%, 90%, 95%, 97%, 98%, 99%, or 100% homology or identity to HAPs ID NO: 68, and a pharmaceutically acceptable excipient, carrier, or diluent. In some cases, the polypeptide does not comprise a signal sequence of HAPs ID NO: 68. Described herein is a composition that may comprise a polypeptide comprising at least about 85%, 90%, 95%, 97%, 98%, 99%, or 100% homology or identity to HAPs ID NO: 72, and a pharmaceutically acceptable excipient, carrier, or diluent. In some cases, the polypeptide does not comprise a signal sequence of HAPs ID NO: 72.

Described herein is a composition that may comprise comprising 1, a plurality, or 2, 3, 4, 5, 6, 7, 8, 9, 10 or more polypeptides at least about 85%, 90%, 95%, 97%, 98%, 99%, or 100% identical to ADAMTS12, INS-IGF2, AOC1, SOD3, CLU, ITIH1, APLP1, THBS1, COCH, ITIH2, APLP2, THBS3, COL11A1, LAMA1, APOB, TNXB, COL12A1, LAMA2, APOE, VEGFA, COL14A1, LAMA5, APOH, VTN, COL18A1, LAMB1, APP, ZNF207, COL1A1, LAMB2, CCDC80, COL1A2, LTF, CFH, COL2A1, MATN2, CLEC3B, COL3A1, MDK, COL25A1, COL5A1, MST1, COL5A3, COL5A2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, RPL29, BMP7, and combinations thereof; and a pharmaceutically acceptable excipient, carrier, or diluent. Described herein is a composition that may comprise comprising a plurality of polypeptides at least about 85%, 90%, 95%, 97%, 98%, 99%, or 100% identical to CTGF, THBS1, THBS2, THBS3, HGFAC, IGFBP3, IGFBP5, IGFBP7, IGFBP4, SFRP1, STC1, STC2, IGFBP2; and a pharmaceutically acceptable excipient, carrier, or diluent.

Described herein is a composition that may comprise consisting essentially of 1, a plurality, or 2, 3, 4, 5, 6, 7, 8, 9, 10 or more polypeptides at least about 85%, 90%, 95%, 97%, 98%, 99%, or 100% identical to ADAMTS12, INS-IGF2, AOC1, SOD3, CLU, ITIH1, APLP1, THBS1, COCH, ITIH2, APLP2, THBS3, COL11A1, LAMA1, APOB, TNXB, COL12A1, LAMA2, APOE, VEGFA, COL14A1, LAMA5, APOH, VTN, COL18A1, LAMB1, APP, ZNF207, COL1A1, LAMB2, CCDC80, COL1A2, LTF, CFH, COL2A1, MATN2, CLEC3B, COL3A1, MDK, COL25A1, COL5A1, MST1, COL5A3, COL5A2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, BMP7, and RPL29; and a pharmaceutically acceptable excipient, carrier, or diluent. Described herein is a composition that may comprise consisting essentially of any 1, 2, 3, 4, 5, 6, 7, 8, 9, 10 or more polypeptides at least about 85%, 90%, 95%, 97%, 98%, 99%, or 100% identical to CTGF, THBS1, THBS2, THBS3, HGFAC, IGFBP3, IGFBP5, IGFBP7, IGFBP4, SFRP1, STC1, STC2, IGFBP2; and a pharmaceutically acceptable excipient, carrier, or diluent.

In certain embodiments, compositions comprising HAPs do not comprise fibroblast growth factors (FGF). In certain embodiments, compositions comprising HAPs do not comprise fibroblast growth factor 2 (FGF2). In certain embodiments, compositions comprising HAPs do not comprise FGF19, Angiogenin, BTC, IL-13 R alpha 2, Siglec-5/CD170, IL-15, APJ, IGFBP-2, Chordin-Like 1, GASP-1/WFIKKNRP, MFRP, IL-10 R alpha, Chem R23, HB-EGF, FGF-6, HGF, IL-16, IL-7 R alpha, TRAIL R3/TNFRSF10C, BMP-6, IL-1 F9/IL-1 H1, IL-1 beta, Kremen-2, TRAIL R4/TNFRSF10D, CXCR1/IL-8 RA, Ck beta 8-1/CCL23, Beta-catenin, FGF-13 1B, TRAIL/TNFSF10, CCL14/HCC-1/HCC-3, or FGF-4, or a combination thereof.

In certain aspects, heparin-associated binding polypeptides and compositions of heparin-associated binding polypeptides herein comprise polypeptides that increase the proliferation of muscle cell precursors, and/or increase their differentiation into muscle cells. The HAPs increase proliferation of a muscle cell precursor by at least about 20%, 30%, 40%, 50%, or 100% compared to a muscle cell precursor not treated with the heparin-associated binding polypeptide. The HAPs increase proliferation of a myoblast by at least about 20%, 30%, 40%, 50%, 100%, 200%, or 500% compared to a myoblast not treated with the heparin-associated binding polypeptide. The myoblast is a human myoblast cell line. The myoblast is a mouse myoblast cell line (e.g., C2C12). Proliferation can be measured by BrdU or EdU incorporation, which can be quantified using suitable methods such as, by way of non-limiting embodiment, microscopy, flow cytometry, or ELISA.

The HAPs increase differentiation and/or fusion of a muscle cell precursor by at least about 50%, 75%, 100%, 200%, or 500% compared to a muscle cell precursor not treated with the heparin-associated binding polypeptide. The HAPs increase differentiation of a myoblast by at least about 50%, 75%, or 100% compared to a myoblast not treated with the heparin-associated binding polypeptide. The myoblast is a human myoblast cell line. The myoblast is a mouse myoblast cell line (e.g., C2C12). Differentiation can be measured and/or quantified by eMyHC staining, which detects fusion of a myoblast or muscle cell precursor. This staining can be quantified, for example, by microscopy or flow cytometry.

HAPs that increase muscle or connective tissue cell precursor proliferation and/or differentiation are useful in methods of treating muscle or connective tissue disorders. These disorders can arise from the normal aging process, injury related to trauma or physical exertion, genetic predispositions, or incident to other disease states.

Heparin-associated binding polypeptides that are useful for increasing muscle cell precursor differentiation or proliferation are described herein, and in certain embodiments comprise Vitronectin (VTN), Stanniocalcin-2 (STC2), Periostin (POSTN), Agrin (AGRN), Fibroblast growth factor (FGF17, also known as Fibroblast growth factor 13 or FGF13), Thrombospondin 2 (THBS2), follistatin (FST), Thrombospondin 4 (THBS4), Thrombospondin 1 (THBS1), Insulin-like growth factor 2 (IGF2), Bone morphogenic protein 7 (BMP7), or Interleukin 15 (IL-15), or any combination thereof. In certain embodiments, any one, two, three, four, or five of VTN, STC2, AGRN, THBS2, or FST are present in a composition useful for increasing muscle cell precursor proliferation or muscle cell differentiation. In certain embodiments, any one, two, three, four, five, six, seven, or eight of VTN, POSTN, FGF17, THBS2, THBS1, IL-15, IGF2, and THBS4 are present in a composition useful for increasing muscle cell precursor proliferation or muscle cell differentiation.

In certain embodiments, a heparin-associated binding polypeptide composition may comprise Vitronectin (VTN). VTN may be further included in the composition with any one, two, three, four, five, six, seven, eight, nine, or all polypeptides selected from STC2, AGRN, POSTN, FGF17, THBS2, FST, THBS1, IL-15, IGF2, and THBS4. The composition may comprise VTN and STC2. The composition may comprise VTN and AGRN. The composition may comprise VTN and THBS2. The composition may comprise VTN and FST. The composition may comprise VTN and POSTN. The composition may comprise VTN and FGF17. The composition may comprise VTN and THBS4. The composition may comprise VTN and THBS1. The composition may comprise VTN and IGF2. The composition may comprise VTN and IL-15. Human VTN is disclosed in HAPs ID NO: 1. The VTN of the heparin-associated binding polypeptide composition may comprise an amino acid sequence that is at least about 80%, 85%, 90%, 95%, 97%, 98%, 99%, or 100% identical to HAPs ID NO: 1 or amino acids 20-478 of HAPs ID NO: 1. The VTN polypeptide lacks a secretory leader sequence, e.g., amino acids 1-19 of HAPs ID NO: 1. The VTN polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. The VTN polypeptide may comprise one or more additional modifications to increase stability. The VTN polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The VTN polypeptide is fused with an Fc region of an immunoglobulin or with serum albumin. The VTN polypeptide is present in a concatemer with one, two, three, four or more distinct polypeptides selected from STC2, AGRN, THBS2, THBS1, THBS4, FGF17, POSTN, IGF2, IL-15 and FST. The VTN polypeptide is present in a concatemer with one, two, three, four or more distinct polypeptides selected from POSTN, FGF17, THBS2, THBS1, IGF2, IL-15, and THBS4. The VTN polypeptide is present in a concatemer with one, two, three, four, or more distinct VTN polypeptides. The VTN polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the VTN polypeptide is prepared recombinantly in an expression system (e.g., bacteria, yeast, mammalian, insect). In some cases, the VTN polypeptide is prepared by chemical synthesis.

In certain embodiments, a heparin-associated binding polypeptide composition may comprise Periostin (POSTN). POSTN may be further included in the composition with any one, two, three, four, five, six, or all polypeptides selected from VTN, FGF17, THBS2, THBS1, IGF2, IL-15, and THBS4. The composition may comprise POSTN and VTN. The composition may comprise POSTN and FGF17. The composition may comprise POSTN and THBS2. The composition may comprise POSTN and THBS4. The composition may comprise POSTN and THBS1. The composition may comprise POSTN and IGF2. The composition may comprise POSTN and IL-15. Human POSTN is disclosed in HAPs ID NO: 6. The POSTN of the heparin-associated binding polypeptide composition may comprise an amino acid sequence that is at least about 80%, 85%, 90%, 95%, 97%, 98%, 99%, or 100% identical to HAPs ID NO: 6 or amino acids 22-836 of HAPs ID NO: 6. The POSTN polypeptide lacks a secretory leader sequence, e.g., amino acids 1-21 of HAPs ID NO: 6. The POSTN polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. The POSTN polypeptide may comprise one or more additional modifications to increase stability. The POSTN polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The POSTN polypeptide is fused with an Fc region of an immunoglobulin or with serum albumin. The POSTN polypeptide is present in a concatemer with one, two, three, four or more distinct polypeptides selected from VTN, FGF17, THBS2, THBS1, IGF2, IL-15, BMP7, and THBS4. The POSTN polypeptide is present in a concatemer with one, two, three, four, or more distinct POSTN polypeptides. The POSTN polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the POSTN polypeptide is prepared recombinantly in an expression system (e.g., bacteria, yeast, mammalian, insect). In some cases, the POSTN polypeptide is prepared by chemical synthesis.

In certain embodiments, a heparin-associated binding polypeptide composition may comprise Fibroblast growth factor (FGF17). FGF17 may be further included in the composition with any one, two, three, four, five, six, seven, or all polypeptides selected from VTN, POSTN, THBS2, THBS1, IL-15, IGF2, BMP7, and THBS4. The composition may comprise FGF17 and VTN. The composition may comprise FGF17 and POSTN. The composition may comprise FGF17 and THBS2. The composition may comprise FGF17 and THBS4. The composition may comprise FGF17 and THBS1. The composition may comprise FGF17 and IGF2. The composition may comprise FGF17 and IL-15. Human FGF17 is disclosed in HAPs ID NO: 7. The FGF17 of the heparin-associated binding polypeptide composition may comprise an amino acid sequence that is at least about 80%, 85%, 90%, 95%, 97%, 98%, 99%, or 100% identical to HAPs ID NO: 7, or amino acids 23-216 of HAPs ID NO: 7. The FGF17 polypeptide lacks a secretory leader sequence, e.g., amino acids 1-22 of HAPs ID NO: 7. The FGF17 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 amino acids from the N-terminal or C-terminal end of the polypeptide, including increments therein. The FGF17 polypeptide may comprise one or more additional modifications to increase stability. The FGF17 polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The FGF17 polypeptide is fused with an Fc region of an immunoglobulin or with serum albumin. The FGF17 polypeptide is present in a concatemer with one, two, three, four or more distinct polypeptides selected from VTN, POSTN, THBS2, THBS1, IGF2, IL-15, BMP7, and THBS4. The FGF17 polypeptide is present in a concatemer with one, two, three, four, or more distinct FGF17 polypeptides. The FGF17 polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the FGF17 polypeptide is prepared recombinantly in an expression system (e.g., bacteria, yeast, mammalian, insect). In some cases, the FGF17 polypeptide is prepared by chemical synthesis.

In certain embodiments, a heparin-associated binding polypeptide composition may comprise Stanniocalcin-2 (STC2). STC-2 may be further included in the composition with any one, two, three, four or more polypeptides selected from VTN, AGRN, THBS2, THBS1, THBS4, POSTN, FGF17, IGF2, IL-15, BMP7, and FST. The composition may comprise STC2 and VTN. The composition may comprise STC2 and AGRN. The composition may comprise STC2 and THBS2. The composition may comprise STC2 and FST. Human STC2 is disclosed in HAPs ID NO: 2. The STC2 of the heparin-associated binding polypeptide composition may comprise an amino acid sequence that is at least about 80%, 85%, 90%, 95%, 97%, 98%, 99%, or 100% identical to HAPs ID NO: 2. The STC2 polypeptide lacks a secretory leader sequence. The STC2 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. The STC2 polypeptide may comprise one or more additional modifications to increase stability. The STC2 polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The STC2 polypeptide is fused with an Fc region of an immunoglobulin or with serum albumin. The STC2 polypeptide is present in a concatemer with one, two, three, four or more distinct polypeptides selected from VTN, AGRN, THBS2, THBS1, THBS4, FGF17, POSTN, IGF2, BMP7, IL15, and FST. The STC2 polypeptide is present in a concatemer with one, two, three, four, or more distinct STC2 polypeptides. The STC2 polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the STC-2 polypeptide is prepared recombinantly in an expression system (e.g., bacteria, yeast, mammalian, insect). In some cases, the STC-2 polypeptide is prepared by chemical synthesis.

In certain embodiments, a heparin-associated binding polypeptide composition may comprise Agrin (AGRN). AGRN may be further included in the composition with any one, two, three, four or more polypeptides selected from VTN, STC2, THBS2, THBS1, THBS4, FGF17, POSTN, IGF2, IL-15, BMP7, and FST. The composition may comprise AGRN and VTN. The composition may comprise AGRN and STC2. The composition may comprise AGRN and THBS2. The composition may comprise AGRN and FST. Human AGRN is disclosed in HAPs ID NO: 3. The AGRN of the heparin-associated binding polypeptide composition may comprise an amino acid sequence that is at least about 80%, 85%, 90%, 95%, 97%, 98%, 99%, or 100% identical to HAPs ID NO: 3. The AGRN polypeptide lacks a secretory leader sequence. The AGRN polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 amino acids from the N-terminal or C-terminal end of the polypeptide, including increments therein. The AGRN polypeptide may comprise one or more additional modifications to increase stability. The AGRN polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The AGRN polypeptide is fused with an Fc region of an immunoglobulin or with serum albumin. The AGRN polypeptide is present in a concatemer with one, two, three, or four other distinct polypeptides selected from VTN, STC2, THBS2, THBS1, THBS4, FGF17, POSTN, IGF2, IL-15, and FST. The AGRN polypeptide is present in a concatemer with one, two, three, four, or more distinct AGRN polypeptides. The AGRN polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the AGRN polypeptide is prepared recombinantly in an expression system (e.g., bacteria, yeast, mammalian, insect). In some cases, the AGRN polypeptide is prepared by chemical synthesis.

In certain embodiments, a heparin-associated binding polypeptide composition may comprise Thrombospondin 2 (THBS2). THBS2 may be further included in the composition with any one, two, three, four, five, six or all polypeptides selected from VTN, STC2, AGRN, THBS1, IL-15, IGF2, and FST. The composition may comprise THBS2 and VTN. The composition may comprise THBS2 and STC2. The composition may comprise THBS2 and AGRN. The composition may comprise THBS2 and FST. The composition may comprise AGRN and FST. The composition may comprise THBS2 and THBS1. The composition may comprise THBS2 and IGF2. The composition may comprise THBS2 and IL-15. THBS2 may be further included in the composition with any one, two, three, four, five, six, or all polypeptides selected from VTN, POSTN, FGF17, THBS1, IL-15, IGF2, and THBS4. The composition may comprise THBS2 and VTN. The composition may comprise THBS2 and POSTN. The composition may comprise THBS2 and FGF17. The composition may comprise THBS2 and THBS4. The composition may comprise FGF17 and THBS4. Human THBS2 is disclosed in HAPs ID NO: 4. The THBS2 of the heparin-associated binding polypeptide composition may comprise an amino acid sequence that is at least about 80%, 85%, 90%, 95%, 97%, 98%, 99%, or 100% identical to HAPs ID NO: 4 or amino acids 19-1,172 of HAPs ID NO: 4. The THBS2 polypeptide lacks a secretory leader sequence, e.g., amino acids 1-18 of HAPs ID NO: 4. The THBS2 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 amino acids from the N-terminal or C-terminal end of the polypeptide, including increments therein. The THBS2 polypeptide may comprise one or more additional modifications to increase stability. The THBS2 polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The THBS2 polypeptide is fused with an Fc region of an immunoglobulin or with serum albumin. The THBS2 polypeptide is present in a concatemer with one, two, three, four or more distinct polypeptides selected from VTN, STC2, AGRN, THBS1, THBS4, FGF17, POSTN, IGF2, IL-15, and FST. The THBS2 polypeptide is present in a concatemer with one, two, three, four or more distinct polypeptides selected from VTN, POSTN, THBS1, IGF2, IL-15, FGF17, and THBS4. The THBS2 polypeptide is present in a concatemer with one, two, three, four, or more distinct THBS2 polypeptides. The THBS2 polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the THBS2 polypeptide is prepared recombinantly in an expression system (e.g., bacteria, yeast, mammalian, insect). In some cases, the THBS2 polypeptide is prepared by chemical synthesis.

In certain embodiments, a heparin-associated binding polypeptide composition may comprise Thrombospondin 4 (THBS4). THBS4 may be further included in the composition with any one, two, three, four, five, six or all polypeptides selected from VTN, POSTN, FGF17, THBS1, IL-15, IGF2, and THBS2. The composition may comprise THBS4 and VTN. The composition may comprise THBS4 and POSTN. The composition may comprise THBS4 and FGF17. The composition may comprise THBS4 and THBS2. The composition may comprise THBS4 and THBS1. The composition may comprise THBS4 and IL-15. The composition may comprise THBS4 and IGF2. Human THBS4 is disclosed in HAPs ID NO: 8. The THBS4 of the heparin-associated binding polypeptide composition may comprise an amino acid sequence that is at least about 80%, 85%, 90%, 95%, 97%, 98%, 99%, or 100% identical to HAPs ID NO: 8 or amino acids 27-961 of HAPs ID NO: 8. The THBS4 polypeptide lacks a secretory leader sequence, e.g., amino acids 1-26 of HAPs ID NO: 8. The THBS4 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. The THBS4 polypeptide may comprise one or more additional modifications to increase stability. The THBS4 polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The THBS4 polypeptide is fused with an Fc region of an immunoglobulin or with serum albumin. The THBS4 polypeptide is present in a concatemer with one, two, three, four or more distinct polypeptides selected from VTN, POSTN, FGF17, THBS1, IGF2, IL-15, and THBS2. The THBS4 polypeptide is present in a concatemer with one, two, three, four, or more distinct THBS4 polypeptides. The THBS4 polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the THBS4 polypeptide is prepared recombinantly in an expression system (e.g., bacteria, yeast, mammalian, insect). In some cases, the THBS4 polypeptide is prepared by chemical synthesis.

In certain embodiments, a heparin-associated binding polypeptide composition may comprise follistatin (FST). FST may be further included in the composition with any one, two, three, four or more polypeptides selected from VTN, STC2, AGRN, THBS1, THBS4, FGF17, POSTN, IGF2, IL-15, and THBS2. The composition may comprise FST and VTN. The composition may comprise FST and STC2. The composition may comprise FST and AGRN. The composition may comprise FST and THBS2. Human FST is disclosed in HAPs ID NO: 5. The FST of the heparin-associated binding polypeptide composition may comprise an amino acid sequence that is at least about 80%, 85%, 90%, 95%, 97%, 98%, 99%, or 100% identical to HAPs ID NO: 5. The FST polypeptide lacks a secretory leader sequence. The FST polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. The FST polypeptide may comprise one or more additional modifications to increase stability. The FST polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The FST polypeptide is fused with an Fc region of an immunoglobulin or with serum albumin. The FST polypeptide is present in a concatemer with one, two, three, four or more distinct polypeptides selected from VTN, STC2, AGRN, THBS1, THBS4, FGF17, POSTN, IGF2, IL-15, BMP7, and THBS2. The FST polypeptide is present in a concatemer with one, two, three, four, or more distinct FST polypeptides. The FST polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA).

In certain embodiments, a heparin-associated binding polypeptide composition may comprise Thrombospondin 1 (THBS1). THSB1 may be further included in the composition with any one, two, three, four, five, six, seven, eight or all polypeptides selected from VTN, STC2, AGRN, THBS4, FGF17, POSTN, IGF2, IL-15, BMP7, and THBS2. The composition may comprise THSB1 and VTN. The composition may comprise THSB1 and STC2. The composition may comprise THSB1 and AGRN. The composition may comprise THSB1 and THBS2. The composition may comprise THSB1 and THBS4. The composition may comprise THSB1 and FGF17. The composition may comprise THSB1 and POSTN. The composition may comprise THSB1 and IGF2. The composition may comprise THSB1 and IL-15. Human THSB1 is disclosed in HAPs ID NO: 9. The THSB1 of the heparin-associated binding polypeptide composition may comprise an amino acid sequence that is at least about 80%, 85%, 90%, 95%, 97%, 98%, 99%, or 100% identical to HAPs ID NO: 9 or amino acids 19-1170 of HAPs ID NO: 9. The THSB1 polypeptide lacks a secretory leader sequence. The THSB1 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. The THSB1 polypeptide may comprise one or more additional modifications to increase stability. The THSB1 polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The THSB1 polypeptide is fused with an Fc region of an immunoglobulin or with serum albumin. The THSB1 polypeptide is present in a concatemer with one, two, three, four or more distinct polypeptides selected from VTN, STC2, AGRN, THBS4, FGF17, POSTN, IGF2, IL-15, BMP7, and THBS2. The THSB1 polypeptide is present in a concatemer with one, two, three, four, or more distinct THSB1 polypeptides. The THSB1 polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the THBS1 polypeptide is prepared recombinantly in an expression system (e.g., bacteria, yeast, mammalian, insect). In some cases, the THBS1 polypeptide is prepared by chemical synthesis.

In certain embodiments, a heparin-associated binding polypeptide composition may comprise Interleukin-15 (IL-15). IL-15 may be further included in the composition with any one, two, three, four or more polypeptides selected from VTN, STC2, AGRN, THBS1, THBS4, IGF2, POSTN, FGF17, BMP7, and THBS2. The composition may comprise IL-15 and VTN. The composition may comprise IL-15 and STC2. The composition may comprise IL-15 and AGRN. The composition may comprise IL-15 and THBS2. The composition may comprise IL-15 and THBS1. The composition may comprise IL-15 and THBS4. The composition may comprise IL-15 and IGF2. The composition may comprise IL-15 and POSTN. The composition may comprise IL-15 and FGF17. The composition may comprise IL-15 and THBS1. Human IL-15 is disclosed in HAPs ID NO: 10. The IL-15 of the heparin-associated binding polypeptide composition may comprise an amino acid sequence that is at least about 80%, 85%, 90%, 95%, 97%, 98%, 99%, or 100% identical to HAPs ID NO: 10 or amino acids 49-162 of HAPs ID NO: 10. The IL-15 polypeptide lacks a secretory leader sequence. The IL-15 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. The IL-15 polypeptide may comprise one or more additional modifications to increase stability. The IL-15 polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The IL-15 polypeptide is fused with an Fc region of an immunoglobulin or with serum albumin. The IL-15 polypeptide is present in a concatemer with one, two, three, four or more distinct polypeptides selected from VTN, STC2, AGRN, THBS1, THBS4, FGF17, POSTN, IGF2, BMP7, and THBS2. The IL-15 polypeptide is present in a concatemer with one, two, three, four, or more distinct IL-15 polypeptides. The IL-15 polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the IL-15 polypeptide is prepared recombinantly in an expression system (e.g., bacteria, yeast, mammalian, insect). In some cases, the IL-15 polypeptide is prepared by chemical synthesis.

In certain embodiments, a heparin-associated binding polypeptide composition may comprise Insulin-like growth factor 2 (IGF2). IGF2 may be further included in the composition with any one, two, three, four or more polypeptides selected from VTN, STC2, AGRN, THBS1, THBS4, POSTN, FGF17, IL-15, BMP7, and THBS2. The composition may comprise IGF2 and VTN. The composition may comprise IGF2 and STC2. The composition may comprise IGF2 and AGRN. The composition may comprise IGF2 and THBS2. The composition may comprise IGF2 and THBS1. The composition may comprise IGF2 and THBS4. The composition may comprise IGF2 and IL-15. The composition may comprise IGF2 and POSTN. The composition may comprise IGF2 and FGF17. Human IGF2 is disclosed in HAPs ID NO: 11. The IGF2 of the heparin-associated binding polypeptide composition may comprise an amino acid sequence that is at least about 80%, 85%, 90%, 95%, 97%, 98%, 99%, or 100% identical to HAPs ID NO: 11 or amino acids 25-91 of HAPs ID NO: 11. The IGF2 polypeptide lacks a secretory leader sequence. The IGF2 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. The IGF2 polypeptide may comprise one or more additional modifications to increase stability. The IGF2 polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The IGF2 polypeptide is fused with an Fc region of an immunoglobulin or with serum albumin. The IGF2 polypeptide is present in a concatemer with one, two, three, four or more distinct polypeptides selected from VTN, STC2, AGRN, THBS1, THBS4, FGF17, POSTN, IL-15, BMP7, and THBS2. The IGF2 polypeptide is present in a concatemer with one, two, three, four, or more distinct IGF2 polypeptides. The IGF2 polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the IGF2 polypeptide is prepared recombinantly in an expression system (e.g., bacteria, yeast, mammalian, insect). In some cases, the IGF2 polypeptide is prepared by chemical synthesis.

In certain embodiments, a heparin-associated binding polypeptide composition may comprise Bone Morphogenic Protein 7 (BMP7). BMP7 may be further included in the composition with any one, two, three, four or more polypeptides selected from VTN, STC2, AGRN, THBS1, THBS4, POSTN, FGF17, IL-15, IGF2, and THBS2. The composition may comprise IGF2 or FGF-17. The composition may comprise IGF2 and STC2. The composition may comprise IGF2 and AGRN. The composition may comprise IGF2 and THBS2. The composition may comprise IGF2 and THBS1. The composition may comprise IGF2 and THBS4. The composition may comprise IGF2 and IL-15. The composition may comprise IGF2 and POSTN. The composition may comprise IGF2 and FGF17. Human BMP7 is disclosed in HAPs ID NO: 72. The BMP7 of the heparin-associated binding polypeptide composition may comprise an amino acid sequence that is at least about 80%, 85%, 90%, 95%, 97%, 98%, 99%, or 100% identical to HAPs ID NO: 11 or amino acids 25-91 of HAPs ID NO: 11. The BMP7 polypeptide lacks a secretory leader sequence. The BMP7 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. The BMP7 polypeptide may comprise one or more additional modifications to increase stability. The BMP7 polypeptide is covalently conjugated to polyethylene glycol, polypropylene glycol, polyoxyalkylenes, or copolymers of polyethylene glycol, polysialic acid, glycolic acid, or polypropylene glycol. The HAP is fused or conjugated to another protein to increase stability and or bioavailability. The BMP7 polypeptide is fused with an Fc region of an immunoglobulin or with serum albumin. The BMP7 polypeptide is present in a concatemer with one, two, three, four or more distinct polypeptides selected from VTN, STC2, AGRN, THBS1, THBS4, FGF17, POSTN, IL-15, IGF2, and THBS2. The BMP7 polypeptide is present in a concatemer with one, two, three, four, or more distinct BMP7 polypeptides. The BMP7 polypeptide is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA). In some cases, the BMP7 polypeptide is prepared recombinantly in an expression system (e.g., bacteria, yeast, mammalian, insect). In some cases, the BMP7 polypeptide is prepared by chemical synthesis.

The heparin-associated binding polypeptide composition may comprise any two polypeptides selected from VTN, POSTN, FGF17, THBS2, THBS1, IGF2, IL-15, BMP7, and THBS4. The composition may comprise THBS1. The composition may comprise THBS2. The composition may comprise THBS4. The composition may comprise FGF17. The composition may comprise VTN. The composition may comprise POSTN. The composition may comprise IGF2. The composition may comprise IL-15. The composition may comprise VTN and POSTN. The composition may comprise VTN and FGF17. The composition may comprise VTN and THBS2. The composition may comprise VTN and THBS1. The composition may comprise VTN and IGF2. The composition may comprise VTN and IL-15. The composition may comprise VTN and THBS4. The composition may comprise POSTN and FGF17. The composition may comprise POSTN and THBS2. The composition may comprise POSTN and THBS1. The composition may comprise POSTN and IGF2. The composition may comprise POSTN and IL-15. The composition may comprise POSTN and THBS4. The composition may comprise FGF17 and THBS2. The composition may comprise FGF17 and THBS1. The composition may comprise FGF17 and IGF2. The composition may comprise FGF17 and IL-15. The composition may comprise FGF17 and THBS4. The composition may comprise THBS2 and THBS1. The composition may comprise THBS2 and IGF2. The composition may comprise THBS2 and IL-15. The composition may comprise THBS2 and THBS4. The composition may comprise THBS1 and IGF2. The composition may comprise THBS1 and IL-15. The composition may comprise THBS1 and THBS4. The composition may comprise IGF2 and IL-15. The composition may comprise IGF2 and THBS4. The composition may comprise IL-15 and THBS4.

The heparin-associated binding polypeptide composition comprising any two polypeptides selected from VTN, POSTN, FGF17, THBS2, THBS1, IGF2, IL-15, BMP7, and THBS4, may comprise one or more additional modifications to increase stability. In certain embodiments, one or more of the HAPs is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the HAPs is fused or conjugated to another protein to increase stability and or bioavailability. In certain embodiments, one or more of the HAPs is fused with an Fc region of an immunoglobulin or with serum albumin. In certain embodiments, one or more of the HAPs is present in a concatemer with one, two, three, or four other distinct polypeptides selected from THBS2, VTN, POSTN, FGF17, THBS1, IGF2, IL-15, BMP7, and THBS4. In certain embodiments, one or more of the HAPs is present in a concatemer with one, two, three, four, or more distinct polypeptides. In certain embodiments, one or more of the HAPs is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA).

The heparin-associated binding polypeptide composition may comprise any three polypeptides selected from VTN, POSTN, FGF17, THBS2, THBS1, IGF2, IL-15, BMP7, and THBS4. The composition may comprise THBS1. The composition may comprise THBS2. The composition may comprise THBS4. The composition may comprise FGF17. The composition may comprise VTN. The composition may comprise POSTN. The composition may comprise IGF2. The composition may comprise IL-15. The composition may comprise IGF2, THBS2, and THBS4. The composition may comprise IL-15, THBS2, and THBS4. The composition may comprise THBS2 and THBS4. The composition may comprise THBS2, THBS4, and VTN. The composition may comprise THBS2, THBS4, and ANOS1. The composition may comprise THBS2, THBS4, and IL-15. The composition may comprise THBS2, THBS4, and IGF2. The composition may comprise THBS1 and FGF17. The composition may comprise THBS2 and VTN. The composition may comprise THBS1 and VTN. The composition may comprise THBS1 and THBS2. The composition may comprise THBS2 and FGF17. The composition may comprise THBS1 and THBS4. The composition may comprise VTN and FGF17. The composition may comprise THBS4 and VTN. The composition may comprise THBS4 and FGF17.

The composition may comprise VTN, POSTN, and FGF17. The composition may comprise VTN, POSTN, and THBS2. The composition may comprise VTN, POSTN, and FGF17. The composition may comprise VTN, POSTN, THBS2. The composition may comprise VTN, POSTN, and THBS1. The composition may comprise VTN, POSTN, IGF2. The composition may comprise VTN, POSTN, and IL-15. The composition may comprise VTN, POSTN, and THBS4.

The composition may comprise VTN, FGF17, and THBS2. The composition may comprise VTN, FGF17, and POSTN. The composition may comprise VTN, FGF17, and THBS2. The composition may comprise VTN, FGF17, and THBS1. The composition may comprise VTN, FGF17, and IGF2. The composition may comprise VTN, FGF17, and IL-15. The composition may comprise VTN, FGF17, and THBS4.

The composition may comprise VTN, THBS2, and POSTN. The composition may comprise VTN, THBS2, and FGF17. The composition may comprise VTN, THBS2, and THBS1. The composition may comprise VTN, THBS2, and IGF2. The composition may comprise VTN, THBS2, and IL-15. The composition may comprise VTN, THBS2, and THBS4.

The composition may comprise VTN, THBS1, and POSTN. The composition may comprise VTN, THBS1, and FGF17. The composition may comprise VTN, THBS1, and THBS2. The composition may comprise VTN, THBS1, and IGF2. The composition may comprise VTN, THBS1, and IL-15. The composition may comprise VTN, THBS1, and THBS4.

The composition may comprise VTN, IGF2, and POSTN. The composition may comprise VTN, IGF2, and FGF17. The composition may comprise VTN, IGF2, and THBS2. The composition may comprise VTN, IGF2, and THBS1. The composition may comprise VTN, IGF2, and IL-15. The composition may comprise VTN, IGF2, and THBS4.

The composition may comprise VTN, IL-15, and POSTN. The composition may comprise VTN, IL-15, and FGF17. The composition may comprise VTN, IL-15, and THBS2. The composition may comprise VTN, IL-15, and THBS1. The composition may comprise VTN, IL-15, and IGF2. The composition may comprise VTN, IL-15, and THBS4.

The composition may comprise VTN, TBHS4, and POSTN. The composition may comprise VTN, TBHS4, and FGF17. The composition may comprise VTN, TBHS4, and THBS2. The composition may comprise VTN, TBHS4, and THBS1. The composition may comprise VTN, TBHS4, and IGF2. The composition may comprise VTN, TBHS4, and IL-15.

The composition may comprise POSTN, FGF17, and VTN. The composition may comprise POSTN, FGF17, and TBHS2. The composition may comprise POSTN, FGF17, and THBS1. The composition may comprise POSTN, FGF17, and IGF2. The composition may comprise POSTN, FGF17, and IL-15. The composition may comprise POSTN, FGF17, and THBS4.

The composition may comprise POSTN, THBS2, and VTN. The composition may comprise POSTN, THBS2, and FGF17. The composition may comprise POSTN, THBS2, and THBS1. The composition may comprise POSTN, THBS2, and IGF2. The composition may comprise POSTN, THBS2, and IL-15. The composition may comprise POSTN, THBS2, and THBS4.

The composition may comprise POSTN, THBS1, and VTN. The composition may comprise POSTN, THBS1, and FGF17. The composition may comprise POSTN, THBS1, and THBS2. The composition may comprise POSTN, THBS1, and IGF2. The composition may comprise POSTN, THBS1, and IL-15. The composition may comprise POSTN, THBS1, and THBS4.

The composition may comprise POSTN, IGF2, and VTN. The composition may comprise POSTN, IGF2, and FGF17. The composition may comprise POSTN, IGF2, and THBS2. The composition may comprise POSTN, IGF2, and THBS1. The composition may comprise POSTN, IGF2, and IL-15. The composition may comprise POSTN, IGF2, and THBS4.

The composition may comprise POSTN, IL-15, and VTN. The composition may comprise POSTN, IL-15, and FGF17. The composition may comprise POSTN, IL-15, and THBS2. The composition may comprise POSTN, IL-15, and THBS1. The composition may comprise POSTN, IL-15, and IGF2. The composition may comprise POSTN, IL-15, and THBS4.

The composition may comprise POSTN, THBS4, and VTN. The composition may comprise POSTN, THBS4, and FGF17. The composition may comprise POSTN, THBS4, and THBS2. The composition may comprise POSTN, THBS4, and THBS1. The composition may comprise POSTN, THBS4, and IGF2. The composition may comprise POSTN, THBS4, and IL-15.

The composition may comprise FGF17, THBS2, and VTN. The composition may comprise FGF17, THBS2, and POSTN. The composition may comprise FGF17, THBS2, and THBS1. The composition may comprise FGF17, THBS2, and IGF2. The composition may comprise FGF17, THBS2, and IL-15. The composition may comprise FGF17, THBS2, and THBS4.

The composition may comprise FGF17, THBS1, and VTN. The composition may comprise FGF17, THBS1, and POSTN. The composition may comprise FGF17, THBS1, and THBS2. The composition may comprise FGF17, THBS1, and IGF2. The composition may comprise FGF17, THBS1, and IL-15. The composition may comprise FGF17, THBS1, and THBS4.

The composition may comprise FGF17, IGF2, and VTN. The composition may comprise FGF17, IGF2, and POSTN. The composition may comprise FGF17, IGF2, and THBS2. The composition may comprise FGF17, IGF2, and THBS1. The composition may comprise FGF17, IGF2, and IL-15. The composition may comprise FGF17, IGF2, and THBS4.

The composition may comprise FGF17, IL-15, and VTN. The composition may comprise FGF17, IL-15, and POSTN. The composition may comprise FGF17, IL-15, and THBS2. The composition may comprise FGF17, IL-15, and THBS1. The composition may comprise FGF17, IL-15, and IGF2. The composition may comprise FGF17, IL-15, and THBS4.

The composition may comprise FGF17, THBS4, and VTN. The composition may comprise FGF17, THBS4, and POSTN. The composition may comprise FGF17, THBS4, and THBS2. The composition may comprise FGF17, THBS4, and THBS1. The composition may comprise FGF17, THBS4, and IGF2. The composition may comprise FGF17, THBS4, and IL-15.

The composition may comprise THBS2, THBS1, and VTN. The composition may comprise THBS2, THBS1, and POSTN. The composition may comprise THBS2, THBS1, and FGF17. The composition may comprise THBS2, THBS1, and IGF2. The composition may comprise THBS2, THBS1, and IL-15. The composition may comprise THBS2, THBS1, and THBS4.

The composition may comprise THBS2, IGF2, and VTN. The composition may comprise THBS2, IGF2, and POSTN. The composition may comprise THBS2, IGF2, and FGF17. The composition may comprise THBS2, IGF2, and THBS1. The composition may comprise THBS2, IGF2, and IL-15. The composition may comprise THBS2, IGF2, and THBS4.

The composition may comprise THBS2, IL-15, and VTN. The composition may comprise THBS2, IL-15, and POSTN. The composition may comprise THBS2, IL-15, and FGF17. The composition may comprise THBS2, IL-15, and THBS1. The composition may comprise THBS2, IL-15, and IGF2. The composition may comprise THBS2, IL-15, and THBS4.

The composition may comprise THBS2, THBS4, and VTN. The composition may comprise THBS2, THBS4, and POSTN. The composition may comprise THBS2, THBS4, and FGF17. The composition may comprise THBS2, THBS4, and THBS1. The composition may comprise THBS2, THBS4, and IGF2. The composition may comprise THBS2, THBS4, and IL-15.

The composition may comprise THBS1, IGF2, and VTN. The composition may comprise THBS1, IGF2, and POSTN. The composition may comprise THBS1, IGF2, and FGF17. The composition may comprise THBS1, IGF2, and THBS2. The composition may comprise THBS1, IGF2, and IL-15. The composition may comprise THBS1, IGF2, and THBS4.

The composition may comprise THBS1, IL-15, and VTN. The composition may comprise THBS1, IL-15, and POSTN. The composition may comprise THBS1, IL-15, and FGF17. The composition may comprise THBS1, IL-15, and THBS2. The composition may comprise THBS1, IL-15, and IGF2. The composition may comprise THBS1, IL-15, and THBS4.

The composition may comprise THBS1, and THBS4, and VTN. The composition may comprise THBS1, and THBS4, and POSTN. The composition may comprise THBS1, and THBS4, and FGF17. The composition may comprise THBS1, and THBS4, and THBS2. The composition may comprise THBS1, and THBS4, and IGF2. The composition may comprise THBS1, and THBS4, and IL-15.

The composition may comprise IGF2, IL-15, and VTN. The composition may comprise IGF2, IL-15, and POSTN. The composition may comprise IGF2, IL-15, and FGF17. The composition may comprise IGF2, IL-15, and THBS2. The composition may comprise IGF2, IL-15, and THBS1. The composition may comprise IGF2, IL-15, and THBS4.

The composition may comprise IGF2, THBS4, and VTN. The composition may comprise IGF2, THBS4, and POSTN. The composition may comprise IGF2, THBS4, and FGF17. The composition may comprise IGF2, THBS4, and THBS2. The composition may comprise IGF2, THBS4, and THBS1. The composition may comprise IGF2, THBS4, and IL-15.

The composition may comprise IL-15, and THBS4, and VTN. The composition may comprise IL-15, and THBS4, and POSTN. The composition may comprise IL-15, and THBS4, and FGF17. The composition may comprise IL-15, and THBS4, and THBS2. The composition may comprise IL-15, and THBS4, and THBS1. The composition may comprise IL-15, and THBS4, and IGF2.

The composition may comprise VTN, POSTN, and FGF17. The composition may comprise VTN, POSTN, and THBS2. The composition may comprise VTN, POSTN, and THBS4. The composition may comprise VTN, FGF17, and POSTN. The composition may comprise VTN, FGF17, and THBS2. The composition may comprise VTN, FGF17, and THBS4. The composition may comprise VTN, THBS2, and POSTN. The composition may comprise VTN, THBS2, and FGF17. The composition may comprise VTN, THBS2, and THBS4. The composition may comprise VTN, THBS4, and POSTN. The composition may comprise VTN, THBS4, and FGF17. The composition may comprise VTN, THBS4, and THBS2. The composition may comprise POSTN, VTN, and THBS4. The composition may comprise POSTN, VTN, and FGF17. The composition may comprise POSTN, VTN, and THBS2. The composition may comprise POSTN, FGF17, and THBS4. The composition may comprise POSTN, FGF17, and VTN. The composition may comprise POSTN, FGF17, and THBS2. The composition may comprise POSTN, THBS2, and THBS4. The composition may comprise POSTN, THBS2, and VTN. The composition may comprise POSTN, THBS2, and FGF17. The composition may comprise POSTN, THBS4, and THBS2. The composition may comprise POSTN, THBS4, and VTN. The composition may comprise POSTN, THBS4, and FGF17. The composition may comprise FGF17, VTN, and THBS2. The composition may comprise FGF17, VTN, and THBS4. The composition may comprise FGF17, VTN, and POSTN. The composition may comprise FGF17, POSTN, and THBS2. The composition may comprise FGF17, POSTN, and THBS4. The composition may comprise FGF17, POSTN, and VTN. The composition may comprise FGF17, THBS2, and POSTN. The composition may comprise FGF17, THBS2, and THBS4. The composition may comprise FGF17, THBS2, and VTN. The composition may comprise FGF17, THBS4, and POSTN. The composition may comprise FGF17, THBS4, and THBS2. The composition may comprise FGF17, THBS4, and VTN. The composition may comprise THBS2, VTN, and POSTN. The composition may comprise THBS2, VTN, and FGF17. The composition may comprise THBS2, VTN, and THBS4. The composition may comprise THBS2, POSTN, and VTN. The composition may comprise THBS2, POSTN, and FGF17. The composition may comprise THBS2, POSTN, and THBS4. The composition may comprise THBS2, FGF17, and VTN. The composition may comprise THBS2, FGF17, and POSTN. The composition may comprise THBS2, FGF17, and THBS4. The composition may comprise THBS2, THBS4, and VTN. The composition may comprise THBS2, THBS4, and POSTN. The composition may comprise THBS2, THBS4, and FGF17. The composition may comprise THBS4, VTN, and THBS2. The composition may comprise THBS4, VTN, and POSTN. The composition may comprise THBS4, VTN, and FGF17. The composition may comprise THBS4, POSTN, and THBS2. The composition may comprise THBS4, POSTN, and VTN. The composition may comprise THBS4, POSTN, and FGF17. The composition may comprise THBS4, FGF17, and THBS2. The composition may comprise THBS4, FGF17, and VTN. The composition may comprise THBS4, FGF17, and POSTN. The composition may comprise THBS4, THBS2, and FGF17. The composition may comprise THBS4, THBS2, and VTN. The composition may comprise THBS4, THBS2, and POSTN.

The heparin-associated binding polypeptide composition comprising any three polypeptides selected from VTN, POSTN, FGF17, THBS2, THBS1, IGF2, IL-15, BMP7, and THBS4, may comprise one or more additional modifications to increase stability. In certain embodiments, one or more of the HAPs is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the HAPs is fused or conjugated to another protein to increase stability and or bioavailability. In certain embodiments, one or more of the HAPs is fused with an Fc region of an immunoglobulin or with serum albumin. In certain embodiments, one or more of the HAPs is present in a concatemer with one, two, three, or four other distinct polypeptides selected from THBS2, VTN, POSTN, FGF17, THBS1, IGF2, IL-15, BMP7, and THBS4. In certain embodiments, one or more of the HAPs is present in a concatemer with one, two, three, four, or more distinct polypeptides. In certain embodiments, one or more of the HAPs is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA).

The heparin-associated binding polypeptide composition may comprise any four polypeptides selected from VTN, POSTN, FGF17, BMP7, THBS1, IGF2, IL-15, THBS2, and THBS4. The composition may comprise BMP7, VTN, POSTN, and FGF17. The composition may comprise BMP7, VTN, POSTN, and THBS2. The composition may comprise BMP7, VTN, POSTN, and THBS4. The composition may comprise BMP7, VTN, FGF17, and POSTN. The composition may comprise BMP7, VTN, FGF17, and THBS2. The composition may comprise BMP7, VTN, FGF17, and THBS4. The composition may comprise BMP7, VTN, THBS2, and POSTN. The composition may comprise BMP7, VTN, THBS2, and FGF17. The composition may comprise BMP7, VTN, THBS2, and THBS4. The composition may comprise BMP7, VTN, THBS4, and POSTN. The composition may comprise BMP7, VTN, THBS4, and FGF17. The composition may comprise BMP7, VTN, THBS4, and THBS2. The composition may comprise BMP7, POSTN, VTN, and THBS4. The composition may comprise BMP7, POSTN, VTN, and FGF17. The composition may comprise BMP7, POSTN, VTN, and THBS2. The composition may comprise BMP7, POSTN, FGF17, and THBS4. The composition may comprise BMP7, POSTN, FGF17, and VTN. The composition may comprise BMP7, POSTN, FGF17, and THBS2. The composition may comprise BMP7, POSTN, THBS2, and THBS4. The composition may comprise BMP7, POSTN, THBS2, and VTN. The composition may comprise BMP7, POSTN, THBS2, and FGF17. The composition may comprise BMP7, POSTN, THBS4, and THBS2. The composition may comprise BMP7, POSTN, THBS4, and VTN. The composition may comprise BMP7, POSTN, THBS4, and FGF17. The composition may comprise BMP7, FGF17, VTN, and THBS2. The composition may comprise BMP7, FGF17, VTN, and THBS4. The composition may comprise BMP7, FGF17, VTN, and POSTN. The composition may comprise BMP7, FGF17, POSTN, and THBS2. The composition may comprise BMP7, FGF17, POSTN, and THBS4. The composition may comprise BMP7, FGF17, POSTN, and VTN. The composition may comprise BMP7, FGF17, THBS2, and POSTN. The composition may comprise BMP7, FGF17, THBS2, and THBS4. The composition may comprise BMP7, FGF17, THBS2, and VTN. The composition may comprise BMP7, FGF17, THBS4, and POSTN. The composition may comprise BMP7, FGF17, THBS4, and THBS2. The composition may comprise BMP7, FGF17, THBS4, and VTN. The composition may comprise BMP7, THBS2, VTN, and POSTN. The composition may comprise BMP7, THBS2, VTN, and FGF17. The composition may comprise BMP7, THBS2, VTN, and THBS4. The composition may comprise BMP7, THBS2, POSTN, and VTN. The composition may comprise BMP7, THBS2, POSTN, and FGF17. The composition may comprise BMP7, THBS2, POSTN, and THBS4. The composition may comprise BMP7, THBS2, FGF17, and VTN. The composition may comprise BMP7, THBS2, FGF17, and POSTN. The composition may comprise BMP7, THBS2, FGF17, and THBS4. The composition may comprise BMP7, THBS2, THBS4, and VTN. The composition may comprise BMP7, THBS2, THBS4, and POSTN. The composition may comprise BMP7, THBS2, THBS4, and FGF17. The composition may comprise BMP7, THBS4, VTN, and THBS2. The composition may comprise BMP7, THBS4, VTN, and POSTN. The composition may comprise BMP7, THBS4, VTN, and FGF17. The composition may comprise BMP7, THBS4, POSTN, and THBS2. The composition may comprise BMP7, THBS4, POSTN, and VTN. The composition may comprise BMP7, THBS4, POSTN, and FGF17. The composition may comprise BMP7, THBS4, FGF17, and THBS2. The composition may comprise BMP7, THBS4, FGF17, and VTN. The composition may comprise BMP7, THBS4, FGF17, and POSTN. The composition may comprise BMP7, THBS4, THBS2, and FGF17. The composition may comprise BMP7, THBS4, THBS2, and VTN. The composition may comprise BMP7, THBS4, THBS2, and POSTN. The composition may comprise VTN, POSTN, FGF17, and THBS2. The composition may comprise VTN, POSTN, FGF17, and THBS4. The composition may comprise VTN, POSTN, THBS2, and FGF17. The composition may comprise VTN, POSTN, THBS2, and THBS4. The composition may comprise VTN, POSTN, THBS4, and FGF17. The composition may comprise VTN, POSTN, THBS4, and THBS2. The composition may comprise VTN, FGF17, POSTN, and THBS4. The composition may comprise VTN, FGF17, POSTN, and THBS2. The composition may comprise VTN, FGF17, THBS2, and THBS4. The composition may comprise VTN, FGF17, THBS2, and POSTN. The composition may comprise VTN, FGF17, THBS4, and THBS2. The composition may comprise VTN, FGF17, THBS4, and POSTN. The composition may comprise VTN, THBS2, POSTN, and FGF17. The composition may comprise VTN, THBS2, POSTN, and THBS4. The composition may comprise VTN, THBS2, FGF17, and POSTN. The composition may comprise VTN, THBS2, FGF17, and THBS4. The composition may comprise VTN, THBS2, THBS4, and POSTN. The composition may comprise VTN, THBS2, THBS4, and FGF17. The composition may comprise VTN, THBS4, POSTN, and THBS2. The composition may comprise VTN, THBS4, POSTN, and FGF17. The composition may comprise VTN, THBS4, FGF17, and THBS2. The composition may comprise VTN, THBS4, FGF17, and POSTN. The composition may comprise VTN, THBS4, THBS2, and FGF17. The composition may comprise VTN, THBS4, THBS2, and POSTN. The composition may comprise POSTN, VTN, THBS4, and FGF17. The composition may comprise POSTN, VTN, THBS4, and THBS2. The composition may comprise POSTN, VTN, FGF17, and THBS4. The composition may comprise POSTN, VTN, FGF17, and THBS2. The composition may comprise POSTN, VTN, THBS2, and THBS4. The composition may comprise POSTN, VTN, THBS2, and FGF17. The composition may comprise POSTN, FGF17, THBS4, and THBS2. The composition may comprise POSTN, FGF17, THBS4, and VTN. The composition may comprise POSTN, FGF17, VTN, and THBS2. The composition may comprise POSTN, FGF17, VTN, and THBS4. The composition may comprise POSTN, FGF17, THBS2, and VTN. The composition may comprise POSTN, FGF17, THBS2, and THBS4. The composition may comprise POSTN, THBS2, THBS4, and VTN. The composition may comprise POSTN, THBS2, THBS4, and FGF17. The composition may comprise POSTN, THBS2, VTN, and THBS4. The composition may comprise POSTN, THBS2, VTN, and FGF17. The composition may comprise POSTN, THBS2, FGF17, and THBS4. The composition may comprise POSTN, THBS2, FGF17, and VTN. The composition may comprise POSTN, THBS4, THBS2, and FGF17. The composition may comprise POSTN, THBS4, THBS2, and VTN. The composition may comprise POSTN, THBS4, VTN, and FGF17. The composition may comprise POSTN, THBS4, VTN, and THBS2. The composition may comprise POSTN, THBS4, FGF17, and VTN. The composition may comprise POSTN, THBS4, FGF17, and THBS2. The composition may comprise FGF17, VTN, THBS2, and THBS4. The composition may comprise FGF17, VTN, THBS2, and POSTN. The composition may comprise FGF17, VTN, THBS4, and THBS2. The composition may comprise FGF17, VTN, THBS4, and POSTN. The composition may comprise FGF17, VTN, POSTN, and THBS2. The composition may comprise FGF17, VTN, POSTN, and THBS4. The composition may comprise FGF17, POSTN, THBS2, and VTN. The composition may comprise FGF17, POSTN, THBS2, and THBS4. The composition may comprise FGF17, POSTN, THBS4, and VTN. The composition may comprise FGF17, POSTN, THBS4, and THBS2. The composition may comprise FGF17, POSTN, VTN, and THBS4. The composition may comprise FGF17, POSTN, VTN, and THBS2. The composition may comprise FGF17, THBS2, POSTN, and THBS4. The composition may comprise FGF17, THBS2, POSTN, and VTN. The composition may comprise FGF17, THBS2, THBS4, and POSTN. The composition may comprise FGF17, THBS2, THBS4, and VTN. The composition may comprise FGF17, THBS2, VTN, and POSTN. The composition may comprise FGF17, THBS2, VTN, and THBS4. The composition may comprise FGF17, THBS4, POSTN, and VTN. The composition may comprise FGF17, THBS4, POSTN, and THBS2. The composition may comprise FGF17, THBS4, THBS2, and VTN. The composition may comprise FGF17, THBS4, THBS2, and POSTN. The composition may comprise FGF17, THBS4, VTN, and THBS2. The composition may comprise FGF17, THBS4, VTN, and POSTN. The composition may comprise THBS2, VTN, POSTN, and FGF17. The composition may comprise THBS2, VTN, POSTN, and THBS4. The composition may comprise THBS2, VTN, FGF17, and POSTN. The composition may comprise THBS2, VTN, FGF17, and THBS4. The composition may comprise THBS2, VTN, THBS4, and POSTN. The composition may comprise THBS2, VTN, THBS4, and FGF17. The composition may comprise THBS2, POSTN, VTN, and THBS4. The composition may comprise THBS2, POSTN, VTN, and FGF17. The composition may comprise THBS2, POSTN, FGF17, and THBS4. The composition may comprise THBS2, POSTN, FGF17, and VTN. The composition may comprise THBS2, POSTN, THBS4, and FGF17. The composition may comprise THBS2, POSTN, THBS4, and VTN. The composition may comprise THBS2, FGF17, VTN, and POSTN. The composition may comprise THBS2, FGF17, VTN, and THBS4. The composition may comprise THBS2, FGF17, POSTN, and VTN. The composition may comprise THBS2, FGF17, POSTN, and THBS4. The composition may comprise THBS2, FGF17, THBS4, and VTN. The composition may comprise THBS2, FGF17, THBS4, and POSTN. The composition may comprise THBS2, THBS4, VTN, and FGF17. The composition may comprise THBS2, THBS4, VTN, and POSTN. The composition may comprise THBS2, THBS4, POSTN, and FGF17. The composition may comprise THBS2, THBS4, POSTN, and VTN. The composition may comprise THBS2, THBS4, FGF17, and POSTN. The composition may comprise THBS2, THBS4, FGF17, and VTN. The composition may comprise THBS4, VTN, THBS2, and POSTN. The composition may comprise THBS4, VTN, THBS2, and FGF17. The composition may comprise THBS4, VTN, POSTN, and THBS2. The composition may comprise THBS4, VTN, POSTN, and FGF17. The composition may comprise THBS4, VTN, FGF17, and THBS2. The composition may comprise THBS4, VTN, FGF17, and POSTN. The composition may comprise THBS4, POSTN, THBS2, and FGF17. The composition may comprise THBS4, POSTN, THBS2, and VTN. The composition may comprise THBS4, POSTN, VTN, and FGF17. The composition may comprise THBS4, POSTN, VTN, and THBS2. The composition may comprise THBS4, POSTN, FGF17, and VTN. The composition may comprise THBS4, POSTN, FGF17, and THBS2. The composition may comprise THBS4, FGF17, THBS2, and VTN. The composition may comprise THBS4, FGF17, THBS2, and POSTN. The composition may comprise THBS4, FGF17, VTN, and THBS2. The composition may comprise THBS4, FGF17, VTN, and POSTN. The composition may comprise THBS4, FGF17, POSTN, and THBS2. The composition may comprise THBS4, FGF17, POSTN, and VTN. The composition may comprise THBS4, THBS2, FGF17, and POSTN. The composition may comprise THBS4, THBS2, FGF17, and VTN. The composition may comprise THBS4, THBS2, VTN, and POSTN. The composition may comprise THBS4, THBS2, VTN, and FGF17. The composition may comprise THBS4, THBS2, POSTN, and VTN. The composition may comprise THBS4, THBS2, POSTN, and FGF17.

The composition may comprise VTN, POSTN, FGF17, and a polypeptide comprising BMP7, VTN, POSTN, FGF17, THBS2, THBS1, IGF2, IL-15, or THBS4. The composition may comprise VTN, POSTN, THBS2, and a polypeptide comprising BMP7, VTN, POSTN, FGF17, THBS2, THBS1, IGF2, IL-15, or THBS4. The composition may comprise VTN, POSTN, FGF17, and a polypeptide comprising BMP7, VTN, POSTN, FGF17, THBS2, THBS1, IGF2, IL-15, or THBS4. The composition may comprise VTN, POSTN, THBS2, and a polypeptide comprising BMP7, VTN, POSTN, FGF17, THBS2, THBS1, IGF2, IL-15, or THBS4. The composition may comprise VTN, POSTN, THBS1, and a polypeptide comprising BMP7, VTN, POSTN, FGF17, THBS2, THBS1, IGF2, IL-15, or THBS4. The composition may comprise VTN, POSTN, IGF2, and a polypeptide comprising BMP7, VTN, POSTN, FGF17, THBS2, THBS1, IGF2, IL-15, or THBS4. The composition may comprise VTN, POSTN, IL-15, and a polypeptide comprising BMP7, VTN, POSTN, FGF17, THBS2, THBS1, IGF2, IL-15, or THBS4. The composition may comprise VTN, POSTN, THBS4, and a polypeptide comprising BMP7, VTN, POSTN, FGF17, THBS2, THBS1, IGF2, IL-15, or THBS4.

The composition may comprise VTN, FGF17, THBS2, and a polypeptide comprising BMP7, VTN, POSTN, FGF17, THBS2, THBS1, IGF2, IL-15, or THBS4. The composition may comprise VTN, FGF17, POSTN, and a polypeptide comprising BMP7, VTN, POSTN, FGF17, THBS2, THBS1, IGF2, IL-15, or THBS4. The composition may comprise VTN, FGF17, THBS2, and a polypeptide comprising BMP7, VTN, POSTN, FGF17, THBS2, THBS1, IGF2, IL-15, or THBS4. The composition may comprise VTN, FGF17, THBS1, and a polypeptide comprising BMP7, VTN, POSTN, FGF17, THBS2, THBS1, IGF2, IL-15, or THBS4. The composition may comprise VTN, FGF17, IGF2, and a polypeptide comprising BMP7, VTN, POSTN, FGF17, THBS2, THBS1, IGF2, IL-15, or THBS4. The composition may comprise VTN, FGF17, IL-15, and a polypeptide comprising BMP7, VTN, POSTN, FGF17, THBS2, THBS1, IGF2, IL-15, or THBS4. The composition may comprise VTN, FGF17, THBS4, and a polypeptide comprising BMP7, VTN, POSTN, FGF17, THBS2, THBS1, IGF2, IL-15, or THBS4.

The composition may comprise VTN, THBS2, POSTN, and a polypeptide comprising BMP7, VTN, POSTN, FGF17, THBS2, THBS1, IGF2, IL-15, or THBS4. The composition may comprise VTN, THBS2, FGF17, and a polypeptide comprising BMP7, VTN, POSTN, FGF17, THBS2, THBS1, IGF2, IL-15, or THBS4. The composition may comprise VTN, THBS2, THBS1, and a polypeptide comprising BMP7, VTN, POSTN, FGF17, THBS2, THBS1, IGF2, IL-15, or THBS4. The composition may comprise VTN, THBS2, IGF2, and a polypeptide comprising BMP7, VTN, POSTN, FGF17, THBS2, THBS1, IGF2, IL-15, or THBS4. The composition may comprise VTN, THBS2, IL-15, and a polypeptide comprising BMP7, VTN, POSTN, FGF17, THBS2, THBS1, IGF2, IL-15, or THBS4. The composition may comprise VTN, THBS2, THBS4, and a polypeptide comprising BMP7, VTN, POSTN, FGF17, THBS2, THBS1, IGF2, IL-15, or THBS4.

The composition may comprise VTN, THBS1, POSTN, and a polypeptide comprising BMP7, VTN, POSTN, FGF17, THBS2, THBS1, IGF2, IL-15, or THBS4. The composition may comprise VTN, THBS1, FGF17, and a polypeptide comprising BMP7, VTN, POSTN, FGF17, THBS2, THBS1, IGF2, IL-15, or THBS4. The composition may comprise VTN, THBS1, THBS2, and a polypeptide comprising BMP7, VTN, POSTN, FGF17, THBS2, THBS1, IGF2, IL-15, or THBS4. The composition may comprise VTN, THBS1, IGF2, and a polypeptide comprising BMP7, VTN, POSTN, FGF17, THBS2, THBS1, IGF2, IL-15, or THBS4. The composition may comprise VTN, THBS1, IL-15, and a polypeptide comprising BMP7, VTN, POSTN, FGF17, THBS2, THBS1, IGF2, IL-15, or THBS4. The composition may comprise VTN, THBS1, THBS4, and a polypeptide comprising BMP7, VTN, POSTN, FGF17, THBS2, THBS1, IGF2, IL-15, or THBS4.

The composition may comprise VTN, IGF2, POSTN, and a polypeptide comprising BMP7, VTN, POSTN, FGF17, THBS2, THBS1, IGF2, IL-15, or THBS4. The composition may comprise VTN, IGF2, FGF17, and a polypeptide comprising BMP7, VTN, POSTN, FGF17, THBS2, THBS1, IGF2, IL-15, or THBS4. The composition may comprise VTN, IGF2, THBS2, and a polypeptide comprising BMP7, VTN, POSTN, FGF17, THBS2, THBS1, IGF2, IL-15, or THBS4. The composition may comprise VTN, IGF2, THBS1, and a polypeptide comprising BMP7, VTN, POSTN, FGF17, THBS2, THBS1, IGF2, IL-15, or THBS4. The composition may comprise VTN, IGF2, IL-15, and a polypeptide comprising BMP7, VTN, POSTN, FGF17, THBS2, THBS1, IGF2, IL-15, or THBS4. The composition may comprise VTN, IGF2, THBS4, and a polypeptide comprising BMP7, VTN, POSTN, FGF17, THBS2, THBS1, IGF2, IL-15, or THBS4.

The composition may comprise VTN, IL-15, POSTN, and a polypeptide comprising BMP7, VTN, POSTN, FGF17, THBS2, THBS1, IGF2, IL-15, or THBS4. The composition may comprise VTN, IL-15, FGF17, and a polypeptide comprising BMP7, VTN, POSTN, FGF17, THBS2, THBS1, IGF2, IL-15, or THBS4. The composition may comprise VTN, IL-15, THBS2, and a polypeptide comprising BMP7, VTN, POSTN, FGF17, THBS2, THBS1, IGF2, IL-15, or THBS4. The composition may comprise VTN, IL-15, THBS1, and a polypeptide comprising BMP7, VTN, POSTN, FGF17, THBS2, THBS1, IGF2, IL-15, or THBS4. The composition may comprise VTN, IL-15, IGF2, and a polypeptide comprising BMP7, VTN, POSTN, FGF17, THBS2, THBS1, IGF2, IL-15, or THBS4. The composition may comprise VTN, IL-15, THBS4, and a polypeptide comprising BMP7, VTN, POSTN, FGF17, THBS2, THBS1, IGF2, IL-15, or THBS4.

The composition may comprise VTN, TBHS4, POSTN, and a polypeptide comprising BMP7, VTN, POSTN, FGF17, THBS2, THBS1, IGF2, IL-15, or THBS4. The composition may comprise VTN, TBHS4, FGF17, and a polypeptide comprising BMP7, VTN, POSTN, FGF17, THBS2, THBS1, IGF2, IL-15, or THBS4. The composition may comprise VTN, TBHS4, THBS2, and a polypeptide comprising BMP7, VTN, POSTN, FGF17, THBS2, THBS1, IGF2, IL-15, or THBS4. The composition may comprise VTN, TBHS4, THBS1, and a polypeptide comprising BMP7, VTN, POSTN, FGF17, THBS2, THBS1, IGF2, IL-15, or THBS4. The composition may comprise VTN, TBHS4, IGF2, and a polypeptide comprising BMP7, VTN, POSTN, FGF17, THBS2, THBS1, IGF2, IL-15, or THBS4. The composition may comprise VTN, TBHS4, IL-15, and a polypeptide comprising BMP7, VTN, POSTN, FGF17, THBS2, THBS1, IGF2, IL-15, or THBS4.

The composition may comprise POSTN, FGF17, VTN, and a polypeptide comprising BMP7, VTN, POSTN, FGF17, THBS2, THBS1, IGF2, IL-15, or THBS4. The composition may comprise POSTN, FGF17, TBHS2, and a polypeptide comprising BMP7, VTN, POSTN, FGF17, THBS2, THBS1, IGF2, IL-15, or THBS4. The composition may comprise POSTN, FGF17, THBS1, and a polypeptide comprising BMP7, VTN, POSTN, FGF17, THBS2, THBS1, IGF2, IL-15, or THBS4. The composition may comprise POSTN, FGF17, IGF2, and a polypeptide comprising BMP7, VTN, POSTN, FGF17, THBS2, THBS1, IGF2, IL-15, or THBS4. The composition may comprise POSTN, FGF17, IL-15, and a polypeptide comprising BMP7, VTN, POSTN, FGF17, THBS2, THBS1, IGF2, IL-15, or THBS4. The composition may comprise POSTN, FGF17, THBS4, and a polypeptide comprising BMP7, VTN, POSTN, FGF17, THBS2, THBS1, IGF2, IL-15, or THBS4.

The composition may comprise POSTN, THBS2, VTN, and a polypeptide comprising BMP7, VTN, POSTN, FGF17, THBS2, THBS1, IGF2, IL-15, or THBS4. The composition may comprise POSTN, THBS2, FGF17, and a polypeptide comprising BMP7, VTN, POSTN, FGF17, THBS2, THBS1, IGF2, IL-15, or THBS4. The composition may comprise POSTN, THBS2, THBS1, and a polypeptide comprising BMP7, VTN, POSTN, FGF17, THBS2, THBS1, IGF2, IL-15, or THBS4. The composition may comprise POSTN, THBS2, IGF2, and a polypeptide comprising BMP7, VTN, POSTN, FGF17, THBS2, THBS1, IGF2, IL-15, or THBS4. The composition may comprise POSTN, THBS2, IL-15, and a polypeptide comprising BMP7, VTN, POSTN, FGF17, THBS2, THBS1, IGF2, IL-15, or THBS4. The composition may comprise POSTN, THBS2, THBS4, and a polypeptide comprising BMP7, VTN, POSTN, FGF17, THBS2, THBS1, IGF2, IL-15, or THBS4.

The composition may comprise POSTN, THBS1, VTN, and a polypeptide comprising BMP7, VTN, POSTN, FGF17, THBS2, THBS1, IGF2, IL-15, or THBS4. The composition may comprise POSTN, THBS1, FGF17, and a polypeptide comprising BMP7, VTN, POSTN, FGF17, THBS2, THBS1, IGF2, IL-15, or THBS4. The composition may comprise POSTN, THBS1, THBS2, and a polypeptide comprising BMP7, VTN, POSTN, FGF17, THBS2, THBS1, IGF2, IL-15, or THBS4. The composition may comprise POSTN, THBS1, IGF2, and a polypeptide comprising BMP7, VTN, POSTN, FGF17, THBS2, THBS1, IGF2, IL-15, or THBS4. The composition may comprise POSTN, THBS1, IL-15, and a polypeptide comprising BMP7, VTN, POSTN, FGF17, THBS2, THBS1, IGF2, IL-15, or THBS4. The composition may comprise POSTN, THBS1, THBS4, and a polypeptide comprising BMP7, VTN, POSTN, FGF17, THBS2, THBS1, IGF2, IL-15, or THBS4.

The composition may comprise POSTN, IGF2, VTN, and a polypeptide comprising BMP7, VTN, POSTN, FGF17, THBS2, THBS1, IGF2, IL-15, or THBS4. The composition may comprise POSTN, IGF2, FGF17, and a polypeptide comprising BMP7, VTN, POSTN, FGF17, THBS2, THBS1, IGF2, IL-15, or THBS4. The composition may comprise POSTN, IGF2, THBS2, and a polypeptide comprising BMP7, VTN, POSTN, FGF17, THBS2, THBS1, IGF2, IL-15, or THBS4. The composition may comprise POSTN, IGF2, THBS1, and a polypeptide comprising BMP7, VTN, POSTN, FGF17, THBS2, THBS1, IGF2, IL-15, or THBS4. The composition may comprise POSTN, IGF2, IL-15, and a polypeptide comprising BMP7, VTN, POSTN, FGF17, THBS2, THBS1, IGF2, IL-15, or THBS4. The composition may comprise POSTN, IGF2, THBS4, and a polypeptide comprising BMP7, VTN, POSTN, FGF17, THBS2, THBS1, IGF2, IL-15, or THBS4.

The composition may comprise POSTN, IL-15, VTN, and a polypeptide comprising BMP7, VTN, POSTN, FGF17, THBS2, THBS1, IGF2, IL-15, or THBS4. The composition may comprise POSTN, IL-15, FGF17, and a polypeptide comprising BMP7, VTN, POSTN, FGF17, THBS2, THBS1, IGF2, IL-15, or THBS4. The composition may comprise POSTN, IL-15, THBS2, and a polypeptide comprising BMP7, VTN, POSTN, FGF17, THBS2, THBS1, IGF2, IL-15, or THBS4. The composition may comprise POSTN, IL-15, THBS1, and a polypeptide comprising BMP7, VTN, POSTN, FGF17, THBS2, THBS1, IGF2, IL-15, or THBS4. The composition may comprise POSTN, IL-15, IGF2, and a polypeptide comprising BMP7, VTN, POSTN, FGF17, THBS2, THBS1, IGF2, IL-15, or THBS4. The composition may comprise POSTN, IL-15, THBS4, and a polypeptide comprising BMP7, VTN, POSTN, FGF17, THBS2, THBS1, IGF2, IL-15, or THBS4.

The composition may comprise POSTN, THBS4, VTN, and a polypeptide comprising BMP7, VTN, POSTN, FGF17, THBS2, THBS1, IGF2, IL-15, or THBS4. The composition may comprise POSTN, THBS4, FGF17, and a polypeptide comprising BMP7, VTN, POSTN, FGF17, THBS2, THBS1, IGF2, IL-15, or THBS4. The composition may comprise POSTN, THBS4, THBS2, and a polypeptide comprising BMP7, VTN, POSTN, FGF17, THBS2, THBS1, IGF2, IL-15, or THBS4. The composition may comprise POSTN, THBS4, THBS1, and a polypeptide comprising BMP7, VTN, POSTN, FGF17, THBS2, THBS1, IGF2, IL-15, or THBS4. The composition may comprise POSTN, THBS4, IGF2, and a polypeptide comprising BMP7, VTN, POSTN, FGF17, THBS2, THBS1, IGF2, IL-15, or THBS4. The composition may comprise POSTN, THBS4, IL-15, and a polypeptide comprising BMP7, VTN, POSTN, FGF17, THBS2, THBS1, IGF2, IL-15, or THBS4.

The composition may comprise FGF17, THBS2, VTN, and a polypeptide comprising BMP7, VTN, POSTN, FGF17, THBS2, THBS1, IGF2, IL-15, or THBS4. The composition may comprise FGF17, THBS2, POSTN, and a polypeptide comprising BMP7, VTN, POSTN, FGF17, THBS2, THBS1, IGF2, IL-15, or THBS4. The composition may comprise FGF17, THBS2, THBS1, and a polypeptide comprising BMP7, VTN, POSTN, FGF17, THBS2, THBS1, IGF2, IL-15, or THBS4. The composition may comprise FGF17, THBS2, IGF2, and a polypeptide comprising BMP7, VTN, POSTN, FGF17, THBS2, THBS1, IGF2, IL-15, or THBS4. The composition may comprise FGF17, THBS2, IL-15, and a polypeptide comprising BMP7, VTN, POSTN, FGF17, THBS2, THBS1, IGF2, IL-15, or THBS4. The composition may comprise FGF17, THBS2, THBS4, and a polypeptide comprising BMP7, VTN, POSTN, FGF17, THBS2, THBS1, IGF2, IL-15, or THBS4.

The composition may comprise FGF17, THBS1, VTN, and a polypeptide comprising BMP7, VTN, POSTN, FGF17, THBS2, THBS1, IGF2, IL-15, or THBS4. The composition may comprise FGF17, THBS1, POSTN, and a polypeptide comprising BMP7, VTN, POSTN, FGF17, THBS2, THBS1, IGF2, IL-15, or THBS4. The composition may comprise FGF17, THBS1, THBS2, and a polypeptide comprising BMP7, VTN, POSTN, FGF17, THBS2, THBS1, IGF2, IL-15, or THBS4. The composition may comprise FGF17, THBS1, IGF2, and a polypeptide comprising BMP7, VTN, POSTN, FGF17, THBS2, THBS1, IGF2, IL-15, or THBS4. The composition may comprise FGF17, THBS1, IL-15, and a polypeptide comprising BMP7, VTN, POSTN, FGF17, THBS2, THBS1, IGF2, IL-15, or THBS4. The composition may comprise FGF17, THBS1, THBS4, and a polypeptide comprising BMP7, VTN, POSTN, FGF17, THBS2, THBS1, IGF2, IL-15, or THBS4.

The composition may comprise FGF17, IGF2, VTN, and a polypeptide comprising BMP7, VTN, POSTN, FGF17, THBS2, THBS1, IGF2, IL-15, or THBS4. The composition may comprise FGF17, IGF2, POSTN, and a polypeptide comprising BMP7, VTN, POSTN, FGF17, THBS2, THBS1, IGF2, IL-15, or THBS4. The composition may comprise FGF17, IGF2, THBS2, and a polypeptide comprising BMP7, VTN, POSTN, FGF17, THBS2, THBS1, IGF2, IL-15, or THBS4. The composition may comprise FGF17, IGF2, THBS1, and a polypeptide comprising BMP7, VTN, POSTN, FGF17, THBS2, THBS1, IGF2, IL-15, or THBS4. The composition may comprise FGF17, IGF2, IL-15, and a polypeptide comprising BMP7, VTN, POSTN, FGF17, THBS2, THBS1, IGF2, IL-15, or THBS4. The composition may comprise FGF17, IGF2, THBS4, and a polypeptide comprising BMP7, VTN, POSTN, FGF17, THBS2, THBS1, IGF2, IL-15, or THBS4.

The composition may comprise FGF17, IL-15, VTN, and a polypeptide comprising BMP7, VTN, POSTN, FGF17, THBS2, THBS1, IGF2, IL-15, or THBS4. The composition may comprise FGF17, IL-15, POSTN, and a polypeptide comprising BMP7, VTN, POSTN, FGF17, THBS2, THBS1, IGF2, IL-15, or THBS4. The composition may comprise FGF17, IL-15, THBS2, and a polypeptide comprising BMP7, VTN, POSTN, FGF17, THBS2, THBS1, IGF2, IL-15, or THBS4. The composition may comprise FGF17, IL-15, THBS1, and a polypeptide comprising BMP7, VTN, POSTN, FGF17, THBS2, THBS1, IGF2, IL-15, or THBS4. The composition may comprise FGF17, IL-15, IGF2, and a polypeptide comprising BMP7, VTN, POSTN, FGF17, THBS2, THBS1, IGF2, IL-15, or THBS4. The composition may comprise FGF17, IL-15, THBS4, and a polypeptide comprising BMP7, VTN, POSTN, FGF17, THBS2, THBS1, IGF2, IL-15, or THBS4.

The composition may comprise FGF17, THBS4, VTN, and a polypeptide comprising BMP7, VTN, POSTN, FGF17, THBS2, THBS1, IGF2, IL-15, or THBS4. The composition may comprise FGF17, THBS4, POSTN, and a polypeptide comprising BMP7, VTN, POSTN, FGF17, THBS2, THBS1, IGF2, IL-15, or THBS4. The composition may comprise FGF17, THBS4, THBS2, and a polypeptide comprising BMP7, VTN, POSTN, FGF17, THBS2, THBS1, IGF2, IL-15, or THBS4. The composition may comprise FGF17, THBS4, THBS1, and a polypeptide comprising BMP7, VTN, POSTN, FGF17, THBS2, THBS1, IGF2, IL-15, or THBS4. The composition may comprise FGF17, THBS4, IGF2, and a polypeptide comprising BMP7, VTN, POSTN, FGF17, THBS2, THBS1, IGF2, IL-15, or THBS4. The composition may comprise FGF17, THBS4, IL-15, and a polypeptide comprising BMP7, VTN, POSTN, FGF17, THBS2, THBS1, IGF2, IL-15, or THBS4.

The composition may comprise THBS2, THBS1, VTN, and a polypeptide comprising BMP7, VTN, POSTN, FGF17, THBS2, THBS1, IGF2, IL-15, or THBS4. The composition may comprise THBS2, THBS1, POSTN, and a polypeptide comprising BMP7, VTN, POSTN, FGF17, THBS2, THBS1, IGF2, IL-15, or THBS4. The composition may comprise THBS2, THBS1, FGF17, and a polypeptide comprising BMP7, VTN, POSTN, FGF17, THBS2, THBS1, IGF2, IL-15, or THBS4. The composition may comprise THBS2, THBS1, IGF2, and a polypeptide comprising BMP7, VTN, POSTN, FGF17, THBS2, THBS1, IGF2, IL-15, or THBS4. The composition may comprise THBS2, THBS1, IL-15, and a polypeptide comprising BMP7, VTN, POSTN, FGF17, THBS2, THBS1, IGF2, IL-15, or THBS4. The composition may comprise THBS2, THBS1, THBS4, and a polypeptide comprising BMP7, VTN, POSTN, FGF17, THBS2, THBS1, IGF2, IL-15, or THBS4.

The composition may comprise THBS2, IGF2, VTN, and a polypeptide comprising BMP7, VTN, POSTN, FGF17, THBS2, THBS1, IGF2, IL-15, or THBS4. The composition may comprise THBS2, IGF2, POSTN, and a polypeptide comprising BMP7, VTN, POSTN, FGF17, THBS2, THBS1, IGF2, IL-15, or THBS4. The composition may comprise THBS2, IGF2, FGF17, and a polypeptide comprising BMP7, VTN, POSTN, FGF17, THBS2, THBS1, IGF2, IL-15, or THBS4. The composition may comprise THBS2, IGF2, THBS1, and a polypeptide comprising BMP7, VTN, POSTN, FGF17, THBS2, THBS1, IGF2, IL-15, or THBS4. The composition may comprise THBS2, IGF2, IL-15, and a polypeptide comprising BMP7, VTN, POSTN, FGF17, THBS2, THBS1, IGF2, IL-15, or THBS4. The composition may comprise THBS2, IGF2, THBS4, and a polypeptide comprising BMP7, VTN, POSTN, FGF17, THBS2, THBS1, IGF2, IL-15, or THBS4.

The composition may comprise THBS2, IL-15, VTN, and a polypeptide comprising BMP7, VTN, POSTN, FGF17, THBS2, THBS1, IGF2, IL-15, or THBS4. The composition may comprise THBS2, IL-15, POSTN, and a polypeptide comprising BMP7, VTN, POSTN, FGF17, THBS2, THBS1, IGF2, IL-15, or THBS4. The composition may comprise THBS2, IL-15, FGF17, and a polypeptide comprising BMP7, VTN, POSTN, FGF17, THBS2, THBS1, IGF2, IL-15, or THBS4. The composition may comprise THBS2, IL-15, THBS1, and a polypeptide comprising BMP7, VTN, POSTN, FGF17, THBS2, THBS1, IGF2, IL-15, or THBS4. The composition may comprise THBS2, IL-15, IGF2, and a polypeptide comprising BMP7, VTN, POSTN, FGF17, THBS2, THBS1, IGF2, IL-15, or THBS4. The composition may comprise THBS2, IL-15, THBS4, and a polypeptide comprising BMP7, VTN, POSTN, FGF17, THBS2, THBS1, IGF2, IL-15, or THBS4.

The composition may comprise THBS2, THBS4, VTN, and a polypeptide comprising BMP7, VTN, POSTN, FGF17, THBS2, THBS1, IGF2, IL-15, or THBS4. The composition may comprise THBS2, THBS4, POSTN, and a polypeptide comprising BMP7, VTN, POSTN, FGF17, THBS2, THBS1, IGF2, IL-15, or THBS4. The composition may comprise THBS2, THBS4, FGF17, and a polypeptide comprising BMP7, VTN, POSTN, FGF17, THBS2, THBS1, IGF2, IL-15, or THBS4. The composition may comprise THBS2, THBS4, THBS1, and a polypeptide comprising BMP7, VTN, POSTN, FGF17, THBS2, THBS1, IGF2, IL-15, or THBS4. The composition may comprise THBS2, THBS4, IGF2, and a polypeptide comprising BMP7, VTN, POSTN, FGF17, THBS2, THBS1, IGF2, IL-15, or THBS4. The composition may comprise THBS2, THBS4, IL-15, and a polypeptide comprising BMP7, VTN, POSTN, FGF17, THBS2, THBS1, IGF2, IL-15, or THBS4.

The composition may comprise THBS1, IGF2, VTN, and a polypeptide comprising BMP7, VTN, POSTN, FGF17, THBS2, THBS1, IGF2, IL-15, or THBS4. The composition may comprise THBS1, IGF2, POSTN, and a polypeptide comprising BMP7, VTN, POSTN, FGF17, THBS2, THBS1, IGF2, IL-15, or THBS4. The composition may comprise THBS1, IGF2, FGF17, and a polypeptide comprising BMP7, VTN, POSTN, FGF17, THBS2, THBS1, IGF2, IL-15, or THBS4. The composition may comprise THBS1, IGF2, THBS2, and a polypeptide comprising BMP7, VTN, POSTN, FGF17, THBS2, THBS1, IGF2, IL-15, or THBS4. The composition may comprise THBS1, IGF2, IL-15, and a polypeptide comprising BMP7, VTN, POSTN, FGF17, THBS2, THBS1, IGF2, IL-15, or THBS4. The composition may comprise THBS1, IGF2, THBS4, and a polypeptide comprising BMP7, VTN, POSTN, FGF17, THBS2, THBS1, IGF2, IL-15, or THBS4.

The composition may comprise THBS1, IL-15, VTN, and a polypeptide comprising BMP7, VTN, POSTN, FGF17, THBS2, THBS1, IGF2, IL-15, or THBS4. The composition may comprise THBS1, IL-15, POSTN, and a polypeptide comprising BMP7, VTN, POSTN, FGF17, THBS2, THBS1, IGF2, IL-15, or THBS4. The composition may comprise THBS1, IL-15, FGF17, and a polypeptide comprising BMP7, VTN, POSTN, FGF17, THBS2, THBS1, IGF2, IL-15, or THBS4. The composition may comprise THBS1, IL-15, THBS2, and a polypeptide comprising BMP7, VTN, POSTN, FGF17, THBS2, THBS1, IGF2, IL-15, or THBS4. The composition may comprise THBS1, IL-15, IGF2, and a polypeptide comprising BMP7, VTN, POSTN, FGF17, THBS2, THBS1, IGF2, IL-15, or THBS4. The composition may comprise THBS1, IL-15, THBS4, and a polypeptide comprising BMP7, VTN, POSTN, FGF17, THBS2, THBS1, IGF2, IL-15, or THBS4.

The composition may comprise THBS1, and THBS4, VTN, and a polypeptide comprising BMP7, VTN, POSTN, FGF17, THBS2, THBS1, IGF2, IL-15, or THBS4. The composition may comprise THBS1, THBS4, POSTN, and a polypeptide comprising BMP7, VTN, POSTN, FGF17, THBS2, THBS1, IGF2, IL-15, or THBS4. The composition may comprise THBS1, THBS4, FGF17, and a polypeptide comprising BMP7, VTN, POSTN, FGF17, THBS2, THBS1, IGF2, IL-15, or THBS4. The composition may comprise THBS1, THBS4, THBS2, and a polypeptide comprising BMP7, VTN, POSTN, FGF17, THBS2, THBS1, IGF2, IL-15, or THBS4. The composition may comprise THBS1, THBS4, IGF2, and a polypeptide comprising BMP7, VTN, POSTN, FGF17, THBS2, THBS1, IGF2, IL-15, or THBS4. The composition may comprise THBS1, THBS4, IL-15, and a polypeptide comprising BMP7, VTN, POSTN, FGF17, THBS2, THBS1, IGF2, IL-15, or THBS4.

The composition may comprise IGF2, IL-15, VTN, and a polypeptide comprising BMP7, VTN, POSTN, FGF17, THBS2, THBS1, IGF2, IL-15, or THBS4. The composition may comprise IGF2, IL-15, POSTN, and a polypeptide comprising BMP7, VTN, POSTN, FGF17, THBS2, THBS1, IGF2, IL-15, or THBS4. The composition may comprise IGF2, IL-15, FGF17, and a polypeptide comprising BMP7, VTN, POSTN, FGF17, THBS2, THBS1, IGF2, IL-15, or THBS4. The composition may comprise IGF2, IL-15, THBS2, and a polypeptide comprising BMP7, VTN, POSTN, FGF17, THBS2, THBS1, IGF2, IL-15, or THBS4. The composition may comprise IGF2, IL-15, THBS1, and a polypeptide comprising BMP7, VTN, POSTN, FGF17, THBS2, THBS1, IGF2, IL-15, or THBS4. The composition may comprise IGF2, IL-15, THBS4, and a polypeptide comprising BMP7, VTN, POSTN, FGF17, THBS2, THBS1, IGF2, IL-15, or THBS4.

The composition may comprise IGF2, THBS4, VTN, and a polypeptide comprising BMP7, VTN, POSTN, FGF17, THBS2, THBS1, IGF2, IL-15, or THBS4. The composition may comprise IGF2, THBS4, POSTN, and a polypeptide comprising BMP7, VTN, POSTN, FGF17, THBS2, THBS1, IGF2, IL-15, or THBS4. The composition may comprise IGF2, THBS4, FGF17, and a polypeptide comprising BMP7, VTN, POSTN, FGF17, THBS2, THBS1, IGF2, IL-15, or THBS4. The composition may comprise IGF2, THBS4, THBS2, and a polypeptide comprising BMP7, VTN, POSTN, FGF17, THBS2, THBS1, IGF2, IL-15, or THBS4. The composition may comprise IGF2, THBS4, THBS1, and a polypeptide comprising BMP7, VTN, POSTN, FGF17, THBS2, THBS1, IGF2, IL-15, or THBS4. The composition may comprise IGF2, THBS4, IL-15, and a polypeptide comprising BMP7, VTN, POSTN, FGF17, THBS2, THBS1, IGF2, IL-15, or THBS4.

The composition may comprise IL-15, THBS4, VTN, and a polypeptide comprising BMP7, VTN, POSTN, FGF17, THBS2, THBS1, IGF2, IL-15, or THBS4. The composition may comprise IL-15, THBS4, POSTN, and a polypeptide comprising BMP7, VTN, POSTN, FGF17, THBS2, THBS1, IGF2, IL-15, or THBS4. The composition may comprise IL-15, THBS4, FGF17, and a polypeptide comprising BMP7, VTN, POSTN, FGF17, THBS2, THBS1, IGF2, IL-15, or THBS4. The composition may comprise IL-15, THBS4, THBS2, and a polypeptide comprising BMP7, VTN, POSTN, FGF17, THBS2, THBS1, IGF2, IL-15, or THBS4. The composition may comprise IL-15, THBS4, THBS1, and a polypeptide comprising BMP7, VTN, POSTN, FGF17, THBS2, THBS1, IGF2, IL-15, or THBS4. The composition may comprise IL-15, THBS4, IGF2, and a polypeptide comprising BMP7, VTN, POSTN, FGF17, THBS2, THBS1, IGF2, IL-15, or THBS4.

The heparin-associated binding polypeptide composition comprising any four polypeptides selected from BMP7, VTN, POSTN, FGF17, THBS2, THBS1, IGF2, IL-15, and THBS4, may comprise one or more additional modifications to increase stability. In certain embodiments, one or more of the HAPs is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the HAPs is fused or conjugated to another protein to increase stability and or bioavailability. In certain embodiments, one or more of the HAPs is fused with an Fc region of an immunoglobulin or with serum albumin. In certain embodiments, one or more of the HAPs is present in a concatemer with one, two, three, four or more distinct polypeptides selected from BMP7, THBS2, VTN, POSTN, FGF17, THBS1, IL-15, IGF2, and THBS4. In certain embodiments, one or more of the HAPs is present in a concatemer with one, two, three, four, or more distinct polypeptides. In certain embodiments, one or more of the HAPs is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA).

The heparin-associated binding polypeptide composition may comprise any three polypeptides selected from VTN, STC2, AGRN, THBS2, and FST. The composition may comprise VTN, STC2, and AGRN. The composition may comprise VTN, STC2, and THBS2. The composition may comprise VTN, STC2, and FST. The composition may comprise VTN, AGRN, and STC2. The composition may comprise VTN, AGRN, and THBS2. The composition may comprise VTN, AGRN, and FST. The composition may comprise VTN, THBS2, and STC2. The composition may comprise VTN, THBS2, and AGRN. The composition may comprise VTN, THBS2, and FST. The composition may comprise VTN, FST, and STC2. The composition may comprise VTN, FST, and AGRN. The composition may comprise VTN, FST, and THBS2. The composition may comprise STC2, VTN, and FST. The composition may comprise STC2, VTN, and AGRN. The composition may comprise STC2, VTN, and THBS2. The composition may comprise STC2, AGRN, and FST. The composition may comprise STC2, AGRN, and VTN. The composition may comprise STC2, AGRN, and THBS2. The composition may comprise STC2, THBS2, and FST. The composition may comprise STC2, THBS2, and VTN. The composition may comprise STC2, THBS2, and AGRN. The composition may comprise STC2, FST, and THBS2. The composition may comprise STC2, FST, and VTN. The composition may comprise STC2, FST, and AGRN. The composition may comprise AGRN, VTN, and THBS2. The composition may comprise AGRN, VTN, and FST. The composition may comprise AGRN, VTN, and STC2. The composition may comprise AGRN, STC2, and THBS2. The composition may comprise AGRN, STC2, and FST. The composition may comprise AGRN, STC2, and VTN. The composition may comprise AGRN, THBS2, and STC2. The composition may comprise AGRN, THBS2, and FST. The composition may comprise AGRN, THBS2, and VTN. The composition may comprise AGRN, FST, and STC2. The composition may comprise AGRN, FST, and THBS2. The composition may comprise AGRN, FST, and VTN. The composition may comprise THBS2, VTN, and STC2. The composition may comprise THBS2, VTN, and AGRN. The composition may comprise THBS2, VTN, and FST. The composition may comprise THBS2, STC2, and VTN. The composition may comprise THBS2, STC2, and AGRN. The composition may comprise THBS2, STC2, and FST. The composition may comprise THBS2, AGRN, and VTN. The composition may comprise THBS2, AGRN, and STC2. The composition may comprise THBS2, AGRN, and FST. The composition may comprise THBS2, FST, and VTN. The composition may comprise THBS2, FST, and STC2. The composition may comprise THBS2, FST, and AGRN. The composition may comprise FST, VTN, and THBS2. The composition may comprise FST, VTN, and STC2. The composition may comprise FST, VTN, and AGRN. The composition may comprise FST, STC2, and THBS2. The composition may comprise FST, STC2, and VTN. The composition may comprise FST, STC2, and AGRN. The composition may comprise FST, AGRN, and THBS2. The composition may comprise FST, AGRN, and VTN. The composition may comprise FST, AGRN, and STC2. The composition may comprise FST, THBS2, and AGRN. The composition may comprise FST, THBS2, and VTN. The composition may comprise FST, THBS2, and STC2. In certain embodiments, one or more of the HAPs comprise one or more additional modifications to increase stability. In certain embodiments, one or more of the HAPs is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the HAP is fused or conjugated to another protein to increase stability and or bioavailability. In certain embodiments, one or more of the HAPs is fused with an Fc region of an immunoglobulin or with serum albumin. In certain embodiments, one or more of the HAPs is present in a concatemer with one, two, three, four or more distinct polypeptides selected from THBS2, VTN, STC2, AGRN, or FST. In certain embodiments, one or more of the HAPs is present in a concatemer with one, two, three, four, or more distinct polypeptides. In certain embodiments, one or more of the HAPs is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA).

The heparin-associated binding polypeptide composition may comprise any four polypeptides selected from VTN, STC2, AGRN, THBS2, and FST. The composition may comprise VTN, STC2, AGRN, and THBS2. The composition may comprise VTN, STC2, AGRN, and FST. The composition may comprise VTN, STC2, THBS2, and AGRN. The composition may comprise VTN, STC2, THBS2, and FST. The composition may comprise VTN, STC2, FST, and AGRN. The composition may comprise VTN, STC2, FST, and THBS2. The composition may comprise VTN, AGRN, STC2, and FST. The composition may comprise VTN, AGRN, STC2, and THBS2. The composition may comprise VTN, AGRN, THBS2, and FST. The composition may comprise VTN, AGRN, THBS2, and STC2. The composition may comprise VTN, AGRN, FST, and THBS2. The composition may comprise VTN, AGRN, FST, and STC2. The composition may comprise VTN, THBS2, STC2, and AGRN. The composition may comprise VTN, THBS2, STC2, and FST. The composition may comprise VTN, THBS2, AGRN, and STC2. The composition may comprise VTN, THBS2, AGRN, and FST. The composition may comprise VTN, THBS2, FST, and STC2. The composition may comprise VTN, THBS2, FST, and AGRN. The composition may comprise VTN, FST, STC2, and THBS2. The composition may comprise VTN, FST, STC2, and AGRN. The composition may comprise VTN, FST, AGRN, and THBS2. The composition may comprise VTN, FST, AGRN, and STC2. The composition may comprise VTN, FST, THBS2, and AGRN. The composition may comprise VTN, FST, THBS2, and STC2. The composition may comprise STC2, VTN, FST, and AGRN. The composition may comprise STC2, VTN, FST, and THBS2. The composition may comprise STC2, VTN, AGRN, and FST. The composition may comprise STC2, VTN, AGRN, and THBS2. The composition may comprise STC2, VTN, THBS2, and FST. The composition may comprise STC2, VTN, THBS2, and AGRN. The composition may comprise STC2, AGRN, FST, and THBS2. The composition may comprise STC2, AGRN, FST, and VTN. The composition may comprise STC2, AGRN, VTN, and THBS2. The composition may comprise STC2, AGRN, VTN, and FST. The composition may comprise STC2, AGRN, THBS2, and VTN. The composition may comprise STC2, AGRN, THBS2, and FST. The composition may comprise STC2, THBS2, FST, and VTN. The composition may comprise STC2, THBS2, FST, and AGRN. The composition may comprise STC2, THBS2, VTN, and FST. The composition may comprise STC2, THBS2, VTN, and AGRN. The composition may comprise STC2, THBS2, AGRN, and FST. The composition may comprise STC2, THBS2, AGRN, and VTN. The composition may comprise STC2, FST, THBS2, and AGRN. The composition may comprise STC2, FST, THBS2, and VTN. The composition may comprise STC2, FST, VTN, and AGRN. The composition may comprise STC2, FST, VTN, and THBS2. The composition may comprise STC2, FST, AGRN, and VTN. The composition may comprise STC2, FST, AGRN, and THBS2. The composition may comprise AGRN, VTN, THBS2, and FST. The composition may comprise AGRN, VTN, THBS2, and STC2. The composition may comprise AGRN, VTN, FST, and THBS2. The composition may comprise AGRN, VTN, FST, and STC2. The composition may comprise AGRN, VTN, STC2, and THBS2. The composition may comprise AGRN, VTN, STC2, and FST. The composition may comprise AGRN, STC2, THBS2, and VTN. The composition may comprise AGRN, STC2, THBS2, and FST. The composition may comprise AGRN, STC2, FST, and VTN. The composition may comprise AGRN, STC2, FST, and THBS2. The composition may comprise AGRN, STC2, VTN, and FST. The composition may comprise AGRN, STC2, VTN, and THBS2. The composition may comprise AGRN, THBS2, STC2, and FST. The composition may comprise AGRN, THBS2, STC2, and VTN. The composition may comprise AGRN, THBS2, FST, and STC2. The composition may comprise AGRN, THBS2, FST, and VTN. The composition may comprise AGRN, THBS2, VTN, and STC2. The composition may comprise AGRN, THBS2, VTN, and FST. The composition may comprise AGRN, FST, STC2, and VTN. The composition may comprise AGRN, FST, STC2, and THBS2. The composition may comprise AGRN, FST, THBS2, and VTN. The composition may comprise AGRN, FST, THBS2, and STC2. The composition may comprise AGRN, FST, VTN, and THBS2. The composition may comprise AGRN, FST, VTN, and STC2. The composition may comprise THBS2, VTN, STC2, and AGRN. The composition may comprise THBS2, VTN, STC2, and FST. The composition may comprise THBS2, VTN, AGRN, and STC2. The composition may comprise THBS2, VTN, AGRN, and FST. The composition may comprise THBS2, VTN, FST, and STC2. The composition may comprise THBS2, VTN, FST, and AGRN. The composition may comprise THBS2, STC2, VTN, and FST. The composition may comprise THBS2, STC2, VTN, and AGRN. The composition may comprise THBS2, STC2, AGRN, and FST. The composition may comprise THBS2, STC2, AGRN, and VTN. The composition may comprise THBS2, STC2, FST, and AGRN. The composition may comprise THBS2, STC2, FST, and VTN. The composition may comprise THBS2, AGRN, VTN, and STC2. The composition may comprise THBS2, AGRN, VTN, and FST. The composition may comprise THBS2, AGRN, STC2, and VTN. The composition may comprise THBS2, AGRN, STC2, and FST. The composition may comprise THBS2, AGRN, FST, and VTN. The composition may comprise THBS2, AGRN, FST, and STC2. The composition may comprise THBS2, FST, VTN, and AGRN. The composition may comprise THBS2, FST, VTN, and STC2. The composition may comprise THBS2, FST, STC2, and AGRN. The composition may comprise THBS2, FST, STC2, and VTN. The composition may comprise THBS2, FST, AGRN, and STC2. The composition may comprise THBS2, FST, AGRN, and VTN. The composition may comprise FST, VTN, THBS2, and STC2. The composition may comprise FST, VTN, THBS2, and AGRN. The composition may comprise FST, VTN, STC2, and THBS2. The composition may comprise FST, VTN, STC2, and AGRN. The composition may comprise FST, VTN, AGRN, and THBS2. The composition may comprise FST, VTN, AGRN, and STC2. The composition may comprise FST, STC2, THBS2, and AGRN. The composition may comprise FST, STC2, THBS2, and VTN. The composition may comprise FST, STC2, VTN, and AGRN. The composition may comprise FST, STC2, VTN, and THBS2. The composition may comprise FST, STC2, AGRN, and VTN. The composition may comprise FST, STC2, AGRN, and THBS2. The composition may comprise FST, AGRN, THBS2, and VTN. The composition may comprise FST, AGRN, THBS2, and STC2. The composition may comprise FST, AGRN, VTN, and THBS2. The composition may comprise FST, AGRN, VTN, and STC2. The composition may comprise FST, AGRN, STC2, and THBS2. The composition may comprise FST, AGRN, STC2, and VTN. The composition may comprise FST, THBS2, AGRN, and STC2. The composition may comprise FST, THBS2, AGRN, and VTN. The composition may comprise FST, THBS2, VTN, and STC2. The composition may comprise FST, THBS2, VTN, and AGRN. The composition may comprise FST, THBS2, STC2, and VTN. The composition may comprise FST, THBS2, STC2, and AGRN. In certain embodiments, one or more of the HAPs comprise one or more additional modifications to increase stability. In certain embodiments, one or more of the HAPs is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the HAPs is fused or conjugated to another protein to increase stability and or bioavailability. In certain embodiments, one or more of the HAPs is fused with an Fc region of an immunoglobulin or with serum albumin. In certain embodiments, one or more of the HAPs is present in a concatemer with one, two, three, or four other distinct polypeptides selected from THBS2, VTN, STC2, AGRN, or FST. In certain embodiments, one or more of the HAPs is present in a concatemer with one, two, three, four, or more distinct polypeptides. In certain embodiments, one or more of the HAPs is included in a composition comprising a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA).

In some embodiments, a composition herein may comprise polypeptide 1 and one or more polypeptides from Table 2. In some embodiments, a composition herein may comprise polypeptide 2 and one or more polypeptides from Table 2. In some embodiments, a composition herein may comprise polypeptide 3 and one or more polypeptides from Table 2. In some embodiments, a composition herein may comprise polypeptide 4 and one or more polypeptides from Table 2. In some embodiments, a composition herein may comprise polypeptide 5 and one or more polypeptides from Table 2. In some embodiments, a composition herein may comprise polypeptide 6 and one or more polypeptides from Table 2. In some embodiments, a composition herein may comprise polypeptide 7 and one or more polypeptides from Table 2. In some embodiments, a composition herein may comprise polypeptide 8 and one or more polypeptides from Table 2. In some embodiments, a composition herein may comprise polypeptide 9 and one or more polypeptides from Table 2. In some embodiments, a composition herein may comprise polypeptide 10 and one or more polypeptides from Table 2. In some embodiments, a composition herein may comprise polypeptide 11 and one or more polypeptides from Table 2. In some embodiments, a composition herein may comprise polypeptide 12 and one or more polypeptides from Table 2. In some embodiments, a composition herein may comprise polypeptide 13 and one or more polypeptides from Table 2. In some embodiments, a composition herein may comprise polypeptide 14 and one or more polypeptides from Table 2. In some embodiments, a composition herein may comprise polypeptide 15 and one or more polypeptides from Table 2. In some embodiments, a composition herein may comprise polypeptide 16 and one or more polypeptides from Table 2. In some embodiments, a composition herein may comprise polypeptide 17 and one or more polypeptides from Table 2. In some embodiments, a composition herein may comprise polypeptide 18 and one or more polypeptides from Table 2. In some embodiments, a composition herein may comprise polypeptide 19 and one or more polypeptides from Table 2. In some embodiments, a composition herein may comprise polypeptide 21 and one or more polypeptides from Table 2. In some embodiments, a composition herein may comprise polypeptide 22 and one or more polypeptides from Table 2. In some embodiments, a composition herein may comprise polypeptide 23 and one or more polypeptides from Table 2. In some embodiments, a composition herein may comprise polypeptide 24 and one or more polypeptides from Table 2. In some embodiments, a composition herein may comprise polypeptide 25 and one or more polypeptides from Table 2. In some embodiments, a composition herein may comprise polypeptide 26 and one or more polypeptides from Table 2. In some embodiments, a composition herein may comprise polypeptide 27 and one or more polypeptides from Table 2. In some embodiments, a composition herein may comprise polypeptide 28 and one or more polypeptides from Table 2. In some embodiments, a composition herein may comprise polypeptide 29 and one or more polypeptides from Table 2. In some embodiments, a composition herein may comprise polypeptide 30 and one or more polypeptides from Table 2. In some embodiments, a composition herein may comprise polypeptide 31 and one or more polypeptides from Table 2. In some embodiments, a composition herein may comprise polypeptide 32 and one or more polypeptides from Table 2. In some embodiments, a composition herein may comprise polypeptide 33 and one or more polypeptides from Table 2. In some embodiments, a composition herein may comprise polypeptide 34 and one or more polypeptides from Table 2. In some embodiments, a composition herein may comprise polypeptide 35 and one or more polypeptides from Table 2. In some embodiments, a composition herein may comprise polypeptide 36 and one or more polypeptides from Table 2. In some embodiments, a composition herein may comprise polypeptide 37 and one or more polypeptides from Table 2. In some embodiments, a composition herein may comprise polypeptide 38 and one or more polypeptides from Table 2. In some embodiments, a composition herein may comprise polypeptide 39 and one or more polypeptides from Table 2. In some embodiments, a composition herein may comprise polypeptide 40 and one or more polypeptides from Table 2. In some embodiments, a composition herein may comprise polypeptide 41 and one or more polypeptides from Table 2. In some embodiments, a composition herein may comprise polypeptide 42 and one or more polypeptides from Table 2. In some embodiments, a composition herein may comprise polypeptide 43 and one or more polypeptides from Table 2. In some embodiments, a composition herein may comprise polypeptide 44 and one or more polypeptides from Table 2. In some embodiments, a composition herein may comprise polypeptide 45 and one or more polypeptides from Table 2. In some embodiments, a composition herein may comprise polypeptide 46 and one or more polypeptides from Table 2. In some embodiments, a composition herein may comprise polypeptide 47 and one or more polypeptides from Table 2. In some embodiments, a composition herein may comprise polypeptide 48 and one or more polypeptides from Table 2. In some embodiments, a composition herein may comprise polypeptide 49 and one or more polypeptides from Table 2. In some embodiments, a composition herein may comprise polypeptide 50 and one or more polypeptides from Table 2. In some embodiments, a composition herein may comprise polypeptide 51 and one or more polypeptides from Table 2. In some embodiments, a composition herein may comprise polypeptide 52 and one or more polypeptides from Table 2. In some embodiments, a composition herein may comprise polypeptide 53 and one or more polypeptides from Table 2. In some embodiments, a composition herein may comprise polypeptide 54 and one or more polypeptides from Table 2. In some embodiments, a composition herein may comprise polypeptide 55 and one or more polypeptides from Table 2. In some embodiments, a composition herein may comprise polypeptide 56 and one or more polypeptides from Table 2. In some embodiments, a composition herein may comprise polypeptide 57 and one or more polypeptides from Table 2. In some embodiments, a composition herein may comprise polypeptide 58 and one or more polypeptides from Table 2. In some embodiments, a composition herein may comprise polypeptide 59 and one or more polypeptides from Table 2. In some embodiments, a composition herein may comprise polypeptide 60 and one or more polypeptides from Table 2. In some embodiments, a composition herein may comprise polypeptide 61 and one or more polypeptides from Table 2. In some embodiments, a composition herein may comprise polypeptide 62 and one or more polypeptides from Table 2. In some embodiments, a composition herein may comprise polypeptide 63 and one or more polypeptides from Table 2. In some embodiments, a composition herein may comprise polypeptide 64 and one or more polypeptides from Table 2. In some embodiments, a composition herein may comprise polypeptide 65 and one or more polypeptides from Table 2. In some embodiments, a composition herein may comprise polypeptide 66 and one or more polypeptides from Table 2. In some embodiments, a composition herein may comprise polypeptide 67 and one or more polypeptides from Table 2. In some embodiments, a composition herein may comprise polypeptide 68 and one or more polypeptides from Table 2. In some embodiments, a composition herein may comprise polypeptide 69 and one or more polypeptides from Table 2. In some embodiments, a composition herein may comprise polypeptide 70 and one or more polypeptides from Table 2. In some embodiments, a composition herein may comprise polypeptide 71 and one or more polypeptides from Table 2. In some embodiments, a composition herein may comprise polypeptide 72 and one or more polypeptides from Table 2. In some embodiments, a composition herein may comprise polypeptide 73 and one or more polypeptides from Table 2. In some embodiments, a composition herein may comprise polypeptide 74 and one or more polypeptides from Table 2. In some embodiments, a composition herein may comprise polypeptide 75 and one or more polypeptides from Table 2. In some embodiments, a composition herein may comprise polypeptide 76 and one or more polypeptides from Table 2. In some embodiments, a composition herein may comprise polypeptide 77 and one or more polypeptides from Table 2. In some embodiments, a composition herein may comprise polypeptide 78 and one or more polypeptides from Table 2. In some embodiments, a composition herein may comprise polypeptide 79 and one or more polypeptides from Table 2. In some embodiments, a composition herein may comprise polypeptide 80 and one or more polypeptides from Table 2. In some embodiments, a composition herein may comprise polypeptide 81 and one or more polypeptides from Table 2. In some embodiments, a composition herein may comprise polypeptide 82 and one or more polypeptides from Table 2. In some embodiments, a composition herein may comprise polypeptide 83 and one or more polypeptides from Table 2. In some embodiments, a composition herein may comprise polypeptide 84 and one or more polypeptides from Table 2. In some embodiments, a composition herein may comprise polypeptide 85 and one or more polypeptides from Table 2. In some embodiments, a composition herein may comprise polypeptide 86 and one or more polypeptides from Table 2. In some embodiments, a composition herein may comprise polypeptide 87 and one or more polypeptides from Table 2. In some embodiments, a composition herein may comprise polypeptide 88 and one or more polypeptides from Table 2. In some embodiments, a composition herein may comprise polypeptide 89 and one or more polypeptides from Table 2. In some embodiments, a composition herein may comprise polypeptide 90 and one or more polypeptides from Table 2. In some embodiments, a composition herein may comprise polypeptide 91 and one or more polypeptides from Table 2. In some embodiments, a composition herein may comprise polypeptide 92 and one or more polypeptides from Table 2. In some embodiments, a composition herein may comprise polypeptide 93 and one or more polypeptides from Table 2. In some embodiments, a composition herein may comprise polypeptide 94 and one or more polypeptides from Table 2. In some embodiments, a composition herein may comprise polypeptide 95 and one or more polypeptides from Table 2. In some embodiments, a composition herein may comprise polypeptide 96 and one or more polypeptides from Table 2. In some embodiments, a composition herein may comprise polypeptide 97 and one or more polypeptides from Table 2. In some embodiments, a composition herein may comprise polypeptide 98 and one or more polypeptides from Table 2. In some embodiments, a composition herein may comprise polypeptide 99 and one or more polypeptides from Table 2. In some embodiments, a composition herein may comprise polypeptide 100 and one or more polypeptides from Table 2.

In some embodiments, a composition herein may comprise polypeptide 101 and one or more polypeptides from Table 2. In some embodiments, a composition herein may comprise polypeptide 102 and one or more polypeptides from Table 2. In some embodiments, a composition herein may comprise polypeptide 103 and one or more polypeptides from Table 2. In some embodiments, a composition herein may comprise polypeptide 104 and one or more polypeptides from Table 2. In some embodiments, a composition herein may comprise polypeptide 105 and one or more polypeptides from Table 2. In some embodiments, a composition herein may comprise polypeptide 106 and one or more polypeptides from Table 2. In some embodiments, a composition herein may comprise polypeptide 107 and one or more polypeptides from Table 2. In some embodiments, a composition herein may comprise polypeptide 108 and one or more polypeptides from Table 2. In some embodiments, a composition herein may comprise polypeptide 109 and one or more polypeptides from Table 2. In some embodiments, a composition herein may comprise polypeptide 110 and one or more polypeptides from Table 2. In some embodiments, a composition herein may comprise polypeptide 111 and one or more polypeptides from Table 2. In some embodiments, a composition herein may comprise polypeptide 112 and one or more polypeptides from Table 2. In some embodiments, a composition herein may comprise polypeptide 113 and one or more polypeptides from Table 2. In some embodiments, a composition herein may comprise polypeptide 114 and one or more polypeptides from Table 2. In some embodiments, a composition herein may comprise polypeptide 115 and one or more polypeptides from Table 2. In some embodiments, a composition herein may comprise polypeptide 116 and one or more polypeptides from Table 2. In some embodiments, a composition herein may comprise polypeptide 117 and one or more polypeptides from Table 2. In some embodiments, a composition herein may comprise polypeptide 118 and one or more polypeptides from Table 2. In some embodiments, a composition herein may comprise polypeptide 119 and one or more polypeptides from Table 2. In some embodiments, a composition herein may comprise polypeptide 121 and one or more polypeptides from Table 2. In some embodiments, a composition herein may comprise polypeptide 122 and one or more polypeptides from Table 2. In some embodiments, a composition herein may comprise polypeptide 123 and one or more polypeptides from Table 2. In some embodiments, a composition herein may comprise polypeptide 124 and one or more polypeptides from Table 2. In some embodiments, a composition herein may comprise polypeptide 125 and one or more polypeptides from Table 2. In some embodiments, a composition herein may comprise polypeptide 126 and one or more polypeptides from Table 2. In some embodiments, a composition herein may comprise polypeptide 127 and one or more polypeptides from Table 2. In some embodiments, a composition herein may comprise polypeptide 128 and one or more polypeptides from Table 2. In some embodiments, a composition herein may comprise polypeptide 129 and one or more polypeptides from Table 2. In some embodiments, a composition herein may comprise polypeptide 130 and one or more polypeptides from Table 2. In some embodiments, a composition herein may comprise polypeptide 131 and one or more polypeptides from Table 2. In some embodiments, a composition herein may comprise polypeptide 132 and one or more polypeptides from Table 2. In some embodiments, a composition herein may comprise polypeptide 133 and one or more polypeptides from Table 2. In some embodiments, a composition herein may comprise polypeptide 134 and one or more polypeptides from Table 2. In some embodiments, a composition herein may comprise polypeptide 135 and one or more polypeptides from Table 2. In some embodiments, a composition herein may comprise polypeptide 136 and one or more polypeptides from Table 2. In some embodiments, a composition herein may comprise polypeptide 137 and one or more polypeptides from Table 2. In some embodiments, a composition herein may comprise polypeptide 138 and one or more polypeptides from Table 2. In some embodiments, a composition herein may comprise polypeptide 139 and one or more polypeptides from Table 2. In some embodiments, a composition herein may comprise polypeptide 140 and one or more polypeptides from Table 2. In some embodiments, a composition herein may comprise polypeptide 141 and one or more polypeptides from Table 2. In some embodiments, a composition herein may comprise polypeptide 142 and one or more polypeptides from Table 2. In some embodiments, a composition herein may comprise polypeptide 143 and one or more polypeptides from Table 2. In some embodiments, a composition herein may comprise polypeptide 144 and one or more polypeptides from Table 2. In some embodiments, a composition herein may comprise polypeptide 145 and one or more polypeptides from Table 2. In some embodiments, a composition herein may comprise polypeptide 146 and one or more polypeptides from Table 2. In some embodiments, a composition herein may comprise polypeptide 147 and one or more polypeptides from Table 2. In some embodiments, a composition herein may comprise polypeptide 148 and one or more polypeptides from Table 2. In some embodiments, a composition herein may comprise polypeptide 149 and one or more polypeptides from Table 2. In some embodiments, a composition herein may comprise polypeptide 150 and one or more polypeptides from Table 2. In some embodiments, a composition herein may comprise polypeptide 151 and one or more polypeptides from Table 2. In some embodiments, a composition herein may comprise polypeptide 152 and one or more polypeptides from Table 2. In some embodiments, a composition herein may comprise polypeptide 153 and one or more polypeptides from Table 2. In some embodiments, a composition herein may comprise polypeptide 154 and one or more polypeptides from Table 2. In some embodiments, a composition herein may comprise polypeptide 155 and one or more polypeptides from Table 2. In some embodiments, a composition herein may comprise polypeptide 156 and one or more polypeptides from Table 2. In some embodiments, a composition herein may comprise polypeptide 157 and one or more polypeptides from Table 2. In some embodiments, a composition herein may comprise polypeptide 158 and one or more polypeptides from Table 2. In some embodiments, a composition herein may comprise polypeptide 159 and one or more polypeptides from Table 2. In some embodiments, a composition herein may comprise polypeptide 160 and one or more polypeptides from Table 2. In some embodiments, a composition herein may comprise polypeptide 161 and one or more polypeptides from Table 2. In some embodiments, a composition herein may comprise polypeptide 162 and one or more polypeptides from Table 2. In some embodiments, a composition herein may comprise polypeptide 163 and one or more polypeptides from Table 2. In some embodiments, a composition herein may comprise polypeptide 164 and one or more polypeptides from Table 2. In some embodiments, a composition herein may comprise polypeptide 165 and one or more polypeptides from Table 2. In some embodiments, a composition herein may comprise polypeptide 166 and one or more polypeptides from Table 2. In some embodiments, a composition herein may comprise polypeptide 167 and one or more polypeptides from Table 2. In some embodiments, a composition herein may comprise polypeptide 168 and one or more polypeptides from Table 2. In some embodiments, a composition herein may comprise polypeptide 169 and one or more polypeptides from Table 2. In some embodiments, a composition herein may comprise polypeptide 170 and one or more polypeptides from Table 2. In some embodiments, a composition herein may comprise polypeptide 171 and one or more polypeptides from Table 2. In some embodiments, a composition herein may comprise polypeptide 172 and one or more polypeptides from Table 2. In some embodiments, a composition herein may comprise polypeptide 173 and one or more polypeptides from Table 2. In some embodiments, a composition herein may comprise polypeptide 174 and one or more polypeptides from Table 2. In some embodiments, a composition herein may comprise polypeptide 175 and one or more polypeptides from Table 2. In some embodiments, a composition herein may comprise polypeptide 176 and one or more polypeptides from Table 2. In some embodiments, a composition herein may comprise polypeptide 177 and one or more polypeptides from Table 2. In some embodiments, a composition herein may comprise polypeptide 178 and one or more polypeptides from Table 2. In some embodiments, a composition herein may comprise polypeptide 179 and one or more polypeptides from Table 2. In some embodiments, a composition herein may comprise polypeptide 180 and one or more polypeptides from Table 2. In some embodiments, a composition herein may comprise polypeptide 181 and one or more polypeptides from Table 2. In some embodiments, a composition herein may comprise polypeptide 182 and one or more polypeptides from Table 2. In some embodiments, a composition herein may comprise polypeptide 183 and one or more polypeptides from Table 2. In some embodiments, a composition herein may comprise polypeptide 184 and one or more polypeptides from Table 2. In some embodiments, a composition herein may comprise polypeptide 185 and one or more polypeptides from Table 2. In some embodiments, a composition herein may comprise polypeptide 186 and one or more polypeptides from Table 2. In some embodiments, a composition herein may comprise polypeptide 187 and one or more polypeptides from Table 2. In some embodiments, a composition herein may comprise polypeptide 188 and one or more polypeptides from Table 2. In some embodiments, a composition herein may comprise polypeptide 189 and one or more polypeptides from Table 2. In some embodiments, a composition herein may comprise polypeptide 190 and one or more polypeptides from Table 2. In some embodiments, a composition herein may comprise polypeptide 191 and one or more polypeptides from Table 2. In some embodiments, a composition herein may comprise polypeptide 192 and one or more polypeptides from Table 2. In some embodiments, a composition herein may comprise polypeptide 193 and one or more polypeptides from Table 2. In some embodiments, a composition herein may comprise polypeptide 194 and one or more polypeptides from Table 2. In some embodiments, a composition herein may comprise polypeptide 195 and one or more polypeptides from Table 2. In some embodiments, a composition herein may comprise polypeptide 196 and one or more polypeptides from Table 2. In some embodiments, a composition herein may comprise polypeptide 197 and one or more polypeptides from Table 2. In some embodiments, a composition herein may comprise polypeptide 198 and one or more polypeptides from Table 2. In some embodiments, a composition herein may comprise polypeptide 199 and one or more polypeptides from Table 2. In some embodiments, a composition herein may comprise polypeptide 200 and one or more polypeptides from Table 2.

In some embodiments, a composition herein may comprise polypeptide 201 and one or more polypeptides from Table 2. In some embodiments, a composition herein may comprise polypeptide 202 and one or more polypeptides from Table 2. In some embodiments, a composition herein may comprise polypeptide 203 and one or more polypeptides from Table 2. In some embodiments, a composition herein may comprise polypeptide 204 and one or more polypeptides from Table 2. In some embodiments, a composition herein may comprise polypeptide 205 and one or more polypeptides from Table 2. In some embodiments, a composition herein may comprise polypeptide 206 and one or more polypeptides from Table 2. In some embodiments, a composition herein may comprise polypeptide 207 and one or more polypeptides from Table 2. In some embodiments, a composition herein may comprise polypeptide 208 and one or more polypeptides from Table 2. In some embodiments, a composition herein may comprise polypeptide 209 and one or more polypeptides from Table 2. In some embodiments, a composition herein may comprise polypeptide 210 and one or more polypeptides from Table 2. In some embodiments, a composition herein may comprise polypeptide 211 and one or more polypeptides from Table 2. In some embodiments, a composition herein may comprise polypeptide 212 and one or more polypeptides from Table 2. In some embodiments, a composition herein may comprise polypeptide 213 and one or more polypeptides from Table 2. In some embodiments, a composition herein may comprise polypeptide 214 and one or more polypeptides from Table 2. In some embodiments, a composition herein may comprise polypeptide 215 and one or more polypeptides from Table 2. In some embodiments, a composition herein may comprise polypeptide 216 and one or more polypeptides from Table 2. In some embodiments, a composition herein may comprise polypeptide 217 and one or more polypeptides from Table 2. In some embodiments, a composition herein may comprise polypeptide 218 and one or more polypeptides from Table 2. In some embodiments, a composition herein may comprise polypeptide 219 and one or more polypeptides from Table 2. In some embodiments, a composition herein may comprise polypeptide 221 and one or more polypeptides from Table 2. In some embodiments, a composition herein may comprise polypeptide 222 and one or more polypeptides from Table 2. In some embodiments, a composition herein may comprise polypeptide 223 and one or more polypeptides from Table 2. In some embodiments, a composition herein may comprise polypeptide 224 and one or more polypeptides from Table 2. In some embodiments, a composition herein may comprise polypeptide 225 and one or more polypeptides from Table 2. In some embodiments, a composition herein may comprise polypeptide 226 and one or more polypeptides from Table 2. In some embodiments, a composition herein may comprise polypeptide 227 and one or more polypeptides from Table 2. In some embodiments, a composition herein may comprise polypeptide 228 and one or more polypeptides from Table 2. In some embodiments, a composition herein may comprise polypeptide 229 and one or more polypeptides from Table 2. In some embodiments, a composition herein may comprise polypeptide 230 and one or more polypeptides from Table 2. In some embodiments, a composition herein may comprise polypeptide 231 and one or more polypeptides from Table 2. In some embodiments, a composition herein may comprise polypeptide 232 and one or more polypeptides from Table 2. In some embodiments, a composition herein may comprise polypeptide 233 and one or more polypeptides from Table 2. In some embodiments, a composition herein may comprise polypeptide 234 and one or more polypeptides from Table 2. In some embodiments, a composition herein may comprise polypeptide 235 and one or more polypeptides from Table 2. In some embodiments, a composition herein may comprise polypeptide 236 and one or more polypeptides from Table 2. In some embodiments, a composition herein may comprise polypeptide 237 and one or more polypeptides from Table 2. In some embodiments, a composition herein may comprise polypeptide 238 and one or more polypeptides from Table 2. In some embodiments, a composition herein may comprise polypeptide 239 and one or more polypeptides from Table 2. In some embodiments, a composition herein may comprise polypeptide 240 and one or more polypeptides from Table 2. In some embodiments, a composition herein may comprise polypeptide 241 and one or more polypeptides from Table 2. In some embodiments, a composition herein may comprise polypeptide 242 and one or more polypeptides from Table 2. In some embodiments, a composition herein may comprise polypeptide 243 and one or more polypeptides from Table 2. In some embodiments, a composition herein may comprise polypeptide 244 and one or more polypeptides from Table 2. In some embodiments, a composition herein may comprise polypeptide 245 and one or more polypeptides from Table 2. In some embodiments, a composition herein may comprise polypeptide 246 and one or more polypeptides from Table 2. In some embodiments, a composition herein may comprise polypeptide 247 and one or more polypeptides from Table 2. In some embodiments, a composition herein may comprise polypeptide 248 and one or more polypeptides from Table 2. In some embodiments, a composition herein may comprise polypeptide 249 and one or more polypeptides from Table 2. In some embodiments, a composition herein may comprise polypeptide 250 and one or more polypeptides from Table 2. In some embodiments, a composition herein may comprise polypeptide 251 and one or more polypeptides from Table 2. In some embodiments, a composition herein may comprise polypeptide 252 and one or more polypeptides from Table 2. In some embodiments, a composition herein may comprise polypeptide 253 and one or more polypeptides from Table 2. In some embodiments, a composition herein may comprise polypeptide 254 and one or more polypeptides from Table 2. In some embodiments, a composition herein may comprise polypeptide 255 and one or more polypeptides from Table 2. In some embodiments, a composition herein may comprise polypeptide 256 and one or more polypeptides from Table 2. In some embodiments, a composition herein may comprise polypeptide 257 and one or more polypeptides from Table 2. In some embodiments, a composition herein may comprise polypeptide 258 and one or more polypeptides from Table 2. In some embodiments, a composition herein may comprise polypeptide 259 and one or more polypeptides from Table 2. In some embodiments, a composition herein may comprise polypeptide 260 and one or more polypeptides from Table 2. In some embodiments, a composition herein may comprise polypeptide 261 and one or more polypeptides from Table 2. In some embodiments, a composition herein may comprise polypeptide 262 and one or more polypeptides from Table 2. In some embodiments, a composition herein may comprise polypeptide 263 and one or more polypeptides from Table 2. In some embodiments, a composition herein may comprise polypeptide 264 and one or more polypeptides from Table 2. In some embodiments, a composition herein may comprise polypeptide 265 and one or more polypeptides from Table 2. In some embodiments, a composition herein may comprise polypeptide 266 and one or more polypeptides from Table 2. In some embodiments, a composition herein may comprise polypeptide 267 and one or more polypeptides from Table 2. In some embodiments, a composition herein may comprise polypeptide 268 and one or more polypeptides from Table 2. In some embodiments, a composition herein may comprise polypeptide 269 and one or more polypeptides from Table 2. In some embodiments, a composition herein may comprise polypeptide 270 and one or more polypeptides from Table 2. In some embodiments, a composition herein may comprise polypeptide 271 and one or more polypeptides from Table 2. In some embodiments, a composition herein may comprise polypeptide 272 and one or more polypeptides from Table 2. In some embodiments, a composition herein may comprise polypeptide 273 and one or more polypeptides from Table 2. In some embodiments, a composition herein may comprise polypeptide 274 and one or more polypeptides from Table 2. In some embodiments, a composition herein may comprise polypeptide 275 and one or more polypeptides from Table 2. In some embodiments, a composition herein may comprise polypeptide 276 and one or more polypeptides from Table 2. In some embodiments, a composition herein may comprise polypeptide 277 and one or more polypeptides from Table 2. In some embodiments, a composition herein may comprise polypeptide 278 and one or more polypeptides from Table 2. In some embodiments, a composition herein may comprise polypeptide 279 and one or more polypeptides from Table 2. In some embodiments, a composition herein may comprise polypeptide 280 and one or more polypeptides from Table 2. In some embodiments, a composition herein may comprise polypeptide 281 and one or more polypeptides from Table 2. In some embodiments, a composition herein may comprise polypeptide 282 and one or more polypeptides from Table 2. In some embodiments, a composition herein may comprise polypeptide 283 and one or more polypeptides from Table 2. In some embodiments, a composition herein may comprise polypeptide 284 and one or more polypeptides from Table 2. In some embodiments, a composition herein may comprise polypeptide 285 and one or more polypeptides from Table 2. In some embodiments, a composition herein may comprise polypeptide 286 and one or more polypeptides from Table 2. In some embodiments, a composition herein may comprise polypeptide 287 and one or more polypeptides from Table 2. In some embodiments, a composition herein may comprise polypeptide 288 and one or more polypeptides from Table 2. In some embodiments, a composition herein may comprise polypeptide 289 and one or more polypeptides from Table 2. In some embodiments, a composition herein may comprise polypeptide 290 and one or more polypeptides from Table 2. In some embodiments, a composition herein may comprise polypeptide 291 and one or more polypeptides from Table 2. In some embodiments, a composition herein may comprise polypeptide 292 and one or more polypeptides from Table 2. In some embodiments, a composition herein may comprise polypeptide 293 and one or more polypeptides from Table 2. In some embodiments, a composition herein may comprise polypeptide 294 and one or more polypeptides from Table 2. In some embodiments, a composition herein may comprise polypeptide 295 and one or more polypeptides from Table 2. In some embodiments, a composition herein may comprise polypeptide 296 and one or more polypeptides from Table 2. In some embodiments, a composition herein may comprise polypeptide 297 and one or more polypeptides from Table 2. In some embodiments, a composition herein may comprise polypeptide 298 and one or more polypeptides from Table 2. In some embodiments, a composition herein may comprise polypeptide 299 and one or more polypeptides from Table 2. In some embodiments, a composition herein may comprise polypeptide 300 and one or more polypeptides from Table 2.

In some embodiments, a composition herein may comprise polypeptide 401 and one or more polypeptides from Table 2. In some embodiments, a composition herein may comprise polypeptide 402 and one or more polypeptides from Table 2. In some embodiments, a composition herein may comprise polypeptide 403 and one or more polypeptides from Table 2. In some embodiments, a composition herein may comprise polypeptide 404 and one or more polypeptides from Table 2. In some embodiments, a composition herein may comprise polypeptide 405 and one or more polypeptides from Table 2. In some embodiments, a composition herein may comprise polypeptide 406 and one or more polypeptides from Table 2. In some embodiments, a composition herein may comprise polypeptide 407 and one or more polypeptides from Table 2. In some embodiments, a composition herein may comprise polypeptide 408 and one or more polypeptides from Table 2. In some embodiments, a composition herein may comprise polypeptide 409 and one or more polypeptides from Table 2. In some embodiments, a composition herein may comprise polypeptide 410 and one or more polypeptides from Table 2. In some embodiments, a composition herein may comprise polypeptide 411 and one or more polypeptides from Table 2. In some embodiments, a composition herein may comprise polypeptide 412 and one or more polypeptides from Table 2. In some embodiments, a composition herein may comprise polypeptide 413 and one or more polypeptides from Table 2. In some embodiments, a composition herein may comprise polypeptide 414 and one or more polypeptides from Table 2. In some embodiments, a composition herein may comprise polypeptide 415 and one or more polypeptides from Table 2. In some embodiments, a composition herein may comprise polypeptide 416 and one or more polypeptides from Table 2. In some embodiments, a composition herein may comprise polypeptide 417 and one or more polypeptides from Table 2. In some embodiments, a composition herein may comprise polypeptide 418 and one or more polypeptides from Table 2. In some embodiments, a composition herein may comprise polypeptide 419 and one or more polypeptides from Table 2. In some embodiments, a composition herein may comprise polypeptide 421 and one or more polypeptides from Table 2. In some embodiments, a composition herein may comprise polypeptide 422 and one or more polypeptides from Table 2. In some embodiments, a composition herein may comprise polypeptide 423 and one or more polypeptides from Table 2. In some embodiments, a composition herein may comprise polypeptide 424 and one or more polypeptides from Table 2. In some embodiments, a composition herein may comprise polypeptide 425 and one or more polypeptides from Table 2. In some embodiments, a composition herein may comprise polypeptide 426 and one or more polypeptides from Table 2. In some embodiments, a composition herein may comprise polypeptide 427 and one or more polypeptides from Table 2. In some embodiments, a composition herein may comprise polypeptide 428 and one or more polypeptides from Table 2. In some embodiments, a composition herein may comprise polypeptide 429 and one or more polypeptides from Table 2. In some embodiments, a composition herein may comprise polypeptide 430 and one or more polypeptides from Table 2. In some embodiments, a composition herein may comprise polypeptide 431 and one or more polypeptides from Table 2. In some embodiments, a composition herein may comprise polypeptide 432 and one or more polypeptides from Table 2. In some embodiments, a composition herein may comprise polypeptide 433 and one or more polypeptides from Table 2. In some embodiments, a composition herein may comprise polypeptide 434 and one or more polypeptides from Table 2. In some embodiments, a composition herein may comprise polypeptide 435 and one or more polypeptides from Table 2. In some embodiments, a composition herein may comprise polypeptide 436 and one or more polypeptides from Table 2. In some embodiments, a composition herein may comprise polypeptide 437 and one or more polypeptides from Table 2. In some embodiments, a composition herein may comprise polypeptide 438 and one or more polypeptides from Table 2. In some embodiments, a composition herein may comprise polypeptide 439 and one or more polypeptides from Table 2. In some embodiments, a composition herein may comprise polypeptide 440 and one or more polypeptides from Table 2. In some embodiments, a composition herein may comprise polypeptide 441 and one or more polypeptides from Table 2. In some embodiments, a composition herein may comprise polypeptide 442 and one or more polypeptides from Table 2. In some embodiments, a composition herein may comprise polypeptide 443 and one or more polypeptides from Table 2. In some embodiments, a composition herein may comprise polypeptide 444 and one or more polypeptides from Table 2. In some embodiments, a composition herein may comprise polypeptide 445 and one or more polypeptides from Table 2. In some embodiments, a composition herein may comprise polypeptide 446 and one or more polypeptides from Table 2. In some embodiments, a composition herein may comprise polypeptide 447 and one or more polypeptides from Table 2. In some embodiments, a composition herein may comprise polypeptide 448 and one or more polypeptides from Table 2. In some embodiments, a composition herein may comprise polypeptide 449 and one or more polypeptides from Table 2. In some embodiments, a composition herein may comprise polypeptide 450 and one or more polypeptides from Table 2. In some embodiments, a composition herein may comprise polypeptide 451 and one or more polypeptides from Table 2. In some embodiments, a composition herein may comprise polypeptide 452 and one or more polypeptides from Table 2. In some embodiments, a composition herein may comprise polypeptide 453 and one or more polypeptides from Table 2. In some embodiments, a composition herein may comprise polypeptide 454 and one or more polypeptides from Table 2. In some embodiments, a composition herein may comprise polypeptide 455 and one or more polypeptides from Table 2. In some embodiments, a composition herein may comprise polypeptide 456 and one or more polypeptides from Table 2. In some embodiments, a composition herein may comprise polypeptide 457 and one or more polypeptides from Table 2. In some embodiments, a composition herein may comprise polypeptide 458 and one or more polypeptides from Table 2. In some embodiments, a composition herein may comprise polypeptide 459 and one or more polypeptides from Table 2. In some embodiments, a composition herein may comprise polypeptide 460 and one or more polypeptides from Table 2. In some embodiments, a composition herein may comprise polypeptide 461 and one or more polypeptides from Table 2. In some embodiments, a composition herein may comprise polypeptide 462 and one or more polypeptides from Table 2. In some embodiments, a composition herein may comprise polypeptide 463 and one or more polypeptides from Table 2. In some embodiments, a composition herein may comprise polypeptide 464 and one or more polypeptides from Table 2. In some embodiments, a composition herein may comprise polypeptide 465 and one or more polypeptides from Table 2. In some embodiments, a composition herein may comprise polypeptide 466 and one or more polypeptides from Table 2. In some embodiments, a composition herein may comprise polypeptide 467 and one or more polypeptides from Table 2. In some embodiments, a composition herein may comprise polypeptide 468 and one or more polypeptides from Table 2. In some embodiments, a composition herein may comprise polypeptide 469 and one or more polypeptides from Table 2. In some embodiments, a composition herein may comprise polypeptide 470 and one or more polypeptides from Table 2. In some embodiments, a composition herein may comprise polypeptide 471 and one or more polypeptides from Table 2. In some embodiments, a composition herein may comprise polypeptide 472 and one or more polypeptides from Table 2. In some embodiments, a composition herein may comprise polypeptide 473 and one or more polypeptides from Table 2. In some embodiments, a composition herein may comprise polypeptide 474 and one or more polypeptides from Table 2. In some embodiments, a composition herein may comprise polypeptide 475 and one or more polypeptides from Table 2. In some embodiments, a composition herein may comprise polypeptide 476 and one or more polypeptides from Table 2. In some embodiments, a composition herein may comprise polypeptide 477 and one or more polypeptides from Table 2. In some embodiments, a composition herein may comprise polypeptide 478 and one or more polypeptides from Table 2. In some embodiments, a composition herein may comprise polypeptide 479 and one or more polypeptides from Table 2. In some embodiments, a composition herein may comprise polypeptide 480 and one or more polypeptides from Table 2. In some embodiments, a composition herein may comprise polypeptide 481 and one or more polypeptides from Table 2. In some embodiments, a composition herein may comprise polypeptide 482 and one or more polypeptides from Table 2. In some embodiments, a composition herein may comprise polypeptide 483 and one or more polypeptides from Table 2. In some embodiments, a composition herein may comprise polypeptide 484 and one or more polypeptides from Table 2. In some embodiments, a composition herein may comprise polypeptide 485 and one or more polypeptides from Table 2. In some embodiments, a composition herein may comprise polypeptide 486 and one or more polypeptides from Table 2. In some embodiments, a composition herein may comprise polypeptide 487 and one or more polypeptides from Table 2. In some embodiments, a composition herein may comprise polypeptide 488 and one or more polypeptides from Table 2. In some embodiments, a composition herein may comprise polypeptide 489 and one or more polypeptides from Table 2. In some embodiments, a composition herein may comprise polypeptide 490 and one or more polypeptides from Table 2. In some embodiments, a composition herein may comprise polypeptide 491 and one or more polypeptides from Table 2. In some embodiments, a composition herein may comprise polypeptide 492 and one or more polypeptides from Table 2. In some embodiments, a composition herein may comprise polypeptide 493 and one or more polypeptides from Table 2. In some embodiments, a composition herein may comprise polypeptide 494 and one or more polypeptides from Table 2. In some embodiments, a composition herein may comprise polypeptide 495 and one or more polypeptides from Table 2. In some embodiments, a composition herein may comprise polypeptide 496 and one or more polypeptides from Table 2. In some embodiments, a composition herein may comprise polypeptide 497 and one or more polypeptides from Table 2. In some embodiments, a composition herein may comprise polypeptide 498 and one or more polypeptides from Table 2. In some embodiments, a composition herein may comprise polypeptide 499 and one or more polypeptides from Table 2. In some embodiments, a composition herein may comprise polypeptide 500 and one or more polypeptides from Table 2.

In some embodiments, a composition herein may comprise polypeptide 501 and one or more polypeptides from Table 2. In some embodiments, a composition herein may comprise polypeptide 502 and one or more polypeptides from Table 2. In some embodiments, a composition herein may comprise polypeptide 503 and one or more polypeptides from Table 2. In some embodiments, a composition herein may comprise polypeptide 504 and one or more polypeptides from Table 2. In some embodiments, a composition herein may comprise polypeptide 505 and one or more polypeptides from Table 2. In some embodiments, a composition herein may comprise polypeptide 506 and one or more polypeptides from Table 2. In some embodiments, a composition herein may comprise polypeptide 507 and one or more polypeptides from Table 2. In some embodiments, a composition herein may comprise polypeptide 508 and one or more polypeptides from Table 2. In some embodiments, a composition herein may comprise polypeptide 509 and one or more polypeptides from Table 2. In some embodiments, a composition herein may comprise polypeptide 510 and one or more polypeptides from Table 2. In some embodiments, a composition herein may comprise polypeptide 511 and one or more polypeptides from Table 2. In some embodiments, a composition herein may comprise polypeptide 512 and one or more polypeptides from Table 2. In some embodiments, a composition herein may comprise polypeptide 513 and one or more polypeptides from Table 2. In some embodiments, a composition herein may comprise polypeptide 514 and one or more polypeptides from Table 2. In some embodiments, a composition herein may comprise polypeptide 515 and one or more polypeptides from Table 2. In some embodiments, a composition herein may comprise polypeptide 516 and one or more polypeptides from Table 2. In some embodiments, a composition herein may comprise polypeptide 517 and one or more polypeptides from Table 2. In some embodiments, a composition herein may comprise polypeptide 518 and one or more polypeptides from Table 2. In some embodiments, a composition herein may comprise polypeptide 519 and one or more polypeptides from Table 2. In some embodiments, a composition herein may comprise polypeptide 521 and one or more polypeptides from Table 2. In some embodiments, a composition herein may comprise polypeptide 522 and one or more polypeptides from Table 2. In some embodiments, a composition herein may comprise polypeptide 523 and one or more polypeptides from Table 2. In some embodiments, a composition herein may comprise polypeptide 524 and one or more polypeptides from Table 2. In some embodiments, a composition herein may comprise polypeptide 525 and one or more polypeptides from Table 2. In some embodiments, a composition herein may comprise polypeptide 526 and one or more polypeptides from Table 2. In some embodiments, a composition herein may comprise polypeptide 527 and one or more polypeptides from Table 2. In some embodiments, a composition herein may comprise polypeptide 528 and one or more polypeptides from Table 2. In some embodiments, a composition herein may comprise polypeptide 529 and one or more polypeptides from Table 2. In some embodiments, a composition herein may comprise polypeptide 530 and one or more polypeptides from Table 2. In some embodiments, a composition herein may comprise polypeptide 531 and one or more polypeptides from Table 2. In some embodiments, a composition herein may comprise polypeptide 532 and one or more polypeptides from Table 2. In some embodiments, a composition herein may comprise polypeptide 533 and one or more polypeptides from Table 2. In some embodiments, a composition herein may comprise polypeptide 534 and one or more polypeptides from Table 2. In some embodiments, a composition herein may comprise polypeptide 535 and one or more polypeptides from Table 2. In some embodiments, a composition herein may comprise polypeptide 536 and one or more polypeptides from Table 2. In some embodiments, a composition herein may comprise polypeptide 537 and one or more polypeptides from Table 2. In some embodiments, a composition herein may comprise polypeptide 538 and one or more polypeptides from Table 2. In some embodiments, a composition herein may comprise polypeptide 539 and one or more polypeptides from Table 2. In some embodiments, a composition herein may comprise polypeptide 540 and one or more polypeptides from Table 2. In some embodiments, a composition herein may comprise polypeptide 541 and one or more polypeptides from Table 2. In some embodiments, a composition herein may comprise polypeptide 542 and one or more polypeptides from Table 2. In some embodiments, a composition herein may comprise polypeptide 543 and one or more polypeptides from Table 2. In some embodiments, a composition herein may comprise polypeptide 544 and one or more polypeptides from Table 2. In some embodiments, a composition herein may comprise polypeptide 545 and one or more polypeptides from Table 2. In some embodiments, a composition herein may comprise polypeptide 546 and one or more polypeptides from Table 2. In some embodiments, a composition herein may comprise polypeptide 547 and one or more polypeptides from Table 2. In some embodiments, a composition herein may comprise polypeptide 548 and one or more polypeptides from Table 2. In some embodiments, a composition herein may comprise polypeptide 549 and one or more polypeptides from Table 2. In some embodiments, a composition herein may comprise polypeptide 550 and one or more polypeptides from Table 2. In some embodiments, a composition herein may comprise polypeptide 551 and one or more polypeptides from Table 2. In some embodiments, a composition herein may comprise polypeptide 552 and one or more polypeptides from Table 2. In some embodiments, a composition herein may comprise polypeptide 553 and one or more polypeptides from Table 2. In some embodiments, a composition herein may comprise polypeptide 554 and one or more polypeptides from Table 2. In some embodiments, a composition herein may comprise polypeptide 555 and one or more polypeptides from Table 2. In some embodiments, a composition herein may comprise polypeptide 556 and one or more polypeptides from Table 2. In some embodiments, a composition herein may comprise polypeptide 557 and one or more polypeptides from Table 2. In some embodiments, a composition herein may comprise polypeptide 558 and one or more polypeptides from Table 2. In some embodiments, a composition herein may comprise polypeptide 559 and one or more polypeptides from Table 2. In some embodiments, a composition herein may comprise polypeptide 560 and one or more polypeptides from Table 2. In some embodiments, a composition herein may comprise polypeptide 561 and one or more polypeptides from Table 2. In some embodiments, a composition herein may comprise polypeptide 562 and one or more polypeptides from Table 2. In some embodiments, a composition herein may comprise polypeptide 563 and one or more polypeptides from Table 2. In some embodiments, a composition herein may comprise polypeptide 564 and one or more polypeptides from Table 2. In some embodiments, a composition herein may comprise polypeptide 565 and one or more polypeptides from Table 2. In some embodiments, a composition herein may comprise polypeptide 566 and one or more polypeptides from Table 2. In some embodiments, a composition herein may comprise polypeptide 567 and one or more polypeptides from Table 2. In some embodiments, a composition herein may comprise polypeptide 568 and one or more polypeptides from Table 2. In some embodiments, a composition herein may comprise polypeptide 569 and one or more polypeptides from Table 2. In some embodiments, a composition herein may comprise polypeptide 570 and one or more polypeptides from Table 2. In some embodiments, a composition herein may comprise polypeptide 571 and one or more polypeptides from Table 2. In some embodiments, a composition herein may comprise polypeptide 572 and one or more polypeptides from Table 2. In some embodiments, a composition herein may comprise polypeptide 573 and one or more polypeptides from Table 2. In some embodiments, a composition herein may comprise polypeptide 574 and one or more polypeptides from Table 2. In some embodiments, a composition herein may comprise polypeptide 575 and one or more polypeptides from Table 2. In some embodiments, a composition herein may comprise polypeptide 576 and one or more polypeptides from Table 2. In some embodiments, a composition herein may comprise polypeptide 577 and one or more polypeptides from Table 2. In some embodiments, a composition herein may comprise polypeptide 578 and one or more polypeptides from Table 2. In some embodiments, a composition herein may comprise polypeptide 579 and one or more polypeptides from Table 2. In some embodiments, a composition herein may comprise polypeptide 580 and one or more polypeptides from Table 2. In some embodiments, a composition herein may comprise polypeptide 581 and one or more polypeptides from Table 2. In some embodiments, a composition herein may comprise polypeptide 582 and one or more polypeptides from Table 2. In some embodiments, a composition herein may comprise polypeptide 583 and one or more polypeptides from Table 2. In some embodiments, a composition herein may comprise polypeptide 584 and one or more polypeptides from Table 2. In some embodiments, a composition herein may comprise polypeptide 585 and one or more polypeptides from Table 2. In some embodiments, a composition herein may comprise polypeptide 586 and one or more polypeptides from Table 2. In some embodiments, a composition herein may comprise polypeptide 587 and one or more polypeptides from Table 2. In some embodiments, a composition herein may comprise polypeptide 588 and one or more polypeptides from Table 2. In some embodiments, a composition herein may comprise polypeptide 589 and one or more polypeptides from Table 2. In some embodiments, a composition herein may comprise polypeptide 590 and one or more polypeptides from Table 2. In some embodiments, a composition herein may comprise polypeptide 591 and one or more polypeptides from Table 2. In some embodiments, a composition herein may comprise polypeptide 592 and one or more polypeptides from Table 2. In some embodiments, a composition herein may comprise polypeptide 593 and one or more polypeptides from Table 2. In some embodiments, a composition herein may comprise polypeptide 594 and one or more polypeptides from Table 2. In some embodiments, a composition herein may comprise polypeptide 595 and one or more polypeptides from Table 2. In some embodiments, a composition herein may comprise polypeptide 596 and one or more polypeptides from Table 2. In some embodiments, a composition herein may comprise polypeptide 597 and one or more polypeptides from Table 2. In some embodiments, a composition herein may comprise polypeptide 598 and one or more polypeptides from Table 2. In some embodiments, a composition herein may comprise polypeptide 599 and one or more polypeptides from Table 2. In some embodiments, a composition herein may comprise polypeptide 600 and one or more polypeptides from Table 2.

In some embodiments, a composition herein may comprise polypeptide 701 and one or more polypeptides from Table 2. In some embodiments, a composition herein may comprise polypeptide 702 and one or more polypeptides from Table 2. In some embodiments, a composition herein may comprise polypeptide 703 and one or more polypeptides from Table 2. In some embodiments, a composition herein may comprise polypeptide 704 and one or more polypeptides from Table 2. In some embodiments, a composition herein may comprise polypeptide 705 and one or more polypeptides from Table 2. In some embodiments, a composition herein may comprise polypeptide 706 and one or more polypeptides from Table 2. In some embodiments, a composition herein may comprise polypeptide 707 and one or more polypeptides from Table 2. In some embodiments, a composition herein may comprise polypeptide 708 and one or more polypeptides from Table 2. In some embodiments, a composition herein may comprise polypeptide 709 and one or more polypeptides from Table 2. In some embodiments, a composition herein may comprise polypeptide 710 and one or more polypeptides from Table 2. In some embodiments, a composition herein may comprise polypeptide 711 and one or more polypeptides from Table 2. In some embodiments, a composition herein may comprise polypeptide 712 and one or more polypeptides from Table 2. In some embodiments, a composition herein may comprise polypeptide 713 and one or more polypeptides from Table 2. In some embodiments, a composition herein may comprise polypeptide 714 and one or more polypeptides from Table 2. In some embodiments, a composition herein may comprise polypeptide 715 and one or more polypeptides from Table 2. In some embodiments, a composition herein may comprise polypeptide 716 and one or more polypeptides from Table 2. In some embodiments, a composition herein may comprise polypeptide 717 and one or more polypeptides from Table 2. In some embodiments, a composition herein may comprise polypeptide 718 and one or more polypeptides from Table 2. In some embodiments, a composition herein may comprise polypeptide 719 and one or more polypeptides from Table 2. In some embodiments, a composition herein may comprise polypeptide 721 and one or more polypeptides from Table 2. In some embodiments, a composition herein may comprise polypeptide 722 and one or more polypeptides from Table 2. In some embodiments, a composition herein may comprise polypeptide 723 and one or more polypeptides from Table 2. In some embodiments, a composition herein may comprise polypeptide 724 and one or more polypeptides from Table 2. In some embodiments, a composition herein may comprise polypeptide 725 and one or more polypeptides from Table 2. In some embodiments, a composition herein may comprise polypeptide 726 and one or more polypeptides from Table 2. In some embodiments, a composition herein may comprise polypeptide 727 and one or more polypeptides from Table 2. In some embodiments, a composition herein may comprise polypeptide 728 and one or more polypeptides from Table 2. In some embodiments, a composition herein may comprise polypeptide 729 and one or more polypeptides from Table 2. In some embodiments, a composition herein may comprise polypeptide 730 and one or more polypeptides from Table 2. In some embodiments, a composition herein may comprise polypeptide 731 and one or more polypeptides from Table 2. In some embodiments, a composition herein may comprise polypeptide 732 and one or more polypeptides from Table 2. In some embodiments, a composition herein may comprise polypeptide 733 and one or more polypeptides from Table 2. In some embodiments, a composition herein may comprise polypeptide 734 and one or more polypeptides from Table 2. In some embodiments, a composition herein may comprise polypeptide 735 and one or more polypeptides from Table 2. In some embodiments, a composition herein may comprise polypeptide 736 and one or more polypeptides from Table 2. In some embodiments, a composition herein may comprise polypeptide 737 and one or more polypeptides from Table 2. In some embodiments, a composition herein may comprise polypeptide 738 and one or more polypeptides from Table 2. In some embodiments, a composition herein may comprise polypeptide 739 and one or more polypeptides from Table 2. In some embodiments, a composition herein may comprise polypeptide 740 and one or more polypeptides from Table 2. In some embodiments, a composition herein may comprise polypeptide 741 and one or more polypeptides from Table 2. In some embodiments, a composition herein may comprise polypeptide 742 and one or more polypeptides from Table 2. In some embodiments, a composition herein may comprise polypeptide 743 and one or more polypeptides from Table 2. In some embodiments, a composition herein may comprise polypeptide 744 and one or more polypeptides from Table 2. In some embodiments, a composition herein may comprise polypeptide 745 and one or more polypeptides from Table 2. In some embodiments, a composition herein may comprise polypeptide 746 and one or more polypeptides from Table 2. In some embodiments, a composition herein may comprise polypeptide 747 and one or more polypeptides from Table 2. In some embodiments, a composition herein may comprise polypeptide 748 and one or more polypeptides from Table 2. In some embodiments, a composition herein may comprise polypeptide 749 and one or more polypeptides from Table 2. In some embodiments, a composition herein may comprise polypeptide 750 and one or more polypeptides from Table 2. In some embodiments, a composition herein may comprise polypeptide 751 and one or more polypeptides from Table 2. In some embodiments, a composition herein may comprise polypeptide 752 and one or more polypeptides from Table 2. In some embodiments, a composition herein may comprise polypeptide 753 and one or more polypeptides from Table 2. In some embodiments, a composition herein may comprise polypeptide 754 and one or more polypeptides from Table 2. In some embodiments, a composition herein may comprise polypeptide 755 and one or more polypeptides from Table 2. In some embodiments, a composition herein may comprise polypeptide 756 and one or more polypeptides from Table 2. In some embodiments, a composition herein may comprise polypeptide 757 and one or more polypeptides from Table 2. In some embodiments, a composition herein may comprise polypeptide 758 and one or more polypeptides from Table 2. In some embodiments, a composition herein may comprise polypeptide 759 and one or more polypeptides from Table 2. In some embodiments, a composition herein may comprise polypeptide 760 and one or more polypeptides from Table 2. In some embodiments, a composition herein may comprise polypeptide 761 and one or more polypeptides from Table 2. In some embodiments, a composition herein may comprise polypeptide 762 and one or more polypeptides from Table 2. In some embodiments, a composition herein may comprise polypeptide 763 and one or more polypeptides from Table 2. In some embodiments, a composition herein may comprise polypeptide 764 and one or more polypeptides from Table 2. In some embodiments, a composition herein may comprise polypeptide 765 and one or more polypeptides from Table 2. In some embodiments, a composition herein may comprise polypeptide 766 and one or more polypeptides from Table 2. In some embodiments, a composition herein may comprise polypeptide 767 and one or more polypeptides from Table 2. In some embodiments, a composition herein may comprise polypeptide 768 and one or more polypeptides from Table 2. In some embodiments, a composition herein may comprise polypeptide 769 and one or more polypeptides from Table 2. In some embodiments, a composition herein may comprise polypeptide 770 and one or more polypeptides from Table 2. In some embodiments, a composition herein may comprise polypeptide 771 and one or more polypeptides from Table 2. In some embodiments, a composition herein may comprise polypeptide 772 and one or more polypeptides from Table 2. In some embodiments, a composition herein may comprise polypeptide 773 and one or more polypeptides from Table 2. In some embodiments, a composition herein may comprise polypeptide 774 and one or more polypeptides from Table 2. In some embodiments, a composition herein may comprise polypeptide 775 and one or more polypeptides from Table 2. In some embodiments, a composition herein may comprise polypeptide 776 and one or more polypeptides from Table 2. In some embodiments, a composition herein may comprise polypeptide 777 and one or more polypeptides from Table 2. In some embodiments, a composition herein may comprise polypeptide 778 and one or more polypeptides from Table 2. In some embodiments, a composition herein may comprise polypeptide 779 and one or more polypeptides from Table 2. In some embodiments, a composition herein may comprise polypeptide 780 and one or more polypeptides from Table 2. In some embodiments, a composition herein may comprise polypeptide 781 and one or more polypeptides from Table 2. In some embodiments, a composition herein may comprise polypeptide 782 and one or more polypeptides from Table 2. In some embodiments, a composition herein may comprise polypeptide 783 and one or more polypeptides from Table 2. In some embodiments, a composition herein may comprise polypeptide 784 and one or more polypeptides from Table 2. In some embodiments, a composition herein may comprise polypeptide 785 and one or more polypeptides from Table 2. In some embodiments, a composition herein may comprise polypeptide 786 and one or more polypeptides from Table 2. In some embodiments, a composition herein may comprise polypeptide 787 and one or more polypeptides from Table 2. In some embodiments, a composition herein may comprise polypeptide 788 and one or more polypeptides from Table 2. In some embodiments, a composition herein may comprise polypeptide 789 and one or more polypeptides from Table 2. In some embodiments, a composition herein may comprise polypeptide 790 and one or more polypeptides from Table 2. In some embodiments, a composition herein may comprise polypeptide 791 and one or more polypeptides from Table 2. In some embodiments, a composition herein may comprise polypeptide 792 and one or more polypeptides from Table 2. In some embodiments, a composition herein may comprise polypeptide 793 and one or more polypeptides from Table 2. In some embodiments, a composition herein may comprise polypeptide 794 and one or more polypeptides from Table 2. In some embodiments, a composition herein may comprise polypeptide 795 and one or more polypeptides from Table 2. In some embodiments, a composition herein may comprise polypeptide 796 and one or more polypeptides from Table 2. In some embodiments, a composition herein may comprise polypeptide 797 and one or more polypeptides from Table 2. In some embodiments, a composition herein may comprise polypeptide 798 and one or more polypeptides from Table 2. In some embodiments, a composition herein may comprise polypeptide 799 and one or more polypeptides from Table 2. In some embodiments, a composition herein may comprise polypeptide 800 and one or more polypeptides from Table 2.

In some embodiments, a composition herein may comprise polypeptide 801 and one or more polypeptides from Table 2. In some embodiments, a composition herein may comprise polypeptide 802 and one or more polypeptides from Table 2. In some embodiments, a composition herein may comprise polypeptide 803 and one or more polypeptides from Table 2. In some embodiments, a composition herein may comprise polypeptide 804 and one or more polypeptides from Table 2. In some embodiments, a composition herein may comprise polypeptide 805 and one or more polypeptides from Table 2. In some embodiments, a composition herein may comprise polypeptide 806 and one or more polypeptides from Table 2. In some embodiments, a composition herein may comprise polypeptide 807 and one or more polypeptides from Table 2. In some embodiments, a composition herein may comprise polypeptide 808 and one or more polypeptides from Table 2. In some embodiments, a composition herein may comprise polypeptide 809 and one or more polypeptides from Table 2. In some embodiments, a composition herein may comprise polypeptide 810 and one or more polypeptides from Table 2. In some embodiments, a composition herein may comprise polypeptide 811 and one or more polypeptides from Table 2. In some embodiments, a composition herein may comprise polypeptide 812 and one or more polypeptides from Table 2. In some embodiments, a composition herein may comprise polypeptide 813 and one or more polypeptides from Table 2. In some embodiments, a composition herein may comprise polypeptide 814 and one or more polypeptides from Table 2. In some embodiments, a composition herein may comprise polypeptide 815 and one or more polypeptides from Table 2. In some embodiments, a composition herein may comprise polypeptide 816 and one or more polypeptides from Table 2. In some embodiments, a composition herein may comprise polypeptide 817 and one or more polypeptides from Table 2. In some embodiments, a composition herein may comprise polypeptide 818 and one or more polypeptides from Table 2. In some embodiments, a composition herein may comprise polypeptide 819 and one or more polypeptides from Table 2. In some embodiments, a composition herein may comprise polypeptide 821 and one or more polypeptides from Table 2. In some embodiments, a composition herein may comprise polypeptide 822 and one or more polypeptides from Table 2. In some embodiments, a composition herein may comprise polypeptide 823 and one or more polypeptides from Table 2. In some embodiments, a composition herein may comprise polypeptide 824 and one or more polypeptides from Table 2. In some embodiments, a composition herein may comprise polypeptide 825 and one or more polypeptides from Table 2. In some embodiments, a composition herein may comprise polypeptide 826 and one or more polypeptides from Table 2. In some embodiments, a composition herein may comprise polypeptide 827 and one or more polypeptides from Table 2. In some embodiments, a composition herein may comprise polypeptide 828 and one or more polypeptides from Table 2. In some embodiments, a composition herein may comprise polypeptide 829 and one or more polypeptides from Table 2. In some embodiments, a composition herein may comprise polypeptide 830 and one or more polypeptides from Table 2. In some embodiments, a composition herein may comprise polypeptide 831 and one or more polypeptides from Table 2. In some embodiments, a composition herein may comprise polypeptide 832 and one or more polypeptides from Table 2. In some embodiments, a composition herein may comprise polypeptide 833 and one or more polypeptides from Table 2. In some embodiments, a composition herein may comprise polypeptide 834 and one or more polypeptides from Table 2. In some embodiments, a composition herein may comprise polypeptide 835 and one or more polypeptides from Table 2. In some embodiments, a composition herein may comprise polypeptide 836 and one or more polypeptides from Table 2. In some embodiments, a composition herein may comprise polypeptide 837 and one or more polypeptides from Table 2. In some embodiments, a composition herein may comprise polypeptide 838 and one or more polypeptides from Table 2. In some embodiments, a composition herein may comprise polypeptide 839 and one or more polypeptides from Table 2. In some embodiments, a composition herein may comprise polypeptide 840 and one or more polypeptides from Table 2. In some embodiments, a composition herein may comprise polypeptide 841 and one or more polypeptides from Table 2. In some embodiments, a composition herein may comprise polypeptide 842 and one or more polypeptides from Table 2. In some embodiments, a composition herein may comprise polypeptide 843 and one or more polypeptides from Table 2. In some embodiments, a composition herein may comprise polypeptide 844 and one or more polypeptides from Table 2. In some embodiments, a composition herein may comprise polypeptide 845 and one or more polypeptides from Table 2. In some embodiments, a composition herein may comprise polypeptide 846 and one or more polypeptides from Table 2. In some embodiments, a composition herein may comprise polypeptide 847 and one or more polypeptides from Table 2. In some embodiments, a composition herein may comprise polypeptide 848 and one or more polypeptides from Table 2. In some embodiments, a composition herein may comprise polypeptide 849 and one or more polypeptides from Table 2. In some embodiments, a composition herein may comprise polypeptide 850 and one or more polypeptides from Table 2. In some embodiments, a composition herein may comprise polypeptide 851 and one or more polypeptides from Table 2. In some embodiments, a composition herein may comprise polypeptide 852 and one or more polypeptides from Table 2. In some embodiments, a composition herein may comprise polypeptide 853 and one or more polypeptides from Table 2. In some embodiments, a composition herein may comprise polypeptide 854 and one or more polypeptides from Table 2. In some embodiments, a composition herein may comprise polypeptide 855 and one or more polypeptides from Table 2. In some embodiments, a composition herein may comprise polypeptide 856 and one or more polypeptides from Table 2. In some embodiments, a composition herein may comprise polypeptide 857 and one or more polypeptides from Table 2. In some embodiments, a composition herein may comprise polypeptide 858 and one or more polypeptides from Table 2. In some embodiments, a composition herein may comprise polypeptide 859 and one or more polypeptides from Table 2. In some embodiments, a composition herein may comprise polypeptide 860 and one or more polypeptides from Table 2. In some embodiments, a composition herein may comprise polypeptide 861 and one or more polypeptides from Table 2. In some embodiments, a composition herein may comprise polypeptide 862 and one or more polypeptides from Table 2. In some embodiments, a composition herein may comprise polypeptide 863 and one or more polypeptides from Table 2. In some embodiments, a composition herein may comprise polypeptide 864 and one or more polypeptides from Table 2. In some embodiments, a composition herein may comprise polypeptide 865 and one or more polypeptides from Table 2. In some embodiments, a composition herein may comprise polypeptide 866 and one or more polypeptides from Table 2. In some embodiments, a composition herein may comprise polypeptide 867 and one or more polypeptides from Table 2. In some embodiments, a composition herein may comprise polypeptide 868 and one or more polypeptides from Table 2. In some embodiments, a composition herein may comprise polypeptide 869 and one or more polypeptides from Table 2. In some embodiments, a composition herein may comprise polypeptide 870 and one or more polypeptides from Table 2. In some embodiments, a composition herein may comprise polypeptide 871 and one or more polypeptides from Table 2. In some embodiments, a composition herein may comprise polypeptide 872 and one or more polypeptides from Table 2. In some embodiments, a composition herein may comprise polypeptide 873 and one or more polypeptides from Table 2. In some embodiments, a composition herein may comprise polypeptide 874 and one or more polypeptides from Table 2. In some embodiments, a composition herein may comprise polypeptide 875 and one or more polypeptides from Table 2. In some embodiments, a composition herein may comprise polypeptide 876 and one or more polypeptides from Table 2.

In some cases, the one or more polypeptides from Table 2 may comprise IL-15. In some cases, the one of more polypeptides from Table 2 may comprise BMP7. In some cases, the one or more polypeptides from Table 2 may comprise THBS4. In some cases, the one or more polypeptides from Table 2 may comprise POSTN. In some cases, the one or more polypeptides from Table 2 may comprise THBS1. In some cases, the one or more polypeptides from Table 2 may comprise THBS2. In some cases, the one or more polypeptides from Table 2 may comprise VTN. In some cases, the one or more polypeptides from Table 2 may comprise FGF17. In some cases, the one or more polypeptides from Table 2 may comprise IGF2. In some cases, the one or more polypeptides from Table 2 may comprise polypeptide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, or 876. In some cases, one or a plurality of the polypeptides of the composition are HAPs. In some cases, one or a plurality of the polypeptides of the composition are mitogenic and/or fusion promoting polypeptides. In certain embodiments, one or more of the polypeptides of the composition comprise one or more additional modifications to increase stability. In certain embodiments, one or more of the polypeptides is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the polypeptides is fused or conjugated to another protein to increase stability and or bioavailability. In certain embodiments, one or more of the polypeptides is fused with an Fc region of an immunoglobulin or with serum albumin. In certain embodiments, one or more of the polypeptides is present in a concatemer with one, two, three, four or more distinct polypeptides selected from Table 2 and/or Table 1. In certain embodiments, one or more of the polypeptides is present in a concatemer with one, two, three, four, or more distinct polypeptides. In certain embodiments, one or more of the polypeptides is included in the composition with a biodegradable or bioabsorbable carrier that promotes polypeptide stability. The biodegradable or bioabsorbable carrier may comprise polylactic acid (PLA), polyglycolic acid (PGA), or Poly(D,L-lactic-coglycolic-acid) (PLGA). The biodegradable or bioabsorbable carrier may comprise polylactic acid (PLA). The biodegradable or bioabsorbable carrier may comprise polyglycolic acid (PGA). The biodegradable or bioabsorbable carrier may comprise Poly(D,L-lactic-coglycolic-acid) (PLGA).

The HAPs increase the mitogenic (e.g., proliferative capacity) of a somatic cell that is a tissue cell or a tissue precursor, such as: a muscle cell, a muscle precursor cell, a tenocyte, a tenocyte precursor cell, a chondrocyte, a chondrocyte precursor, a mesenchymal stem cell, or a fibroblast. The cell can be a precursor cell derived from any mammal, such as, monkeys, apes, dogs, cats, horses, rats, mice, or humans. The precursor cell is a human precursor cell. The HAPs increase the proliferative capacity of a mouse myoblast by at least about 1.5-fold, about 2-fold, about 3-fold, or about 4-fold as measured by BrdU or EdU incorporation.

### Therapeutic Indications

In certain aspects, HAPs and compositions comprising HAPs, described herein, are useful for treating diseases and disorders that involve soft-tissue injury, degradation, or destruction. Aging disorders that result in the deterioration and loss of muscle tissue are such soft-tissue disorders. Sarcopenia, for example, is the degenerative loss of skeletal muscle mass quality, and strength associated with aging. Injuries that result in acute muscle damage are other such disorders. The disorders include muscle ruptures, strains, and contusions. A rupture is a separating of the muscle tissues. Muscle strains are contraction-induced injuries in which muscle fibers tear due to extensive mechanical stress, and can be classified as a grade I, II, or III. Muscle contusions are muscle hematomas. Muscle injury can also be caused by non-mechanical stresses such as cachexia. Cachexia may be caused by malnutrition, cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), Crohn's disease, untreated/severe type 1 diabetes mellitus, anorexia nervosa, chemotherapy, muscular dystrophy or other genetic diseases which cause immobility, and hormonal deficiencies. Certain disorders that are weaknesses of specific muscles such as dysphagia or facioscapulohumeral muscular dystrophy may also be treated by the polypeptides described herein. Additional soft-tissues disorders that may be treated using the HAPs described herein are those that inflict injury to the tendons, ligaments or cartilage. The muscle wasting disease is a muscular dystrophy. The muscular dystrophy may comprise myotonic muscular dystrophy, Duchenne muscular dystrophy, Becker muscular dystrophy, Limb-girdle muscular dystrophy, facioscapulohumeral muscular dystrophy, congenital, muscular dystrophy, oculopharyngeal muscular dystrophy, or distal muscular dystrophy. The muscular dystrophy is Becker muscular dystrophy. The HAPs useful for treating a soft-tissue disorder comprise any one, two, three, four, or five HAPs selected from Vitronectin (VTN), Periostin (POSTN), Fibroblast growth factor (FGF17), Thrombospondin 2 (THBS2), Thrombospondin 4 (THBS4), Thrombospondin 1 (THBS1), Insulin-like growth factor 2 (IGF2), Bone morphogenic protein 7 (BMP7), and Interleukin 15 (IL-15). The HAPs useful for treating a soft-tissue disorder comprise any one, two, three, four, or five HAPs selected from Vitronectin (VTN), Stanniocalcin-2 (STC2), Agrin (AGRN), Thrombospondin 2 (THBS2), follistatin (FST), Periostin (POSTN), Fibroblast growth factor (FGF17), Thrombospondin 4 (THBS4), Thrombospondin 1 (THBS1), Insulin-like growth factor 2 (IGF2), and Interleukin 15 (IL-15).

The HAPs and compositions comprising HAPs, described herein, are for use in treating an individual with an aging disorder, a muscle wasting disorder, a muscle injury, an injury to a connective tissue, or an injury to a non-muscle soft-tissue, or any combination thereof. The aging disorder is sarcopenia. The muscle wasting disorder is cachexia. The cachexia is a result of a cancer. The cachexia is a result of AIDS. The injury is a muscle injury. The muscle wasting is atrophy do to limb immobilization or disuse. The muscle injury is a strain or a tear. The muscle injury is a Grade III strain. In certain embodiments, sarcopenia contributes to the incidence of the muscle injury. The injury is ligament damage. The ligament damage is a rupture or a tear. The injury is tendon damage. The tendon damage is a rupture or a tear. The injury is cartilage damage. The HAPs comprise any one, two, three, four, five or more HAPs selected from Vitronectin (VTN), Periostin (POSTN), Fibroblast growth factor (FGF17), Thrombospondin 2 (THBS2), Thrombospondin 4 (THBS4), Thrombospondin 1 (THBS1), Insulin-like growth factor 2 (IGF2), Bone morphogenic protein 7 (BMP7), and Interleukin 15 (IL-15). The HAPs comprise any one, two, three, four, five or more HAPs selected from Vitronectin (VTN), Stanniocalcin-2 (STC2), Agrin (AGRN), Thrombospondin 2 (THBS2), and follistatin (FST).

The HAPs and compositions comprising HAPs, described herein, are for use in a method of treating myositis. The myositis may comprise dermatomyositis, polymyositis, necrotizing myopathy (also called necrotizing autoimmune myopathy or immune-mediated necrotizing myopathy), juvenile myositis, or sporadic inclusion-body myositis. The HAPs comprise any one, two, three, four, five or more HAPs selected from Vitronectin (VTN), Periostin (POSTN), Fibroblast growth factor (FGF17), Thrombospondin 2 (THBS2), Thrombospondin 4 (THBS4), Thrombospondin 1 (THBS1), Insulin-like growth factor 2 (IGF2), Bone morphogenic protein 7 (BMP7), and Interleukin 15 (IL-15). The HAPs comprise any one, two, three, four, or five HAPs selected from Vitronectin (VTN), Stanniocalcin-2 (STC2), Agrin (AGRN), Thrombospondin 2 (THBS2), and follistatin (FST).

The HAPs and compositions comprising HAPs, described herein, are for use in a method of treating cartilage related-disorders. The cartilage related disorder may be due to tears, injuries, or wear. The cartilage-associated disease may be osteoarthritis, osteochondritis dissecans, achondroplasia, or degenerative cartilage lesions. The HAPs comprise any one, two, three, four, five, six, or more HAPs selected from Vitronectin (VTN), Periostin (POSTN), Fibroblast growth factor (FGF17), Thrombospondin 2 (THBS2), Thrombospondin 4 (THBS4), Thrombospondin 1 (THBS1), Insulin-like growth factor 2 (IGF2), Bone Morphogenic Protein 7 (BMP7), and Interleukin 15 (IL-15). The HAPs comprise any one, two, three, four, or five HAPs selected from Vitronectin (VTN), Stanniocalcin-2 (STC2), Agrin (AGRN), Thrombospondin 2 (THBS2), and follistatin (FST).

The HAPs and compositions comprising HAPs, described herein, are for use in a method of increasing proliferation or promoting survival of a cell associated with soft-tissue damage. The HAPs described herein are useful in a method of increasing proliferation or promoting survival of any one or more of a muscle cell, a muscle precursor cell, a tenocyte, a tenocyte precursor cell, a chondrocyte, a chondrocyte precursor cell, a mesenchymal stem cell, or a fibroblast. The HAPs comprise any one, two, three, four, five or more HAPs selected from Vitronectin (VTN), Periostin (POSTN), Fibroblast growth factor (FGF17), Thrombospondin 2 (THBS2), Thrombospondin 4 (THBS4), Thrombospondin 1 (THBS1), Insulin-like growth factor 2 (IGF2), Bone morphogenic protein 7 (BMP7), and Interleukin 15 (IL-15). The HAPs comprise any one, two, three, four, or five HAPs selected from Vitronectin (VTN), Stanniocalcin-2 (STC2), Agrin (AGRN), Thrombospondin 2 (THBS2), and follistatin (FST).

The HAPs compositions described herein can be administered separately or as a mixture of 2, 3, 4, 5, 6, 7, 8, 9, 10 or more heparin-binding or HAPs for the treatment of any disorder associated with muscle or soft-tissue.

In certain aspects, a method of treating a disease or condition, such as those described herein, in a subject in need thereof may comprise administering to the subject a composition comprising a polypeptide of Table 2. In some embodiments, the polypeptide of Table 2 is a polypeptide of Table 1. In some embodiments, the composition may comprise a pharmaceutically acceptable excipient, such as described herein. In some embodiments, the disease or condition may comprise an aging disorder, muscle wasting disorder, muscle injury, or injury to connective tissue, or a combination thereof. In some embodiments, the aging disorder may comprise sarcopenia. In some embodiments, the muscle wasting disorder may comprise muscular dystrophy. In some embodiments, the muscle wasting is a result of obesity. The muscle wasting is the result of a metabolic disorder. In some cases the metabolic disorder is diabetes. In some cases the diabetes is Type 2 Diabetes. In some embodiments, muscle wasting is a result of disease progression. In some embodiments, muscle wasting is a result of therapeutic treatment. In some embodiments, the muscle wasting is cachexia. In some embodiments, the therapeutic polypeptide promotes fusion of myocytes.

In some embodiments, the polypeptide is a heparin-associated binding polypeptide as described herein. In some embodiments, the polypeptide is a mitogenic and/or fusion promoting polypeptide as described herein. In some embodiments, a composition comprising a plurality of heparin-associated binding polypeptides as described herein is administered. In some embodiments, a composition comprising a plurality of mitogenic and/or fusion promoting polypeptides as described herein is administered.

In some embodiments, the polypeptide has been recombinantly produced. In some embodiments, the polypeptide has been produced in a mammalian cell culture. The polypeptide has been produced in a mammalian cell and the mammalian cell is a human cell. In some cases the human cell is a human embryonic kidney-derived epithelial cell (e.g., HEK293 cells). In some embodiments, the mammalian cell culture is a mouse myeloma cell culture. In some embodiments, the mammalian cell culture is a Chinese Hamster Ovary (CHO) cell culture. In some embodiments, the polypeptide has been produced in a non-mammalian cell culture, e.g., in bacteria, yeast, or insect cells. The polypeptide has been purified from a human biological sample. In some cases, the human biological sample is human plasma. In some embodiments, the composition is formulated for administration by injection to the subject. In some embodiments, the composition may comprise one or more polypeptides having at least about 90% homology to a sequence selected from HAPs ID NOS: 1-44, 55, 56, and 58-72. In some embodiments, the composition may comprise polypeptide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, or 876, or any combination thereof.

In some embodiments, the polypeptide may comprise VTN. In some cases, the VTN may comprise a polypeptide comprising at least about 90% homology or identity to HAPs ID NO: 1. In some cases, the VTN may comprise a polypeptide comprising at least about 90% homology or identity to amino acids 20-478 of HAPs ID NO: 1. In some cases, the VTN is purified from human plasma.

In some embodiments, the polypeptide may comprise POSTN. In some cases, the POSTN may comprise a polypeptide comprising at least about 90% homology or identity to HAPs ID NO: 6. In some cases, the POSTN may comprise a polypeptide comprising at least about 90% homology or identity to amino acids 22-836 of HAPs ID NO: 6. In some cases, the POSTN is expressed in a mouse myeloma cell line.

In some embodiments, the polypeptide may comprise FGF17. In some cases, the FGF17 may comprise a polypeptide comprising at least about 90% homology or identity to HAPs ID NO: 7. In some cases, the FGF17 may comprise a polypeptide comprising at least about 90% homology or identity to amino acids 23-216 of HAPs ID NO: 7. In some cases, the FGF17 is expressed in a bacterial cell. In some embodiments, the bacterial cell is E. coli.

In some embodiments, the polypeptide may comprise THBS2. In some cases, the THBS2 may comprise a polypeptide comprising at least about 90% homology or identity to HAPs ID NO: 4. In some cases, the THBS2 may comprise a polypeptide comprising at least about 90% homology or identity to amino acids 19-1172 of HAPs ID NO: 4. In some cases, the THBS2 is expressed in a mouse myeloma cell line.

In some embodiments, the polypeptide may comprise THBS4. In some cases, the THBS4 may comprise a polypeptide comprising at least about 90% homology or identity to HAPs ID NO: 8. In some cases, the THBS4 may comprise a polypeptide comprising at least about 90% homology or identity to amino acids 27-961 of HAPs ID NO: 8. In some cases, the THBS4 is expressed in Chinese hamster ovary cell.

In some embodiments, the polypeptide may comprise IGF2. In some cases, the IGF2 may comprise a polypeptide comprising at least about 90% homology or identity to HAPs ID NO: 11. In some cases, the IGF2 may comprise a polypeptide comprising at least about 90% homology or identity to amino acids 25-91 of HAPs ID NO: 11. In some cases, the IGF2 is expressed in a bacterial cell. In some embodiments, the bacterial cell is E. coli.

In some embodiments, the polypeptide may comprise IL-15. In some cases, the IL-15 may comprise a polypeptide comprising at least about 90% homology or identity to HAPs ID NO: 10. In some cases, the IL-15 may comprise a polypeptide comprising at least about 90% homology or identity to amino acids 49-162 of HAPs ID NO: 10. In some cases, the IL-15 is expressed in a bacterial cell. In some embodiments, the bacterial cell is E. coli.

In some embodiments, the polypeptide may comprise THBS1. In some cases, the THBS1 may comprise a polypeptide comprising at least about 90% homology or identity to HAPs ID NO: 9. In some cases, the THBS1 may comprise a polypeptide comprising at least about 90% homology or identity to amino acids 19-1170 of HAPs ID NO: 9. In some cases, the THBS1 is expressed in a mouse myeloma cell line.

In some embodiments, the polypeptide may comprise BMP7. In some cases, the BMP7 may comprise a polypeptide comprising at least about 90% homology or identity to HAPs ID NO: 72. In some cases, the BMP7 may comprise a polypeptide comprising at least about 90% homology or identity to HAPs ID NO: 72. In some cases, the BMP7 is expressed in a mouse myeloma cell line.

In some embodiments, the polypeptide may comprise IL-15, and the composition further may comprise THBS2. In some embodiments, the polypeptide may comprise IL-15, and the composition further may comprise THBS4. In some embodiments, the polypeptide may comprise THBS4, and the composition further may comprise THBS2. In some embodiments, the polypeptide may comprise IL-15, and the composition further may comprise THBS2 and THBS4.

In some embodiments, the polypeptide may comprise IGF2, and the composition further may comprise THBS2. In some embodiments, the polypeptide may comprise IGF2, and the composition further may comprise THBS4. In some embodiments, the polypeptide may comprise THBS4, and the composition further may comprise THBS2. In some embodiments, the polypeptide may comprise IGF2, and the composition further may comprise THBS2 and THBS4.

### Schedules Routes of Administration and Amounts

The HAPs can be administered by any suitable route such as, for example, subcutaneous, intravenous, or intramuscular. The HAPs are administered on a suitable dosage schedule, for example, weekly, twice weekly, monthly, twice monthly, once every three weeks, or once every four weeks. The HAPs can be administered in any therapeutically effective amount. The therapeutically acceptable amount is about 0.001 mg/kg to about 1 mg/kg. The therapeutically acceptable amount is about 0.001 mg/kg to about 0.002 mg/kg, about 0.001 mg/kg to about 0.005 mg/kg, about 0.001 mg/kg to about 0.01 mg/kg, about 0.001 mg/kg to about 0.02 mg/kg, about 0.001 mg/kg to about 0.05 mg/kg, about 0.001 mg/kg to about 0.1 mg/kg, about 0.001 mg/kg to about 0.2 mg/kg, about 0.001 mg/kg to about 0.5 mg/kg, about 0.001 mg/kg to about 1 mg/kg, about 0.002 mg/kg to about 0.005 mg/kg, about 0.002 mg/kg to about 0.01 mg/kg, about 0.002 mg/kg to about 0.02 mg/kg, about 0.002 mg/kg to about 0.05 mg/kg, about 0.002 mg/kg to about 0.1 mg/kg, about 0.002 mg/kg to about 0.2 mg/kg, about 0.002 mg/kg to about 0.5 mg/kg, about 0.002 mg/kg to about 1 mg/kg, about 0.005 mg/kg to about 0.01 mg/kg, about 0.005 mg/kg to about 0.02 mg/kg, about 0.005 mg/kg to about 0.05 mg/kg, about 0.005 mg/kg to about 0.1 mg/kg, about 0.005 mg/kg to about 0.2 mg/kg, about 0.005 mg/kg to about 0.5 mg/kg, about 0.005 mg/kg to about 1 mg/kg, about 0.01 mg/kg to about 0.02 mg/kg, about 0.01 mg/kg to about 0.05 mg/kg, about 0.01 mg/kg to about 0.1 mg/kg, about 0.01 mg/kg to about 0.2 mg/kg, about 0.01 mg/kg to about 0.5 mg/kg, about 0.01 mg/kg to about 1 mg/kg, about 0.02 mg/kg to about 0.05 mg/kg, about 0.02 mg/kg to about 0.1 mg/kg, about 0.02 mg/kg to about 0.2 mg/kg, about 0.02 mg/kg to about 0.5 mg/kg, about 0.02 mg/kg to about 1 mg/kg, about 0.05 mg/kg to about 0.1 mg/kg, about 0.05 mg/kg to about 0.2 mg/kg, about 0.05 mg/kg to about 0.5 mg/kg, about 0.05 mg/kg to about 1 mg/kg, about 0.1 mg/kg to about 0.2 mg/kg, about 0.1 mg/kg to about 0.5 mg/kg, about 0.1 mg/kg to about 1 mg/kg, about 0.2 mg/kg to about 0.5 mg/kg, about 0.2 mg/kg to about 1 mg/kg, or about 0.5 mg/kg to about 1 mg/kg. The therapeutically acceptable amount is about 0.001 mg/kg, about 0.002 mg/kg, about 0.005 mg/kg, about 0.01 mg/kg, about 0.02 mg/kg, about 0.05 mg/kg, about 0.1 mg/kg, about 0.2 mg/kg, about 0.5 mg/kg, or about 1 mg/kg. The therapeutically acceptable amount is at least about 0.001 mg/kg, about 0.002 mg/kg, about 0.005 mg/kg, about 0.01 mg/kg, about 0.02 mg/kg, about 0.05 mg/kg, about 0.1 mg/kg, about 0.2 mg/kg, or about 0.5 mg/kg. The therapeutically acceptable amount is at most about 0.002 mg/kg, about 0.005 mg/kg, about 0.01 mg/kg, about 0.02 mg/kg, about 0.05 mg/kg, about 0.1 mg/kg, about 0.2 mg/kg, about 0.5 mg/kg, or about 1 mg/kg. The therapeutically acceptable amount is about 0.1 mg/kg to about 50 mg/kg. The therapeutically acceptable amount is about 0.1 mg/kg to about 0.2 mg/kg, about 0.1 mg/kg to about 0.5 mg/kg, about 0.1 mg/kg to about 1 mg/kg, about 0.1 mg/kg to about 2 mg/kg, about 0.1 mg/kg to about 5 mg/kg, about 0.1 mg/kg to about 10 mg/kg, about 0.1 mg/kg to about 20 mg/kg, about 0.1 mg/kg to about 50 mg/kg, about 0.2 mg/kg to about 0.5 mg/kg, about 0.2 mg/kg to about 1 mg/kg, about 0.2 mg/kg to about 2 mg/kg, about 0.2 mg/kg to about 5 mg/kg, about 0.2 mg/kg to about 10 mg/kg, about 0.2 mg/kg to about 20 mg/kg, about 0.2 mg/kg to about 50 mg/kg, about 0.5 mg/kg to about 1 mg/kg, about 0.5 mg/kg to about 2 mg/kg, about 0.5 mg/kg to about 5 mg/kg, about 0.5 mg/kg to about 10 mg/kg, about 0.5 mg/kg to about 20 mg/kg, about 0.5 mg/kg to about 50 mg/kg, about 1 mg/kg to about 2 mg/kg, about 1 mg/kg to about 5 mg/kg, about 1 mg/kg to about 10 mg/kg, about 1 mg/kg to about 20 mg/kg, about 1 mg/kg to about 50 mg/kg, about 2 mg/kg to about 5 mg/kg, about 2 mg/kg to about 10 mg/kg, about 2 mg/kg to about 20 mg/kg, about 2 mg/kg to about 50 mg/kg, about 5 mg/kg to about 10 mg/kg, about 5 mg/kg to about 20 mg/kg, about 5 mg/kg to about 50 mg/kg, about 10 mg/kg to about 20 mg/kg, about 10 mg/kg to about 50 mg/kg, or about 20 mg/kg to about 50 mg/kg. The therapeutically acceptable amount is about 0.1 mg/kg, about 0.2 mg/kg, about 0.5 mg/kg, about 1 mg/kg, about 2 mg/kg, about 5 mg/kg, about 10 mg/kg, about 20 mg/kg, or about 50 mg/kg. The therapeutically acceptable amount is at least about 0.1 mg/kg, about 0.2 mg/kg, about 0.5 mg/kg, about 1 mg/kg, about 2 mg/kg, about 5 mg/kg, about 10 mg/kg, or about 20 mg/kg. The therapeutically acceptable amount is at most about 0.2 mg/kg, about 0.5 mg/kg, about 1 mg/kg, about 2 mg/kg, about 5 mg/kg, about 10 mg/kg, about 20 mg/kg, or about 50 mg/kg.

### Nucleic Acids

TABLE 3
Nucleic acid sequences.

| Protein | Size | | Protein | mRNA | | |
|---|---|---|---|---|---|---|
| Name | (AA) | UniProtKB | accession# | accession# | Gene ID | SEQ ID |
| FGF17 | 216 | O60258.1 | NP_003858.1 | NM_003867.4 | 8822 | 45 |
| THBS1 | 1170 | P07996.2 | NP_003237.2 | NM_003246.4 | 7057 | 46 |
| THBS2 | 1172 | P35442.2 | NP_003238.2 | NM_003247.3 | 7058 | 47 |
| THBS4 | 961 | P35443.2 | NP_003239.2 | NM_003248.6 | 7060 | 48 |
| IGF2 | 180 | P01344.1 | NP_000603.1 | NM_000612.6 | 3481 | 49 |
| IL15 | 162 | P40933.1 | NP_000576.1 | NM_000585.5 | 3600 | 50 |
| IGFBP7 | 282 | Q16270.1 | NP_001544.1 | NM_001553.3 | 3490 | 51 |
| VTN | 478 | P04004 | NP_000629.3 | NM_000638.4 | 7448 | 52 |
| POSTN | 836 | Q15063.2 | NP_006466.2 | NM_006475.3 | 10631 | 53 |
| PDGFRL | 375 | Q15198.1 | NP_006198.1 | NM_006207.2 | 5157 | 54 |
| ANOS1 | 3730 | P23352 | NP_000207.2 | NM_000216.4 | 3730 | 57 |
| BMP7 | 431 | P18075 | NP_001710.1 | NM_001719.3 | 655 | 73 |

Described herein is a composition that may comprise nucleic acids that encode the HAPs described herein. The nucleic acids are exogenous. The nucleic acid is a plasmid. The nucleic acid is a viral vector. The viral vector is an adenovirus, lentivirus, retrovirus, adeno-associated virus, or vaccinia virus. The nucleic acid may comprise RNA. The nucleic acid encodes any of the polypeptides listed in Table 1 or Table 2, or VTN, STC2, AGRN, POSTN, FGF17, THBS2, FST, THBS1, IL-15, IGF2, THBS4, or BMP7. The nucleic acid encodes any one or more polypeptides described herein. Nucleic acids according to this description can comprise additional nucleic acid sequences sufficient to propagate the vector or express a polypeptide encoded by the vector. The nucleic acid may comprise a universal promoter, such as the CMV promoter, or an inducible promoter system such as a TET_{ON}, TET_{OFF} or GAL4. The nucleic acid is expressed via a tissue specific promoter or one compatible with a eukaryotic or prokaryotic cellular expression system. The nucleic acid can further comprise a sequence encoding a suitable purification tag (e.g., HIS-tag, V5, FLAG, MYC).

### Production of Heparin-Associated or Heparin-Binding Polypeptides

Once a polypeptide is determined as a heparin-associated or heparin-binding polypeptide it can be purified or synthesized in any suitable manner. A nucleic acid encoding the polypeptide can be cloned into a suitable vector and expressed in a suitable cellular system. The cellular system is a prokaryotic cell system. The cellular system is a eukaryotic cell system. The cellular system is a mammalian cell system. The supernatants from such an expression system can be subjected to one or more purification steps involving centrifugation, ultracentrifugation, filtration, diafiltration, tangential-flow filtration, dialysis, chromatography (e.g., cation exchange, ion exchange, hydrophobic interaction, reverse phase, affinity, or size exclusion). The polypeptides can be purified to an extent suitable for human administration. Additionally, polypeptides can be synthesized for inclusion in a formulation to be administered to a human subject. The polypeptides can be produced by a suitable peptide synthesis method, such as solid-phase synthesis.

### Master Cell Bank and Transgenic Cells

Described herein is a master cell bank comprising a cell that may comprise a nucleic acid encoding one or more HAPs integrated into its genome creating a transgenic cell-line. The master cell bank may comprise a plurality of cells that each comprise a nucleic acid encoding a HAP. The nucleic acid is maintained extrachromosomally on a plasmid or yeast artificial chromosome. The nucleic acid is integrated into a chromosomal location. The cell is a yeast cell. The yeast is Pichia pastoris or Saccharomyces cerevisiae. The cell is a mammalian cell. The mammalian cell is a 293T cell or derivative thereof (e.g., 293T-Rex). The cell is a bacterial cell.

The transgenic mammalian, yeast, or bacterial cell is a master cell bank that may comprise a cryopreservative suitable for freezing to at least about −80° or below. The master cell bank may comprise glycerol at between about 10 and about 30%, and is suitable for long-term storage at about −80° or below. The master cell bank can preserve a transgenic mammalian, yeast, or bacterial strain for at least 1, 2, 3, 4, 5, 6, 7, 8, 9, 10 or more years.

### Pharmaceutically Acceptable Excipients, Carriers, and Diluents

The HAP(s) described herein can be administered in a pharmaceutical composition that may comprise one or more pharmaceutically acceptable excipients, carriers, or diluents. The exact components can differ based upon the preferred route of administration. The excipients used in a pharmaceutical composition can provide additional function to the polypeptide by making the polypeptide suitable for a particular route of administration (e.g., intravenous, topical, subcutaneous, or intramuscular), increasing polypeptide stability, increasing penetration of a desired tissue (e.g., muscle or skin), increasing residence time at particular site, increasing solubility, enhancing the efficacy of the polypeptide, and/or reducing inflammatory reactions coincident with administration.

The HAP(s) described herein are included in a pharmaceutical composition with a solubilizing emulsifying, or dispersing agent. The solubilizing agent can allow high-concentration solutions of HAPs that exceed at least about 2 mg/mL, 5 mg/mL, 10 mg/mL, 15 mg/mL, or 20 mg/mL. Carbomers in an aqueous pharmaceutical composition serve as emulsifying agents and viscosity modifying agents. The pharmaceutically acceptable excipient may comprise or consists of a carbomer. The carbomer may comprise or consists of carbomer 910, carbomer 934, carbomer 934P, carbomer 940, carbomer 941, carbomer 1342, or combinations thereof. Cyclodextrins in an aqueous pharmaceutical composition serve as solubilizing and stabilizing agents. The pharmaceutically acceptable excipient may comprise or consists of a cyclodextrin. The cyclodextrin may comprise or consists of alpha cyclodextrin, beta cyclodextrin, gamma cyclodextrin, or combinations thereof. Lecithin in a pharmaceutical composition may serve as a solubilizing agent. The solubilizing agent may comprise or consists of lecithin. Poloxamers in a pharmaceutical composition serve as emulsifying agents, solubilizing agents, and dispersing agents. The pharmaceutically acceptable excipient may comprise or consists of a poloxamer. The poloxamer may comprise or consists of poloxamer 124, poloxamer 188, poloxamer 237, poloxamer 338, poloxamer 407, or combinations thereof. Polyoxyethylene sorbitan fatty acid esters in a pharmaceutical composition serve as emulsifying agents, solubilizing agents, surfactants, and dispersing agents. The pharmaceutically acceptable excipient may comprise or consists of a polyoxyethylene sorbitan fatty acid ester. The polyoxyethylene sorbitan fatty acid ester may comprise or consists of polysorbate 20, polysorbate 21, polysorbate 40, polysorbate 60, polysorbate 61, polysorbate 65, polysorbate 80, polysorbate 81, polysorbate 85, polysorbate 120, or combinations thereof. Polyoxyethylene stearates in a pharmaceutical composition serve as emulsifying agents, solubilizing agents, surfactants, and dispersing agents. The pharmaceutically acceptable excipient may comprise or consists of a polyoxyethylene stearate. The polyoxyethylene stearate may comprise or consists of polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 100 stearate, polyoxyl 150 stearate, polyoxyl 4 distearate, polyoxyl 8 distearate, polyoxyl 12 distearate, polyoxyl 32 distearate, polyoxyl 150 distearate, or combinations thereof. Sorbitan esters in a pharmaceutical composition serve as emulsifying agents, solubilizing agents, and non-ionic surfactants, and dispersing agents. The pharmaceutically acceptable excipient may comprise or consists of a sorbitan ester. The sorbitan ester may comprise or consists of sorbitan laurate, sorbitan oleate, sorbitan palmitate, sorbitan stearate, sorbitan trioleate, sorbitan sesquioleate, or combinations thereof. Solubility may be achieved with a protein carrier. The protein carrier may comprise recombinant human albumin.

The HAP(s) of the current disclosure are formulated to increase stability. Polypeptides in aqueous formulations may require stabilization to prevent degradation. The stabilizer may comprise pH buffers, salts, amino acids, polyols/disaccharides/polysaccharides, liposomes, surfactants, antioxidants, reducing agents, or chelating agents. The stabilizer may comprise or consists of a polyol/non-reducing sugar. The non-reducing sugar may comprise or consists of sucrose, mannitol, trehalose, raffinose, stachyose, xylitol, starch, verbascose, or combinations thereof. Polypeptides can be encapsulated in liposomes to increase stability. The stabilizer may comprise or consists of liposomes. The liposomes comprise or consists of ipalmitoylphosphatidylcholine (DPPC) liposomes, phosphatidylcholine:cholesterol (PC:Chol) (70:30) liposomes, or dipalmitoylphosphatidylcholine: dipalmitoylphosphatidylserine (DPPC:DPPS) liposomes (70:30). Non-ionic surfactants can increase the stability of a polypeptide. The stabilizer may comprise or consists of a non-ionic surfactant. The non-ionic surfactant may comprise or consists of polysorbates (e.g., poly sorbate 80, poly sorbate 20), alkylsaccharides alkyl ethers and alkyl glyceryl ethers, polyoxyethelene (4) lauryl ether; polyoxyethylene cetyl ethers, polyoxyethylene stearyl ethers, sorbitan fatty acid esters, polyoxyethylene fatty acid esters, or combinations thereof The polypeptide is formulated with a protein surfactant, such as recombinant human serum albumin as a stabilizer. Antioxidants or reducing agents can increase the stability of a polypeptide. The stabilizer may comprise or consists of an antioxidant or reducing agent. The reducing agent may comprise or consists of dithiothreitol, ethylenediaminetetraacetic acid, 2-Mercaptoethanol, Tris(2-carboxyethyl)phosphine hydrochloride, Tris(hydroxypropyl)phosphine, or combinations thereof. The antioxidant may comprise or consists of methionine, ascorbic acid, citric acid, alpha tocopherol, sodium bisulfite, ascorbyl palmitate, erythorbic acid, or combinations thereof. Chelating agents can stabilize polypeptides by reducing the activity of proteases. The stabilizer may comprise or consists of a chelating agent. The chelating agent may comprise or consists of ethylenediaminetetraacetic acid (EDTA), ethylene glycol-bis(O-aminoethyl ether)-N,N,N′,N′-tetraacetic acid (EGTA), metal complexes (e.g. Zn-protein complexes), or combinations thereof. Buffer agents can stabilize polypeptides by reducing the acid hydrolysis of polypeptides. The stabilizer may comprise or consists of a buffer agent. The buffer agent may comprise or consists sucrose octa-sulfate, ammonium carbonate, ammonium phosphate, boric acid, sodium citrate, potassium citrate, lactic acid, 3-(N-morpholino)propanesulfonic acid (MOPS), 2-(N-morpholino)ethanesulfonic acid (MES), hydroxymethylaminomethane (Tris), calcium carbonate, calcium phosphate or combinations thereof.

The HAP(s) also may be entrapped in or associated with microcapsules prepared, for example, by coacervation techniques or by interfacial polymerization (for example, hydroxymethylcellulose or gelatin-microcapsules and poly-(methylmethacylate) microcapsules, respectively), in colloidal delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The HAP(s) of the current disclosure may be formulated or delivered with an anti-inflammatory agent. The anti-inflammatory agent may comprise or consists of a corticosteroid. The corticosteroid may comprise or consists of hydrocortisone, cortisone, ethamethasoneb (Celestone), prednisone (Prednisone Intensol), prednisolone (Orapred, Prelone), triamcinolone (Aristospan Intra-Articular, Aristospan Intralesional, Kenalog), methylprednisolone (Medrol, Depo-Medrol, Solu-Medrol), or dexamethasone (Dexamethasone Intensol). In certain emboidments, the anti-inflammatory may comprise or consists of a non-steroidal anti-inflammatory (NSAID). The NSAID may comprise or consists of aspirin, celecoxib, diclofenac, diflunisal, etodolac, ibuprofen, indomethacin, ketoprofen, ketorolac, nabumetone, naproxen, oxaprozin, piroxicam, salsalate, sulindac, or tolmetin.

The HAP(s) of the current disclosure are included in a pharmaceutical composition suitable for intravenous administration comprising one or more pharmaceutically acceptable excipients, carriers, and diluents. The polypeptides of the current disclosure are administered suspended in a sterile solution. The solution is one commonly used for administration of biological formulations, and may comprise, for example, about 0.9% NaCl or about 5% dextrose. The solution further may comprise one or more of buffers, for example, acetate, citrate, histidine, succinate, phosphate, potassium phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine, histidine, leucine, or arginine; antioxidants, for example, ascorbic acid, methionine; or chelating agents, for example, EDTA, or EGTA.

The HAP(s) of the current disclosure are included in a pharmaceutical composition suitable for intramuscular or subcutaneous administration comprising one or more pharmaceutically acceptable excipients, carriers, and diluents. Formulations suitable for intramuscular or subcutaneous injection can include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include ethanol, polyols (inositol, propyleneglycol, polyethylene-glycol, glycerol, cremophor and the like) and suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

The HAP(s) of the current disclosure are formulated for topical administration as a cream, gel, paste, ointment, or emulsion. Excipients in a cream, gel, paste, ointment, or emulsion can comprise gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches.

The excipient used with the HAP(s) described herein will allow for storage, formulation, or administration of highly concentrated formulations. A highly concentrated HAP(s) may comprise at least about 1, 2, 3, 4, 5, 6, 7, 8, 9, 10, 15, 20, 25, 20, 25, 40, 45, 50 or more milligrams per milliliter.

The polypeptides of the current disclosure are shipped/stored lyophilized and reconstituted before administration. Lyophilized HAP formulations may comprise a bulking agent such as, mannitol, sorbitol, sucrose, trehalose, and dextran 40. The lyophilized formulation can be contained in a vial comprised of glass. The HAPs when formulated, whether reconstituted or not, can be buffered at a certain pH, generally less than 7.0. The pH can be between 4.5 and 6.5, 4.5 and 6.0, 4.5 and 5.5, 4.5 and 5.0, or 5.0 and 6.0.

### EXAMPLES

The following illustrative examples are representative of embodiments of the compositions and methods described herein and are not meant to be limiting in any way.

### Example 1—Isolation of Heparin-Associated Polypeptides

hESC secretome collection (differentiated vs undifferentiated): Human embryonic or induced pluripotent stem cells (H1, H9, H7 lines, and 2 iPSC lines derived from 1 healthy young adult female (18-25 years) and 1 aged female (greater than 65 year) donor), were cultured in triplicate on 10 cm plates on diluted Matrigel (1:30), in mTeSR-1 (Stem Cell Technologies), for a total media volume of 10 mL per plate. Another triplicate set of hPSCs/iPSCs were cultured on 10 cm plates and differentiated after plating in mTeSR-1 by changing the medium to DMEM/F12 with 10% Bovine Growth Serum (Hyclone), and culturing for an additional 7 days. hPSCs/iPSCs and differentiated hPSCs/iPSCs (6 plates in total) were washed twice with Opti-MEM (Gibco) and then cultured in Opti-MEM for 16 hours. 10 ml media was then collected per plate as hPSCs/iPSCs—secretome or differentiated hPSCs/iPSCs—secretome containing media. Media was spun for 5 min at 1000 g and transferred to new tubes to remove cell debris, aliquoted and flash frozen at 2 mL per plate as 0.5 mL aliquots and stored at −80 C; remaining 8 mL/plate was used immediately for heparin-associated protein purification.

### Heparin-Associated Protein Purification

10 mL of Heparin-Agarose Type I Beads (H 6508, Sigma Aldrich) was washed with molecular grade water and preconditioned in 1 mL OptiMEM as recommended by manufacturer. 8-9 ml secretome containing media was incubated with 1 ml Heparin-Agarose Beads for 2 hours shaking at 4° C. to allow binding. Remaining heparin-depleted hPSCs/iPSCs-conditioned medium or differentiated hPSCs/iPSCs-conditioned medium was aliquoted in 15 mL tubes, flash frozen and stored at −80 C to serve as negative controls for efficacy testing. Protein bound heparin beads were washed twice via a 10-minute incubation at 4° C. in 1 mL sterile PBS+0.05% Tween. Proteins were eluted twice for 15 minutes at 4° C. in 400 μl of elution buffer A (Eluted-A) (0.01M HEPES pH 7.5+1.5M NaCl+0.1% BSA) per 10 cm plate for the first two plates, or elution buffer B (Eluted-B) lacking BSA (0.01M HEPES pH 7.5+1.5M NaCl) for the 3rd 10 cm plate, to collect proteins in a total of 800 μl of elute per original plate. The proteins were desalted by diffusion dialysis (3500 MWCO) (by a 2-hour dialysis shaking at 4 C in 500 ml McCoy's 5A Medium or similar tissue culture medium (Gibco) followed by overnight (not more than 16 hours) dialysis shaking at 4 C in 200 ml OptiMEM (Gibco). The collected eluate was aliquoted in appropriately capped tubes, flash frozen and stored at −80 C.

### Secretome Heparin-Associated Fraction Validation Assays:

BCA assay (Pierce) was performed for total protein yield in the eluate using 2 ul per sample in triplicate according to manufacturer's instructions from each sample.

SDS-PAGE Silver Stain/SDS-PAGE Coomassie was performed for protein integrity and rough MW analysis (loading <5-10 μg per lane for each sample).

### Mouse Myoblast Proliferation Assay

Reduced regeneration from an individual's tissue progenitor cells is a hallmark of aging, therefore assays that measure mitogenic capacity in tissue progenitor cells serve as a read-out for potential success of any given heparin-associated polypeptide (HAP) as a regenerative factor. Measuring the increased proliferation rate of treated mouse or human muscle progenitor cells will provide good basis for potentially therapeutic regenerative factors for treating individuals who have suffered illness, injury, or who possess genetic or developmental defects leading to premature tissue loss, wasting, or weakening. As a control, the assay will also be performed on proteins purified from differentiated cells, which result in no in myoblast proliferation, cultured in medium conditioned by differentiated cells, or purified heparin-associated fractions.

Mouse muscle progenitor cells (early passage myoblasts) were cultured and expanded in mouse growth medium: Ham's F-10 (Gibco), 20% Bovine Growth Serum (Hyclone), 5 ng/mL FGF2 and 1% penicillin-streptomycin on Matrigel coated plates (1:300 matrigel: PBS), at 37° C. and 5% CO2. For experimental conditions, cells were plated at 40,000 cells/well on Matrigel coated 8-well chamber slides in 250-500 μL medium per well (1:100 matrigel: PBS) in mouse fusion medium: DMEM (Gibco)+2% horse serum (Hyclone). One hour after plating, mouse myoblasts were treated with 50% respective medias:

TABLE 4
8-well Chamber Slide A: Eluted HAPs from H9/H7 hPSCs and 2
iPSC lines - 4 slides total, 1 for each cell line tested.

| | | | |
|---|---|---|---|
| Fusion | 50% FM/ | 50% FM/50% | 50% FM/50% |
| Media (FM) | 50% Eluted-A | Differentiated | Differentiated |
| (250 μL) | Heparin- | hPSC- | hPSC- |
| | associated | conditioned | conditioned |
| | proteins | OptiMem | OptiMem |
| 50% FM | 50% FM/50% | 50% FM/50% | 50% FM/ |
| (125 μL)/ | hPSC- | Heparin- | 50% Eluted-B |
| 50% Growth | conditioned | depleted | Heparin- |
| Media | OptiMEM | hESC- | associated |
| (125 μL) | | conditioned | proteins |
| | | OptiMEM | (no BSA) |

TABLE 5
Assay for eluted heparin-associated proteins

| | | | |
|---|---|---|---|
| (Control). 50% | 50% FM/ | 50% FM/50% | 50% FM/ |
| FM/ | 50% Eluted-A | Differentiated | 50% Eluted-B |
| 50% OptiMEM | Heparin- | hPSC-conditioned | Heparin- |
| | associated | OptiMem | associated |
| | proteins | | proteins (no BSA) |

TABLE 6
8-well Chamber Slide B: Eluted Heparin-
associated Protein Serial Dilution

| | | | |
|---|---|---|---|
| Fusion Media | 50% FM/ | 75% FM/25% | 75% FM/12.5% |
| (FM) | 50% Eluted-A | Eluted-A | Eluted-A |
| (250 μL) | Heparin- | Heparin- | Heparin- |
| | associated | associated | associated |
| | protein | protein | protein |
| 81.25% FM/ | 84.375% FM/ | 98.44% FM/ | 75% FM/ |
| 6.25% Eluted- | 3.125% Eluted- | 1.56% Eluted- | 25% hPSC- |
| A Heparin- | A Heparin- | A Heparin- | conditioned |
| associated | associated | associated | OptiMEM |
| protein | protein | protein | |

Mouse myoblasts were cultured for 24 hours in the above conditions, at 37° C. in 10% CO₂ incubator. BrdU (300 μM) in DMSO was added for 2 hours prior to fixation with cold 70% ethanol and stored at 4° C. until staining.

### Quantifying Regenerative Index

Following permeabilization in PBS+0.25% Triton X-100, antigen retrieval was performed via a 10-minute 4 N HCl treatment followed by PBS washes. Primary staining was performed overnight at 4° C. in PBS+2% FBS. Primary antibodies include: a species-specific monoclonal antibody for mouse anti-embryonic Myosin Heavy Chain (eMyHC, hybridoma clone 1.652, Developmental Studies Hybridoma Bank) and Rat-anti-BrdU (Abcam Inc. ab6326). Secondary staining with fluorophore-conjugated, species-specific antibodies (Donkey anti-Rat-488, #712-485-150; Donkey anti-Mouse-488, #715-485-150; all secondary antibodies from Jackson ImmunoResearch) was performed for 1 hour at room temperature at a 1:500 dilution in PBS+2% FBS. Nuclei are visualized by Hoechst staining.

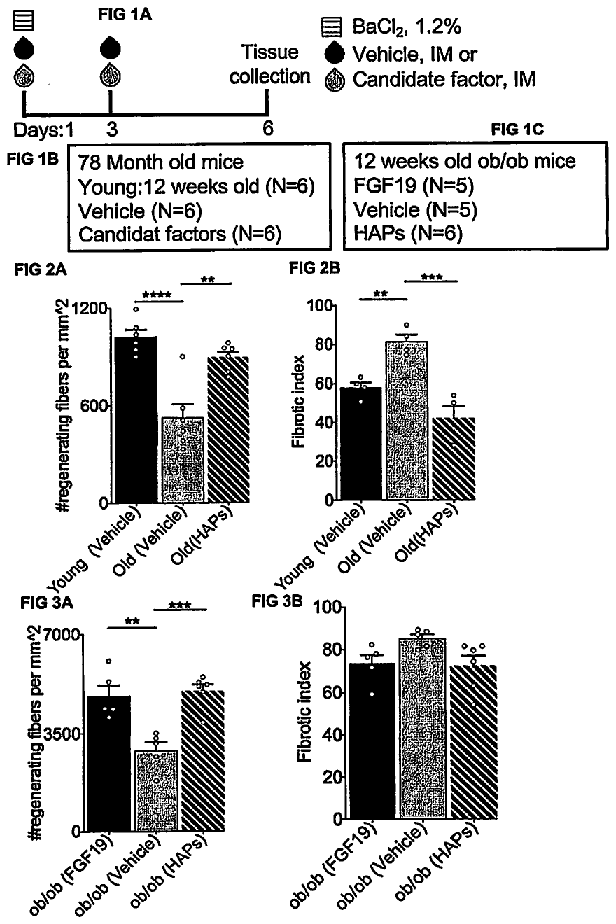

In one experiment, old mice were injured with BaCl₂ and injected with the HAPs. After 5 days, mice were euthanized, and the muscles were analyzed for regeneration. As depicted in FIG. 1, the muscles of old mice that had received an injection of the heparin-binding proteins had a higher regenerative index than the muscles of the untreated old mice.

For cell quantification, 5 images per well were collected at 20× in each of the channels as well as DIC to achieve at least 2000 imaged cells per condition. Using the Hoechst stain to tally cell number, the percent of cells positive for BrdU and eMyHC were tabulated and reported.

Human muscle progenitor cells (myoblasts) were similarly activated to proliferate when conditioned with hPSC-secreted heparin-associated proteins. Proliferation assays were performed on human myoblasts to test protein candidate factors for enhanced precursor cell activity in an in vitro screening assay. Conditions for culturing human muscle cells were optimized to reflect the slower rate of growth and differentiation of human muscle cells, where early passage human myoblasts were cultured for 72 hours with daily medium changes rather than 24 hours, and pulsed for 4 hours with BrdU instead of 2 hours.

### Example 2—Characterization of the Protein Components of the Heparin Bead Binding hPSC Secretomes

### Protein Quantification

The protein concentration in the eluted sample was determined using the bicinchoninic acid (BCA) protein assay (Thermo Fisher Scientific, Waltham, MA). The protocol was performed as follows: A volume containing 100 μg protein was extracted and disulfide bonds were reduced with 5 mM tris-(2-carboxyethyl)-phosphine (TCEP), at room temperature for 25 min, and alkylated with 10 mM iodoacetamide at room temperature for 30 min in the dark). Excess iodoacetamide was quenched with 15 mM dithiothreitol (room temperature, 15 min in the dark). At this point the samples were split, with 20 μg analyzed immediately via SDS-PAGE Silver Stain, 20 μg saved for SDS-PAGE Coomassie stained gel band analysis, and 60 μg proceeded to in-solution mass spectrometry sample preparation.

### Quantify the Size Distribution of Proteins

Silver staining provides a sensitive, rapid, low cost way to survey the complexity and general molecular weight distribution of the proteins in a complex mixture. By running a matched sample treated to remove glycans, the presence of this PTM common secreted proteins can be determined by the resulting shift in apparent molecular weight. Additional rounds of selective glycosylation reactions can then be run to gain insight into the identity and structure of glycan modifications on proteins of interest. Five micrograms of sample can be removed and treated with Protein Deglycosylation Mix II (NEB) to remove all N-linked and simple O-linked glycans as well as some complex O-linked glycans, which can be visualized by molecular weight shifts relative to an untreated control on a silver stained SDS-PAGE gel.

A 4-12% acrylamide gel (BioRad) in 1×MOPS buffer was loaded gel with samples (>0.20 ug/lane) and ladder (as per manufacturer's instructions), run at 200V for 45 minutes or until sample front neared the bottom of the gel, and incubated in 50% methanol/50% LC grade water >1 hour. Stain solution was prepared adding a solution of 0.8 g AgNO3 in 4 mL LC grade H₂O dropwise into a solution of 1 mL 0.36% NaOH+1.4 mL 14.8M ammonium hydroxide under constant stirring followed by the addition of LC grade water to a final volume of 100 mL. Gel staining proceeded by incubating gel in stain solution for 15 minutes, before washing twice with LC grade water, allowing 5-8 minutes of incubation per wash step. The silver stain was developed by incubation in a solution of 0.25 mL 1% citric acid+25 uL 37% formaldehyde in 50 mL LC grade water for 10-15 minutes in the dark (or until desired density was achieved). Developer solution was removed, and the gel washed with LC grade water to slow development for an imaging series, or development was stopped by incubation in a solution of 45% methanol, 10% acetic acid.

### In-Solution Mass Spectrometry Sample Preparation

Methanol-chloroform precipitation was performed prior to protease digestion (a standard trichloroacetic acid-based precipitation protocol would be substituted here if protein yield from the heparin bead eluates are below 25 μg total). In brief, four parts neat methanol was added to each sample and vortexed, one part chloroform was added to the sample and vortexed, and three parts water was added to the sample and vortexed. The sample was centrifuged at 4,000 RPM for 15 min at room temperature and subsequently washed twice with 100% methanol, prior to air-drying. Samples were resuspended in 50 mM HEPES pH 8.5 and digested at room temperature for 12 hrs with LysC protease at a 100:1 protein-to-protease ratio. Trypsin was then added at a 100:1 protein-to-protease ratio and the reaction was incubated 6 hours at 37° C. Peptide concentrations in the digests were measured using the Quantitative Colorimetric Peptide assay kit (Pierce). From each sample 10 μg of peptide digestion solution was taken and enzymatic activity quenched with formic acid to a final pH of <2 before de-salting via C-18 Stagetips, using a standard formic acid/acetonitrile buffer system. Stagetips were eluted directly into autosampler vials in a buffer of 70% acetonitrile and 1% formic acid, dried in a vacuum concentrator, and stored at −80 C until being resuspended to ˜1 ug/μl of Buffer A (typically ˜0.2% formic acid, 5% acetonitrile) for mass spectrometry analysis.

### SDS-PAGE Coomassie and In-Gel Band Mass Spectrometry Sample Preparation

A gel-based sample preparation pipeline may be employed if the abundance distribution of the sample is heavily skewed, or where only a few species of proteins account for a substantial majority of the molecules in the sample. This size-based separation method has been shown to effectively improve depth of proteomic coverage in biochemically purified protein mixtures.

Briefly, the protocol begins by running >20 μg per lane of sample out on an SDS-PAGE as in the Silver Stain method above, staining and destaining by Coomassie as per manufacturer's instructions, excising sections of the gel containing potentially interesting proteins, and cutting excised gel sections into 1 mm×1 mm squares. Ensure gel pieces are at neutral pH by adding 50-100 μl 100 mM Ammonium bicarbonate, let sit for 10 minutes and discard. Wash gel pieces with 100-150 μl 50 mM Ammonium bicarbonate/50% acetonitrile for 10 minutes, vortexing every 5 minutes to dehydrate. Depending on intensity of stain, repeat step 9 until the gel pieces are clear. Discard solution phase and dry samples in speed vac for 5-10 minutes. To digest proteins add 5 pmol sequencing grade trypsin (Promega Corp.) in 50 mM Ammonium bicarbonate and 0.02% Protease Max to each sample and incubate overnight in 37° C. on a shaking heatblock. Spin down samples at 1000G for 2 minutes, pull off all liquid, and transfer to a glass autosampler vial. Add 40-50 μl 1% formic acid, 66% acetonitrile 33% 100 mM Ammonium bicarbonate and incubate for 10 minutes at 37° C. to increase peptide release from gel. Spin at 10,000G for 2 minutes to pellet insoluble protein or detergent degradation production. Extract all solution being sure to avoid pellet areas and combine into autosampler vial. Speed vac total combined extracts to dryness and store at −80 C until being resuspended to ˜1 μg/μl of Buffer A (typically ˜0.2% formic acid, 5% acetonitrile) for mass spectrometry analysis.

nHPLC-MS2 Instrumentation and Analysis

Two, 3-hr gradients were collected per sample using an Orbitrap Fusion instrument coupled to a Waters liquid chromatography (LC) pump (Thermo Fisher Scientific). Peptides are fractionated on a 100 μm inner diameter microcapillary column packed with ˜25 cm of Accucore 150 resin (1.2 μm, 150 Å, ThermoFisher Scientific). For each analysis, 1 μg per sample was loaded onto the column. Peptides were separated using a 3 hr gradient of 6 to 46% acetonitrile in 0.2% formic acid at a flow rate of ˜400 nL/min. Instrument settings for the Orbitrap fusion were as follows: FTMS1 resolution (120,000), ITMS2 isolation window (0.4 m/z), ITMS2 max ion time (120 ms), ITMS2 AGC (2E4), ITMS2 CID energy (35%), dynamic exclusion window (90 sec). A TOP10 method was used where each FTMS1 scan was used to select up to 10 FTMS2 precursors for interrogation by HCD-MS2 with readout in the orbitrap.

### Data Analysis

Resulting mass spectra were searched using commercially available analysis software (e.g., Byonic) against a human database publicly available from Uniprot which was concatenated with common contaminants and reversed sequences of the human and contaminant proteins as decoys for FDR determination. Searches restricted the precursor ion tolerance to 20 ppm, and product ion tolerance window was set to 0.5 m/z. Searches allowed up to two missed cleavages, including static carbamidomethylation of cysteine residues (+57.021 Da) and variable oxidation of methionine residues (+15.995 Da). Additional variable modifications may be included, particularly glycosylations, based on the results of the gel shift assay following de-glycosylation treatment or the preview search PTM scan. Results were filtered to a 1% FDR at the protein level per sample.

### Example 3—In Vitro Screening of Stem Cell Secreted Factors

A deeper understanding of a given protein factor's contribution to the regenerative effects of the pool of heparin-associated hPSC secretome will be gained by screening against an established panel of assays for cellular age. Assays include measurements of reactive oxygen species (ROS) production or tolerance, cytoplasmically and in the mitochondria, telomerase activity, measurements of proteostasis capacity via lysosomal, autophagy, and proteasomal routes, epigenetic re-patterning, and cellular energy balance (e.g., ATP/ADP and NAD/NADH ratios). Many of these assays leverage the high-throughput automated microscopy described above to make these measurements in a variety of cell types, including fibroblast, endothelial cells, mesenchymal stem cells, and chondrocytes. Collectively these metrics can inform both the pathway and the mechanisms by which the heparin-associated hPSC secretome or its individual components enact their regenerative effects.

To begin screening and validating potential regenerative protein factors protein coding sequences will be collected from a publicly available source, such as used for the proteomics analysis (e.g., UniProt). The sequence for each of the proteins will be used to construct a DNA sequence encoding the proteins. The sequences are then each cloned into a plasmid vector system tailored for inducible or constitutive high-copy expression (in mammalian or prokaryotic settings). Alternatively, such a plasmid vector system may be designed in silico. Such a plasmid can be transformed into a pool of cells where the encoded protein was transiently expressed from the plasmid. Alternatively, the gene of interest could be incorporated in the genomes of a pool of cells (e.g. lentiviral transduction for mammalian cells or homologous recombination for S. cerevisiae) to create stable cell lines for recombinant protein production.

To de-bulk the target factor list and validate recombinant expression for factor production, a viable route would be to express the construct in a human cell line (like 293T-REx), which exploits: 1) that proteins of interest naturally purify themselves during the secretion process; and 2) will be processed in a natural context, potentially preserving important post translational processing steps. T-REx cells will be grown on 10 cm plates to ˜50% confluence in DMEM with 10% Bovine Growth Serum (Hyclone), 2 mM L-glutamine, and 1% Pen-Strep before initiating translation of a target protein of interest for 48 hr. The media would be collected, spun at 2,000 g to purify, and the supernatant used for heparin-associated enrichment of target factors in mouse myoblast regeneration assays.

### Machine Learning Classifier

By combining and statistically comparing the information from the Regenerative index assay, the Panel of Cellular Age Makers, the Proteomics we can create deep feature vectors for each protein factor, the pool of all factors (from each repeat of the assays), and the negative control pool (from each repeat of the assays). Treating the pool of all factors (or known factors such as FGF-2) as True Positives, and the negative control pool (or known non-functional proteins such as BSA) as True Negatives a supervised clustering algorithm can be trained to classify protein factors. Using a standard 10-fold cross validation scheme to assess the relative accuracy, recall, and confusion matrix graphs of the output of various algorithms' outputs (eg, Naive Bayes, Support Vector Machine, Linear Regression, or Random Forest) trained classifier most likely to successfully distinguish proteins with regenerative potential from the set of target factors can be selected. Target factors (or tested combinations) can then be rank ordered by the probability they derive from the regenerative set compared to the null set. A number of the top scoring target factors (or tested combinations) will then be selected for GMP-grade production for in vivo and in vitro validation.

Based on the complexity of the original heparin-associated fraction of the hPSC secretome and the limits to which individual proteins can recapitulate the activity of the whole pool, we will test combinations of factors as well. In the simplest approach, we would combine the 293T-REx secretome containing media from two or more cell lines each producing a given factor, and test their combined regenerative efficacy across a range of concentrations in an isobologram analysis using the regenerative index from the Myoblast Regeneration assay.

### Example 4—In Vivo Testing of Stem Cell Secreted Factors

There are two main aspects of muscle degeneration with aging, acute loss following trauma and chronic wasting (sarcopenia), and both of them will be tested. As the therapeutic approach to each case is expected to be different two arms for the in vivo validation is envisioned to specifically test each use-case for the factors as therapeutics in humans. The following Acute Injury Model and Sarcopenia/Chronic Administration Model for the most promising proteins emerging from the machine learning classifier can be carried out.

### Acute Injury Model

Animals were kept under standard animal husbandry condition. Animals were fed standard chow, and have ad libitum access to food and water. Temperature were kept at 22° C. and 12 h light/12 h dark cycles. Animals were acclimated prior to study initiation. The experimental groups were: C57BL/6J male mice, N=18; Young: 12-13 weeks old (3-month-old) mice, n=6; Aged: 77-78 weeks old (18-month-old) mice, n=12. This design was used to test any single factor identified and validated in in vitro assays or complex mixtures of 2 or more factors, as shown in FIG. 1.

TABLE 7
Experimental design of acute injury model

| | | | Dose | Adminis- | Blood | Tissue |
|---|---|---|---|---|---|---|
| Group | | Test | (mg/kg) | tration | collection | collection |
| Young, | 6 | vehicle | n/a | i.m, q.d; into | terminal | TA/GA |
| Vehicle | | | | injured | | muscles into |
| Aged, | 6 | vehicle | n/a | muscle | | Tissue-TEK |
| Vehicle | | | | site on day | | OCT; Brain, |
| Aged, | 6 | Test | 0.1 | 0, and 2 | | liver, heart, |
| Test | | factor | | | | and lung into |
| factor | | | | | | 4% PFA |

On Day 0, mice were weighed and underwent muscle injury with focal injection of barium chloride (BaCl₂, 14 μL, 1.2% w/v in saline, Sigma-Aldrich) in the Tibialis anterior (TA; Day 0) of both the right and left hindlegs. Injections of vehicle or factor A (0.1 mg/kg) were administered intramuscularly (i.m) 2 h following the BaCl₂ into the TA injured hindleg sites, and again 48 hours later on day 2 (i.m.) into the TA injured hindleg sites. Also on day 2, BaCl₂ (BaCl₂, 14 μL, 1.2% w/v in saline, Sigma-Aldrich) was injected into the Gastrocnemius (GA, Day 2, i.m.) muscles of both right and left hind legs. Injections of vehicle or a factor were sequentially administered (i.m.) following the BaCl₂ into the TA hindleg sites post-injury, and again 48 hours later on day 4 (i.m.) into the GA injured hind leg sites. Bromodeoxyuridine (BrdU) was be administered (100 mg/kg, i.p.) once daily for 3 days, day 2-4, before sacrifice to label proliferating cells.

On day 5, animals were sacrificed, and animal weight recorded followed by collecting 0.5 ml of terminal blood via cardiac puncture which was processed to plasma and stored at 80° C. We then dissected the skin from the GA/TA muscles of each hind leg and took photos (prior to excision). After excision of exclusively the GA or TA muscle, excised tissue was photographed, weighed, then immersed in Tissue-TEK OCT and rapidly frozen in chilled isopentane bath precooled in liquid nitrogen and stored at −80 C. Cryosectioning and H&E were performed to ensure muscle injury site was appropriately visualized. A diagram of the time points for these experiments can be seen in FIG. 2A. Muscle tissue composition from new skeletal muscle fibers, fibrotic tissue, and adipose (fat), were measured. Muscle regeneration, as defined as the number of number of new myofibers with centrally located nuclei per millimeter, fibrosis as defined as the area of fibrotic scarring, size of the fibers, as defined as the width and area, adipose tissue, as defined by the amount of fat surrounding the muscle, were measured to assess level of regeneration.

Young muscle regeneration after acute focal injury had the highest regenerative index (measured as the number of new myofibers with centrally located nuclei per millimeter squared). Aged muscle regeneration following acute injuries performed on average 30-50% less than young muscle regeneration, in part due to the reduction of mitogenic muscle precursor/progenitor cell activity (FIG. 2B). It was predicted that systemic treatment of aged animals, injured animals, animals with genetic diseases causing muscle wasting, or animals injured by radiation or other tissue damaging treatment, the HAPs, individually or in various combinations, will improve skeletal muscle regeneration by 20-50%, approaching comparable levels to young healthy animals. It was also predicted that the composition of fibrous scar tissue and fat tissue will be reduced to levels comparable to younger animals by treatment with the HAPs. And indeed, treatment with the HAPs improved the skeletal muscle regeneration of sarcopenic mice to levels indistinguishable from the young by both the number of new myoblasts and the reduction in fibrous scar tissue (FIG. 2B).

### Sarcopenia/Chronic Administration Model

After arrival, animals will be kept under standard animal husbandry condition. Animals will be fed standard chow, and have ad libitum access to food and water. Temperature will be kept at 22° C. and 12 h light/12 h dark cycles. Animals will be acclimated prior to study initiation, including any in vivo assay acclimation, if necessary. The experimental design was C57BL/6J male mice, N=18; Young: 12-13 weeks old (3-month-old) mice, n=6; Aged: 77-78 weeks old (18-month-old) mice, n=12. This design can be used to test any single factor identified and validated in in vitro assays or complex mixtures of 2 or more factors.

TABLE 8
Design of sarcopenia/chronic administration model

| | | | | Adminis- | | Blood | Tissue |
|---|---|---|---|---|---|---|---|
| Group | n | Test | (g/kg) | tration | In vivo assay | collection | collection |
| Young, | 6 | vehicle | n/a | i.p, q.d; on | 2 sets of: Animal | terminal | TA/GA |
| Vehicle | | | | day −8 to +5 | weight, grip strength, | | muscles into |
| Aged | 6 | vehicle | n/a | | running wheel | | Tissue-TEK |
| Vehicle | | | | | performance; | | OCT; Brain, |
| Aged, | 6 | Test | 0.1 | | horizontal | | liver, heart, |
| Test | | factor | | | bar; 1 set of: | | and lung into |
| factor | | | | | In capacitance | | 4% PFA |

On Day 0, mice will have the following in vivo healthspan measurements will be performed over 1 day as a baseline for age-based parameters: Weight, running wheel performance, grip strength, and horizontal bar. Each assay should be run for 4 trials per assay per animal. These healthspan assays will be repeated on day −1. After one day of rest on day −9, mice will begin 1× daily injections (0.1 mg/kg) of vehicle or factor A for the remainder of the experiment until sacrifice (days −8 to +5, 13 days of dosing). On day −4, 6 days after dosing begins, mice will undergo a repeat of the healthspan assays. On day 0, 5 days prior to sacrifice, mice will undergo muscle injury with focal injection of BaCl₂ (BaCl₂, 14 μL, 1.2% w/v in saline, Sigma-Aldrich) in the Tibialis anterior (TA; Day 0) of the right hindleg only. On day 2, the Gastrocnemius (GA; Day 2) muscle of the right hind leg will then receive BaCl₂ (BaCl₂, 14 μL, 1.2% w/v in saline, Sigma-Aldrich). BrdU will be administered (100 mg/kg, i.p.) once daily for 3 days, day 2-4, before sacrifice. On day +5, prior to take-down, the animals will have an in vivo in capacitance assay run. On day +5, animals will be sacrificed, and animal weight recorded. Collect 0.5 ml of blood via cardiac puncture, process to plasma and store plasma samples at 80° C. will then dissect the skin from the GA/TA muscles of each hind leg and take photos (prior to excision). After excision of exclusively the GA or TA muscle, weigh the muscles, then place the muscles in chilled isopentane bath precooled in liquid nitrogen and stored at −80 C. Perform cryosectioning and H&E, ensuring muscle injury site is appropriately visualized. Carefully excise the inguinal white adipose tissue (WAT) and weigh tissue. Discard WAT post-weighing.

Collected brain, liver, heart and lung can be post-fixed in 4% PFA for 72 hours, after 72 hours, transferred into 30% sucrose in 1×PBS and stored at −4° C. (brain, liver, heart, lung).

Muscle tissue composition, from new skeletal muscle fibers, fibrotic tissue, and adipose (fat), will be measured. Muscle regeneration, as defined as the number of number of new myofibers with centrally located nuclei per millimeter, fibrosis, as defined as the area of fibrotic scarring, size of the fibers, as defined as the width and area, adipose tissue, as defined by the amount of fat surrounding the muscle, will be measured to assess level of regeneration. Weights of the animals during the duration of treatment with HAP(s), as well as healthspan assays including performance on a running wheel (speed, distance, duration), grip strength, and performance on a horizontal bar will take into account the phenotypic outcomes of treatment of the aged animals systemically with the HAPs for sarcopenia.

The horizontal bar test is performed as described previously (Malinowska et al. 2010) at 8 months (n=6 WT, n=7 MPS IIIB) and 10 months (n=3 WT, n=4 MPS IIIB) of age. In brief, a 300-mm metal wire, 2 mm in diameter, was secured between two posts 320 mm above a padded surface. The mouse was allowed to grip the center of the wire and the time to fall or reach the side was recorded, and after 2 min the test was stopped. Crossing the bar in x seconds was scored as 240-x, remaining on the bar was scored as 120, and falling off the bar after y seconds was recorded as the value of y. The test was repeated three times as a practice run followed by a 10-min rest prior to three tests where the score was recorded.

Young muscle regeneration after acute focal injury has the highest regenerative index (measured as the number of new myofibers with centrally located nuclei per millimeter squared). Aged muscle regeneration following acute injuries performs on average 30-50% less than young muscle regeneration, in part due to the reduction of mitogenic muscle precursor/progenitor cell activity. We predict systemic treatment of aged animals, injured animals, animals with genetic diseases causing muscle wasting, or animals injured by radiation or other tissue damaging treatment, the HAPs, individually or in various combinations, will improve skeletal muscle regeneration by 20-50%, approaching comparable levels to young healthy animals. We also predict the composition of fibrous scar tissue and fat tissue will be reduced to levels comparable to younger animals by treatment with the HAPs.

Animals will also have better healthspan outcomes: reduced weight, fat composition, scar tissue around muscles, increased running speed, duration, and distance, increased grip strength, and enhanced performance on the horizontal bar test.

### Genetically Obese Muscle Dystrophy Model

Genetically obese (ob/ob) mice were injected with BaCl₂ on day 0 in the TA muscle. 3 mice were treated with vehicle only, 3 mice were injected with the hPSC factors and 3 mice were treated with FGF19 (positive control) on day 0 and day 2. On day 5, the mice were euthanized, the TA muscles perfused with PBS, and dissected, as depicted in FIG. 3A. Muscles were then analyzed for regenerative index and fibrotic index, as described in Example 1. Mice that had been treated with either the hPSC factors or FGF19 had a significant increase in the regenerative index and a significant decrease in the fibrotic index when compared to untreated mice, as depicted in FIG. 3B.

### Methods of Testing Muscle Strength, Endurance and Function

Forelimb and Both limb grip strength test: After 30 min acclimation, the mice are introduced to the grip strength meter. For forelimb grip strength, the mice held by the tail are allowed to grasp the grip bar with only its forelimbs. For both limb measurements the mice are placed on the grid and allowed to grasp the grid with both limbs. The force generated by each mouse is calculated as the average of 5-6 measurements.

Limb endurance test: Mice are allowed to discover and acclimate the rodent treadmill environment through 2 training sessions of 10 min each at 10 m/min on separate days prior to the endurance test. For the endurance test, mice are placed in the individual lanes of the rodent treadmill. The speed is gradually increased at 2 m/min until exhaustion is reached. Exhaustion is defined as a mouse staying on a grill electrified to deliver a shock of 2 Hz, intensity 5 for 3-5 seconds.

In vivo tetanic force measurement: Mice will be under anesthesia using regulated delivery of isoflurane during the whole process. Following anesthetization, the animal is placed onto a heated chamber with the foot is secured on the foot pedal of an Aurora force transducer. The 2 electrodes are placed specifically to stimulate the sciatic nerve. The force generated by the ankle torsion of the animal's hind limb, as opposed to direct force is measured in response to a series of stimulation that includes 20, 50, 70, 100, 150 and 200 Hz.

In situ tetanic force measurement: This experiment is performed using Aurora force measurement. Mice are under anesthesia during the whole process. A small incision in the skin around the Anterior Tibialis exposes the Achilles tendon which is connected via surgical suture to the Aurora force transducer through a hook. The force generated by the muscle in response to a series of stimulation that includes 20, 50, 70, 100, 150 and 200 Hz by 2 electrodes placed on the anterior tibialis is recorded.

### Example 5-Additional Tests for Pro-Regenerative Factors

Mechanistic insight into a given HAP factor's pathway of action will be gained by establishing and screening against a panel of assays for cellular age. Assays include measurements of reactive oxygen species (ROS) production or tolerance cytoplasmically and in the mitochondria, telomerase activity, measurements of proteostasis capacity via lysosomal, autophagy, and proteasomal routes, epigenetic re-patterning, and cellular energy balance (e.g., ATP/ADP and NAD/NADH ratios). Many of these assay leverage high-throughput automated microscopy to make these measurements in a variety of cell types, including fibroblast, endothelial cells, mesenchymal stem cells, and chondrocytes. Collectively these metrics can inform both the pathway and the mechanisms by which the heparin-associated hPSC secretome or its individual components enact their regenerative effects. These deep profile vectors can be crucial for approaching combinations of factors rationally, and for machine learning predictions.

To test the cellular effects of secretomes toward reversing the hallmarks of aging, high-throughput automated imaging and quantification of single cells to achieve deep population level statistical power can be employed. Cellular component state profiles of Young, Aged, and Aged+Treatment in human fibroblasts and epithelial cells, myoblasts, mesenchymal stem cells, chondrocytes, and neural progenitor cells will be compared. Some examples of tests and methods include:

Epigenetic reprogramming: repressive mark H3K9me3, the heterochromatin-associated protein HP1γ, nuclear lamina support protein LAP2α

Nuclear membrane Folding/Blebbing: immunofluorescence of the nuclear membrane protein Lamin A/C

Proteolytic Activity: Cleavage of fluorescent-tagged chymotrypsin like substrate corresponds to proteasome 20S core particle activity. Wells are first stained with PrestoBlue Cell Viability dye (Life Technologies) for 10 minutes. Well signals are read using a TECAN fluorescence plate reader as a measure of cell count. Then cells are washed with HBSS/Ca/Mg before switching to original media containing the chymotrypsin like fluorogenic substrate LLVY-R110 (Sigma) which is cleaved by the proteasome 20S core particle. Cells are then incubated at 37° C. in 5% CO2 for 2 hours before signals are again read on the TECAN fluorescence plate reader. Readings are then normalized by PrestoBlue cell count.

Formation of autophagosomes: Autophagosome number and volume are measured by staining with CellTracker Deep Red (Sigma). The cells are then incubated at 37° C. in 5% CO2 for 20 minutes, washed 2 times using HBSS/Ca/Mg, and stained for 15 minutes using CellTracker Deep Red cell labeling dye. Cells are then switched to HBSS/Ca/Mg for single cell imaging using the Operetta High Content Imaging System (Perkin Elmer).

Energy Metabolism: ATP in the cells is measured using colorimetric assay using an ATP assay kit (ab83355; Abcam, Cambridge, MA) following manufacturer's instructions. Cells are washed in cold phosphate buffered saline and homogenized and centrifuged to collect the supernatant. The samples are loaded with assay buffer in triplicate. ATP reaction mix and background control (50 μL) is added to the wells of a 96 well plate and incubated for 30 min in dark. The average change in absorbance at 570 nm is used to estimate of the intracellular ATP concentration relative to the standard curve.

Mitochondrial Activity: To measure Mitochondria Membrane Potential, cells are washed twice with Ham's F10 (no serum or pen/strep). Subsequently, MuSCs are stained with MitoTracker Green FM (ThermoFisher, M7514) and DAPI for 30 minutes at 37° C., washed three times with Ham's F10, and analyzed using a BD FACSAria III flow cytometer. To measure Mitochondrial ROS Measurement. Cells are washed with HBSS/Ca/Mg and then switched to HBSS/Ca/Mg containing MitoSOX (Thermo), a live cell permeant flurogenic dye that is selectively targeted to mitochondria and fluoresces when oxidized by superoxide. Cells are incubated for 10 minutes at 37° C. in 5% C02. Cells are then washed twice with HBSS/Ca/Mg, and stained for 15 minutes using CellTracker Deep Red. Finally, cells are imaged in fresh HBSS/Ca/Mg using the Operetta High Content Imaging System (Perkin Elmer).

Deregulated Nutrient Sensing: levels of SIRT1 are measured.

Senescence: Senescence-associated beta-galactosidase staining is measured in cells washed twice with PBS then fixed with 15% Paraformaldehyde in PBS for 6 minutes. Cells are rinsed 3 times with PBS before staining with X-gal chromogenic substrate, which is cleaved by endogenous Beta galactosidase. Plates are kept in the staining solution, Parafilmed, to prevent from drying out, and incubated overnight at 37° C. with ambient CO2. The next day, cells are washed again with PBS before switching to a 70% glycerol solution for imaging under a Leica brightfield microscope.

Secretome of the cells: Mass-Spec or O-Link for inflammatory cytokines profiles.

Soft Tissue Deposition: Immunofluorescence for SOX9, MMP3, MMP13, and COL2A1 expression, the decrease of which is characterized by cartilage loss, pain, cleft-lip, and joint destruction.

### Example 6—Identification of Pro-Regenerative Factors by Mass Spectroscopy

Factors enriched in the secretome of undifferentiated hPSCs can be determined by Mass spectroscopy. A schematic of a type of mass spectroscopy experiment employed herein is shown in FIG. 3A.

Five confluent, 15 cm plates of cells per biological replicate were washed with OptiMEM—a basal, synthetic medium-, and then incubated in OptiMEM for 16 hours, yielding roughly 100 ml of media. The media, now containing secreted factors, was collected, cells and cell debris removed by centrifugation, and flash frozen for storage at −80 C until processing. The target factors were enriched via affinity purification for heparin binding using heparin-agarose bead columns. Heparin-agarose beads (Sigma) were washed with water twice, and once with OptiMEM (minus phenol red), before incubating with factor containing culture media for 2 h at 4° C. shaking at 100 rpm. The ratio of bead slurry (˜50% beads) to media can be effective at 1:10, 1:20, 1:30, 1:40, and 1:50. Heparin-agarose beads were then collected into a column by centrifugation in an Amicon Pro Purification System column set in a 50 ml conical tube at 1000 g for 5 min, washed with 10× column volumes of PBS+0.05% tween at 4° C. twice. Factors were eluted via two repeats of the following: addition of a high salt solution (1.5M NaCl, 0.01M HEPES, pH 7.2, at ratio of 0.4 ml elution buffer per milliliter of bead slurry), incubated at 4° C. for 10 minutes at 100 rpm, and centrifugation at 1000 g for 5 min into a fresh collection tube.

Protein concentration in the eluted fraction was assayed by silver stain densitometry, and a BCA assay against standard curves for bovine serum albumin. Protein disulfide bonds were reduced by incubation in 5 mM tris-(2-carboxyethyl)-phosphine (TCEP) for 25 min, and the free cysteines alkylated with 10 mM iodoacetamide at room temperature for 30 min in the dark. Excess iodoacetamide was quenched with 15 mM dithiotreitol during a 15 min incubation. The eluates from all samples were then further purified by protein precipitation using trichloroacetic acid, prior to resuspending in digest buffer and 16 hr of digestion using a mixture of modified Trypsin and LysC to yield peptides predominantly with terminal arginine or lysine residues. The resulting peptide concentration were measured using a quantitative colorimetric peptide assay (Promega), and equimolar amounts of peptides from each biological replicate labeled at their free amines with tandem mass tags (TMT) using manufacturer recommended conditions before mixing the peptides. The mixed sample was desalted via reverse phase separation on a C18 StageTip prior to analysis via nHPLC-SPSMS3 on a Fusion Lumos (Thermo Fisher). A TOP10 method was used to select up to 10 MS2 precursors for identification by CID-MS2 analyzed in the ion trap. For synchronous precursor selection of up to 10 ion windows, the FTMS3 isolation window was 0.4 m/z, max ion time 150 ms, automatic gain control 1.5E5, and FTMS3 resolution was 50,000. Resulting spectra were searched using commercial MS analysis software against the Uniprot human database (2018) protein sequences (Swiss-Prot and TrEMBL) concatenated with their reversed sequences as decoys for FDR determination, appended to common contaminant sequences. Searches restricted the precursor ion tolerance to 50 ppm and the product ion tolerance window to 0.9 m/z (or 50 ppm), allowed no more than two missed cleavages, included static modification of lysine residues, arginine residues and peptide N-termini with TMT tags (+229.163 Da), static carbamidomethylation of cysteine residues (+57.021 Da), and variable oxidation of methionine residues (+15.995 Da).

Results were filtered to a 1% FDR at the peptide and then protein level using the target-decoy strategy. Peptides were assigned to protein groups, and individual proteins by the parsimony principle. Proteins were quantified by summing reporter ion intensities across all PSMs with greater than 70% of their spectral intensity deriving from matched ions and a summed signal to noise intensity greater than 200, normalizing channel level intensities, and computing the percent contribution of a given channel to the total signal. These values were then used for additional statistical modelling of differential abundance.

Heparin-associated proteins from undifferentiated and differentiated supernatants generated distinct sets of secreted factors. Combined results from such experiments are summarized in Table 2 shown previously herein, by the gene name, UniProt ID, Entrez Gene ID, and Ensembl ID.

### Example 7—Validation of Pro-Regenerative Factors In Vitro Using High-Throughput Imaging

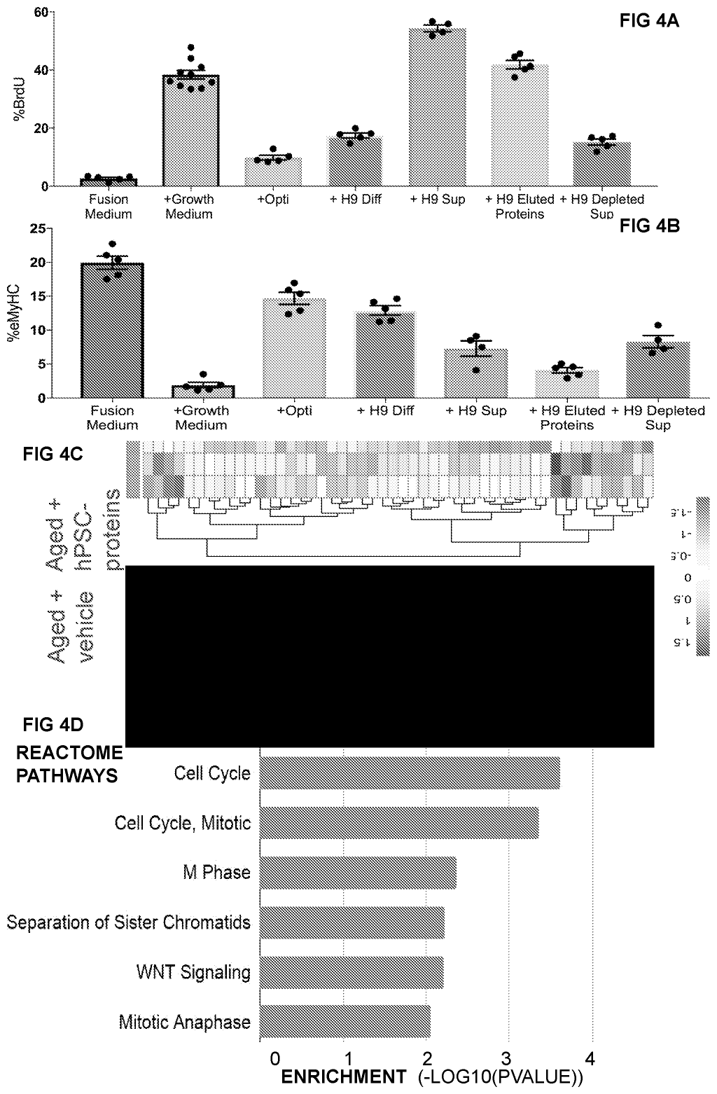

Mass spectroscopy can define candidate pro-regenerative factors, however, as shown in example 6, these experiments can generate large amounts of data that need to be further validated in relevant in vitro and in vivo models. The use of high-throughput imaging can help define individual factors and mixtures of factors that possess regenerative potential. Mouse muscle progenitor cells can be cultured with BrdU or Edu, in the presence or absence of specific potential pro-regenerative factors, and the degree of proliferation determined using high-throughput microscopy. BrdU or Edu staining indicates proliferation, while embryonic Myosin Heavy-Chain (eMyHC) staining indicates terminal differentiation of the progenitor cells. FIGS. 4A-4B show an example of data generated using high-throughput imaging.

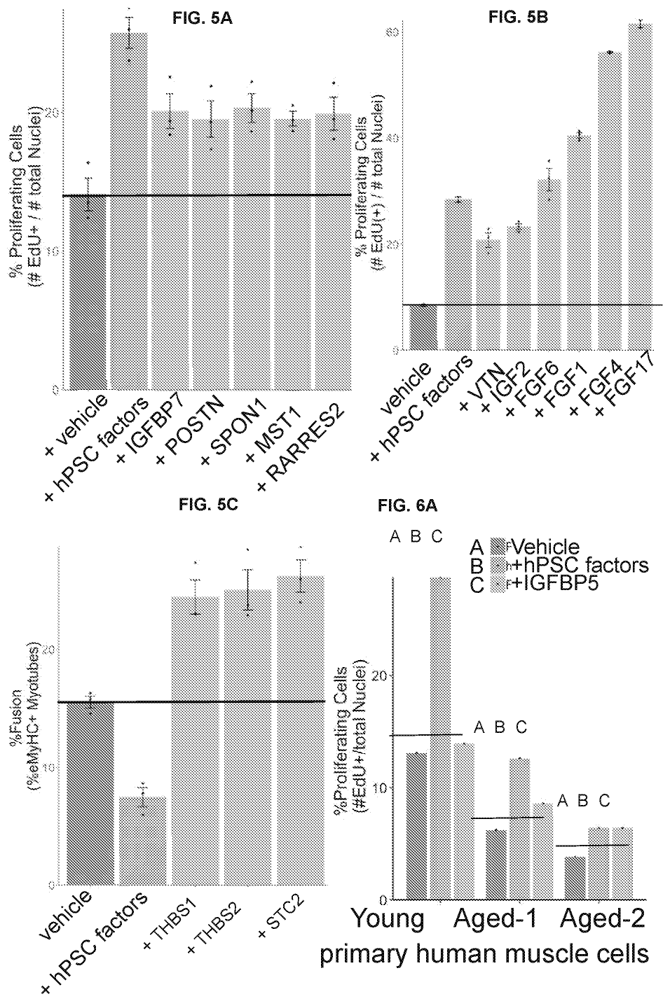

Many of the factors detailed in Table 2 were tested individually for their ability to promote proliferation and/or fusion of mouse muscle progenitor cells in vitro. FIGS. 5A-5B show that there was a significant increase in the percent of proliferating cells of injury-activated primary mouse myoblasts grown in vitro for the hPSC factors, IGFBP7, POSTN, SPON1, MST1, RARES2, VTN, FGF1, IGF2, FGF4, FGF6, and FGF7, when compared to untreated cells. FIG. 5C shows that treatment with THBS1, THBS2, and STC2 resulted in a significant increase in the percent of fusion in injury activated primary mouse myoblasts, compared to untreated cells.

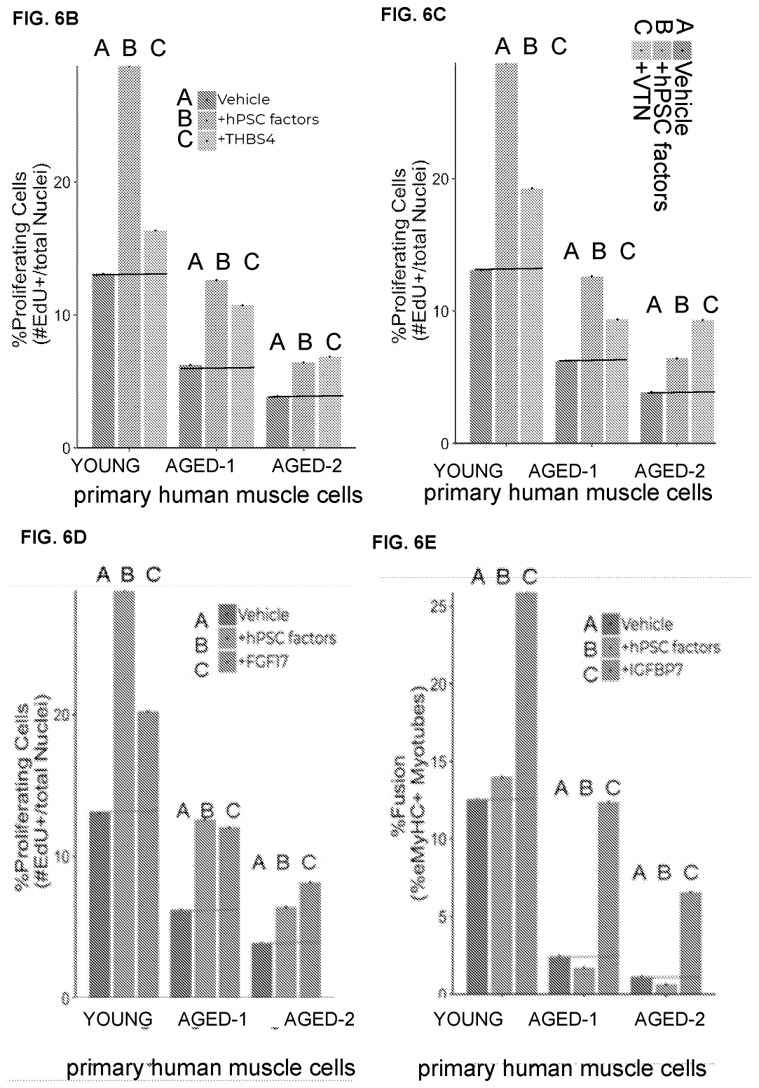

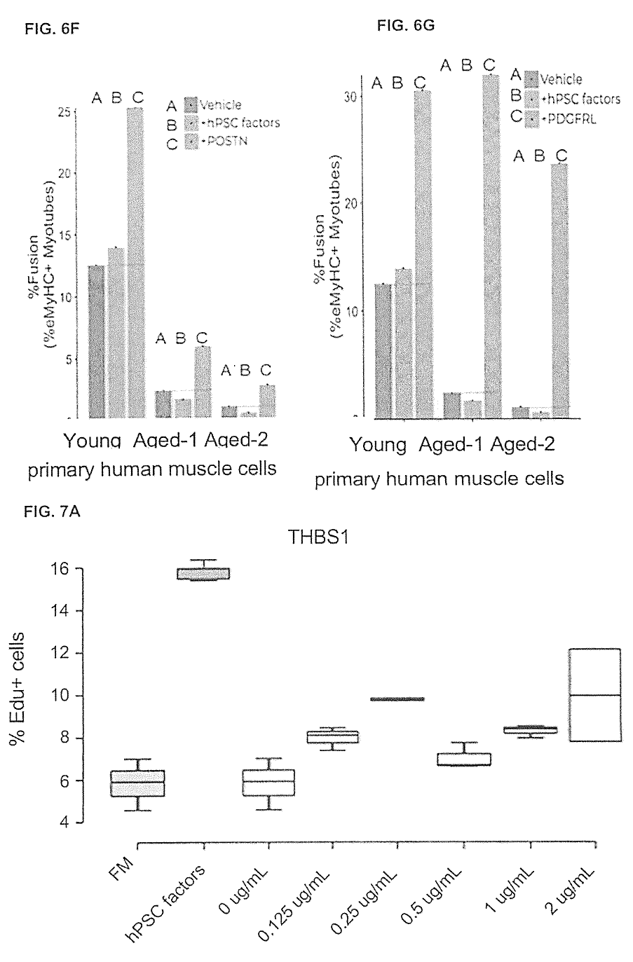

The factors were also able to affect proliferation and/or fusion in both young (18 years) and old (69 years) injury-activated primary human myoblasts. FIG. 6A shows that treatment with IGFBP5 at 1 μg/mL resulted in an increase in the percent of proliferating cells in both young and aged human myoblasts, compared to untreated myoblasts. FIG. 6B shows that treatment with THBS4 at 1 μg/mL resulted in an increase in the percent of proliferating cells in both young and aged human myoblasts, compared to untreated myoblasts. FIG. 6C shows that treatment with VTN at 10 μg/mL resulted in an increase in the percent of proliferating cells in both young and aged human myoblasts, compared to untreated myoblasts. FIG. 6D shows that treatment with FGF17 at 250 ng/mL resulted in an increase in the percent of proliferating cells in both young and aged human myoblasts, compared to untreated myoblasts. FIG. 6E shows that treatment with IGFBP7 T 500 NG/Ml resulted in an increase in the percent of fusion in both young and aged human myoblasts, compared to untreated myoblasts. FIG. 6F shows that treatment with 1 μg/mL of POSTN resulted in an increase in the percent of fusion in both young and aged human myoblasts, compared to untreated myoblasts. FIG. 6G shows that treatment with 5 μg/mL of PDGFRL resulted in an increase in the percent of fusion in both young and aged human myoblasts, compared to untreated myoblasts.

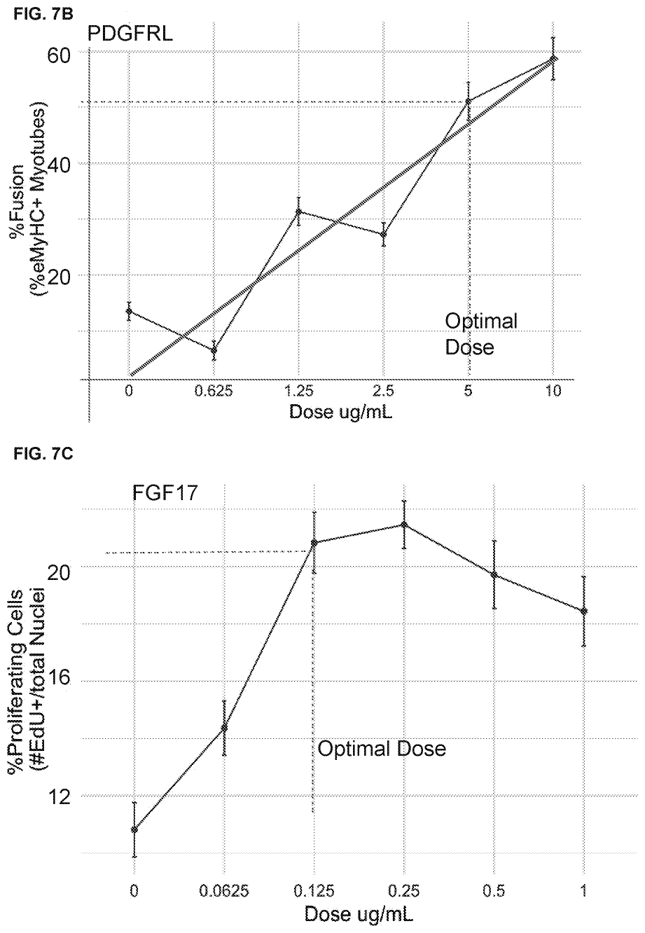

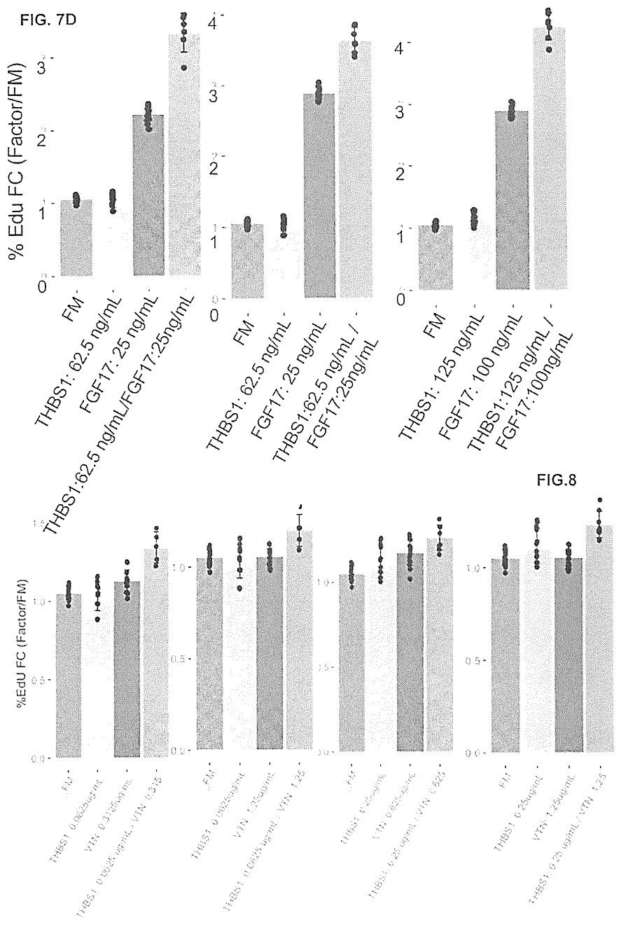

FIG. 7 shows an example of the dose dependent increasing cellular fusion of mouse myoblasts cultured with a HAP. In this case Platelet derived growth factor-like (PDGFRL) proteins were applied at 625 ng/mL, 1250 ng/mL, 2500 ng/mL, 5000 ng/mL, and 10000 ng/mL.

The effect of the candidate factors on myogenic activity was assayed in biological triplicate across a range of concentrations centered around expected physiological levels by adding each factor to mouse myoblasts for 48 hours or human myoblasts for 72 hours with daily media changes (DMEM +2% horse serum) and a second pulse of factors. After 24 hours, cells were pulsed for 2-5 hours with EdU (30 uM), ethanol fixed, stained with Hoescht 3342, immunostained for proliferation—as measured by the percent of cells staining positive for EdU (% EdU)−, and immunostained for differentiation—as measured by the increase in cellular area staining positive for embryonic myosin heavy chain (% eMyHC) relative to the negative controls, which received media and vehicle only. Wells were imaged on a Keyence BZ-100 at 4×, the images quantified in Cell Profiler, and the statistics were computed in R. FIGS. 8A-8B show an example of the proliferation dose response for two of the factors tested. Results for additional factors are summarized below in Table 9.

TABLE 9
Effect of individual factors on mouse myoblast growth and fusion

| | | Proliferation | | | Fusion | |
|---|---|---|---|---|---|---|
| | | (¹ = % EdU or ² = nuclei counts) | | | (% eMyHC) | |
| | | | Effect size | Effect | Effect size | Effect |
| | | Concentration | (fold-change | significance | (fold-change | significance |
| Factor | Effect | (ug/mL) | to control) | (p-value) | to control) | (p-value) |
| AGRN | Proliferation² | 2.5 | 1.14 | 0.01008 | 0.46 | n.s. |
| APOB | Proliferation¹ | 0.1 | 1.28 | 0.008202 | 1.01 | n.s. |
| BMP7 | Proliferation¹ | 0.025 | 1.71 | 0.001448 | 0.17 | n.s. |
| BTC | Proliferation¹/ | 0.5 | 1.4 | 0.03395 | 1.7 | n.s. |
| | Fusion | | | | | |
| CHRDL1 | Proliferation¹ | 2.5 | 1.62 | 0.004249 | 0.85 | n.s. |
| CLEC3A | Proliferation¹ | 0.04 | 1.35 | 0.005224 | 1.09 | n.s. |
| FGF1 | Proliferation¹ | 0.5 | 2.89 | 0.005553 | 0.84 | n.s. |
| FGF17 | Proliferation¹ | 0.5 | 4.50 | 0.01809 | 0.09 | n.s. |
| FGF4 | Proliferation¹ | 0.5 | 4.01 | 0.01363 | 0.25 | n.s. |
| FGF6 | Proliferation¹/ | 1 | 3.8 | 0.007639 | 1.85 | 0.02367 |
| | Fusion | | | | | |
| HGF | Proliferation¹ | 0.5 | 2.04 | 1.56E−05 | 0.39 | n.s. |
| IGF2 | Proliferation¹ | 2 | 1.66 | 0.0239 | 1.46 | n.s. |
| IGF2 | Proliferation² | 0.03 | 2.28 | 0.0004336 | — | — |
| IGFBP2 | Proliferation¹ | 1 | 1.97 | 0.04376 | 1.03 | n.s. |
| IGFBP7 | Proliferation¹/ | 1 | 1.23 | 0.01132 | 1.05 | 0.04213 |
| | Fusion | | | | | |
| IL15 | Proliferation¹/ | 0.5 | 1.85 | 0.004579 | 2.03 | 0.02273 |
| | Fusion | | | | | |
| MST1 | Proliferation¹ | 1 | 1.36 | 0.03868 | 1.25 | n.s. |
| NOV | Proliferation² | 5 | 1.26 | 0.03359 | 0.65 | n.s. |
| NTS | Proliferation¹ | 0.2 | 1.29 | 0.006777 | 1.1 | n.s. |
| PAMR1 | Fusion | 0.4 | 0.88 | n.s. | 1.27 | 0.002882 |
| PDGFD | Proliferation¹ | 0.1 | 1.47 | 3.94E−02 | 0.97 | n.s. |
| PLAT | Proliferation² | 2.2 | 1.27 | 0.02239 | 0.68 | n.s. |
| POSTN | Fusion | 0.25 | 0.65 | n.s. | 1.5 | 0.002679 |
| THBS1 | Proliferation¹/ | 1 | 1.26 | 0.003634 | 1.39 | 0.0008755 |
| | Fusion | | | | | |
| THBS2 | Proliferation¹/ | 1 | 1.54 | n.s. | 1.34 | 3.94E−05 |
| | Fusion | | | | | |
| THBS4 | Proliferation¹/ | 2 | 1.87 | 0.0163 | 1.23 | 0.0004685 |
| | Fusion | | | | | |
| VTN | Proliferation¹ | 5 | 1.56 | 0.01231 | 1.01 | n.s. |
| | | Proliferation | | | Fusion | |
| | | (¹ = % EdU or ² = nuclei counts) | | | (% eMyHC) | |
| | | | | Effect | | Effect |
| | | | Effect size | significance | Effect size | significance |
| | | Concentration | (fold-change | (p-value, | (fold-change | (p-value, |
| Factor | Effect | (ug/mL) | to control) | t-test) | to control) | t-test) |
| AGRN | Proliferation² | 2.5 | 1.14 | 0.01008 | 0.46 | n.s. |
| APOB | Proliferation¹ | 0.1 | 1.28 | 0.008202 | 1.01 | n.s. |
| BMP7 | Proliferation¹ | 0.025 | 1.71 | 0.001448 | 0.17 | n.s. |
| BTC | Proliferation¹/ | 0.5 | 1.4 | 0.03395 | 1.7 | n.s. |
| | Fusion | | | | | |
| CHRDL1 | Proliferation¹ | 2.5 | 1.62 | 0.004249 | 0.85 | n.s. |
| CLEC3A | Proliferation¹ | 0.04 | 1.35 | 0.005224 | 1.09 | n.s. |
| FGF1 | Proliferation¹ | 0.5 | 2.89 | 0.005553 | 0.84 | n.s. |
| FGF17 | Proliferation¹ | 0.5 | 4.50 | 0.01809 | 0.09 | n.s. |
| FGF4 | Proliferation¹ | 0.5 | 4.01 | 0.01363 | 0.25 | n.s. |
| FGF6 | Proliferation¹/ | 1 | 3.8 | 0.007639 | 1.85 | 0.02367 |
| | Fusion | | | | | |
| HGF | Proliferation¹ | 0.5 | 2.04 | 1.56E−05 | 0.39 | n.s. |
| IGF2 | Proliferation¹ | 2 | 1.66 | 0.0239 | 1.46 | n.s. |
| IGF2 | Proliferation² | 0.03 | 2.28 | 0.0004336 | — | — |
| IGFBP2 | Proliferation¹ | 1 | 1.97 | 0.04376 | 1.03 | n.s. |
| IGFBP7 | Proliferation¹/ | 1 | 1.23 | 0.01132 | 1.05 | 0.04213 |
| | Fusion | | | | | |
| IL15 | Proliferation¹/ | 0.5 | 1.85 | 0.004579 | 2.03 | 0.02273 |
| | Fusion | | | | | |
| MST1 | Proliferation¹ | 1 | 1.36 | 0.03868 | 1.25 | n.s. |
| NOV | Proliferation² | 5 | 1.26 | 0.03359 | 0.65 | n.s. |
| NTS | Proliferation¹ | 0.2 | 1.29 | 0.006777 | 1.1 | n.s. |
| PAMR1 | Fusion | 0.4 | 0.88 | n.s. | 1.27 | 0.002882 |
| PDGFD | Proliferation¹ | 0.1 | 1.47 | 3.94E−02 | 0.97 | n.s. |
| PLAT | Proliferation² | 2.2 | 1.27 | 0.02239 | 0.68 | n.s. |
| POSTN | Fusion | 0.25 | 0.65 | n.s. | 1.5 | 0.002679 |
| THBS1 | Proliferation¹/ | 1 | 1.26 | 0.003634 | 1.39 | 0.0008755 |
| | Fusion | | | | | |
| THBS2 | Proliferation¹/ | 1 | 1.54 | n.s. | 1.34 | 3.94E−05 |
| | Fusion | | | | | |
| THBS4 | Proliferation¹/ | 2 | 1.87 | 0.0163 | 1.23 | 0.0004685 |
| | Fusion | | | | | |
| VTN | Proliferation¹ | 5 | 1.56 | 0.01231 | 1.01 | n.s. |

The effect of the combination of candidate factors on myogenic activity was assayed in biological triplicate across a range of concentrations centered around expected physiological levels by adding each factor to mouse myoblasts for 48 hours or human myoblasts for 72 hours with daily media changes (DMEM +200 horse serum) and a second pulse of factors. After 24 hours, cells were pulsed for 2-5 hours with EdU (30 uM), ethanol fixed, stained with Hoescht 3342, immunostained for proliferation—as measured by the percent of cells staining positive for EdU (% EdU)−, and immunostained for differentiation—as measured by the increase in cellular area staining positive for embryonic myosin heavy chain (% eMyHC) relative to the negative controls, which received media, individual factors, or vehicle only. Wells were imaged on a Keyence BZ-100 at 4×, the images quantified in Cell Profiler, and the statistics were computed in R. FIGS. 8C-SM show examples of the proliferation dose response for two or more of the factors tested individually and as a combination to test for synergy. Statistical metrics for increased myogenetic activity from the pair of factors relative to the controls are summarized below in Tables 10-14. Each polypeptide was produced using the method listed in Table 1. The magnitude of the combinations' effects relative to control (FM—negative control, HAPs—positive control) is shown. The Combination Index (CI) for synergy was calculated using the Highest Single Agent (HAS) model due to the linear dose responses for the individual factor, e.g. FIG. 8B. Tables 10-15 show additional data regarding the synergistic effects of the HAPs in both mice and human myoblasts.

TABLE 10
Synergistic combinations of HAPs

| Factor 1 | | Factor 2 | | Factor 3 | | | CI |
|---|---|---|---|---|---|---|---|
| Name | ug/mL | Name | ug/mL | Name | ug/mL | p-value | (HSA) |
| THBS2 | 0.125 | THBS4 | 0.25 | VTN | 1 | <0.05 | 0.886 |
| THBS2 | 0.125 | THBS4 | 0.25 | ANOS1 | 1 | <0.005 | 0.532 |
| THBS2 | 0.125 | THBS4 | 0.25 | | | <0.005 | 0.633 |
| THBS1 | 0.0625 | FGF17 | 0.025 | | | <0.005 | 0.667 |
| THBS1 | 0.125 | FGF17 | 0.1 | | | <0.005 | 0.679 |
| THBS2 | 0.125 | THBS4 | 0.25 | IL-15 | 1 | <0.005 | 0.680 |
| THBS2 | 0.125 | THBS4 | 0.25 | IGF2 | 0.05 | <0.05 | 0.733 |
| THBS2 | 0.0625 | VTN | 1.25 | | | <0.005 | 0.763 |
| THBS2 | 0.125 | VTN | 1.25 | | | <0.005 | 0.763 |
| THBS2 | 0.125 | VTN | 0.3125 | | | <0.005 | 0.771 |
| THBS2 | 0.0625 | THBS4 | 0.125 | | | <0.005 | 0.772 |
| THBS2 | 0.125 | THBS4 | 0.0625 | | | <0.005 | 0.779 |
| THBS1 | 0.0625 | FGF17 | 0.1 | | | <0.005 | 0.796 |
| THBS1 | 0.0625 | VTN | 0.315 | | | <0.005 | 0.840 |
| THBS2 | 0.0625 | THBS4 | 0.25 | | | <0.005 | 0.850 |
| THBS1 | 0.25 | THBS2 | 0.25 | | | <0.05 | 0.859 |
| THBS2 | 0.0625 | FGF17 | 0.1 | | | <0.005 | 0.871 |
| THBS2 | 0.0625 | VTN | 0.625 | | | <0.005 | 0.876 |
| THBS2 | 0.125 | THBS4 | 0.125 | | | <0.005 | 0.878 |
| THBS1 | 0.0625 | VTN | 1.25 | | | <0.05 | 0.880 |
| THBS1 | 0.25 | THBS4 | 0.0625 | | | <0.005 | 0.888 |
| THBS2 | 0.0625 | FGF17 | 0.025 | | | <0.005 | 0.890 |
| THBS1 | 0.0625 | THBS2 | 0.0625 | | | <0.05 | 0.900 |
| THBS1 | 0.25 | VTN | 1.25 | | | <0.005 | 0.907 |
| THBS2 | 0.125 | FGF17 | 0.025 | | | <0.005 | 0.913 |
| VTN | 0.3125 | FGF17 | 0.025 | | | <0.05 | 0.915 |
| THBS4 | 0.25 | VTN | 0.3125 | | | <0.05 | 0.922 |
| THBS2 | 0.125 | FGF17 | 0.1 | | | <0.005 | 0.923 |
| THBS1 | 0.25 | VTN | 0.625 | | | <0.005 | 0.930 |
| THBS2 | 0.0625 | THBS4 | 0.0625 | | | <0.05 | 0.942 |
| THBS4 | 0.25 | FGF17 | 0.1 | | | <0.005 | 0.945 |
| THBS4 | 0.0625 | VTN | 0.625 | | | <0.05 | 0.950 |
| VTN | 0.625 | FGF17 | 0.1 | | | <0.005 | 0.952 |
| THBS4 | 0.25 | VTN | 1.25 | | | <0.05 | 0.954 |

TABLE 11
Additional data regarding synergistic combinations of HAPs (mouse myoblasts)

| | | | Single | | | | Combo | | | |
|---|---|---|---|---|---|---|---|---|---|---|
| | | | Dose | | | | Dose | | | |
| | Single | Single | % EdU | | | | % EdU | | | |
| | Dose @ | Dose | Fold | Single | Combo | Combo | Fold | Combo | | |
| Factor | saturation | % of | Change | Dose | Dose | Dose % | Change | Dose | | |
| Name | (ug/mL) | EB | to FM | % EdU | (ug/mL) | of EB | to FM | % EdU | CI_type | CI_Value |
| THBS1 | 2 | 50% | 1.70 | 15% | 0.0625 | 64% | 1.16 | 14% | HSA | 0.679 |
| FGF17 | 0.5 | 239% | 4.50 | 69% | 0.1 | 148% | 2.89 | 33% | | |
| combo | | | | | | 163% | 3.63 | 29% | BI | 0.194 |
| THBS2 | 2 | 50% | 1.25 | 13% | 0.125 | 46% | 0.88 | 10% | HSA | 0.633 |
| THBS4 | 2 | 40% | 1.87 | 10% | 0.25 | 53% | 1.11 | 12% | | |
| Combo | | | | | | 71% | 1.39 | 17% | BI | 0.729 |
| THBS1 | 2 | 50% | 1.70 | 15% | 0.0625 | 44.08% | 0.75 | 10.63% | HSA | 0.852 |
| VTN | 10 | 50% | 1.50 | 16% | 10 | 59.18% | 1.01 | 14.27% | | |
| Combo | | | | | | 69.47% | 1.19 | 16.75% | BI | 0.845 |
| THBS2 | 1 | 50% | 1.54 | 13% | 0.125 | 31.08% | 0.85 | 7.86% | HSA | 0.680 |
| THBS4 | 2 | 40% | 1.87 | 10% | 0.25 | 29.33% | 0.80 | 7.41% | | |
| IL15 | 0.5 | 52% | 1.85 | | 1 | 47.25% | 1.28 | 11.90% | BI | 1.239 |
| Combo | | | | | | 57.91% | 1.66 | 16.21% | | |
| THBS2 | 1 | 50% | 1.54 | 13% | 0.125 | 45.08% | 0.79 | 9.64% | HSA | 0.886 |
| THBS4 | 2 | 40% | 1.87 | 10% | 0.25 | 61.15% | 1.08 | 13.08% | | |
| VTN | 10 | 50% | 1.50 | 16% | 10 | 54.46% | 0.94 | 12.67% | BI | 1.650 |
| combo | | | | | | 69.02% | 1.22 | 14.76% | | |
| THBS2 | 1 | 50% | 1.54 | 13% | 0.125 | 58.62% | 0.86 | 15.23% | HSA | 0.733 |
| THBS4 | 2 | 40% | 1.87 | 10% | 0.25 | 62.99% | 0.93 | 16.37% | | |
| IGF2 | 0.03 | 89% | 2.28 | | 0.5 | 85.78% | 1.27 | 22.29% | BI | 1.184 |
| combo | | | | | | 116.95% | 1.72 | 30.39% | | |
| FGF17 | 0.5 | 50% | 1.70 | 15% | 0.0125 | 49.74% | 1.05 | 12.10 | HSA | 0.725 |
| BMP7 | 0.025 | 117% | 1.71 | 18% | 0.0125 | 90.39% | 1.91 | 21.98 | | |
| combo | | | | | | 124.73% | 2.63 | 30.33 | BI | 0.363 |
| IGF2 | 0.03 | 89% | 2.28 | 23% | 0.012 | 41.54 | 1.69 | 8.04 | HSA | 0.73 |
| BMP7 | 0.025 | 117% | 1.71 | 18% | 0.0125 | 112.33 | 4.39 | 21.73 | | |
| combo | | | | | | 153.16 | 6.22 | 29.63 | BI | −0.23 |

TABLE 12
Additional data regarding synergistic combinations of HAPs (mouse myoblasts)

| | | | | | | Combo Dose | |
|---|---|---|---|---|---|---|---|
| | Single Dose | Single Dose | Single Dose | Combo Dose | Combo Dose | % EdU Fold | Combo Dose |
| Factor | (ug/mL) | % of Eb | % EdU | (ug/mL) | % of EB | Change to FM | % EdU |
| THBS1 | 2 | 50% | 15% | 0.125 | 64% | 1.16 | 14% |
| FGF17 | 0.5 | 239% | 69% | 0.1 | 148% | 2.89 | 33% |
| Combo | | | | | 163% | 3.63 | 29% |
| THBS2 | 2 | 50% | 13% | 0.125 | 46% | 0.88 | 10% |
| THBS4 | 2 | 40% | 10% | 0.25 | 53% | 1.11 | 12% |
| Combo | | | | | 71% | 1.39 | 17% |
| THBS1 | 2 | 50% | 15% | 0.0625 | 44.08% | 0.75 | 10.63% |
| VTN | 10 | 50% | 16% | 10 | 59.18% | 1.01 | 14.27% |
| Combo | | | | | 69.47% | 1.19 | 16.75% |
| THBS2 | 2 | 50% | 13% | 0.125 | 31.08% | 0.85 | 7.86% |
| THBS4 | 2 | 40% | 10% | 0.25 | 29.33% | 0.8 | 7.41% |
| IL15 | 0.5 | 52% | 12.50% | 1 | 47.25% | 1.28 | 11.90% |
| Combo | | | | | 57.91% | 1.66 | 16.21% |
| THBS2 | 2 | 50% | 13% | 0.125 | 45.08% | 0.79 | 9.64% |
| THBS4 | 2 | 40% | 10% | 0.25 | 61.15% | 1.08 | 13.08% |
| VTN | 10 | 50% | 16% | 1 | 54.46% | 0.94 | 12.67% |
| Combo | | | | | 69.02% | 1.22 | 14.76% |

TABLE 13
Additional data regarding combinations of proteins with synergistic regenerative
effects in human myoblasts as measured by nuclei counts per well

| | Single dose | | | | | Combo dose | | | | |
|---|---|---|---|---|---|---|---|---|---|---|
| | | | Nuclei | | | | | Nuclei | | |
| | | | Counts | | | | | Counts | | |
| | @ | | Fold | | | | % of EB | Fold | | |
| Factor | saturation | % of | Change | Nuclei | | Dose | (8x EB | Change | Nuclei | HSA |
| Name | (ug/mL) | EB | to FM | Counts | | (ug/mL) | @ 856) | to FM | Counts | CI |
| THBS2 | 2 | 82.21 | 1.16 | 5000 | THBS2/ | 0.5/0.5 | 187.85 | 1.13 | 1608 | |
| | | | | | THBS4 | | | | | |
| THBS4 | 1 | 58.90 | 0.83 | 3582 | IGF2 | 0.1 | 219.45 | 1.32 | 1879 | |
| IGF2 | 1 | 120.84 | 1.07 | 5532 | THBS2/ | | 239.08 | 1.44 | 2047 | 0.92 |
| | | | | | THBS4/ | | | | | |
| | | | | | IGF2 | | | | | |
| THBS2 | 2 | 82.21 | 1.16 | 5000 | THBS2/ | 0.5/0.5 | 187.85 | 1.13 | 1608 | |
| | | | | | THBS4 | | | | | |
| THBS4 | 1 | 58.90 | 0.83 | 3582 | IGF2 | 0.25 | 229.09 | 1.38 | 1961 | |
| IGF2 | 1 | 120.84 | 1.07 | 5532 | THBS2/ | | 278.39 | 1.67 | 2383 | 0.82 |
| | | | | | THBS4/ | | | | | |
| | | | | | IGF2 | | | | | |
| THBS2 | 2 | 82.21 | 1.16 | 5000 | THBS2/ | 0.5/0.5 | 187.85 | 1.13 | 1608 | |
| | | | | | THBS4 | | | | | |
| THBS4 | 1 | 58.90 | 0.83 | 3582 | IGF2 | 0.5 | 236.57 | 1.42 | 2025 | |
| IGF2 | 1 | 120.84 | 1.07 | 5532 | THBS2/ | | 291.41 | 1.75 | 2495 | 0.81 |
| | | | | | THBS4/ | | | | | |
| | | | | | IGF2 | | | | | |
| THBS2 | 2 | 82.21 | 1.16 | 5000 | THBS2/ | 0.125/0.125 | 192.52 | 1.16 | 1648 | |
| | | | | | THBS4 | | | | | |
| THBS4 | 1 | 58.90 | 0.83 | 3582 | IL15 | 0.125 | 202.22 | 1.22 | 1731 | |
| IL15 | 0.5 | 117.58 | 1.04 | 5383 | THBS2/ | | 234.40 | 1.41 | 2007 | 0.86 |
| | | | | | THBS4/ | | | | | |
| | | | | | IL15 | | | | | |

TABLE 14
Additional data regarding synergistic combinations of HAPs (human myoblasts)

| | | | Single | | | | Combo | | |
|---|---|---|---|---|---|---|---|---|---|
| | | | Dose | | | | Dose | | |
| | Single | Single | % EdU | | | Combo | % EdU | | |
| | Dose @ | Dose | Fold | Single | Combo | Dose | Fold | Combo | |
| Factor | saturation | % of | Change | Dose | Dose | % of | Change | Dose | HSA |
| Name | (ug/mL) | EB | to FM | % EdU | (ug/mL) | EB | to FM | % EdU | CI |
| THBS1 | 7.5 | 98.93 | 1.61 | 10.99 | 0.5 | 78.22 | 0.85 | 8.95 | |
| FGF17 | 0.25 | 222.09 | 3.62 | 24.67 | 0.05 | 121.74 | 1.33 | 13.93 | |
| Combo | | | | | | 157.29 | 1.72 | 18 | 0.77 |
| THBS2 | 7.5 | 112.8469 | 1.2 | 12.53448 | 0.125 | 57.09 | 0.95 | 7.82 | |
| THBS4 | 0.5 | 126.7002 | 2.07 | 14.07323 | 0.25 | 62.67 | 1.04 | 8.59 | |
| IL15 | 0.1 | 67.81 | 1.13 | 9.29 | 0.1 | 67.81 | 1.13 | 9.29 | |
| Combo | | | | | | 69 | 1.15 | 9.46 | 0.98 |

TABLE 15
Additional data regarding synergistic combinations of HAPs (human myoblasts)

| | | | Single | | | | | Combo | | |
|---|---|---|---|---|---|---|---|---|---|---|
| | | | Dose | | | | | Dose | | |
| | Single | Single | % EdU | | | | Combo | % EdU | Combo | |
| | Dose @ | Dose | Fold | Single | | Combo | Dose | Fold | Dose | |
| Factor | saturation | % of | Change | Dose | | Dose | % of | Change | Dose | HSA |
| Name | (ug/mL) | EB | to FM | % EdU | | (ug/mL) | EB | to FM | % EdU | CI |
| THBS1 | 7.5 | 98.93 | 1.61 | 10.99 | THBS1 | 0.5 | 78.22 | 0.85 | 8.95 | |
| FGF17 | 0.25 | 222.09 | 3.62 | 24.67 | FGF17 | 0.05 | 121.74 | 1.33 | 13.93 | |
| | | | | | THBS1/ | 0.5/0.05 | 157.29 | 1.72 | 18.00 | 0.77 |
| | | | | | FGF17 | | | | | |
| THBS1 | 7.5 | — | 1.30 | 12.46 | THBS1 | 0.125 | | 0.989 | 11.80 | |
| FGF17 | 1 | — | 1.50 | 14.80 | FGF17 | 0.025 | | 1.09 | 12.80 | |
| | | | | | THBS1/ | 0.125/0.025 | | 1.281 | 14.30 | 0.85 |
| | | | | | FGF17 | | | | | |

### Example 8—BMP7 Induces Myoblast Proliferation

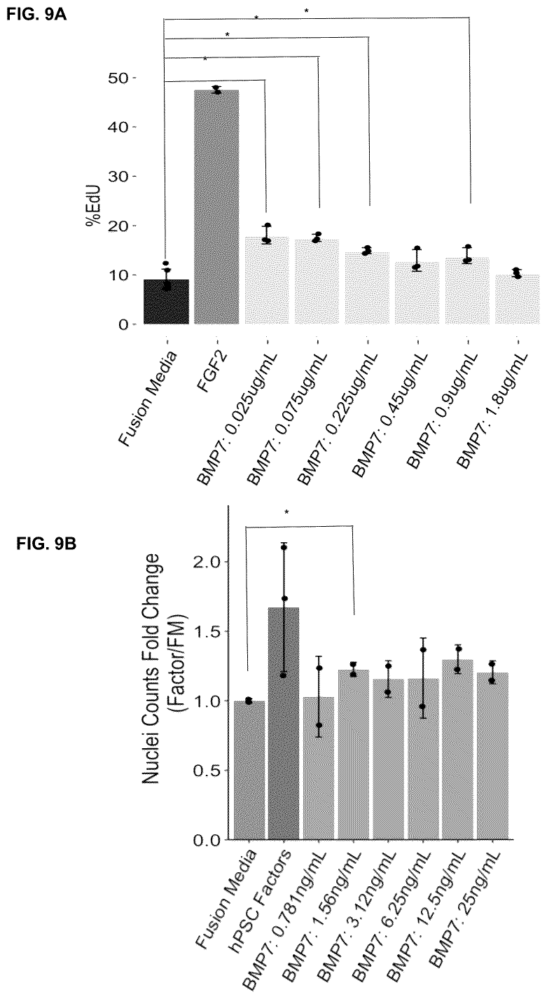

Mouse myoblast cells were cultured for 48 hours in the presence of BMP 7 at either 0.025 μg/mL, 0.075 μg/mL, 0.22 μg/mL, 0.45 μg/mL, 0.45 μg/mL, 0.9 μg/mL, 1.8 μg/mL, or vehicle only. Fresh media and BMP7 was added every 24 hours. After 48 hours, the cells were stained for EdU, Hoescht and eMyHC using methods similar to those in Example 1. Representative images of the images are seen in FIG. 9A. BMP7 treatment resulted in increased proliferation in the mouse myoblasts, as seen by the increase in EdU positive cells. The number of EdU positive nuclei was quantified, and this information is displayed in FIG. 9B and Table 16. Most doses of BMP7 resulted in an increase in the percent of EdU positive nuclei when compared to myoblasts treated with the vehicle alone. In particular, treatment with 0.025 μg/mL, 0.075 μg/ml, 0.2255 μg/mL, and 0.9 μg/ul resulted in a significant change in the percentage of EdU positive nuclei, indicating an increase in myoblast proliferation. Significance was determined by a Welch's one-tailed t-test with a p value <0.05.

TABLE 16
BMP7-induced proliferation in mouse myoblasts

| BMP7 | % EdU | p-value |
|---|---|---|
| Vehicle | 10.56 | — |

| | | | |
|---|---|---|---|
| 0.025 | ug/mL | 18.08 | 1.21E−07 |
| 0.075 | μg/mL | 17.53 | 8.05E−07 |
| 0.2255 | μg/mL | 14.93 | 3.37E−03 |
| 0.45 | μg/mL | 12.93 | n.s. |
| 0.9 | μg/mL | 13.91 | 4.99E−02 |
| 1.8 | μg/mL | 10.37 | n.s. |

BMP7 treatment also induced proliferation of human myoblasts. Human myoblasts were cultured with BMP7 at a dose of either 0.78 ng/ml, 1.56 ng/ml, 3.12 ng/ml, 6.25 ng/mL. 12.5 ng/ml, or 25 ng/mL for 72 hours. Media and BMP7 was changed every 24 hours. After 72 hours, cells were stained and imaged for EdU as described in Example 1. The number of EdU positive nuclei was quantified and this information is displayed in FIG. 9C and Table 17 However, only the 1.56 ng/mL dose of BMP7 resulted in a significant increase in myoblast proliferation compared to cells treated with vehicle alone.

TABLE 17
BMP7-induced proliferation in human myoblasts

| | Nuclei | |
|---|---|---|
| Human Myoblast | Counts | p-value |
| Vehicle | 2083.5 | — |

| | | | |
|---|---|---|---|
| 0.78 | ng/mL | 2144.5 | n.s. |
| 1.56 | ng/mL | 2552.5 | 0.04 |
| 3.12 | ng/mL | 2408 | n.s. |
| 6.25 | ng/mL | 2422.5 | n.s. |
| 12.5 | ng/mL | 2706 | n.s. |
| 25 | ng/mL | 2509 | n.s. |

### Example 9—IGF2 and BMP7 Combination Treatment is More Potent than the Single Factors Individually

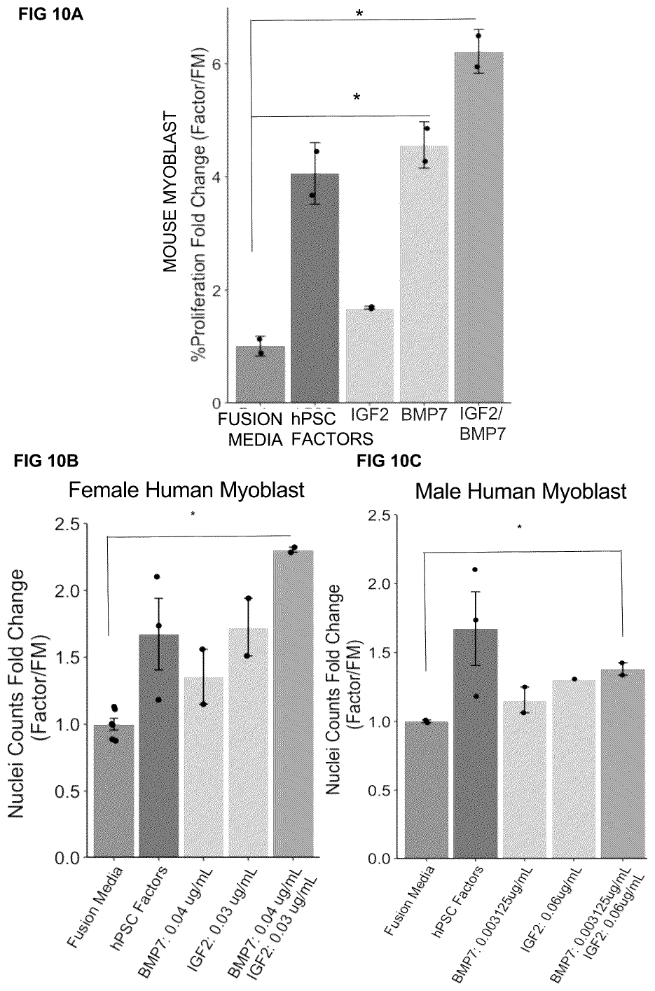

Mouse myoblasts were cultured in media and with either PBS(vehicle), hPSC, IGF2, BMP7, or IGF2 and BMP7 combined. Fresh media and factors were added every 24 hours. After 48 hours, cells were stained and imaged for EdU as described in Example 1. The percent of EdU positive nuclei were quantified, as shown in FIG. 10A and Table 18. Compared to untreated cells, treatment with hPSC factors, BMP7, and BMP7/IGF2 resulted in a significant fold-change in proliferation. However, the combination treatment of BMP7/IGF2 resulted in the greatest fold-change of all treatment conditions, with a fold change of 6.22.

TABLE 18
BMP7-and IGF2-induced proliferation in mouse myoblasts

| Factor | | % EdU Fold | |
|---|---|---|---|
| Name | (ug/mL) | Change | P-value |
| IGF2 | 0.012 | 1.69 | n.s. |
| BMP7 | 0.006 | 4.56 | 1.39E−03 |
| Combined | | 6.22 | 3.82E−06 |

Female and male myoblasts were cultured in media and with added factors. The factors consisted of either PBS (vehicle only), hPSC factors, IGF2, BMP7, or a combination of IGF2 and BMP7. After 72 hours, cells were stained and imaged for EdU as described in Example 1. The number of EdU positive nuclei was quantified in female myoblasts, application of either the hPSC factors or the combined IGF2 and BMP7 resulted in a significant increase in the number of EdU positive nuclei, while treatment with either factor alone did not result in a significant increase in proliferation, when compared to cells cultured with vehicle alone, as depicted in FIG. 10B and Table 19.

TABLE 19
BMP7- and IGF2-induced proliferation in human female myoblasts

| Factor | | Nuclei | |
|---|---|---|---|
| Name | (ug/mL) | Counts | P-value |
| IGF2 | 0.03 | 1331 | n.s. |
| BMP7 | 0.04 | 1041 | n.s. |
| Combined | | 1546 | 8.32E−08 |

In human male myoblasts, there was a significant increase in nuclei count fold change when comparing either the hPSC factors or the combined IGF2/BMP7 condition to the fusion media, as depicted in FIG. 10C and Table 20. Treatment with either factor alone did not result in a significant increase compared to untreated cells.

TABLE 20
BMP7 and IGF2 induced proliferation in human male myoblasts

| Factor | | Nuclei | |
|---|---|---|---|
| Name | (ug/mL) | Counts | P-value |
| IGF2 | 0.06 | 2723 | n.s. |
| BMP7 | 0.003 | 2408 | n.s. |
| Combined | | 2875 | 3.12E−02 |

### Example 10—FGF17 and BMP7 Combination Treatment Induced Proliferation in Myoblasts

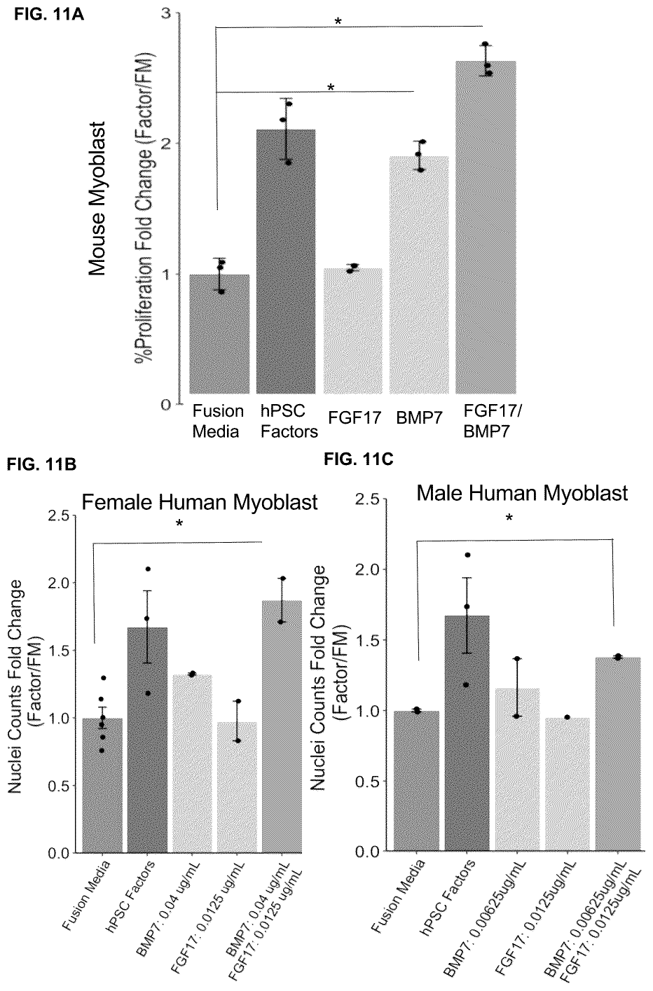

Mouse myoblasts were cultured in media and with either PBS (vehicle), hPSC, IGF2, BMP7, or IGF2 and BMP7 combined. Fresh media and factors were added every 24 hours. After 48 hours, cells were stained and imaged for EdU as described in Example 1. Representative images of the mouse myoblasts treated with the vehicle, hPSC factors, FGF17, BMP7, and the combination FGF17/BMP7 are shown in FIG. 11A. The number of EdU positive nuclei were quantified for each treatment condition, as depicted in FIG. 11B and Table 21. The cells treated with the hPSC factors, BMP7, and the combination treatment had significant increases in the percent fold change. Furthermore, the combination treatment of BMP7 and FGF17 produced the greatest fold-change increase in proliferation, with a fold-change of 2.63 when compared to untreated cells.

TABLE 21
Mouse myoblast proliferation

| Factor | Dose | Fold | |
|---|---|---|---|
| Name | (ug/mL) | Change | P-value |
| FGF17 | 0.012 | 1.05 | n.s. |
| BMP7 | 0.012 | 1.91 | 3.47E−04 |
| Combined | | 2.63 | 3.75E−05 |

Human myoblasts were also cultured with hPSC factors, FGF17, BMP7, and a combination FGF17/BMP7 treatment. The number of EdU positive nuclei were quantified for each treatment condition to assess myoblast proliferation. Culturing female human myoblast cells with either the hPSC factors or the combination FGF17/BMP7 produced a significant difference in the fold change of proliferating myoblasts, as depicted in FIG. 11C and Table 22. The combination factors resulted in a similar fold-change increase as culturing the cells with the hPSC factors. However, culturing the cells with either FGF17 or BMP7 alone did not produce a significant increase in proliferation.

TABLE 22
Human female myoblast proliferation

| | Dose | Nuclei | |
|---|---|---|---|
| Factor Name | (ug/mL) | Counts | P-value |
| FGF17 | 0.0125 | 786 | n.s. |
| BMP7 | 0.04 | 1041 | n.s. |
| Combined | | 1470 | 3.90E−03 |

Culturing male human myoblast wells with the hPSC factors produced the greatest fold change increase in proliferation, compared to cells cultured with PBS, as depicted in FIG. 11D. Culturing male myoblast cells with either FGF17 or BMP7 alone did not produce a significant fold change in proliferation. However, cells that were cultured with a combination of FGF17 and BMP7 had a significant increase in proliferation, as depicted in FIG. 11D and Table 23.

TABLE 23
Male human myoblast proliferation

| | Dose | Nuclei | |
|---|---|---|---|
| Factor Name | (ug/mL) | Counts | P-value |
| FGF17 | 0.0125 | 1985 | n.s. |
| BMP7 | 0.00625 | 2422.5 | n.s. |
| Combo | | 2872.5 | 7.25E−04 |

### Example 11—THBS1 Receptor Expression in Mouse and Human Myoblasts

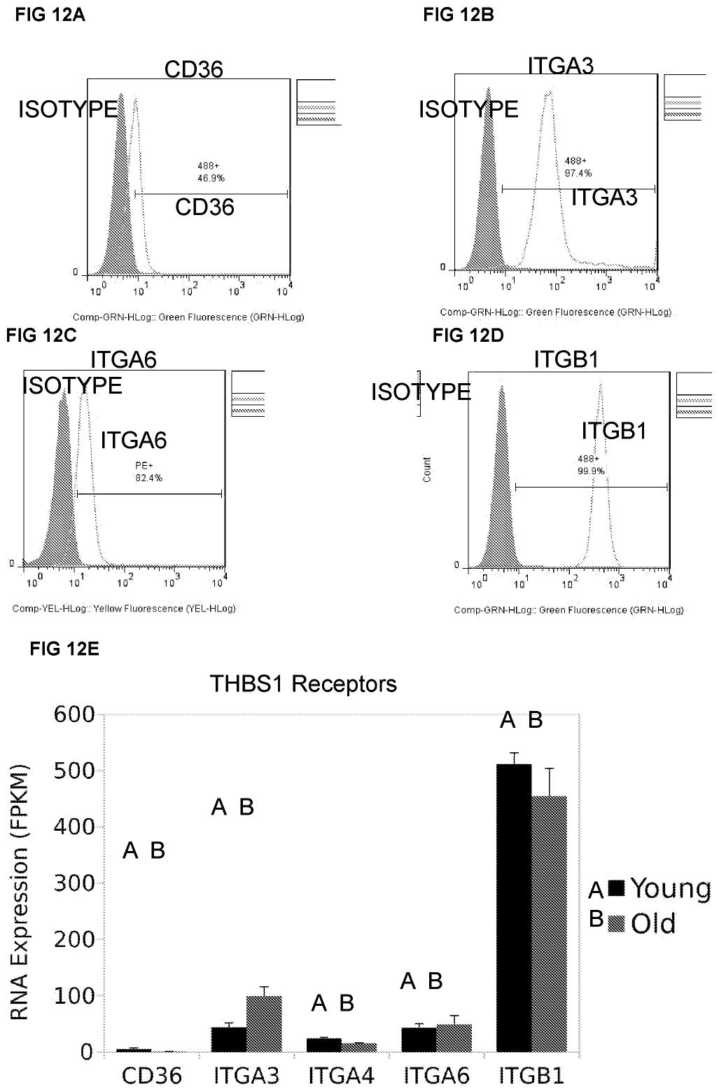

Mouse myoblast cells were cultured in growth media, as described in example 1. After 48 hours, the cells were analyzed via flow cytometry for the presence of THBS1 receptors on the cell surface. As depicted in FIG. 12A and Table 24, 46.9% of the cells were positive for the THBS1 receptor CD36. As depicted in FIG. 12B and Table 24, 97.4% of the cells were positive for the THBS1 receptor ITGA3. As depicted in FIG. 12C and Table 24, 82.4% of the cells were positive for the THBS1 receptor ITGA6. As depicted in FIG. 12D and Table 24, 99.9% of the cells were positive for the THBS1 receptor ITGB1.

TABLE 24
THBS1 receptors on the cell surface of mouse myoblasts

| Receptor | Positive Cells (%) |
|---|---|
| CD36 | 46.9 |
| ITGA3 | 97.4 |
| ITGA6 | 82.4 |
| ITGB1 | 99.9 |

Young and aged human myoblast cells were cultured in fusion media, with fresh media added every 24 hours. After 96 hours, RNA was isolated from each well (RNeasy Mini Kit, Qiagen) and cDNA was obtained by reverse-transcription (High Capacity Reverse Transcription Kit, Thermo Fisher Scientific). Real-time quantitative PCR was performed using QuantStudio3 (Thermo Fisher).

As depicted in FIG. 12E and Table 25, both young and aged myoblasts showed expression of THBS1 receptors. CD36 expression was low in old myoblasts, with a FPKM of 0.871 compared to a FPKM of 5.032 in young myoblasts. ITGA2 expression was higher in old myoblasts than young myoblasts, with a FPKM of 99.259 compared to a FPKM of 44.177. ITGA4 expression was higher in young myoblasts than in old myoblasts, with a FPLKM of 24.348 compared to a FPKM of 15.409. ITGA6 expression was higher in old myoblasts than young myoblasts, with a FPKM of 49.069 compared to a FPKM of 43.355. ITGB1 expression was higher in young myoblasts than in old myoblasts, with a FPKM of 511.270 compared to a FPKM of 454.480.

TABLE 25
Human myoblast cells for receptors

| GeneName | Young | Old | Young_SEM | Old_SEM |
|---|---|---|---|---|
| CD36 | 5.032 | 0.871 | 2.096 | 0.085 |
| ITGA3 | 44.177 | 99.259 | 7.600 | 16.691 |
| ITGA4 | 24.346 | 15.409 | 1.706 | 0.940 |
| ITGA6 | 43.355 | 49.069 | 7.058 | 15.548 |
| ITGB1 | 511.270 | 454.48 | 20.174 | 49.053 |

### Example 12—Receptors for hPSC Factors are Expressed in Human Myoblasts

Young and aged human myoblast cells were cultured in fusion media, with fresh media added every 24 hours. After 96 hours, RNA was isolated from each well (RNeasy Mini Kit, Qiagen) and cDNA was obtained by reverse-transcription (High Capacity Reverse Transcription Kit, Thermo Fisher Scientific). Real-time quantitative PCR was performed using QuantStudio3 (Thermo Fisher).

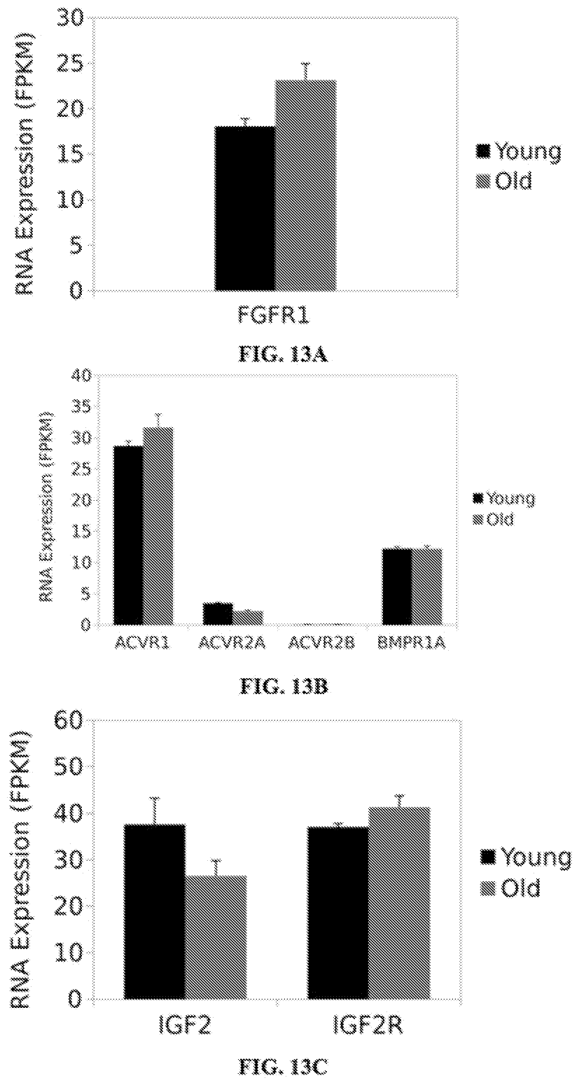

FGF17 was one of the factors present in the hPSC. FGF17 activates the receptor FGFR1. As depicted in Table 26 and FIG. 13A, both young and aged myoblasts expressed FGFR1.

TABLE 26
RNA Expression (in FPKM) of factor
receptors in human myoblasts

| | Young | Old | | |
|---|---|---|---|---|
| GeneName | (n = 6) | (n = 6) | Young_SEM | Old_SEM |
| FGFR1 | 18.069 | 23.122 | 0.843 | 1.836 |
| ACVR1 | 28.705 | 31.643 | 0.746 | 2.070 |
| ACVR2A | 3.508 | 2.190 | 0.088 | 0.191 |
| ACVR2B | 0.083 | 0.129 | 0.016 | 0.018 |
| BMPR1A | 12.270 | 12.240 | 0.299 | 0.393 |
| IGF2 | 37.589 | 26.511 | 5.690 | 3.341 |
| IGF2R | 37.033 | 41.306 | 0.761 | 2.503 |

BMP7 was one of the factors present in the hPSC. BMP7 is capable of interacting with several receptors, including ACVR1, ACVR2A, ACVR2B and BMPR1A. Both young and old myoblasts showed expression of multiple BMP7 receptors, as depicted in FIG. 13B and Table 26. ACVR1 was expressed at the highest levels in myoblasts, with a FPKM of 28.705 in young myoblasts and 31.643 in old myoblasts. BMPR1A had expression levels of 12.270 FPKMs and 12.240 FPKMs in young and old myoblasts, respectively. ACVR2A had expression levels of 3.508 and 2.190 FPKMs in young and old myoblasts respectively. ACVR2B has expression levels of 0.083 and 0.129 FPKMs, respectively.

Another hPSC factor was IGF2. Both IGF2 and IGF2R expression was present in human myoblasts, as depicted in FIG. 13C and Table 26. IGF2 levels were higher in young myoblasts than old myoblasts, with FPKMs of 37.589 and 26.511. respectively. IGF2R was expressed in both young and old myoblasts, with expression levels of 37,033 FPKMs and 41.306 FPKMs, respectively.

### Example 13—Transcriptional Profiling

### Myogenic Gene Profiling for Pro-Regenerative Factors

Expression of myogenic factors Pax7, Myf5, Myod1, and Myog are key indicators of the functional status of muscle progenitor cells. Factors upregulating of Pax7 and Myf5 indicate rejuvenation of proliferative progenitor cells whereas upregulation of Myod1 and Myog are indicative of muscle myofiber regeneration. A read-out of these gene expressions will provide potential success for any given HAP as a regenerative factor. Measuring myogenic genes in mouse or human muscle progenitor cells treated with factors will provide a good characterization of the therapeutic effect for treating individuals who have suffered injury, or who possess genetic or developmental defects leading to premature tissue loss, wasting, or weakening. As a control, the assay will also be performed on proteins purified from differentiated cells, which result in no in myoblast proliferation, cultured in medium conditioned by differentiated cells, or purified heparin-associated fractions.

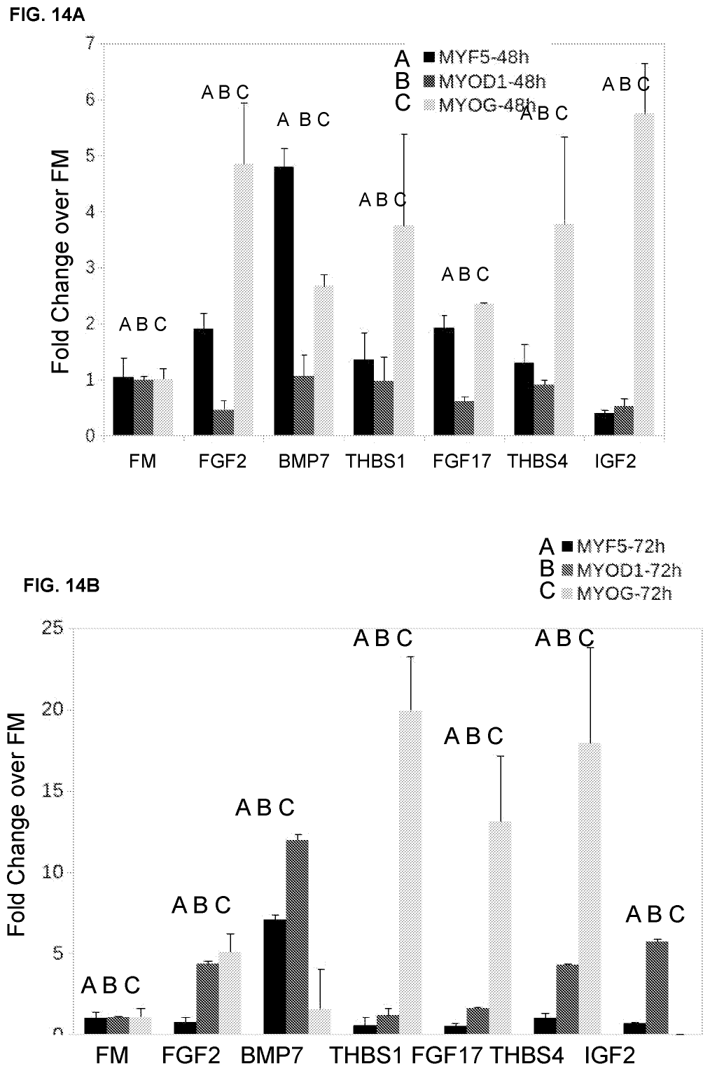

Aged human myoblasts were cultured in well plates. Culturing the cells with the different medias resulted in differential induction of myogenic gene expression. All factors resulted in changes in at least one myogenic receptor gene at 48 hours and 72 hours when compared to cells cultured in fusion media, as depicted in Table 27 and FIGS. 14A-14B. RNA was isolated from each well (RNeasy Mini Kit, Qiagen) and cDNA was obtained by reverse-transcription (High Capacity Reverse Transcription Kit, Thermo Fisher Scientific). Real-time quantitative PCR was performed using QuantStudio3 (Thermo Fisher).

Cells that had been cultured with FGF2 had increases in levels of both MYF and MYOG, but not MYOD. Cells that had been cultured with BMP7 had increases in levels of MYF5 and MYOG at 48 hours and all 3 receptors at 72 hours. Cells that had been cultured with THBS1 had increases in levels of MYF5 and MYOG at 48 hours and in levels of MYOD and MYOG at 72 hours. Cells that had been cultured with FGF17 had increases in levels of MYF5 and MYOG at 48 hours, and increases in levels of MYOD and MYOG at 72 hours. Cells that had been cultured with THBS4 had increases in levels of MYF5 and MYOG at 48 hours and increases in levels of all 3 factors at 72 hours. Cells that had been cultured with IGF2 had increases in levels of MYOG at 48 hours and levels of MYOD at 72 hours. Values were expressed as fold change compared to vehicle treated controls.

TABLE 27
Fold change increase in myogenic transcription factor expression
in aged human myoblasts cultured with different factors

| | MYF5- | MYOD1- | MYOG- | MYF5- | MYOD1- | MYOG- |
|---|---|---|---|---|---|---|
| Condition | 48 h | 48 h | 48 h | 72 h | 72 h | 72 h |
| FM | 1.04 | 1.001 | 1.013 | 1.023 | 1.055 | 1.092 |
| FGF2 | 1.918 | 0.448 | 4.851 | 0.784 | 4.339 | 5.075 |
| BMP7 | 4.804 | 1.06 | 2.677 | 7.038 | 11.934 | 1.562 |
| THBS1 | 1.354 | 0.968 | 3.735 | 0.605 | 1.174 | 19.906 |
| FGF17 | 1.922 | 0.604 | 2.353 | 0.499 | 1.627 | 13.124 |
| THBS4 | 1.307 | 0.91 | 3.773 | 1.014 | 4.299 | 17.925 |
| IGF2 | 0.409 | 0.519 | 5.756 | 0.708 | 5.723 | 0.018 |

### Myogenic Gene Profiling in Mouse Progenitor Cells

Mouse muscle progenitor cells plated at 10,000 cells/well on Matrigel coated 96-well plates in 100 μL medium per well (1:100 matrigel: PBS) in mouse fusion medium: DMEM (Gibco)+2% horse serum (Hyclone). One hour after plating, mouse myoblasts are treated with respective factors. Mouse myoblasts are analyzed for expression of Pax7, Myf5, Myod1, and Myog to characterize the regenerative effect of treatment with the therapeutic factor. Values were expressed as fold change compared to vehicle treated controls.

TABLE 28
Fold change increase in myogenic transcription factor expression
in mouse muscle progenitor cells cultured with different factors

| | Dose | | | |
|---|---|---|---|---|
| Condition | (ug/mL) | Mean | SD | p-value |
| Gene: | Pax7 | | | |
| FM | 2 | 1.011 | 0.185 | — |
| THBS1 | 2 | 1.045 | 0.105 | n.s. |
| IGF2 | 0.2 | 1.459 | 0.207 | 0.049 |
| THBS4 | 0.5 | 1.511 | 0.567 | n.s |
| FGF17 | 0.5 | 6.118 | 0.920 | 7.05E−04 |
| BMP7 | 0.025 | 8.752 | 1.528 | 9.56E−04 |
| Gene: | Myf5 | | | |
| FM | 2 | 1.002 | 0.071 | — |
| THBS1 | 2 | 0.921 | 0.094 | n.s. |
| THBS4 | 0.2 | 1.365 | 0.319 | n.s. |
| FGF17 | 0.5 | 1.376 | 0.201 | 1.69E−02 |
| IGF2 | 0.5 | 1.383 | 0.051 | 1.66E−03 |
| BMP7 | 0.025 | 40.738 | 5.627 | 6.65E−05 |
| Gene: | Myod1 | | | |
| FM | 2 | 1.002 | 0.086 | — |
| THBS1 | 2 | 0.964 | 0.023 | n.s. |
| THBS4 | 0.2 | 1.073 | 0.062 | n.s. |
| FGF17 | 0.5 | 1.230 | 0.158 | n.s. |
| IGF2 | 0.5 | 1.328 | 0.036 | 6.41E−03 |
| BMP7 | 0.025 | 2.004 | 0.075 | 1.09E−04 |

### Transcriptome Profiling in Human Progenitor Cells

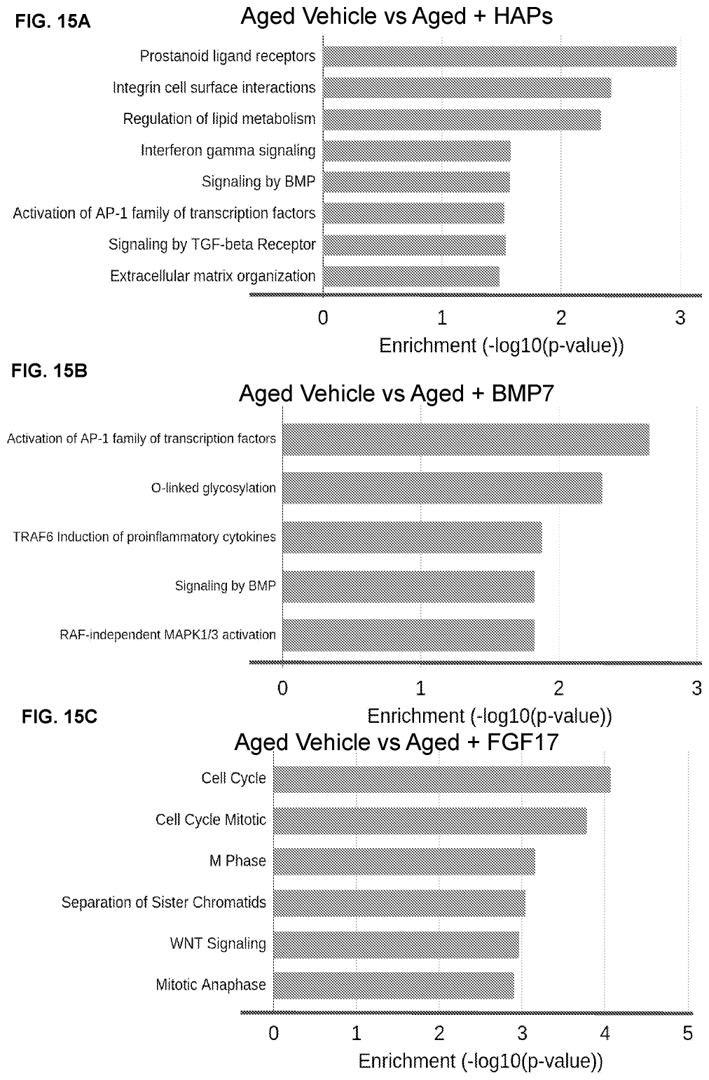

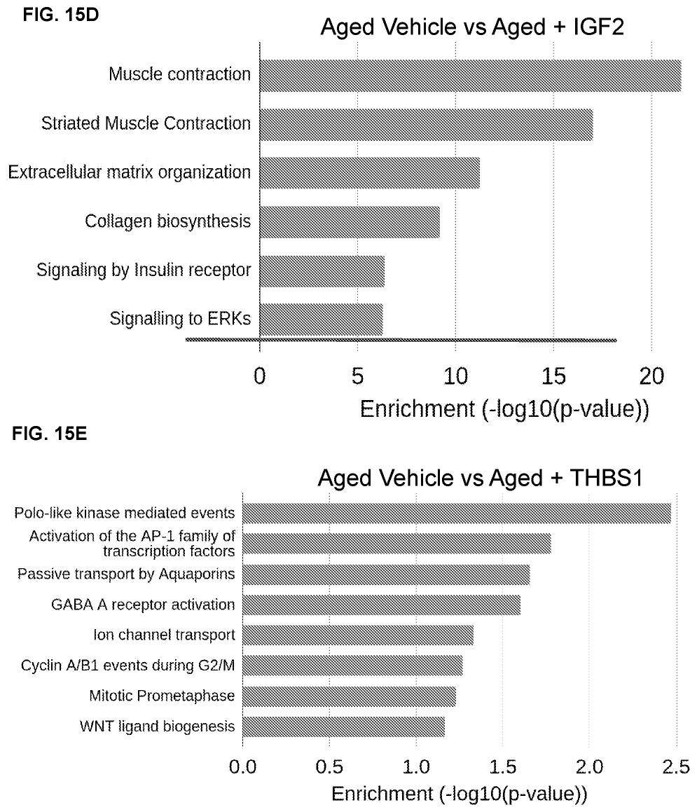

Aged human myoblasts were cultured in well plates. Culturing the cells with the different medias resulted in differential induction of myogenic gene expression (FIG. 15A-E). Differentially enriched genes and pathways driven by heparin-associated proteins (HAPs) cocktail or individual factors including FGF17, IGF2, or BMP7 in aged human muscle cells. Cells were treated with indicated factor every 24 h for 96 h. Total RNA was isolated using the RNeasy Mini Kit (Qiagen) and was further purified via polyA selection. cDNA was then generated and illumina adaptor indexes were ligated to generate RNAseq libraries (NEBNext Ultra). Libraries were sequenced using an Illumina HiSeq 4000 sequencer (Illumina). RNA abundance was obtained by STAR and RSEM software. Expression is normalized to Z-score. n=4 biological replicates. (FIG. 15A) HAPs induce extracellular matrix and cell surface interactions to activate signal cascades promoting proliferative homeostasis. Reactome pathway analysis performed on all upregulated or downregulated DE genes (cutoff=p-value 0.01) discovered upregulation of several categories (Cell Cycle, M Phase, Separation of Sister Chromatids, WNT signaling, and Mitotic Anaphase). Enriched pathways were obtained from GSEA Reactome_2016 gene sets. Enrichment is shown according to the p-value. Positive values are upregulated pathways and negative values are downregulated pathways.

### Example 14—Plasma Membrane Receptor Profiling by Fractionating and Label-Free Mass Spectrometry

Plasma membrane receptor profiling was performed by fractionation and label-free mass spectrometry of aged, primary human myoblasts cultured in well plates in growth media. Cells were harvested by EDTA cold buffer and cell scraping. Fractionation followed the manufacturer's instructions for the Mem-Per Plus kit (ThermoFisher). Cellular fractions were prepared for mass spectrometry analysis as described in example 6 but omitting the TMT labelling aspects of the methods. Samples were analyzed using 2 hr gradients at 4-45% acetonitrile in 1% formic acid over a 20 cm C18 reverse phase columns (Ion Optiks) electrospraying into timsTOF PRO running PASEF, dynamic resampling, Top10 method. Resulting data were analyzed in commercially available software to identify proteins present in the various cellular fractions. Receptors for the HAPs FGF17, THBS1, VTN, and THBS4 were identified on 68-year-old primary human myoblasts, as listed in Table 29

TABLE 29
Receptors detected by label-free mass
spectrometry of fractionating myoblasts

| Gene | Spectral | Matching |
|---|---|---|
| Name | Counts | Ligands |
| EGFR | 5 | FGF-17 |
| FLT1 | 4 | FGF-17 |
| LRP1 | 4 | THBS1 |
| ITGB1 | 11 | THBS1 |
| ITGA6 | 8 | THBS1, VTN, THBS4 |
| ITGA7 | 10 | THBS4 |

### Example 15—In Vivo Testing of hPSC Factors Increases Regenerative Index and Reduces Fibrotic Index in an Acute Injury Model in Aged Mice

FIGS. 15A-15F show that aged mice (18 months) administered isolated, heparin-agarose bead purified hPSC showed an improved regenerative index and reduced fibrotic index. FIG. 15A shows a schematic of the experiment in this example. This experiment showed that hPSC-derived factors improved histological metrics of muscle health and function. As shown in FIG. 15B HAPs isolated from human pluripotent stem cells increased regenerative potential and reduce muscle fibrosis in aged mice subjected to a model of acute muscle injury. As shown in FIG. 15C, there was increased muscle regeneration for injured, aged mouse muscle treated with THBS1 (2 μg/mL) compared to young and vehicle-treated, aged mouse muscle.

FIG. 15D shows the experimental schematic of time-points for dosing and analysis using an acute injury model in aged mice to measure the effects of individual HAPs with fusion enhancing effects in vitro. Squares denote injury inducing intramuscular injection (IM) with Barium Chloride while circles denote administration of treatment or vehicle. FIG. 15E shows the results of the experiment outlined in FIG. 15D. Administration of 20 μl of HAPs PDGFRL (5 μg/mL, p<3.85E-2) and IGFBP7 (1 μg/mL, p<6.63E-3) resulted in improved new fiber formation (regenerative index compared to vehicle treated aged mice. Stars indicate degree of significance from one-way ANOVA tests. FIG. 15F provides representative images of immunofluorescence staining of sectioned mouse muscle (tibialis anterior) demonstrating increased muscle regeneration for injured, aged mouse muscle treated with POSTN (1 μg/mL) or IGFBP7 (1 μg/mL) compared to vehicle-treated, aged mouse muscle.

Other models for in vivo testing of hPSC factors include:

### Disuse-Reload Injury Model

This mouse model is a way to observe muscle atrophy in a non-invasive way by contracting the hind limbs of a mouse and preventing extension and flexion, thus reducing the size and strength. The model will serve as an important measurement of muscle regeneration with biologic candidates.

The hind limb will be immobilized with Cast Tape extended position using sports tape to prevent flexion of the limb. Once the sports tape is in place, a strip of casting tape will be wrapped over the sports tape from the ankle upward, and air dried. The extension of the hind limb should stay rigid in its position for the duration of the study parameters.

The study begins after mice are acclimated and on Day −3, in which mice from all groups will be weighed. Assigned animals will be given daily i.p. injections of Vehicle control or Candidate Biologic for 3 days before undergoing hind limb immobilization on Day 0 for 7 days with continuous daily i.p. injections. Hind limbs will be observed for any adverse effects due to immobilization. On Day 7 of the study, all animals will be sacrificed, and muscle tissue weighed and harvested for further analysis.

### Force Measurement

This study will be used to measure the force of pull in the hind limbs that the animal exerts upon skeletal muscle injury of the tibialis anterior (TA) and gastrocnemius (GA) muscles after injury induction with Barium chloride (BaCl₂). This model will serve to determine which of our biological candidates are efficacious in muscle regeneration.

Skeletal Muscle Injury Induction: Under anesthesia, BaCl₂ will be administered in two sites on the TA and four sites on the GA (as previously described). Hair will be shaved on the left and right hind limbs prior to injection with small animal hair clippers. On Day 0 of the study, the TA muscle will undergo BaCl₂ induced injury on two sites (previously described). On Day 4 of the study, the GA muscle will follow with BaCl₂ induced injury on four sites. Candidate biologic will be administered on Days 0 and 2 in the injury sites of the TA and GA muscles. BrdU will be injected via IP (QD) on days 4-7 to label proliferating muscle precursor and fibrotic cells in order to measure their regenerative potential.

On terminal day 7, animals will be deeply anesthetized, and a force transducer will be used to measure twitch reactions in the hind limbs of each mouse being tested in the study, via a small incision in the TA to a small metal hook. This will be a terminal procedure. Grip strength measurements: the mice will rest on an angled mesh, facing away from the force meter and with its hind limbs at least one-half of the way down the length of the bar. The mouse's tail is pulled directly toward the meter and parallel to the bar. During this procedure, the mouse resists by grasping the mesh with all four limbs. Pulling is continued toward the meter until the hind limbs release.

### Ex Vivo Regenerative Measurement

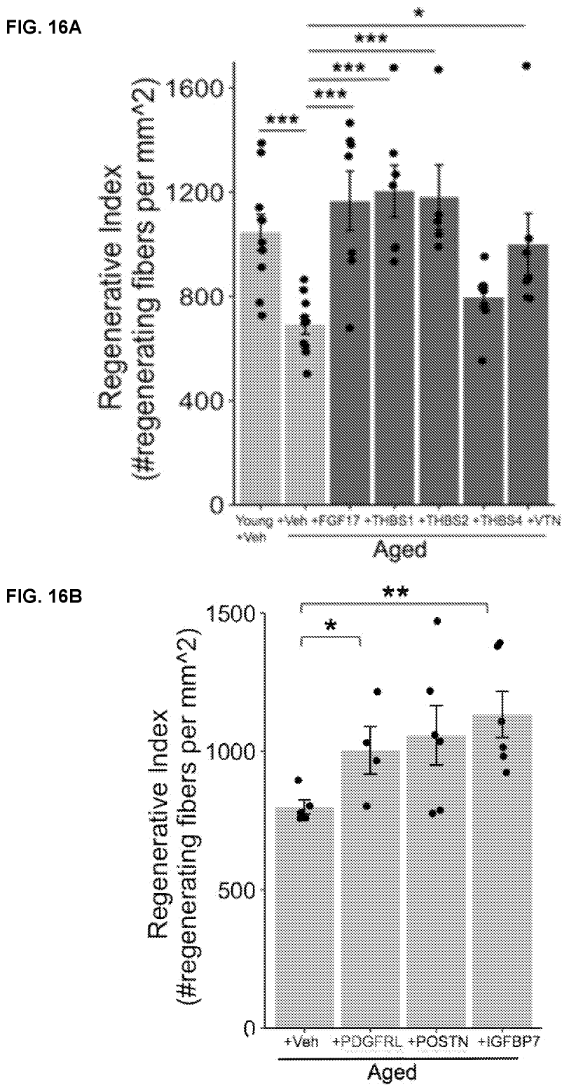

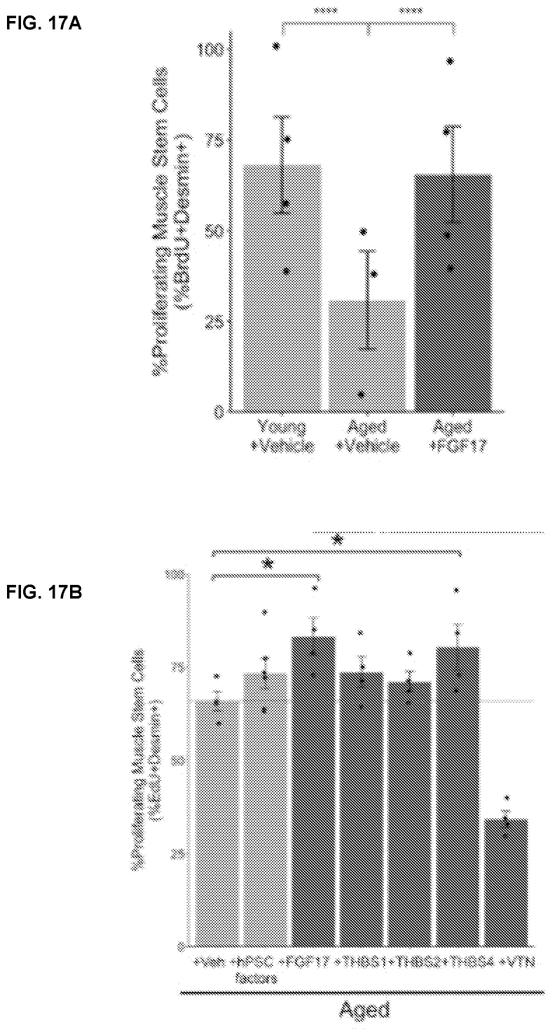

To confirm these data with age matched, primary muscle stem cells, injury-activated satellite cells associated with myofibers will be isolated from young and old muscle by dissecting the muscle groups of interest and dissociating the tissue to single cell suspensions by incubating in digestion medium (250 U/mL Collagenase type II in DMEM medium, buffered with 30 mM HEPES, pH 7.4) at 37 C for 1 hr., triturating the cell suspension, the myofibers were collected by centrifugation and myofibers further digested with 1 U/mL Dispase and 40 U/mL Collagenase type II in 30 mM HEPES at 37 C for 1 hr to free muscle stem cells, as depicted in FIG. 16A. Muscle stem cells can then be plated and cultured growth media containing serum (2-5%) from the same mouse. The regenerative and fusion potential of the cells will then be assayed as described above in in Example 7 and as demonstrated in FIGS. 16B, 16C, 17B, and 17C. This has the advantage of testing the effect of treatment while maintaining the exogenous, often inhibitory extracellular environmental cues contributed by the age appropriate serum.

### Example 16—: Intramuscular Administration of FGF17, THBS1 and IGF2/BMP7 Combo Promote Regeneration of Muscle in a BaCl₂ Injured Old Mouse Model

An old mouse model was used to assess the combination factors regenerative capabilities. On Day 0, 70-week-old mice were weighed and underwent muscle injury with focal injection of barium chloride (BaCl₂, 14 μL, 1.2% w/v in saline, Sigma-Aldrich) in the Tibialis anterior (TA; Day 0) of both the right and left hindlegs. Vehicle or factor A (0.1 mg/kg) were administered intramuscularly (i.m) 2 h following the BaCl₂ into the TA injured hindleg sites, and again 48 hours later on day 3 (i.m.) into the TA injured hindleg sites. Bromodeoxyuridine (BrdU) was administered (100 mg/kg, i.p.) once daily for 3 days, day 2-4, before sacrifice to label proliferating cells. Treatments were administered as listed in Table 30.

TABLE 30
Treatment groups

| Group | Factors | Concentration | |
|---|---|---|---|
| A, n = 5 | FGF17/THBS1 | 100 ng/ml/125 ng/ml | |
| B, n = 5 | FGF17/BMP7 | 12.5 | ng/ml |
| C, n = 4 | BMP7/IGF2 | 25 ng/ml/60 ng/ml | |
| D. n = 4 | FGF17 | 100 | ng/ml |
| E, n = 4 | THBS1 | 125 | ng/ml |
| F, n = 3 | BMP7 | 25 | ng/ml |
| G. n = 4 | IGF2 | 60 | ng/ml |
| H, n = 3 | FGF17 | 500 | ng/ml |
| I, n = 4 | THBS1 | 2000 | ng/ml |

| | | |
|---|---|---|
| J, n = 4 | Vehicle | Saline solution |

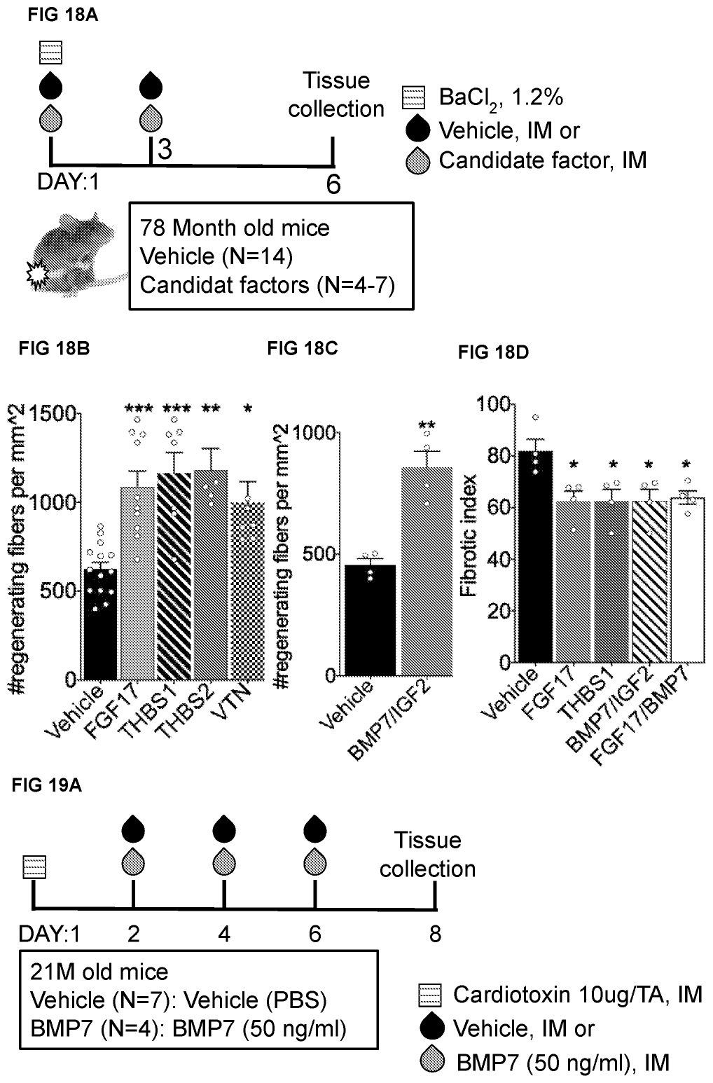

On day 6, animals were sacrificed, and animal weight recorded followed by collecting 0.5 ml of terminal blood via cardiac puncture which was processed to plasma and stored at 80° C. We then dissected the skin from the TA muscles of each hind leg and took photos (prior to excision). After excision of exclusively the TA muscle, excised tissue was photographed, weighed, then placed in chilled isopentane bath precooled in liquid nitrogen and stored at −80 C. Cryosectioning and H&E were performed to ensure the muscle injury site was appropriately visualized. A diagram of the time points for these experiments can be seen in FIG. 18A. Muscle tissue composition from new skeletal muscle fibers, fibrotic tissue, and adipose (fat) were measured. Muscle regeneration, as defined as the number of number of new myofibers with centrally located nuclei per millimeter, fibrosis as defined as the area of fibrotic scarring, size of the fibers, as defined as the width and area, adipose tissue, as defined by the amount of fat surrounding the muscle, were measured to assess level of regeneration.

FIGS. 18B-18C depicts the regenerative index of the treated and untreated muscles. When compared to the untreated (vehicle) group, mice that were treated with FGF17 (group H), THBS1 (group I), and the combination BMP7/IGF7 (group C) showed a significant increase in the regenerative index. When the muscle fibrosis was analyzed, mice that were treated with FGF17 (group H), FGF17/BMP7 (group B), and BMP7/IGF2 (group C) showed a significant decrease in the fibrotic index when compared to untreated mice, as depicted in FIG. 18D. Notably, a combination treatment of BMP7/IGF2 resulted in improved recovery from muscle injury as seen by both an increase in proliferation and a decrease in fibrosis. In this example, combining factors resulted in an improvement in muscle injury compared to untreated muscles.

### Example 17—Systemic Administration of FGF17 Protects Against Dexamethasone Induced Muscle Atrophy

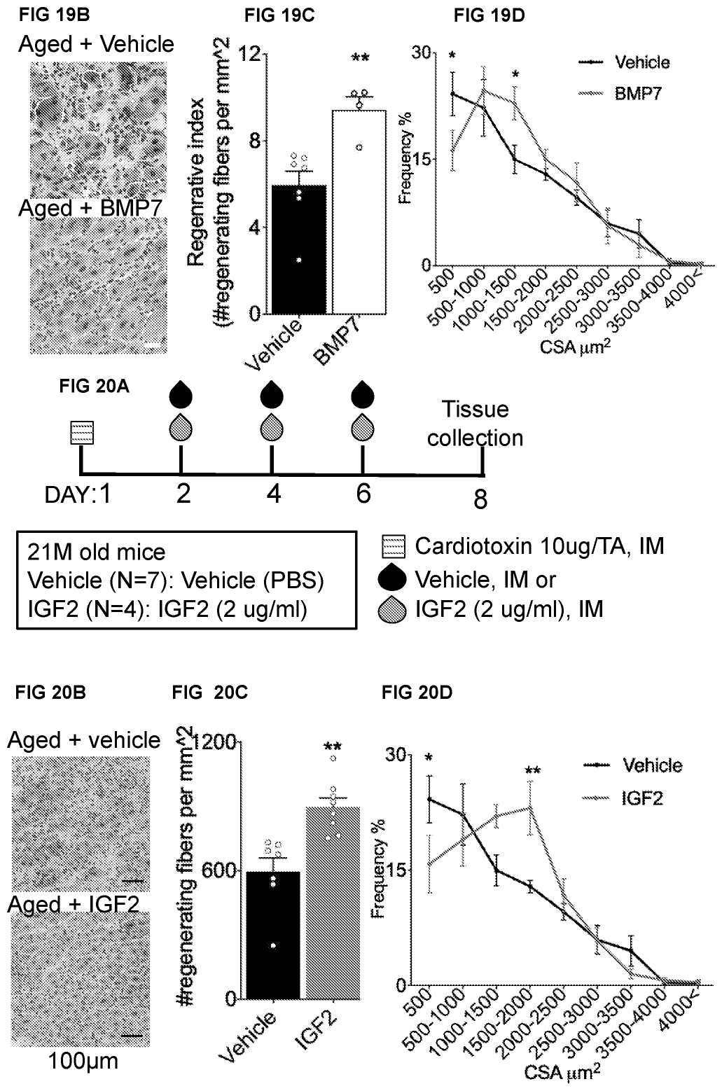

12-week-old mice were injected daily with either vehicle only (n=7), dexamethasone (n=6), or dexamethasone and FGF17 (n=6), as depicted in FIG. 19A. Dexamethasone was injected intraperitoneally at a concentration of 25 mg/kg to induce muscle atrophy. FGF17 was injected subcutaneously at a concentration of 0.5 mg/kg. Forelimb grip strength and both limb grip strength was assessed on days 7, 13, and 19, as described in previous Example 4. At 21 days, mice were euthanized, and the weight of the TA muscle was assessed.

Mice that had been administered dexamethasone had a significant decrease in TA weight when compared to mice that had not received dexamethasone, as depicted in FIG. 19B. Administration of FGF17 resulted in a significant increase of TA weight compared to untreated mice that had received dexamethasone. Both the forelimb specific force and the both limb specific force was significantly reduced in mice with dexamethasone-induced muscle atrophy compared to untreated mice. However, mice that had received both dexamethasone and FGF17 had a significant increase in forelimb specific forms and both limb specific force when compared to mice that received dexamethasone alone, as depicted in FIGS. 19C-19D.

### Example 18—Modelling Treatment of a Muscular Dystrophy with Pro-Regenerative Factors In Vitro Using High-Throughput Imaging

Muscular dystrophies (MD) encompass a variety of muscular degeneration diseases typically due to genetic mutations in genes encoding proteins responsible for forming and stabilizing skeletal muscle. The phenotypic consequence of these genetic mutations is the progressive loss of muscle mass and strength over time, similar to sarcopenia but with different underlying causes. As HAPs provided phenotypic improvements on sarcopenic muscle, we tested for similar improvements in a model for MD.

Many of the factors detailed in Table 2 were tested individually for their ability to promote proliferation and/or fusion of human muscle progenitor cells from a patient with myotonic dystrophy type 1 (hMD)—a muscular dystrophy caused by mutations in the DMPK1 gene.

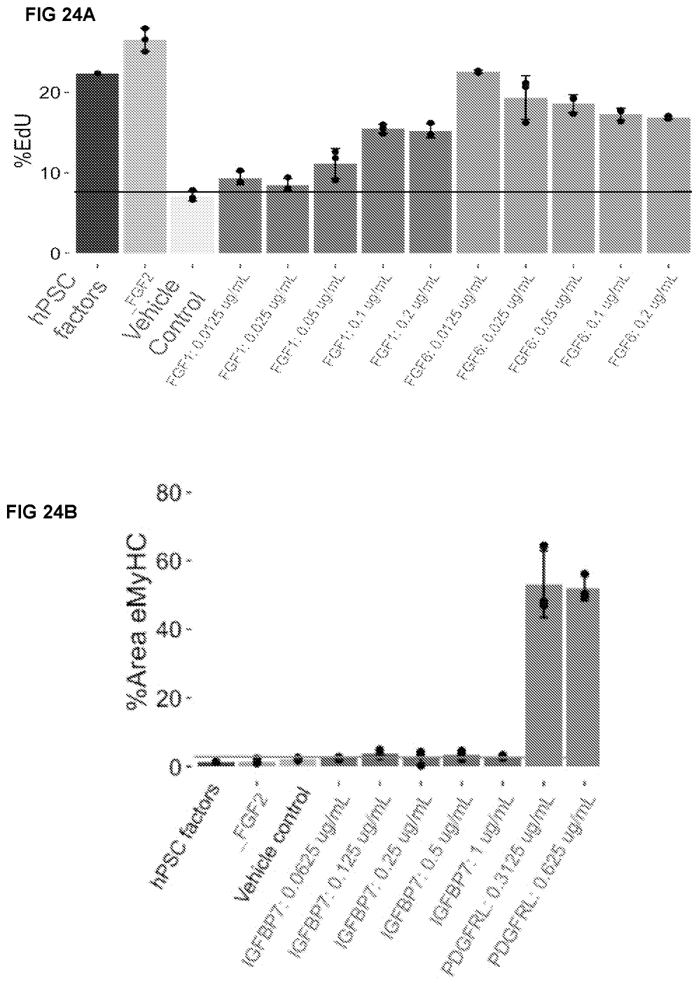

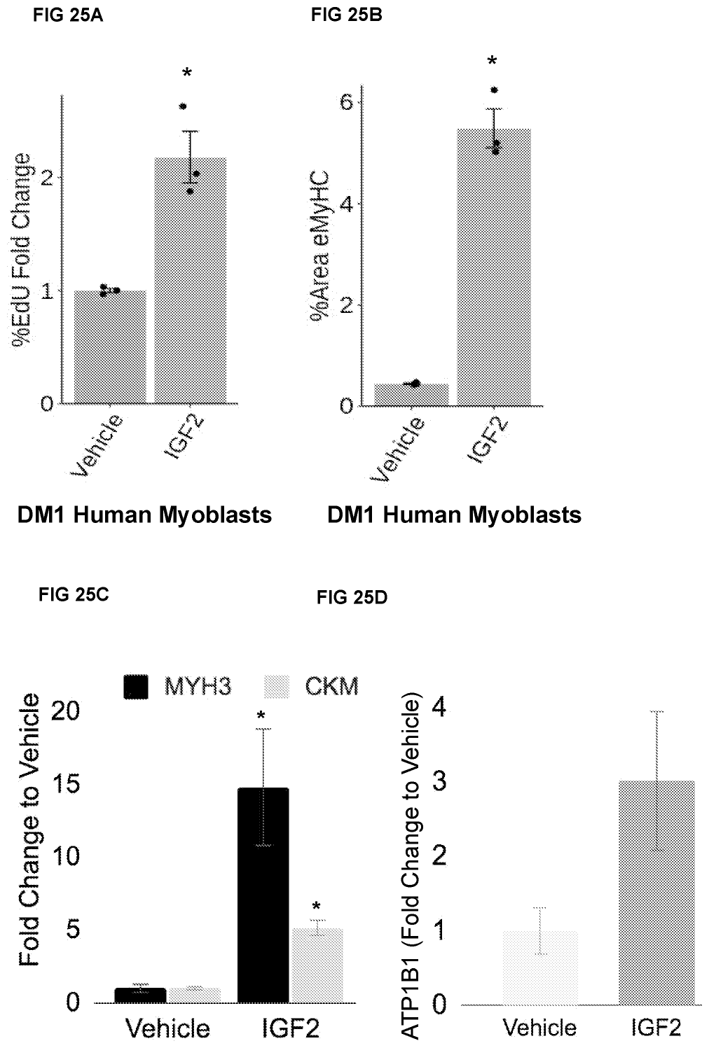

The effect of the candidate factors on myogenic activity was assayed in biological triplicate across a range of concentrations centered around expected physiological levels by adding each factor to hMD myoblasts for 72 hours with daily media changes (DMEM +2% horse serum) and a second pulse of factors at the first media change. After 72 hours, cells were pulsed for 2-5 hours with EdU (30 uM), ethanol fixed, stained with Hoechst 3342, immunostained for proliferation—as measured by the percent of cells staining positive for EdU (% EdU)−, and immunostained for differentiation—as measured by the increase in cellular area staining positive for embryonic myosin heavy chain (% eMyHC) relative to the negative controls, which received media and vehicle only. Wells were imaged on a Keyence BZ-100 at 4×, the images quantified in Cell Profiler, and the statistics were computed in R. FIGS. 24A, 25A and 24B, 25B show examples of the quantitation of the proliferation response and fusion response for several of the factors tested, respectively. Results of those and additional factors are summarized below in Table 31. Transcriptional profiling of these treated cells found IGF2 enhances MYH3, CKM, and ATP1B1 expression in DM1 human myoblast (32 year old caucasian female) cells FIGS. 25C and 25D.

TABLE 31
Effect of individual factors on dystrophic human myoblast growth and fusion

| | | Proliferation (% EdU) | | Fusion (% eMyHC) | |
|---|---|---|---|---|---|
| | | Effect Size | Statistical | Effect Size | Statistical |
| Factor | Concentration | (% relative | Significance | (% relative | Significance |
| Name | (ug/mL) | to −control) | (p-value) | to −control) | (p-value) |
| FGF17 | 0.2 | 208% | <9E−14 | | |
| FGF4 | 0.0125 | 269% | 9.52E−13 | | |
| FGF4 | 0.025 | 244% | 6.83E−12 | | |
| FGF4 | 0.05 | 235% | 7.22E−10 | | |
| FGF4 | 0.1 | 213% | 8.11E−07 | | |
| FGF4 | 0.2 | 184% | 1.46E−02 | | |
| IGF2 | 0.2 | 53% | 2.57E−02 | | |
| FGF1 | 0.05 | 157% | 1.65E−06 | | |
| FGF1 | 0.1 | 217% | 2.87E−06 | | |
| FGF1 | 0.2 | 213% | 1.10E−10 | | |
| FGF6 | 0.0125 | 277% | 1.34E−09 | | |
| FGF6 | 0.025 | 272% | 4.54E−09 | | |
| FGF6 | 0.05 | 261% | 4.75E−08 | | |
| FGF6 | 0.1 | 243% | 1.03E−07 | | |
| FGF6 | 0.2 | 237% | 8.49E−06 | | |
| PDGFRL | 0.03125 | | | 2580% | 1.47E−06 |
| PDGFRL | 0.0625 | | | 2240% | 6.34E−07 |
| PDGFRL | 0.125 | | | 1410% | 4.53E−03 |
| PDGFRL | 0.25 | | | 2570% | 1.86E−08 |
| PDGFRL | 0.5 | | | 3440% | 5.75E−11 |
| IGF2 | 0.2 | 218 | 6.8E−3 | 1200% | 1.9E−4 |

### Example 19—Systemic Administration of Therapeutic Polypeptides Reverses Sarcopenia and Protects from Muscle Injury

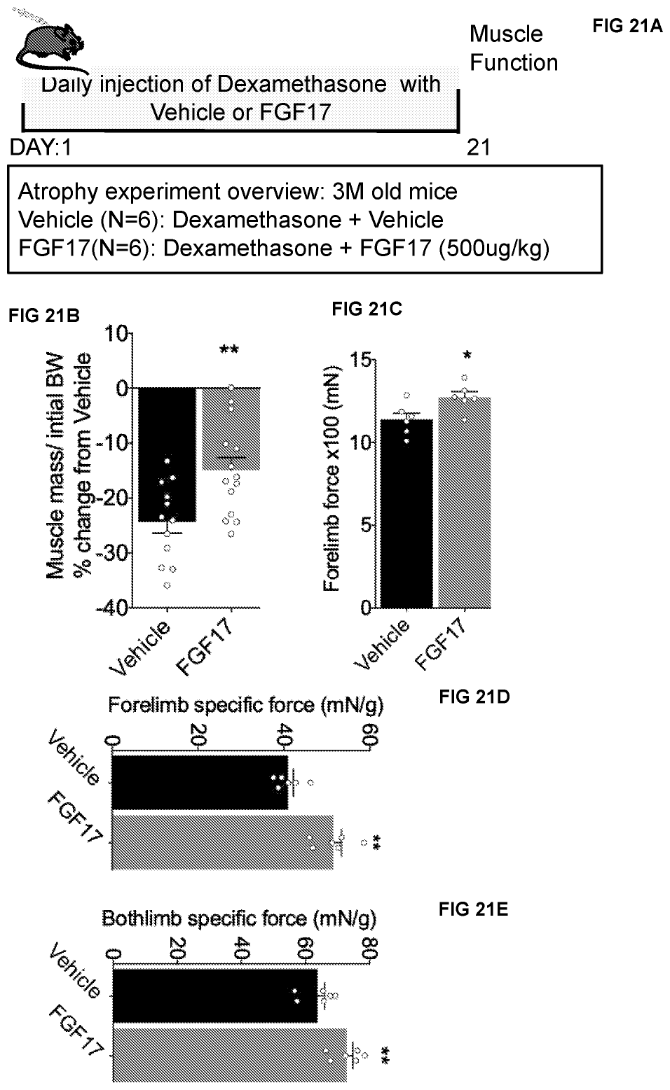

A daily subcutaneous injection of therapeutic polypeptides or vehicle only is administered to 78-week-old mice for 14 days, as depicted in FIG. 21. The therapeutic polypeptides include FGF17 at a concentration of 500 ug/kg, IGF2 at a concentration of 100-1000 ug/kg, and BMP7 at a concentration of 10-100 μg/ug. In some experiments, treatment groups receive a single therapeutic factor while in other experiments, treatment groups receive a combination of factors. At 7 days, muscle function is assessed using forelimb grip strength and both grip strength. On day 12, 13 and 14, groups 1 and 2 are injected with BrdU intraperitoneally. On days 13-15, all mice are assessed for grip strength and an endurance test to determine max distance and max speed and tetanic force, as described in example 4.

At 15 days, mice in groups 1 and 2 are euthanized and the muscles are analyzed for markers of proliferation and fibrosis. At 15 days, an intramuscular injection of 1.2% of BaCl₂ (7 ul/TA) is used to generate chemical injury in the TAs of group 3 and group 4. Mice from groups 3 and 4 continue to receive a therapeutic polypeptide injected subcutaneously on days 15-21. They also receive BrdU injections intraperitoneally on days 19, 20 and 21. On day 21, the TA muscles are tested for in situ tetanic force, using methods described in Example 4. The TA muscles are dissected and assessed for signs of proliferation and fibrosis.

### Example 20—Systemic Administration of Therapeutic Polypeptides Predicted to Reverse Dexamethasone Induced Muscle Atrophy

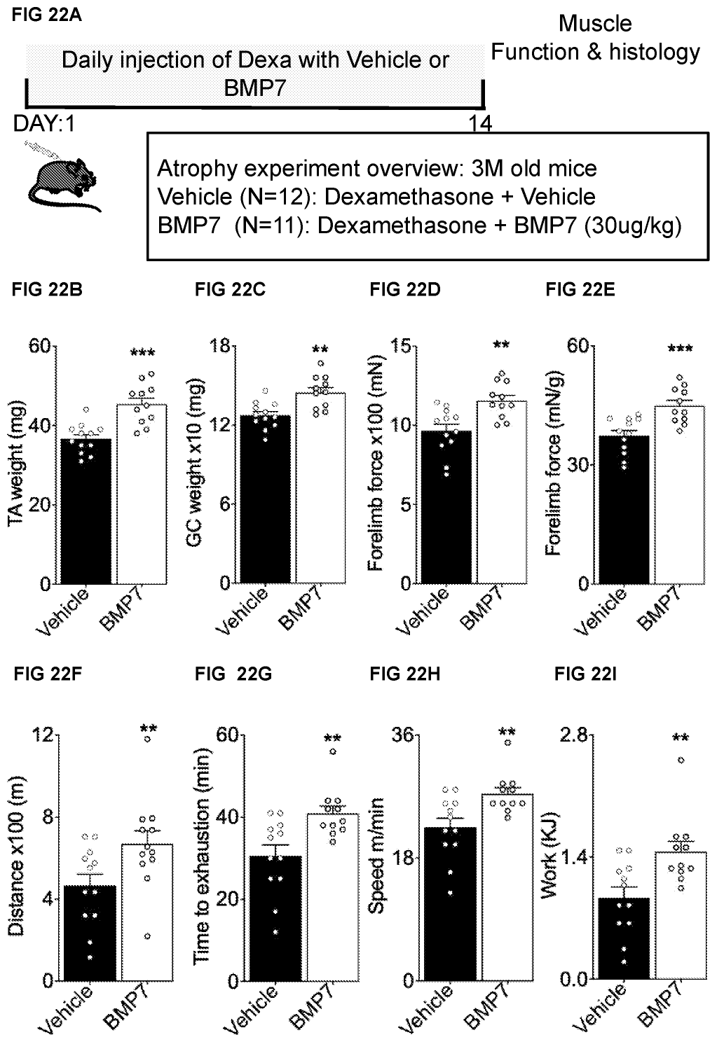

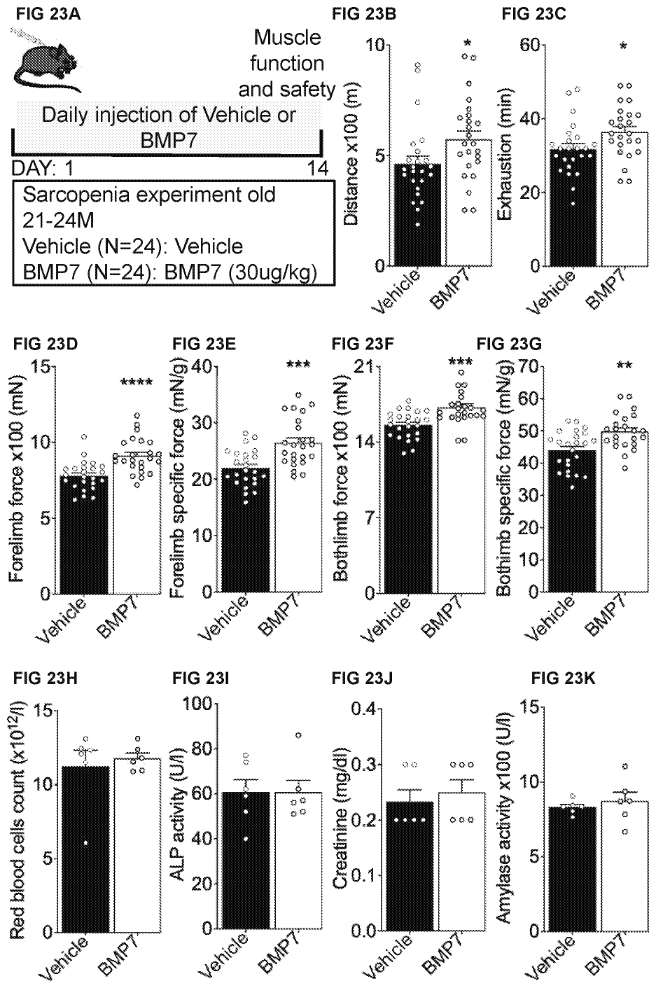

12-week-old mice are divided into 3 treatment groups: group 1 which receives injections only of the vehicle, group 2 which receives injections of dexamethasone, and group 3 which receives injections of dexamethasone and therapeutic peptide. Dexamethasone (25 mg/kg i.p.) is administered for 14 days simultaneously with a subcutaneous injection of therapeutic polypeptides, as depicted in FIG. 22.

At 7 days, mice are assessed for forelimb and both limb grip strength, using the methods described in example 4. At days 13-15, mice are assessed for grip strength, in vivo and in situ tetanic force, and undergo a treadmill endurance test to determine max speed and max distance.

The therapeutic peptides predicted to be effective at reversing muscle atrophy are IGF2 at a concentration of 100-1000 ug/kg, BMP7 at a concentration of 10-100 μg/kg, POSTN at a concentration of 10-1000 μg/kg, IGFBP7 at a concentration of 100-1000 μg/kg, and PDGFRL at a concentration of 10-1000 μg/kg.

### Example 21—Systemic Administration of Therapeutic Polypeptides Predicted to Improve Muscle Atrophy in Genetically Obese Mice

Thirteen-week-old genetically obese mice (ob ob) are injected subcutaneously with a therapeutic peptide for 14 days At day 7, forelimb and both grip strength are measured as described in Example 4. BrdU is injected on days 12, 13 and 14. On days 13, 14 and 15, forelimb and both limb grip strength, in vivo and in situ tetanic force are tested, and an endurance test to determine max distance and max speed is performed using methods described in Example 4. At 14 days, the mice are euthanized, and the TA muscles are dissected. Muscle weight and proliferation is analyzed.

The therapeutic peptides predicted to be effective at reversing muscle atrophy are IGF2 at a concentration of 100-1000 μg/kg, BMP7 at a concentration of 10-100 μg/kg, POSTN at a concentration of 10-1000 μg/kg, IGFBP7 at a concentration of 100-1000 μg/kg, and PDGFRL at a concentration of 10-1000 ug/kg.

### Example 22—Systemic Administration of Therapeutic Polypeptides Predicted to Reverse of Slow Down Dystrophic Features in 70 Weeks Old Mdx Mice

Another class of human myopathies in need of treatment are the genetic abnormality induced muscular dystrophies, among which Duchenne muscular dystrophy is a rare but fatal case. Old genetically dystrophic (mdx) mice (>15 month old) show similar features to the human Duchenne muscular dystrophy (DMD), notably, a decrease in muscle regeneration leading to muscle wasting. Treatment with therapeutic polypeptide described herein can reverse the dystrophic features of old mdx mice. Mice will be fed ad libitum and housed under controlled conditions of lighting (12-hour light/12-hour dark) and temperature (22-24° C.). Mice will be allowed to acclimate for 3 days prior to the initiation of the experiment. During the acclimation period, the weight, Forelimb and both limb grip strength as well as in vivo tetanic force will be assessed to determine the baseline strength of each mouse. 70-week dystrophic mice (mdx) are injected with the therapeutic peptide subcutaneously for 14 days. At day 7, forelimb and both grip strength are measured as described in Example 4. BrdU is injected on days 12, 13 and 14. On days 13, 14 and 15, forelimb and both limb grip strength and tetanic force are tested, and an endurance test to determine max distance and max speed is performed using methods described in Example 4. A The right tibialis anterior and gastrocnemius will be collected, immersed in Tissue-TEK OCT and then flash frozen in chilled isopentane bath precooled in liquid nitrogen and stored at −80 C. Tissue will be sectioned and stained for Laminin to determine the cross-sectional area (CSA) of muscle fibers, for eMyHC to measure new fiber formation and for BrdU to assess the proliferation rate. The left anterior tibialis and gastrocnemius will be collected and flash frozen in liquid nitrogen for molecular analysis that include qPCR and western blot.

The therapeutic peptides predicted to be effective in this model are IGF2 at a concentration of 100-1000 ug/kg, FGF17 at a concentration of 10-100 ug/kg, FGF4 at a concentration of 100-1000 μg/kg, and FGF6 at a concentration of 100-1000 ug/kg.

### Example 23—Systemic Administration of Therapeutic Polypeptides Predicted to Improve the Dystrophic Features in 6-Week-Old Mice

Between 3-6 weeks old, the skeletal muscle of mdx mice undergoes severe necrosis followed by an increase in the activation of satellite cells to promote muscle regeneration. Treatment with therapeutic polypeptide described herein can improve the regeneration process and therefore muscle health. Mice will be fed ad libitum and housed under controlled conditions of lighting (12-hour light/12-hour dark) and temperature (22-24° C.). Mice will be allowed to acclimate for 3 days prior to the initiation of the experiment. During the acclimation period, the weight, Forelimb and both limb grip strength as well as in vivo tetanic force will be assessed to determine the baseline strength of each mouse. 6-week-old dystrophic mice (mdx) are injected with the therapeutic peptide subcutaneously for 14 days. At day 7, forelimb and both grip strength are measured as described in Example 4. BrdU is injected on days 12, 13 and 14. On days 13, 14 and 15, forelimb and both limb grip strength and tetanic force are tested, and an endurance test to determine max distance and max speed is performed using methods described in Example 4.

Mice will be euthanized. The right tibialis anterior and gastrocnemius will be collected, immersed in Tissue-TEK OCT and then flash frozen in chilled isopentane bath precooled in liquid nitrogen and stored at −80 C. Tissue will be sectioned and stained for Laminin to determine the cross-sectional area (CSA) of muscle fibers, for eMyHC to measure new fiber formation and for BrdU to assess the proliferation rate. The left anterior tibialis and gastrocnemius will be collected and flash frozen in liquid nitrogen for molecular analysis that include qPCR and western blot.

The therapeutic peptides predicted to be effective in this model are IGF2 at a concentration of 100-1000 μg/kg, FGF17 at a concentration of 10-100 ug/kg, FGF4 at a concentration of 100-1000 μg/kg, and FGF6 at a concentration of 100-1000 ug/kg.

### Example 24—Treatment for Chondrocyte Proliferation in Cartilage Injury and Osteoarthritis

Cartilage can become damaged as a result of a sudden injury or due to gradual wear and tear (osteoarthritis). Chondrocytes secrete the cartilage matrix and preadipocytes, osteocytes and tenocytes are all cell types associated with cartilage.

Preadipocytes, chondrocytes, osteocytes and tenocytes were cultured in well plates. RNA was isolated from each well (RNeasy Mini Kit, Qiagen) and cDNA was obtained by reverse-transcription (High Capacity Reverse Transcription Kit, Thermo Fisher Scientific). Real-time quantitative PCR was performed using QuantStudio3 (Thermo Fisher).

These cartilage-associated cells expressed receptors for many of hPSC factors Specifically, expression of FGFR1, ACVR1, ITGB1 and IGF2R was detected in cartilage associated cells. Subcutaneous preadipocytes, chondrocytes, osteocytes and tenocytes all expressed these receptors, as depicted in Table 31, indicating that these hEPSC factors may be able to affect cartilage proliferation.

TABLE 31
RNA expression in cartilage associated cells (TPM)

| Cell Type | FGFR1 | ACVR1 | ITGB1 | IGF2R |
|---|---|---|---|---|
| Preadipocyte | 54.92 | 27.11 | 827.15 | 27.083 |
| (Subcutaneous) | | | | |
| Chondrocyte | 152.59 | 34.44 | 971.35 | 47.63 |
| Osteocyte | 202.57 | 39.79 | 290.03 | 83.96 |
| Tenocyte | 167.01 | 33.00 | 994.43 | 23.12 |

### In Vitro Model Screening for Chondrocyte Proliferation in Cartilage Injury and Osteoarthritis

HAPs collectively and FGF17 specifically induced chondrocyte proliferation in healthy adult human chondrocytes as shown in Table 32. Chondrocytes were cultured for 48 h in the presence of HAPs at indicated dose. Fresh media and HAP was added every 24 h. Mean∓S.D. Table 32 of % EdU+ chondrocyte values and p-values by Tukey Honest Significant Difference T-test, n=2-3.

TABLE 32
Chondrocyte proliferation driven by HAPs

| Condition | n | % EdU | sd | adj-p-val |
|---|---|---|---|---|
| Vehicle | 3 | 0.744 | 0.247 | |
| FGF18 | 3 | 6.039 | 1.6370 | 1.29E−03 |
| 1x_HAPs | 3 | 14.542 | 6.092 | 1.95E−02 |
| 2x_HAPs | 3 | 31.037 | 6.097 | 1.28E−04 |
| 4x_HAPs | 3 | 32.009 | 1.197 | 1.01E−04 |

HAPs collectively and FGF17 specifically induced chondrocyte proliferation in healthy adult human chondrocytes as shown in Table 32. Chondrocytes were cultured for 48 h in the presence of HAPs at indicated dose. Fresh media and FGF17 (Table 33) TGFB1 (Table 34), FGF1 (Table 34), or PDGFD (Table 34) was added every 24 h. Mean∓S.D. of % EdU+ chondrocyte values and p-values by Tukey Honest Significant Difference T-test, n=2-3.

TABLE 33
Chondrocyte proliferation driven by FGF17

| Condition | n | % EdU | sd | adj-p-val |
|---|---|---|---|---|
| Vehicle | 3 | 0.744 | 0.247 | |
| FGF18: 0.1 ug/mL | 3 | 6.039 | 1.637 | 1.29E−03 |
| FGF17: 0.1 ug/mL | 2 | 4.675 | 0.198 | 1.92E−04 |
| FGF17: 0.20 ug/mL | 2 | 9.085 | 0.094 | 7.85E−06 |
| FGF17: 0.40 ug/mL | 2 | 16.773 | 0.621 | 5.42E−07 |

TABLE 34
Chondrocyte proliferation driven by
HAPs and pro-regenerative factors

| Condition | N | Percent_EdU | SD | adj-pval |
|---|---|---|---|---|
| Vehicle | 9 | 4.793 | 2.359 | |
| HAPs | 6 | 28.794 | 4.159 | 3.07E−10 |
| TGFB1 | 3 | 17.206 | 1.703 | 4.37E−04 |
| FGF1 | 3 | 15.632 | 0.796 | 2.01E−03 |
| PDGFD | 3 | 44.225 | 2.697 | 2.18E−12 |
| FGF17 | 3 | 26.342 | 7.521 | 1.39E−07 |

### Example 25—Clinical Testing of Pro-Regenerative Factors

The purpose of this study is to determine the safety and tolerability of repeat dosing with multiple dose levels of heparin-associated polypeptide in healthy individuals or individuals diagnosed with sarcopenia, a muscular dystrophy, or recovery from surgery. The muscular dystrophy may be myotonic dystrophy. In addition, this study will generate data on the physical function, skeletal muscle mass and strength resulting from treatment with compositions comprising heparin-associated polypeptides. In addition, this study will generate data on the safety, tolerability, and pharmacokinetics of heparin-associated proteins in older adults with sarcopenia. Individuals will be administered placebo or heparin-associated binding proteins and monitored for 25 weeks of study. The following primary and secondary outcome measures will be assessed:
Primary Outcome Measures: Safety and tolerability as assessed by various measures such as percent of adverse events per study arm.
Secondary Outcome Measures: Plasma Pharmacokinetics (Cmax, Tmax, AUC) [Plasma at 0.5, 1, 1.5, 2, 4, 6, 8, 12 and 24 hrs after dosing.]
Short Physical Performance Battery (SPPB). Change from baseline to week 25.
10-meter walk test. Change from baseline to week 25.
Change in total lean body mass and appendicular skeletal muscle index measured by Dual-energy X-ray Absorptiometry (DEXA) from baseline to week 25.
Inclusion Criteria: Diagnosis of sarcopenia, a muscular dystrophy, or recovery from surgery; Low muscle mass as confirmed by DXA; Low gait speed; SPPB score less than or equal to 9; Weigh at least 35 kg; with adequate dietary intake as determined by patient interview. Independently ambulatory to 10 meters.
Protocol: Patients will be i.v.—administered placebo (5% dextrose solution) or treatment article (in 5% dextrose). Starting on day 1, week 1 and repeated every week (day one of weeks 1 through 25). At the end of week 13 and 25 patients will be assessed by the above methods for improvement. Doses will be selected from a traditional 3+3 design, and selected as the top two-doses that lack dose-limiting toxicity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

TABLE 1
Exemplary Therapeutic Polypeptides

| | HAPs | Uniprot | |
|---|---|---|---|
| Polypeptide | ID NO | ID | Modified Amino Acid Sequences Tested |
| Vitronectin (VTN) | 1 | P04004 | Asp20 to Leu478, purified from Human |
| | | | plasma-derived |
| STC2 | 2 | O76061 | |
| AGRN | 3 | O00468 | |
| Thrombospondin 2 | 4 | P35442 | Gly19 to Ile1172, with a C-terminal 10-His |
| (THBS2) | | | tag (SEQ ID NO: 70), purified from Mouse |
| | | | myeloma cell line, NS0 |
| FST | 5 | P19883 | |
| Periostin (POSTN) | 6 | Q15063 | Asn22 to Gln836, with a C-terminal 6-His tag |
| | | | (SEQ ID NO: 69), purified from Mouse |
| | | | myeloma cell line, NS0 |
| Fibroblast growth | 7 | O60258 | Thr23 to Thr216, purified from E. coli |
| factor 17 | | | |
| (FGF17) | | | |
| Thrombospondin 4 | 8 | P35443 | Ala22 to Asn961, with a C-terminal 10-His |
| (THBS4) | | | tag (SEQ ID NO: 70), purified from Chinese |
| | | | Hamster Ovary cell line |
| Thrombospondin 1 | 9 | P07996 | Asn19-Pro1170, with Thr523 Ala substitution |
| (THBS1) | | | plus 10His tag, purified from mouse myeloma |
| | | | cell line, NS0 |
| Interleukin-15 | 10 | P40933 | Asn49 to Ser162, purified from E. coli |
| Insulin-like growth | 11 | P01344 | Ala25 to Glu91, purified from E. coli |
| factor 2 (IGF2) | | | |
| Fibroblast growth | 12 | P09038 | Pro143 to Ser288, purified E. coli |
| factor 2 (FGF 2) | | | |
| Fibroblast growth | 13 | O95750 | Leu25 to Lys216, purified from E. coli |
| factor 19 (FGF 19) | | | |
| Angiogenin (ANG) | 14 | P03950 | Gln25 to Pro147, purified from E. coli |
| Probetacellulin (BTC) | 15 | P35070 | Asp32 to Tyr111, purified from E. coli |
| Interleukin -13 | 16 | P35225 | Arg21 to Ala228, purified from mouse |
| receptor alpha 2 | | | myeloma cell line, NS0 |
| Siglec-5/CD170 | 17 | O15389 | Glu17 to Thr434, purified from mouse |
| | | | myeloma cell line, NS0 |
| Apelin receptor (APJ) | 18 | P35414 | |
| Insulin-like growth | 19 | P18065 | Glu40 to Gln328, purified from mouse |
| factor-binding protein | | | myeloma cell line, NS0 |
| 2 (IGFBP-2) | | | |
| Chordin-Like 1 | 20 | Q9BU40 | Glu22-Cys450, purified from mouse myeloma |
| (CHRDL1) | | | cell line, NS0 |
| WAP, Kazal, | 21 | Q8TEU8 | Leu35 to His 576, purified from mouse |
| immunoglobulin, | | | myeloma cell line, NS0 |
| Kunitz and NTR | | | |
| domain-containing | | | |
| protein 2 | | | |
| Membrane frizzled- | 22 | Q9BY79 | Ser101 to Pro579, purified from mouse |
| related protein | | | myeloma cell line, NS0 |
| (MFRP) | | | |
| Interleukin -10 | 23 | P22301 | His22 to Asn235, purified from human cell |
| receptor alpha | | | line HEK293 |
| Chemokine like | 24 | Q99788 | |
| receptor 1, Chemerin | | | |
| Receptor 23 (Chem | | | |
| R23) | | | |
| HB-EGF | 25 | Q99075 | Asp63 to Leu148, purified from insect cells |
| fibroblast growth | 26 | P10767 | Gly67 to Ile208, purified from E. coli |
| factor 6 | | | |
| Hepatocyte Growth | 27 | P14210 | Gln32 to Ser728, purified from insect cells |
| Factor | | | |
| Interleukin-16 | 28 | Q14005 | Pro2 to Ser130, purified from E. coli |
| Interleukin-7 receptor | 29 | P16871 | Glu21 to Lys261, purified from mouse |
| alpha | | | myeloma cell line, NS0 |
| Tumor necrosis factor | 30 | O14798 | Ala26 to Ala221, purified from mouse |
| receptor superfamily | | | myeloma cell line, NS0 |
| member 10C | | | |
| Bone morphogenetic | 31 | P22004 | Gln382 to His51, 3 purified from E. coli |
| protein 6 | | | |
| Interleukin-36 gamma | 32 | Q9NZH8 | Ser18 to Asp169, purified from E. coli |
| interleukin-1 receptor | 33 | P18510 | Val2 to Asp155, purified from E. coli |
| antagonist | | | |
| (IL-1RA) | | | |
| Kremen protein 2 | 34 | Q8NCW0 | Gln19 to Ala364, purified from mouse |
| | | | myeloma cell line, NS0 |
| Tumor necrosis factor | 35 | Q9UBN6 | |
| receptor superfamily | | | |
| member 10D | | | |
| C-X-C chemokine | 36 | P25024 | |
| receptor type 1 | | | |
| C-C motif chemokine | 37 | P55773 | |
| 23 | | | |
| Catenin, Beta | 38 | P35222 | |
| Fibroblast growth | 39 | Q92913 | |
| factor 13, 1B | | | |
| Tumor necrosis factor | 40 | P50591 | Glu108 to Leu261, purified from E. coli |
| ligand superfamily | | | |
| member 10 | | | |
| C-C motif chemokine | 41 | Q16627 | Ser35 to Glu111, purified from E. coli |
| 14 | | | |
| Insulin-like growth | 42 | Q16270 | Asp30 to Leu282 with a K95R mutation with |
| factor binding protein | | | an N-terminal 10-His tag (SEQ ID NO: 70), |
| 7 | | | purified from Mouse myeloma cell line, NS0 |
| Fibroblast growth | 43 | P08620 | Ser54 to Leu206, purified from E. coli |
| factor 4 | | | |
| Fibroblast growth | 44 | P55075 | Gln23 to Arg204, purified from E. coli |
| factor 8 | | | |
| PDGFRL | 55 | Q15198 | Gln22 to Ser375, purified from HEK293 cells |
| ANOS1 | 56 | P23352 | Ala25 to Tyr680, purified from CHO cells |
| THBS1 isoform 2 | 58 | P07996-2 | |
| FGF17 isoform 2 | 59 | O60258-2 | |
| POSTN isoform 2 | 60 | Q15063-2 | |
| POSTN isoform 3 | 61 | Q15063-3 | |
| POSTN isoform 4 | 62 | Q15063-4 | |
| POSTN isoform 5 | 63 | Q15063-5 | |
| POSTN isoform 6 | 64 | Q15063-6 | |
| POSTN isoform 7 | 65 | Q15063-7 | |
| IGF2 isoform 2 | 66 | P01344-2 | |
| IGF2 isoform 3 | 67 | P01344-3 | |
| IL15 isoform 2 | 68 | P40933 | |
| Bone morphogenetic | 72 | P18075 | Ser293 to His 431, purified from CHO cells |
| protein 7 (BMP7) | | | |

TABLE 2
Factors enriched in the supernatants of undifferentiated
human pluripotent stem cells.

| | Uniprot | Entrez Gene | | |
|---|---|---|---|---|
| Gene Name | ID | ID | Ensembl ID | Polypeptide No. |
| A1BG | P04217 | 1 | ENSG00000121410 | 1 |
| A2M | P01023 | 2 | ENSG00000175899 | 2 |
| ABCF1 | Q8NE71 | 23 | ENSG00000204574 | 3 |
| ACADVL | P49748 | 37 | ENSG00000072778 | 4 |
| ACLY | P53396 | 47 | ENSG00000131473 | 5 |
| ACP1 | P24666 | 52 | ENSG00000143727 | 6 |
| ACP5 | P13686 | 54 | ENSG00000102575 | 7 |
| ACTG1 | P63261 | 71 | ENSG00000184009 | 8 |
| ACTN1 | P12814 | 87 | ENSG00000072110 | 9 |
| ACTR3 | P61158 | 10096 | ENSG00000115091 | 10 |
| ADAMTS1 | Q9UHI8 | 9510 | ENSG00000154734 | 11 |
| ADAMTS12 | P58397 | 81792 | ENSG00000151388 | 12 |
| ADAMTS19 | Q8TE59 | 171019 | ENSG00000145808 | 13 |
| ADAMTS7 | Q9UKP4 | 11173 | ENSG00000136378 | 14 |
| ADAMTS8 | Q9UP79 | 11095 | ENSG00000134917 | 15 |
| ADRM1 | Q16186 | 11047 | ENSG00000130706 | 16 |
| AEBP1 | Q8IUX7 | 165 | ENSG00000106624 | 17 |
| AFM | P43652 | 173 | ENSG00000079557 | 18 |
| AFP | P02771 | 174 | ENSG00000081051 | 19 |
| AGPS | O00116 | 8540 | ENSG00000018510 | 20 |
| AGRN | O00468 | 375790 | ENSG00000188157 | 21 |
| AGT | P01019 | 183 | ENSG00000135744 | 22 |
| AHCYL2 | Q96HN2 | 23382 | ENSG00000158467 | 23 |
| AHSG | P02765 | 197 | ENSG00000145192 | 24 |
| AIMP1 | Q12904 | 9255 | ENSG00000164022 | 25 |
| ALB | P02768 | 213 | ENSG00000163631 | 26 |
| ALCAM | Q13740 | 214 | ENSG00000170017 | 27 |
| ALDH9A1 | P49189 | 223 | ENSG00000143149 | 28 |
| ALDOA | P04075 | 226 | ENSG00000149925 | 29 |
| ALPL | P05186 | 249 | ENSG00000162551 | 30 |
| AMBP | P02760 | 259 | ENSG00000106927 | 31 |
| ANG | P03950 | 283 | ENSG00000214274 | 32 |
| ANGPTL4 | Q9BY76 | 51129 | ENSG00000167772 | 33 |
| ANOS1 | P23352 | 3730 | ENSG00000011201 | 34 |
| ANXA1 | P04083 | 301 | ENSG00000135046 | 35 |
| ANXA2 | P07355 | 302 | ENSG00000182718 | 36 |
| ANXA2P2 | A6NMY6 | | | 37 |
| AOC1 | P19801 | 26 | ENSG00000002726 | 38 |
| AP2A1 | O95782 | 160 | ENSG00000196961 | 39 |
| AP2A2 | O94973 | 161 | ENSG00000183020 | 40 |
| AP3D1 | O14617 | 8943 | ENSG00000065000 | 41 |
| APLP2 | Q06481 | 334 | ENSG00000084234 | 42 |
| APOA1 | P02647 | 335 | ENSG00000118137 | 43 |
| APOA2 | P02652 | 336 | ENSG00000158874 | 44 |
| APOB | P04114 | 338 | ENSG00000084674 | 45 |
| APOC3 | P02656 | 345 | ENSG00000110245 | 46 |
| APOD | P05090 | 347 | ENSG00000189058 | 47 |
| APOE | P02649 | 348 | ENSG00000130203 | 48 |
| APOH | P02749 | 350 | ENSG00000091583 | 49 |
| APOM | O95445 | 55937 | ENSG00000204444 | 50 |
| ARCN1 | P48444 | 372 | ENSG00000095139 | 51 |
| ARHGEF1 | Q92888 | 9138 | ENSG00000076928 | 52 |
| ARHGEF28 | Q8N1W1 | 64283 | ENSG00000214944 | 53 |
| ARPC1B | O15143 | 10095 | ENSG00000130429 | 54 |
| ARRB1 | P49407 | 408 | ENSG00000137486 | 55 |
| ARSK | Q6UWY0 | 153642 | ENSG00000164291 | 56 |
| ART4 | Q93070 | 420 | ENSG00000111339 | 57 |
| ASNA1 | O43681 | 439 | ENSG00000198356 | 58 |
| ASNS | P08243 | 440 | ENSG00000070669 | 59 |
| ATP6AP2 | O75787 | 10159 | ENSG00000182220 | 60 |
| ATRN | O75882 | 8455 | ENSG00000088812 | 61 |
| AZGP1 | P25311 | 563 | ENSG00000160862 | 62 |
| B3GALT6 | Q96L58 | 126792 | ENSG00000176022 | 63 |
| B3GNT7 | Q8NFL0 | 93010 | ENSG00000156966 | 64 |
| B4GALT1 | P15291 | 2683 | ENSG00000086062 | 65 |
| B4GALT4 | O60513 | 8702 | ENSG00000121578 | 66 |
| B4GAT1 | O43505 | 11041 | ENSG00000174684 | 67 |
| BCAM | P50895 | 4059 | ENSG00000187244 | 68 |
| BGN | P21810 | 633 | ENSG00000182492 | 69 |
| BLVRB | P30043 | 645 | ENSG00000090013 | 70 |
| BMP1 | P13497 | 649 | ENSG00000168487 | 71 |
| BMP7 | P18075 | 655 | ENSG00000101144 | 72 |
| BOC | Q9BWV1 | 91653 | ENSG00000144857 | 73 |
| BRD3 | Q15059 | 8019 | ENSG00000169925 | 74 |
| BSG | P35613 | 682 | ENSG00000172270 | 75 |
| BTBD17 | A6NE02 | 388419 | ENSG00000204347 | 76 |
| BTD | P43251 | 686 | ENSG00000169814 | 77 |
| BZW2 | Q9Y6E2 | 28969 | ENSG00000136261 | 78 |
| C11orf24 | Q96F05 | 53838 | ENSG00000171067 | 79 |
| C1QA | P02745 | 712 | ENSG00000173372 | 80 |
| C1QBP | Q07021 | 708 | ENSG00000108561 | 81 |
| C1QC | P02747 | 714 | ENSG00000159189 | 82 |
| C1QTNF3 | Q9BXJ4 | 114899 | ENSG00000082196 | 83 |
| C1QTNF3- | E9PGA6 | | ENSG00000273294 | 84 |
| AMACR | | | | |
| C1QTNF4 | Q9BXJ3 | 114900 | ENSG00000172247 | 85 |
| C1RL | Q9NZP8 | 51279 | ENSG00000139178 | 86 |
| C1S | P09871 | 716 | ENSG00000182326 | 87 |
| C20orf27 | Q9GZN8 | 54976 | ENSG00000101220 | 88 |
| C3 | P01024 | 718 | ENSG00000125730 | 89 |
| C4A | POCOL4 | 720 | ENSG00000206340 | 90 |
| C4B | A0A140TA29 | | ENSG00000236625 | 91 |
| C4BPA | P04003 | 722 | ENSG00000123838 | 92 |
| C5 | P01031 | 727 | ENSG00000106804 | 93 |
| C7 | P10643 | 730 | ENSG00000112936 | 94 |
| C8B | P07358 | 732 | ENSG00000021852 | 95 |
| C9 | P02748 | 735 | ENSG00000113600 | 96 |
| CA11 | O75493 | 770 | ENSG00000063180 | 97 |
| CALM2 | PODP24 | 801 | ENSG00000143933 | 98 |
| CALR | P27797 | 811 | ENSG00000179218 | 99 |
| CALU | O43852 | 813 | ENSG00000128595 | 100 |
| CAND1 | Q86VP6 | 55832 | ENSG00000111530 | 101 |
| CANT1 | Q8WVQ1 | 124583 | ENSG00000171302 | 102 |
| CANX | P27824 | 821 | ENSG00000127022 | 103 |
| CAPG | P40121 | 822 | ENSG00000042493 | 104 |
| CAPN1 | P07384 | 823 | ENSG00000014216 | 105 |
| CAPZA2 | P47755 | 830 | ENSG00000198898 | 106 |
| CARM1 | Q86X55 | 10498 | ENSG00000142453 | 107 |
| CARS | P49589 | 833 | ENSG00000110619 | 108 |
| CBL | P22681 | 867 | ENSG00000110395 | 109 |
| CBX3 | Q13185 | 11335 | ENSG00000122565 | 110 |
| CCAR2 | Q8N163 | 57805 | ENSG00000158941 | 111 |
| CCBE1 | Q6UXH8 | 147372 | ENSG00000183287 | 112 |
| CCDC80 | Q76M96 | 151887 | ENSG00000091986 | 113 |
| CCK | P06307 | 885 | ENSG00000187094 | 114 |
| CCT2 | P78371 | 10576 | ENSG00000166226 | 115 |
| CCT4 | P50991 | 10575 | ENSG00000115484 | 116 |
| CCT7 | Q99832 | 10574 | ENSG00000135624 | 117 |
| CD5 | P06127 | 921 | ENSG00000110448 | 118 |
| CDC40 | O60508 | 51362 | ENSG00000168438 | 119 |
| CDH1 | P12830 | 999 | ENSG00000039068 | 120 |
| CDH9 | Q9ULB4 | 1007 | ENSG00000113100 | 121 |
| CDON | Q4KMG0 | 50937 | ENSG00000064309 | 122 |
| CDSN | Q15517 | 1041 | ENSG00000137197 | 123 |
| CENPV | Q7Z7K6 | 201161 | ENSG00000166582 | 124 |
| CFB | P00751 | 629 | ENSG00000241253 | 125 |
| CFC1 | P0CG37 | 55997 | ENSG00000136698 | 126 |
| CFD | P00746 | 1675 | ENSG00000197766 | 127 |
| CFH | P08603 | 3075 | ENSG00000000971 | 128 |
| CFI | P05156 | 3426 | ENSG00000205403 | 129 |
| CHAD | O15335 | 1101 | ENSG00000136457 | 130 |
| CHD4 | Q14839 | 1108 | ENSG00000111642 | 131 |
| CHD8 | Q9HCK8 | 57680 | ENSG00000100888 | 132 |
| CHGA | P10645 | 1113 | ENSG00000100604 | 133 |
| CHID1 | Q9BWS9 | 66005 | ENSG00000177830 | 134 |
| CHRDL1 | Q9BU40 | 91851 | ENSG00000101938 | 135 |
| CHST11 | Q9NPF2 | 50515 | ENSG00000171310 | 136 |
| CHST6 | Q9GZX3 | 4166 | ENSG00000183196 | 137 |
| CHSY1 | Q86X52 | 22856 | ENSG00000131873 | 138 |
| CHSY3 | Q70JA7 | 337876 | ENSG00000198108 | 139 |
| CILP2 | Q8IUL8 | 148113 | ENSG00000160161 | 140 |
| CKAP5 | Q14008 | 9793 | ENSG00000175216 | 141 |
| CKMT1A | C9J8F6 | | ENSG00000223572 | 142 |
| CKMT2 | P17540 | 1160 | ENSG00000131730 | 143 |
| CLDN6 | P56747 | 9074 | ENSG00000184697 | 144 |
| CLEC3B | P05452 | 7123 | ENSG00000163815 | 145 |
| CLPX | O76031 | 10845 | ENSG00000166855 | 146 |
| CLSTN3 | Q9BQT9 | 9746 | ENSG00000139182 | 147 |
| CLTC | Q00610 | 1213 | ENSG00000141367 | 148 |
| CLU | P10909 | 1191 | ENSG00000120885 | 149 |
| CNOT1 | A5YKK6 | 23019 | ENSG00000125107 | 150 |
| COCH | O43405 | 1690 | ENSG00000100473 | 151 |
| COL11A1 | P12107 | 1301 | ENSG00000060718 | 152 |
| COL11A2 | P13942 | 1302 | ENSG00000227801 | 153 |
| COL12A1 | Q99715 | 1303 | ENSG00000111799 | 154 |
| COL14A1 | Q05707 | 7373 | ENSG00000187955 | 155 |
| COL16A1 | Q07092 | 1307 | ENSG00000084636 | 156 |
| COL18A1 | P39060 | 80781 | ENSG00000182871 | 157 |
| COL1A1 | P02452 | 1277 | ENSG00000108821 | 158 |
| COL1A2 | P08123 | 1278 | ENSG00000164692 | 159 |
| COL22A1 | Q8NFW1 | 169044 | ENSG00000169436 | 160 |
| COL25A1 | Q9BXS0 | 84570 | ENSG00000188517 | 161 |
| COL26A1 | Q96A83 | 136227 | ENSG00000160963 | 162 |
| COL2A1 | P02458 | 1280 | ENSG00000139219 | 163 |
| COL3A1 | P02461 | 1281 | ENSG00000168542 | 164 |
| COL4A1 | P02462 | 1282 | ENSG00000187498 | 165 |
| COL4A2 | P08572 | 1284 | ENSG00000134871 | 166 |
| COL4A3 | Q01955 | 1285 | ENSG00000169031 | 167 |
| COL4A6 | Q14031 | 1288 | ENSG00000197565 | 168 |
| COL5A1 | P20908 | 1289 | ENSG00000130635 | 169 |
| COL5A2 | P05997 | 1290 | ENSG00000204262 | 170 |
| COL5A3 | P25940 | 50509 | ENSG00000080573 | 171 |
| COL6A1 | P12109 | 1291 | ENSG00000142156 | 172 |
| COL6A2 | P12110 | 1292 | ENSG00000142173 | 173 |
| COL6A3 | P12111 | 1293 | ENSG00000163359 | 174 |
| COL9A2 | Q14055 | 1298 | ENSG00000049089 | 175 |
| COLEC10 | Q9Y6Z7 | 10584 | ENSG00000184374 | 176 |
| COMP | P49747 | 1311 | ENSG00000105664 | 177 |
| COPA | P53621 | 1314 | ENSG00000122218 | 178 |
| COTL1 | Q14019 | 23406 | ENSG00000103187 | 179 |
| CP | P00450 | 1356 | ENSG00000047457 | 180 |
| CPA4 | Q9UI42 | 51200 | ENSG00000128510 | 181 |
| CPE | P16870 | 1363 | ENSG00000109472 | 182 |
| CPN1 | P15169 | 1369 | ENSG00000120054 | 183 |
| CPNE1 | Q99829 | 8904 | ENSG00000214078 | 184 |
| CPVL | Q9H3G5 | 54504 | ENSG00000106066 | 185 |
| CPXM1 | Q96SM3 | 56265 | ENSG00000088882 | 186 |
| CPXM2 | Q8N436 | 119587 | ENSG00000121898 | 187 |
| CPZ | Q66K79 | 8532 | ENSG00000109625 | 188 |
| CRIM1 | Q9NZV1 | 51232 | ENSG00000150938 | 189 |
| CRISPLD1 | Q9H336 | 83690 | ENSG00000121005 | 190 |
| CRLF1 | O75462 | 9244 | ENSG00000006016 | 191 |
| CRYL1 | Q9Y2S2 | 51084 | ENSG00000165475 | 192 |
| CS | O75390 | 1431 | ENSG00000062485 | 193 |
| CSDE1 | O75534 | 7812 | ENSG00000009307 | 194 |
| CSF2RA | P15509 | 1438 | ENSG00000198223 | 195 |
| CST1 | P01037 | 1469 | ENSG00000170373 | 196 |
| CST3 | P01034 | 1471 | ENSG00000101439 | 197 |
| CST4 | P01036 | 1472 | ENSG00000101441 | 198 |
| CTGF | Q5M8T4 | 1490 | | 199 |
| CTNNA1 | P35221 | 1495 | ENSG00000044115 | 200 |
| CTSD | P07339 | 1509 | ENSG00000117984 | 201 |
| CTSV | O60911 | 1515 | ENSG00000136943 | 202 |
| CUL2 | Q13617 | 8453 | ENSG00000108094 | 203 |
| CUL3 | Q13618 | 8452 | ENSG00000036257 | 204 |
| CUL4B | Q13620 | 8450 | ENSG00000158290 | 205 |
| CUTA | O60888 | 51596 | ENSG00000112514 | 206 |
| CXADR | P78310 | 1525 | ENSG00000154639 | 207 |
| CXCL12 | P48061 | 6387 | ENSG00000107562 | 208 |
| CYR61 | Q6FI18 | 3491 | | 209 |
| DAG1 | Q14118 | 1605 | ENSG00000173402 | 210 |
| DARS | P14868 | 1615 | ENSG00000115866 | 211 |
| DBNL | Q9UJU6 | 28988 | ENSG00000136279 | 212 |
| DCD | P81605 | 117159 | ENSG00000161634 | 213 |
| DDOST | P39656 | 1650 | ENSG00000244038 | 214 |
| DDR1 | Q08345 | 780 | ENSG00000137332 | 215 |
| DDX17 | Q92841 | 10521 | ENSG00000100201 | 216 |
| DDX39B | Q13838 | 7919 | ENSG00000215425 | 217 |
| DENND5A | Q6IQ26 | 23258 | ENSG00000184014 | 218 |
| DHFR | P00374 | 1719 | ENSG00000228716 | 219 |
| DHX29 | Q7Z478 | 54505 | ENSG00000067248 | 220 |
| DKK1 | O94907 | 22943 | ENSG00000107984 | 221 |
| DKK3 | Q9UBP4 | 27122 | ENSG00000050165 | 222 |
| DKK4 | Q9UBT3 | 27121 | ENSG00000104371 | 223 |
| DLG3 | Q92796 | 1741 | ENSG00000082458 | 224 |
| DMBT1 | Q9UGM3 | 1755 | ENSG00000187908 | 225 |
| DNAAF5 | Q86Y56 | 54919 | ENSG00000164818 | 226 |
| DNAJB11 | Q9UBS4 | 51726 | ENSG00000090520 | 227 |
| DNAJC3 | Q13217 | 5611 | ENSG00000102580 | 228 |
| DNMT1 | P26358 | 1786 | ENSG00000130816 | 229 |
| DRAXIN | Q8NBI3 | 374946 | ENSG00000162490 | 230 |
| DRG1 | Q9Y295 | 4733 | ENSG00000185721 | 231 |
| DSG2 | Q14126 | 1829 | ENSG00000046604 | 232 |
| ECM1 | Q16610 | 1893 | ENSG00000143369 | 233 |
| EDA | Q92838 | 1896 | ENSG00000158813 | 234 |
| EDIL3 | O43854 | 10085 | ENSG00000164176 | 235 |
| EEF1G | P26641 | 1937 | ENSG00000254772 | 236 |
| EFEMP1 | Q12805 | 2202 | ENSG00000115380 | 237 |
| EFTUD2 | Q15029 | 9343 | ENSG00000108883 | 238 |
| EGFLAM | Q63HQ2 | 133584 | ENSG00000164318 | 239 |
| EIF1AY | O14602 | 9086 | ENSG00000198692 | 240 |
| EIF2B4 | Q9UI10 | 8890 | ENSG00000115211 | 241 |
| EIF2S1 | P05198 | 1965 | ENSG00000134001 | 242 |
| EIF2S2 | P20042 | 8894 | ENSG00000125977 | 243 |
| EIF3A | Q14152 | 8661 | ENSG00000107581 | 244 |
| EIF3C | Q99613 | 8663 | ENSG00000184110 | 245 |
| EIF3F | O00303 | 8665 | ENSG00000175390 | 246 |
| EIF3H | O15372 | 8667 | ENSG00000147677 | 247 |
| EIF3M | Q7L2H7 | 10480 | ENSG00000149100 | 248 |
| EIF5 | P55010 | 1983 | ENSG00000100664 | 249 |
| EIF5B | O60841 | 9669 | ENSG00000158417 | 250 |
| ELAC2 | Q9BQ52 | 60528 | ENSG00000006744 | 251 |
| ELP3 | Q9H9T3 | 55140 | ENSG00000134014 | 252 |
| EMILIN2 | Q9BXX0 | 84034 | ENSG00000132205 | 253 |
| EPHA4 | P54764 | 2043 | ENSG00000116106 | 254 |
| EPHB2 | P29323 | 2048 | ENSG00000133216 | 255 |
| EPHB4 | P54760 | 2050 | ENSG00000196411 | 256 |
| EPRS | P07814 | 2058 | ENSG00000136628 | 257 |
| ERBB3 | P21860 | 2065 | ENSG00000065361 | 258 |
| ERLIN1 | O75477 | 10613 | ENSG00000107566 | 259 |
| ERVMER34-1 | Q9H9K5 | 100288413 | ENSG00000226887 | 260 |
| EXTL2 | Q9UBQ6 | 2135 | ENSG00000162694 | 261 |
| EZR | P15311 | 7430 | ENSG00000092820 | 262 |
| F10 | P00742 | 2159 | ENSG00000126218 | 263 |
| F13A1 | P00488 | 2162 | ENSG00000124491 | 264 |
| F2 | P00734 | 2147 | ENSG00000180210 | 265 |
| F5 | P12259 | 2153 | ENSG00000198734 | 266 |
| FAM129B | Q96TA1 | 64855 | ENSG00000136830 | 267 |
| FAP | Q12884 | 2191 | ENSG00000078098 | 268 |
| FAT1 | Q14517 | 2195 | ENSG00000083857 | 269 |
| FBLN1 | P23142 | 2192 | ENSG00000077942 | 270 |
| FBLN2 | P98095 | 2199 | ENSG00000163520 | 271 |
| FBN1 | P35555 | 2200 | | 272 |
| FBN2 | P35556 | 2201 | ENSG00000138829 | 273 |
| FERMT2 | Q96AC1 | 10979 | ENSG00000073712 | 274 |
| FGB | P02675 | 2244 | ENSG00000171564 | 275 |
| FGF17 | O60258 | 8822 | ENSG00000158815 | 276 |
| FGF2 | P09038 | 2247 | ENSG00000138685 | 277 |
| FGF8 | P55075 | 2253 | ENSG00000107831 | 278 |
| FGF4 | P08620 | 2249 | ENSG00000075388 | 279 |
| FGF6 | P10767 | 2251 | ENSG00000111241 | 280 |
| FGFBP3 | Q8TAT2 | 143282 | ENSG00000174721 | 281 |
| FGFR1 | P11362 | 2260 | ENSG00000077782 | 282 |
| FGFR2 | P21802 | 2263 | ENSG00000066468 | 283 |
| FGFR4 | P22455 | 2264 | ENSG00000160867 | 284 |
| FGFRL1 | Q8N441 | 53834 | ENSG00000127418 | 285 |
| FH | P07954 | 2271 | ENSG00000091483 | 286 |
| FLT1 | P17948 | 2321 | ENSG00000102755 | 287 |
| FN1 | P02751 | 2335 | ENSG00000115414 | 288 |
| FRAS1 | Q86XX4 | 80144 | ENSG00000138759 | 289 |
| FRZB | Q92765 | 2487 | ENSG00000162998 | 290 |
| FST | P19883 | 10468 | ENSG00000134363 | 291 |
| FSTL1 | Q12841 | 11167 | ENSG00000163430 | 292 |
| FUCA2 | Q9BTY2 | 2519 | ENSG00000001036 | 293 |
| FXR1 | P51114 | 8087 | ENSG00000114416 | 294 |
| GALNT1 | Q10472 | 2589 | ENSG00000141429 | 295 |
| GALNT16 | Q8N428 | 57452 | ENSG00000100626 | 296 |
| GALNT2 | Q10471 | 2590 | ENSG00000143641 | 297 |
| GALNT7 | Q86SF2 | 51809 | ENSG00000109586 | 298 |
| GANAB | Q14697 | 23193 | ENSG00000089597 | 299 |
| GARS | P41250 | 2617 | ENSG00000106105 | 300 |
| GBA | P04062 | 2629 | ENSG00000177628 | 301 |
| GC | P02774 | 2638 | ENSG00000145321 | 302 |
| GCNT1 | Q02742 | 2650 | ENSG00000187210 | 303 |
| GDF11 | O95390 | 10220 | ENSG00000135414 | 304 |
| GDF15 | Q99988 | 9518 | ENSG00000130513 | 305 |
| GDF6 | Q6KF10 | 392255 | ENSG00000156466 | 306 |
| GEMIN5 | Q8TEQ6 | 25929 | ENSG00000082516 | 307 |
| GFAP | P14136 | 2670 | ENSG00000131095 | 308 |
| GGH | Q92820 | 8836 | ENSG00000137563 | 309 |
| GLB1 | P16278 | 2720 | ENSG00000170266 | 310 |
| GLG1 | Q92896 | 2734 | ENSG00000090863 | 311 |
| GM2A | P17900 | 2760 | ENSG00000196743 | 312 |
| GNAS | O95467 | 2778 | ENSG00000087460 | 313 |
| GOLM1 | Q8NBJ4 | 51280 | ENSG00000135052 | 314 |
| GOT2 | P00505 | 2806 | ENSG00000125166 | 315 |
| GPC1 | P35052 | 2817 | ENSG00000063660 | 316 |
| GPC3 | P51654 | 2719 | ENSG00000147257 | 317 |
| GPC4 | O75487 | 2239 | ENSG00000076716 | 318 |
| GPI | P06744 | 2821 | ENSG00000105220 | 319 |
| GPRC5B | Q9NZH0 | 51704 | ENSG00000167191 | 320 |
| GPX4 | P36969 | 2879 | ENSG00000167468 | 321 |
| GREM1 | O60565 | 26585 | ENSG00000166923 | 322 |
| GRN | P28799 | 2896 | ENSG00000030582 | 323 |
| GRSF1 | Q12849 | 2926 | ENSG00000132463 | 324 |
| GSN | P06396 | 2934 | ENSG00000148180 | 325 |
| GSPT1 | P15170 | 2935 | ENSG00000103342 | 326 |
| GTF3C3 | Q9Y5Q9 | 9330 | ENSG00000119041 | 327 |
| HABP2 | Q14520 | 3026 | ENSG00000148702 | 328 |
| HADHB | P55084 | 3032 | ENSG00000138029 | 329 |
| HAPLN1 | P10915 | 1404 | ENSG00000145681 | 330 |
| HAPLN3 | Q96S86 | 145864 | | 331 |
| HAPLN4 | Q86UW8 | 404037 | | 332 |
| HARS | P12081 | 3035 | ENSG00000170445 | 333 |
| HBB | P68871 | 3043 | ENSG00000244734 | 334 |
| HBS1L | Q9Y450 | 10767 | ENSG00000112339 | 335 |
| HDGF | P51858 | 3068 | ENSG00000143321 | 336 |
| HDGFL2 | Q7Z4V5 | 84717 | ENSG00000167674 | 337 |
| HDLBP | Q00341 | 3069 | ENSG00000115677 | 338 |
| HGF | P14210 | 3082 | ENSG00000019991 | 339 |
| HGFAC | Q04756 | 3083 | ENSG00000109758 | 340 |
| HIST1H1C | P16403 | 3006 | ENSG00000187837 | 341 |
| HIST1H1E | P10412 | 3008 | ENSG00000168298 | 342 |
| HLA-C | P04222 | | ENSG00000225691 | 343 |
| HMCN1 | Q96RW7 | 83872 | ENSG00000143341 | 344 |
| HMCN2 | Q8NDA2 | | ENSG00000148357 | 345 |
| HMGB1 | P09429 | 3146 | ENSG00000189403 | 346 |
| HMGB2 | P26583 | 3148 | ENSG00000164104 | 347 |
| HMGB3 | O15347 | 3149 | ENSG00000029993 | 348 |
| HMGN1 | P05114 | 3150 | ENSG00000205581 | 349 |
| HMGN5 | P82970 | 79366 | ENSG00000198157 | 350 |
| HNRNPA2B1 | P22626 | 3181 | ENSG00000122566 | 351 |
| HNRNPDL | O14979 | 9987 | ENSG00000152795 | 352 |
| HP | P00738 | 3240 | ENSG00000257017 | 353 |
| HP1BP3 | Q5SSJ5 | 50809 | ENSG00000127483 | 354 |
| HPR | P00739 | 3250 | ENSG00000261701 | 355 |
| HPX | P02790 | 3263 | ENSG00000110169 | 356 |
| HS3ST3A1 | Q9Y663 | 9955 | ENSG00000153976 | 357 |
| HS6ST1 | O60243 | 9394 | ENSG00000136720 | 358 |
| HS6ST2 | Q96MM7 | 90161 | ENSG00000171004 | 359 |
| HSD17B10 | Q99714 | 3028 | ENSG00000072506 | 360 |
| HSD17B4 | P51659 | 3295 | ENSG00000133835 | 361 |
| HSP90AA1 | P07900 | 3320 | ENSG00000080824 | 362 |
| HSP90AB1 | P08238 | 3326 | ENSG00000096384 | 363 |
| HSP90B1 | P14625 | 7184 | ENSG00000166598 | 364 |
| HSPA5 | P11021 | 3309 | ENSG00000044574 | 365 |
| HSPG2 | P98160 | 3339 | ENSG00000142798 | 366 |
| HTRA1 | Q92743 | 5654 | ENSG00000166033 | 367 |
| HYOU1 | Q9Y4L1 | 10525 | ENSG00000149428 | 368 |
| IARS | P41252 | 3376 | ENSG00000196305 | 369 |
| ICAM2 | P13598 | 3384 | ENSG00000108622 | 370 |
| IDE | P14735 | 3416 | ENSG00000119912 | 371 |
| IDH1 | O75874 | 3417 | ENSG00000138413 | 372 |
| IDH2 | P48735 | 3418 | ENSG00000182054 | 373 |
| IGF1 | P05019 | 3479 | ENSG00000017427 | 374 |
| IGF2 | P01344 | 3481 | ENSG00000167244 | 375 |
| IGFBP2 | P18065 | 3485 | ENSG00000115457 | 376 |
| IGFBP3 | P17936 | 3486 | ENSG00000146674 | 377 |
| IGFBP4 | P22692 | 3487 | ENSG00000141753 | 378 |
| IGFBP5 | P24593 | 3488 | ENSG00000115461 | 379 |
| IGFBP6 | P24592 | 3489 | ENSG00000167779 | 380 |
| IGFBP7 | Q16270 | 3490 | ENSG00000163453 | 381 |
| IGFBPL1 | Q8WX77 | 347252 | ENSG00000137142 | 382 |
| IGHA1 | P01876 | | ENSG00000211895 | 383 |
| IGHA2 | P01877 | | ENSG00000211890 | 384 |
| IGHG1 | P01857 | | ENSG00000211896 | 385 |
| IGHG2 | P01859 | | ENSG00000211893 | 386 |
| IGHG4 | P01861 | | ENSG00000211892 | 387 |
| IGHM | P01871 | | ENSG00000211899 | 388 |
| IGKC | P01834 | | | 389 |
| IGKV2-28 | A0A075B6P5 | | ENSG00000244116 | 390 |
| IGKV2D-40 | P01614 | | ENSG00000251039 | 391 |
| IGKV3D-20 | A0A0C4DH25 | | ENSG00000211625 | 392 |
| IGLC2 | P0DOY2 | | ENSG00000211677 | 393 |
| IGLC3 | P0DOY3 | | ENSG00000211679 | 394 |
| IGLV2-11 | P01706 | | ENSG00000211668 | 395 |
| IGSF1 | Q8N6C5 | 3547 | ENSG00000147255 | 396 |
| IGSF10 | Q6WRI0 | 285313 | ENSG00000152580 | 397 |
| ILF2 | Q12905 | 3608 | ENSG00000143621 | 398 |
| INHBA | P08476 | 3624 | ENSG00000122641 | 399 |
| INS | P01308 | 3630 | ENSG00000254647 | 400 |
| INS-IGF2 | F8WCM5 | 723961 | ENSG00000129965 | 401 |
| IPO11 | Q9UI26 | 51194 | ENSG00000086200 | 402 |
| IPO5 | O00410 | 3843 | ENSG00000065150 | 403 |
| IPO8 | O15397 | 10526 | ENSG00000133704 | 404 |
| IQGAP1 | P46940 | 8826 | ENSG00000140575 | 405 |
| ISOC1 | Q96CN7 | 51015 | ENSG00000066583 | 406 |
| ITGAL | P20701 | 3683 | ENSG00000005844 | 407 |
| ITIH1 | P19827 | 3697 | ENSG00000055957 | 408 |
| ITIH2 | P19823 | 3698 | ENSG00000151655 | 409 |
| ITIH3 | Q06033 | 3699 | ENSG00000162267 | 410 |
| ITIH4 | Q14624 | 3700 | ENSG00000055955 | 411 |
| ITIH5 | Q86UX2 | 80760 | ENSG00000123243 | 412 |
| ITLN2 | Q8WWU7 | 142683 | ENSG00000158764 | 413 |
| JCHAIN | P01591 | 3512 | ENSG00000132465 | 414 |
| KARS | Q15046 | 3735 | ENSG00000065427 | 415 |
| KDM1A | O60341 | 23028 | ENSG00000004487 | 416 |
| KMT2A | Q03164 | 4297 | ENSG00000118058 | 417 |
| KNG1 | P01042 | 3827 | ENSG00000113889 | 418 |
| KRT10 | P13645 | 3858 | ENSG00000186395 | 419 |
| KRT14 | P02533 | 3861 | ENSG00000186847 | 420 |
| KRT17 | Q04695 | 3872 | ENSG00000128422 | 421 |
| KRT18 | P05783 | 3875 | ENSG00000111057 | 422 |
| KRT8 | P05787 | 3856 | ENSG00000170421 | 423 |
| LACRT | Q9GZZ8 | 90070 | ENSG00000135413 | 424 |
| LAG3 | P18627 | 3902 | ENSG00000089692 | 425 |
| LAMA1 | P25391 | 284217 | ENSG00000101680 | 426 |
| LAMA2 | P24043 | 3908 | ENSG00000196569 | 427 |
| LAMA5 | O15230 | 3911 | ENSG00000130702 | 428 |
| LAMB1 | P07942 | 3912 | ENSG00000091136 | 429 |
| LAMB2 | P55268 | 3913 | ENSG00000172037 | 430 |
| LAMC1 | P11047 | 3915 | ENSG00000135862 | 431 |
| LARS | Q9P2J5 | 51520 | ENSG00000133706 | 432 |
| LCAT | P04180 | 3931 | ENSG00000213398 | 433 |
| LCN1 | P31025 | 3933 | ENSG00000160349 | 434 |
| LDHA | P00338 | 3939 | ENSG00000134333 | 435 |
| LECT2 | O14960 | 3950 | ENSG00000145826 | 436 |
| LEFTY1 | O75610 | 10637 | ENSG00000243709 | 437 |
| LEFTY2 | O00292 | 7044 | ENSG00000143768 | 438 |
| LEFTYA | | | | 439 |
| LFNG | Q8NES3 | 3955 | ENSG00000106003 | 440 |
| LGALS3BP | Q08380 | 3959 | ENSG00000108679 | 441 |
| LGALS7 | M0R281 | | ENSG00000205076 | 442 |
| LIG3 | P49916 | 3980 | ENSG00000005156 | 443 |
| LINGO1 | Q96FE5 | 84894 | ENSG00000169783 | 444 |
| LIPG | Q9Y5X9 | 9388 | ENSG00000101670 | 445 |
| LMAN2 | Q12907 | 10960 | ENSG00000169223 | 446 |
| LMNA | P02545 | 4000 | ENSG00000160789 | 447 |
| LOXL1 | Q08397 | 4016 | ENSG00000129038 | 448 |
| LOXL2 | Q9Y4K0 | 4017 | ENSG00000134013 | 449 |
| LOXL3 | P58215 | 84695 | ENSG00000115318 | 450 |
| LPL | P06858 | 4023 | ENSG00000175445 | 451 |
| LRG1 | P02750 | 116844 | ENSG00000171236 | 452 |
| LRP1 | Q07954 | 4035 | ENSG00000123384 | 453 |
| LRPAP1 | P30533 | 4043 | ENSG00000163956 | 454 |
| LRRC59 | Q96AG4 | 55379 | ENSG00000108829 | 455 |
| LRRTM4 | Q86VH4 | 80059 | ENSG00000176204 | 456 |
| LSR | Q86X29 | 51599 | ENSG00000105699 | 457 |
| LTBP1 | Q14766 | 4052 | ENSG00000049323 | 458 |
| LTBP4 | Q8N2S1 | 8425 | ENSG00000090006 | 459 |
| LTF | P02788 | 4057 | ENSG00000012223 | 460 |
| LUM | P51884 | 4060 | ENSG00000139329 | 461 |
| LYAR | Q9NX58 | 55646 | ENSG00000145220 | 462 |
| LYZ | P61626 | 4069 | ENSG00000090382 | 463 |
| MANF | P55145 | 7873 | ENSG00000145050 | 464 |
| MAP2K2 | P36507 | 5605 | ENSG00000126934 | 465 |
| MAP4 | P27816 | 4134 | ENSG00000047849 | 466 |
| MAPK1 | P28482 | 5594 | ENSG00000100030 | 467 |
| MASP1 | P48740 | 5648 | ENSG00000127241 | 468 |
| MATN2 | O00339 | 4147 | ENSG00000132561 | 469 |
| MATN3 | O15232 | 4148 | ENSG00000132031 | 470 |
| MATR3 | P43243 | 9782 | ENSG00000015479 | 471 |
| MAZ | P56270 | 4150 | ENSG00000103495 | 472 |
| MBNL1 | Q9NR56 | 4154 | ENSG00000152601 | 473 |
| MCM5 | P33992 | 4174 | ENSG00000100297 | 474 |
| MDH2 | P40926 | 4191 | ENSG00000146701 | 475 |
| MDK | P21741 | 4192 | ENSG00000110492 | 476 |
| MEGF10 | Q96KG7 | 84466 | ENSG00000145794 | 477 |
| MEGF6 | O75095 | 1953 | ENSG00000162591 | 478 |
| METAP2 | P50579 | 10988 | ENSG00000111142 | 479 |
| METTL14 | Q9HCE5 | 57721 | ENSG00000145388 | 480 |
| MFAP2 | P55001 | 4237 | ENSG00000117122 | 481 |
| MFGE8 | Q08431 | 4240 | ENSG00000140545 | 482 |
| MGAT1 | P26572 | 4245 | ENSG00000131446 | 483 |
| MIF | P14174 | 4282 | ENSG00000240972 | 484 |
| MINPP1 | Q9UNW1 | 9562 | ENSG00000107789 | 485 |
| MMP2 | P08253 | 4313 | ENSG00000087245 | 486 |
| MMP9 | P14780 | 4318 | ENSG00000100985 | 487 |
| MSMB | P08118 | 4477 | ENSG00000263639 | 488 |
| MSN | P26038 | 4478 | ENSG00000147065 | 489 |
| MST1 | P26927 | 4485 | ENSG00000173531 | 490 |
| MST1L | Q2TV78 | 11223 | | 491 |
| MTDH | Q86UE4 | 92140 | ENSG00000147649 | 492 |
| MTHFD1 | P11586 | 4522 | ENSG00000100714 | 493 |
| MTHFD2 | P13995 | 10797 | ENSG00000065911 | 494 |
| MXRA5 | Q9NR99 | 25878 | ENSG00000101825 | 495 |
| MYBBP1A | Q9BQG0 | 10514 | ENSG00000132382 | 496 |
| MYL3 | P08590 | 4634 | ENSG00000160808 | 497 |
| MYL4 | P12829 | 4635 | ENSG00000198336 | 498 |
| NAA15 | Q9BXJ9 | 80155 | ENSG00000164134 | 499 |
| NAMPT | P43490 | 10135 | ENSG00000105835 | 500 |
| NASP | P49321 | 4678 | ENSG00000132780 | 501 |
| NCAM1 | P13591 | 4684 | ENSG00000149294 | 502 |
| NCAN | O14594 | 1463 | ENSG00000130287 | 503 |
| NDNF | Q8TB73 | 79625 | ENSG00000173376 | 504 |
| NDST1 | P52848 | 3340 | ENSG00000070614 | 505 |
| NECTIN1 | Q15223 | 5818 | ENSG00000110400 | 506 |
| NECTIN3 | Q9NQS3 | 25945 | ENSG00000177707 | 507 |
| NELL2 | Q99435 | 4753 | ENSG00000184613 | 508 |
| NID1 | P14543 | 4811 | ENSG00000116962 | 509 |
| NID2 | Q14112 | 22795 | ENSG00000087303 | 510 |
| NIPBL | Q6KC79 | 25836 | ENSG00000164190 | 511 |
| NLGN3 | Q9NZ94 | 54413 | ENSG00000196338 | 512 |
| NLGN4Y | Q8NFZ3 | 22829 | ENSG00000165246 | 513 |
| NME1-NME2 | J3KPD9 | | ENSG00000011052 | 514 |
| NMT1 | P30419 | 4836 | ENSG00000136448 | 515 |
| NOLC1 | Q14978 | 9221 | ENSG00000166197 | 516 |
| NOV | A0A024R9J4 | 4856 | | 517 |
| NPC2 | P61916 | 10577 | ENSG00000119655 | 518 |
| NPM3 | O75607 | 10360 | ENSG00000107833 | 519 |
| NPTX1 | Q15818 | 4884 | | 520 |
| NPTX2 | P47972 | 4885 | ENSG00000106236 | 521 |
| NPTXR | O95502 | 23467 | ENSG00000221890 | 522 |
| NRG1 | Q02297 | 3084 | ENSG00000157168 | 523 |
| NRG2 | O14511 | 9542 | ENSG00000158458 | 524 |
| NRP1 | O14786 | 8829 | ENSG00000099250 | 525 |
| NRP2 | O60462 | 8828 | ENSG00000118257 | 526 |
| NSUN5 | Q96P11 | 55695 | ENSG00000130305 | 527 |
| NTS | P30990 | 4922 | ENSG00000133636 | 528 |
| NUBP2 | Q9Y5Y2 | 10101 | ENSG00000095906 | 529 |
| NUCB1 | Q02818 | 4924 | ENSG00000104805 | 530 |
| NUMA1 | Q14980 | 4926 | ENSG00000137497 | 531 |
| NUP155 | O75694 | 9631 | ENSG00000113569 | 532 |
| OAF | Q86UD1 | 220323 | ENSG00000184232 | 533 |
| OLA1 | Q9NTK5 | 29789 | ENSG00000138430 | 534 |
| OLFM2 | O95897 | 93145 | | 535 |
| OLFML2A | Q68BL7 | 169611 | ENSG00000185585 | 536 |
| OLFML3 | Q9NRN5 | 56944 | ENSG00000116774 | 537 |
| ORM1 | P02763 | 5004 | ENSG00000229314 | 538 |
| ORM2 | P19652 | 5005 | ENSG00000228278 | 539 |
| P4HB | P07237 | 5034 | ENSG00000185624 | 540 |
| PACSIN2 | Q9UNF0 | 11252 | ENSG00000100266 | 541 |
| PAFAH1B1 | P43034 | 5048 | ENSG00000007168 | 542 |
| PAIP1 | Q9H074 | 10605 | ENSG00000172239 | 543 |
| PAM | P19021 | 5066 | ENSG00000145730 | 544 |
| PAMR1 | Q6UXH9 | 25891 | ENSG00000149090 | 545 |
| PAPLN | O95428 | 89932 | ENSG00000100767 | 546 |
| PAPPA | Q13219 | 5069 | ENSG00000182752 | 547 |
| PARP1 | P09874 | 142 | ENSG00000143799 | 548 |
| PC | P11498 | 5091 | ENSG00000173599 | 549 |
| PCDH1 | Q08174 | 5097 | ENSG00000156453 | 550 |
| PCLO | Q9Y6V0 | 27445 | ENSG00000186472 | 551 |
| PCOLCE | Q15113 | 5118 | ENSG00000106333 | 552 |
| PCOLCE2 | Q9UKZ9 | 26577 | ENSG00000163710 | 553 |
| PCSK5 | Q92824 | 5125 | ENSG00000099139 | 554 |
| PCSK9 | Q8NBP7 | 255738 | ENSG00000169174 | 555 |
| PDCD6IP | Q8WUM4 | 10015 | ENSG00000170248 | 556 |
| PDGFD | Q9GZP0 | 80310 | ENSG00000170962 | 557 |
| PDGFRL | Q15198 | 5157 | ENSG00000104213 | 558 |
| PDIA3 | P30101 | 2923 | ENSG00000167004 | 559 |
| PDIA4 | P13667 | 9601 | ENSG00000155660 | 560 |
| PDIA5 | Q14554 | 10954 | ENSG00000065485 | 561 |
| PDIA6 | Q15084 | 10130 | ENSG00000143870 | 562 |
| PFAS | O15067 | 5198 | ENSG00000178921 | 563 |
| PFKP | Q01813 | 5214 | ENSG00000067057 | 564 |
| PFN1 | P07737 | 5216 | ENSG00000108518 | 565 |
| PGD | P52209 | 5226 | ENSG00000142657 | 566 |
| PGLYRP2 | Q96PD5 | 114770 | ENSG00000161031 | 567 |
| PHGDH | O43175 | 26227 | ENSG00000092621 | 568 |
| PI16 | Q6UXB8 | 221476 | ENSG00000164530 | 569 |
| PIGR | P01833 | 5284 | ENSG00000162896 | 570 |
| PIP | P12273 | 5304 | ENSG00000159763 | 571 |
| PKDCC | Q504Y2 | 91461 | ENSG00000162878 | 572 |
| PKM | P14618 | 5315 | ENSG00000067225 | 573 |
| PLAT | P00750 | 5327 | ENSG00000104368 | 574 |
| PLAU | P00749 | 5328 | ENSG00000122861 | 575 |
| PLCB3 | Q01970 | 5331 | ENSG00000149782 | 576 |
| PLEC | Q15149 | 5339 | ENSG00000178209 | 577 |
| PLG | P00747 | 5340 | ENSG00000122194 | 578 |
| PLIN4 | Q96Q06 | 729359 | ENSG00000167676 | 579 |
| PLOD1 | Q02809 | 5351 | ENSG00000083444 | 580 |
| PLOD2 | O00469 | 5352 | ENSG00000152952 | 581 |
| PLOD3 | O60568 | 8985 | ENSG00000106397 | 582 |
| PLTP | P55058 | 5360 | ENSG00000100979 | 583 |
| POLL | Q9UGP5 | 27343 | ENSG00000166169 | 584 |
| POMC | P01189 | 5443 | ENSG00000115138 | 585 |
| POSTN | Q15063 | 10631 | ENSG00000133110 | 586 |
| PPIA | P62937 | 5478 | ENSG00000196262 | 587 |
| PPIB | P23284 | 5479 | ENSG00000166794 | 588 |
| PPP1CA | P62136 | 5499 | ENSG00000172531 | 589 |
| PPP1CC | P36873 | 5501 | ENSG00000186298 | 590 |
| PPP2R1A | P30153 | 5518 | ENSG00000105568 | 591 |
| PPT1 | P50897 | 5538 | ENSG00000131238 | 592 |
| PRB3 | Q04118 | | ENSG00000197870 | 593 |
| PRB4 | P10163 | | | 594 |
| PRCP | P42785 | 5547 | ENSG00000137509 | 595 |
| PRDX2 | P32119 | 7001 | ENSG00000167815 | 596 |
| PRDX4 | Q13162 | 10549 | ENSG00000123131 | 597 |
| PRDX5 | P30044 | 25824 | ENSG00000126432 | 598 |
| PRG4 | Q92954 | 10216 | ENSG00000116690 | 599 |
| PRKDC | P78527 | 5591 | ENSG00000253729 | 600 |
| PRMT1 | Q99873 | 3276 | ENSG00000126457 | 601 |
| PRMT5 | O14744 | 10419 | ENSG00000100462 | 602 |
| PROM1 | O43490 | 8842 | ENSG00000007062 | 603 |
| PRPF19 | Q9UMS4 | 27339 | ENSG00000110107 | 604 |
| PRPF40A | O75400 | 55660 | ENSG00000196504 | 605 |
| PRPF4B | Q13523 | 8899 | ENSG00000112739 | 606 |
| PRPF6 | O94906 | 24148 | ENSG00000101161 | 607 |
| PRPF8 | Q6P2Q9 | 10594 | ENSG00000174231 | 608 |
| PRPSAP2 | O60256 | 5636 | ENSG00000141127 | 609 |
| PRR4 | Q16378 | 11272 | ENSG00000111215 | 610 |
| PRSS2 | P07478 | 5645 | ENSG00000275896 | 611 |
| PRSS23 | O95084 | 11098 | ENSG00000150687 | 612 |
| PRSS3 | P35030 | 5646 | ENSG00000010438 | 613 |
| PRTG | Q2VWP7 | 283659 | ENSG00000166450 | 614 |
| PSIP1 | O75475 | 11168 | ENSG00000164985 | 615 |
| PSMB6 | P28072 | 5694 | ENSG00000142507 | 616 |
| PSMD1 | Q99460 | 5707 | ENSG00000173692 | 617 |
| PSMD2 | Q13200 | 5708 | ENSG00000175166 | 618 |
| PSMD5 | Q16401 | 5711 | ENSG00000095261 | 619 |
| PSMD6 | Q15008 | 9861 | ENSG00000163636 | 620 |
| PSMD8 | P48556 | 5714 | ENSG00000099341 | 621 |
| PSME3 | P61289 | 10197 | ENSG00000131467 | 622 |
| PTK2 | Q05397 | 5747 | ENSG00000169398 | 623 |
| PTK7 | Q13308 | 5754 | ENSG00000112655 | 624 |
| PTN | P21246 | 5764 | ENSG00000105894 | 625 |
| PTPRC | P08575 | 5788 | ENSG00000081237 | 626 |
| PTPRD | P23468 | 5789 | ENSG00000153707 | 627 |
| PTPRF | P10586 | 5792 | ENSG00000142949 | 628 |
| PTPRS | Q13332 | 5802 | ENSG00000105426 | 629 |
| PTPRZ1 | P23471 | 5803 | ENSG00000106278 | 630 |
| PUF60 | Q9UHX1 | 22827 | ENSG00000179950 | 631 |
| PXDN | Q92626 | 7837 | ENSG00000130508 | 632 |
| PZP | P20742 | | ENSG00000126838 | 633 |
| QPRT | Q15274 | 23475 | ENSG00000103485 | 634 |
| QSOX1 | O00391 | 5768 | ENSG00000116260 | 635 |
| RAB7A | P51149 | 7879 | ENSG00000075785 | 636 |
| RACK1 | P63244 | 10399 | ENSG00000204628 | 637 |
| RARRES2 | Q99969 | 5919 | ENSG00000106538 | 638 |
| RBMX | P38159 | 27316 | ENSG00000147274 | 639 |
| RBP4 | P02753 | 5950 | ENSG00000138207 | 640 |
| RCC1 | P18754 | 1104 | ENSG00000180198 | 641 |
| RCOR1 | Q9UKL0 | 23186 | ENSG00000089902 | 642 |
| RECQL | P46063 | 5965 | ENSG00000004700 | 643 |
| RELN | P78509 | 5649 | ENSG00000189056 | 644 |
| RNASE1 | P07998 | 6035 | ENSG00000129538 | 645 |
| RNASE4 | P34096 | 6038 | ENSG00000258818 | 646 |
| ROBO1 | Q9Y6N7 | 6091 | ENSG00000169855 | 647 |
| RPL14 | P50914 | 9045 | ENSG00000188846 | 648 |
| RPL18 | Q07020 | 6141 | ENSG00000063177 | 649 |
| RPL19 | P84098 | 6143 | ENSG00000108298 | 650 |
| RPL23A | P62750 | 6147 | ENSG00000198242 | 651 |
| RPL26 | P61254 | 6154 | ENSG00000161970 | 652 |
| RPL29 | P47914 | 6159 | ENSG00000162244 | 653 |
| RPL3 | P39023 | 6122 | ENSG00000100316 | 654 |
| RPL35 | P42766 | 11224 | ENSG00000136942 | 655 |
| RPL4 | P36578 | 6124 | ENSG00000174444 | 656 |
| RPL9 | A0A2R8Y5Y7 | | ENSG00000163682 | 657 |
| RPLP0 | P05388 | 6175 | ENSG00000089157 | 658 |
| RPLP1 | P05386 | 6176 | ENSG00000137818 | 659 |
| RPLP2 | P05387 | 6181 | ENSG00000177600 | 660 |
| RPN2 | P04844 | 6185 | ENSG00000118705 | 661 |
| RPS13 | P62277 | 6207 | ENSG00000110700 | 662 |
| RPS20 | P60866 | 6224 | ENSG00000008988 | 663 |
| RPS23 | P62266 | 6228 | ENSG00000186468 | 664 |
| RPS27A | P62979 | 6233 | ENSG00000143947 | 665 |
| RPS27L | Q71UM5 | 51065 | ENSG00000185088 | 666 |
| RPS3 | P23396 | 6188 | ENSG00000149273 | 667 |
| RRBP1 | Q9P2E9 | 6238 | ENSG00000125844 | 668 |
| RSF1 | Q96T23 | 51773 | ENSG00000048649 | 669 |
| RSL1D1 | O76021 | 26156 | ENSG00000171490 | 670 |
| RTF1 | Q92541 | 23168 | ENSG00000137815 | 671 |
| RTN4 | Q9NQC3 | 57142 | ENSG00000115310 | 672 |
| RTN4RL2 | Q86UN3 | 349667 | ENSG00000186907 | 673 |
| RUVBL2 | Q9Y230 | 10856 | ENSG00000183207 | 674 |
| S100A12 | P80511 | 6283 | ENSG00000163221 | 675 |
| S100A13 | Q99584 | 6284 | ENSG00000189171 | 676 |
| S100A7 | P31151 | 6278 | ENSG00000143556 | 677 |
| S100A8 | P05109 | 6279 | ENSG00000143546 | 678 |
| S100A9 | P06702 | 6280 | ENSG00000163220 | 679 |
| SAP30 | O75446 | 8819 | ENSG00000164105 | 680 |
| SARS | P49591 | 6301 | ENSG00000031698 | 681 |
| SBSN | Q6UWP8 | 374897 | ENSG00000189001 | 682 |
| SCG3 | Q8WXD2 | 29106 | ENSG00000104112 | 683 |
| SCGB2A2 | Q13296 | 4250 | ENSG00000110484 | 684 |
| SCUBE1 | Q8IWY4 | 80274 | ENSG00000159307 | 685 |
| SCUBE3 | Q8IX30 | 222663 | ENSG00000146197 | 686 |
| SDC1 | P18827 | 6382 | ENSG00000115884 | 687 |
| SDC4 | P31431 | 6385 | ENSG00000124145 | 688 |
| SDCBP | O00560 | 6386 | ENSG00000137575 | 689 |
| SDF4 | Q9BRK5 | 51150 | ENSG00000078808 | 690 |
| SEC13 | P55735 | 6396 | ENSG00000157020 | 691 |
| SELENOP | P49908 | 6414 | ENSG00000250722 | 692 |
| SEMA3A | Q14563 | 10371 | ENSG00000075213 | 693 |
| SEMA3F | Q13275 | 6405 | ENSG00000001617 | 694 |
| SEMA4B | Q9NPR2 | 10509 | ENSG00000185033 | 695 |
| SEMA4D | Q92854 | 10507 | ENSG00000187764 | 696 |
| SEMA5A | Q13591 | 9037 | ENSG00000112902 | 697 |
| SEMA6A | Q9H2E6 | 57556 | ENSG00000092421 | 698 |
| SEMA6D | Q8NFY4 | 80031 | ENSG00000137872 | 699 |
| SEMG1 | P04279 | 6406 | ENSG00000124233 | 700 |
| SEPT9 | Q9UHD8 | 10801 | ENSG00000184640 | 701 |
| SERBP1 | Q8NC51 | 26135 | ENSG00000142864 | 702 |
| SERPINA1 | P01009 | 5265 | ENSG00000197249 | 703 |
| SERPINA3 | P01011 | 12 | ENSG00000196136 | 704 |
| SERPINA5 | P05154 | 5104 | ENSG00000188488 | 705 |
| SERPINA7 | P05543 | 6906 | ENSG00000123561 | 706 |
| SERPINB12 | Q96P63 | 89777 | ENSG00000166634 | 707 |
| SERPINB3 | P29508 | 6317 | ENSG00000057149 | 708 |
| SERPINB9 | P50453 | 5272 | ENSG00000170542 | 709 |
| SERPINC1 | P01008 | 462 | ENSG00000117601 | 710 |
| SERPIND1 | P05546 | 3053 | ENSG00000099937 | 711 |
| SERPINE1 | P05121 | 5054 | ENSG00000106366 | 712 |
| SERPINE2 | P07093 | 5270 | ENSG00000135919 | 713 |
| SERPINF1 | P36955 | 5176 | ENSG00000132386 | 714 |
| SERPINF2 | P08697 | 5345 | ENSG00000167711 | 715 |
| SERPING1 | P05155 | 710 | ENSG00000149131 | 716 |
| SERPINH1 | P50454 | 871 | ENSG00000149257 | 717 |
| SERPINI1 | Q99574 | 5274 | ENSG00000163536 | 718 |
| SF3B1 | O75533 | 23451 | ENSG00000115524 | 719 |
| SF3B2 | Q13435 | 10992 | ENSG00000087365 | 720 |
| SF3B3 | Q15393 | 23450 | ENSG00000189091 | 721 |
| SFPQ | P23246 | 6421 | ENSG00000116560 | 722 |
| SFRP1 | Q8N474 | 6422 | ENSG00000104332 | 723 |
| SFRP2 | Q96HF1 | 6423 | ENSG00000145423 | 724 |
| SH2B1 | Q9NRF2 | 25970 | ENSG00000178188 | 725 |
| SHBG | P04278 | 6462 | ENSG00000129214 | 726 |
| SHMT1 | P34896 | 6470 | ENSG00000176974 | 727 |
| SKIV2L2 | L8E9T8 | | | 728 |
| SLC1A5 | Q15758 | 6510 | ENSG00000105281 | 729 |
| SLC2A14 | Q8TDB8 | 144195 | ENSG00000173262 | 730 |
| SLC39A10 | Q9ULF5 | 57181 | ENSG00000196950 | 731 |
| SLIT2 | O94813 | 9353 | | 732 |
| SLIT3 | O75094 | 6586 | ENSG00000184347 | 733 |
| SLPI | P03973 | 6590 | ENSG00000124107 | 734 |
| SLTM | Q9NWH9 | 79811 | ENSG00000137776 | 735 |
| SLURP1 | P55000 | 57152 | ENSG00000126233 | 736 |
| SMARCC1 | Q92922 | 6599 | ENSG00000173473 | 737 |
| SMARCD1 | Q96GM5 | 6602 | ENSG00000066117 | 738 |
| SMARCD2 | Q92925 | 6603 | ENSG00000108604 | 739 |
| SMC1A | Q14683 | 8243 | ENSG00000072501 | 740 |
| SMC3 | Q9UQE7 | 9126 | ENSG00000108055 | 741 |
| SMOC1 | Q9H4F8 | 64093 | ENSG00000198732 | 742 |
| SMOC2 | Q9H3U7 | 64094 | ENSG00000112562 | 743 |
| SMPDL3B | Q92485 | 27293 | ENSG00000130768 | 744 |
| SMR3B | P02814 | 10879 | ENSG00000171201 | 745 |
| SNRPB | P14678 | 6628 | ENSG00000125835 | 746 |
| SNRPD1 | P62314 | 6632 | ENSG00000167088 | 747 |
| SNRPD3 | P62318 | 6634 | ENSG00000100028 | 748 |
| SOD3 | P08294 | 6649 | ENSG00000109610 | 749 |
| SPARC | P09486 | 6678 | ENSG00000113140 | 750 |
| SPINT1 | O43278 | 6692 | ENSG00000166145 | 751 |
| SPINT2 | O43291 | 10653 | ENSG00000167642 | 752 |
| SPOCK1 | Q08629 | 6695 | ENSG00000152377 | 753 |
| SPON1 | Q9HCB6 | 10418 | ENSG00000262655 | 754 |
| SPP1 | P10451 | 6696 | ENSG00000118785 | 755 |
| SRP14 | P37108 | 6727 | ENSG00000140319 | 756 |
| SRPX | P78539 | 8406 | ENSG00000101955 | 757 |
| SRPX2 | O60687 | 27286 | ENSG00000102359 | 758 |
| SRSF1 | Q07955 | 6426 | ENSG00000136450 | 759 |
| SSB | P05455 | 6741 | ENSG00000138385 | 760 |
| SSC5D | A1L4H1 | 284297 | ENSG00000179954 | 761 |
| ST6GAL1 | P15907 | 6480 | ENSG00000073849 | 762 |
| ST6GAL2 | Q96JF0 | 84620 | ENSG00000144057 | 763 |
| STAG1 | Q8WVM7 | 10274 | ENSG00000118007 | 764 |
| STC1 | P52823 | 6781 | ENSG00000159167 | 765 |
| STC2 | O76061 | 8614 | ENSG00000113739 | 766 |
| SUB1 | P53999 | 10923 | ENSG00000113387 | 767 |
| SULF2 | Q8IWU5 | 55959 | ENSG00000196562 | 768 |
| SUMF2 | Q8NBJ7 | 25870 | ENSG00000129103 | 769 |
| SUPT16H | Q9Y5B9 | 11198 | ENSG00000092201 | 770 |
| SUPT6H | Q7KZ85 | 6830 | ENSG00000109111 | 771 |
| SVEP1 | Q4LDE5 | 79987 | ENSG00000165124 | 772 |
| SYNCRIP | O60506 | 10492 | ENSG00000135316 | 773 |
| TAGLN2 | P37802 | 8407 | ENSG00000158710 | 774 |
| TBL1XR1 | Q9BZK7 | 79718 | ENSG00000177565 | 775 |
| TCN2 | P20062 | 6948 | ENSG00000185339 | 776 |
| TCOF1 | Q13428 | 6949 | ENSG00000070814 | 777 |
| TF | P02787 | 7018 | ENSG00000091513 | 778 |
| TFAM | Q00059 | 7019 | ENSG00000108064 | 779 |
| TFPI | P10646 | 7035 | ENSG00000003436 | 780 |
| TFRC | P02786 | 7037 | ENSG00000072274 | 781 |
| TGFB2 | P61812 | 7042 | ENSG00000092969 | 782 |
| TGFBI | Q15582 | 7045 | ENSG00000120708 | 783 |
| THBS1 | P07996 | 7057 | ENSG00000137801 | 784 |
| THBS2 | P35442 | 7058 | ENSG00000186340 | 785 |
| THBS3 | P49746 | 7059 | ENSG00000169231 | 786 |
| THBS4 | P35443 | 7060 | ENSG00000113296 | 787 |
| THOC3 | Q96J01 | 84321 | ENSG00000051596 | 788 |
| THSD7A | Q9UPZ6 | 221981 | ENSG00000005108 | 789 |
| TIMP1 | P01033 | 7076 | ENSG00000102265 | 790 |
| TIMP2 | P16035 | 7077 | ENSG00000035862 | 791 |
| TIMP3 | P35625 | 7078 | ENSG00000100234 | 792 |
| TINAGL1 | Q9GZM7 | 64129 | ENSG00000142910 | 793 |
| TJP1 | Q07157 | 7082 | ENSG00000104067 | 794 |
| TNC | P24821 | 3371 | ENSG00000041982 | 795 |
| TNN | Q9UQP3 | 63923 | | 796 |
| TNXB | P22105 | 7148 | ENSG00000168477 | 797 |
| TOP1 | P11387 | 7150 | ENSG00000198900 | 798 |
| TPM4 | P67936 | 7171 | ENSG00000167460 | 799 |
| TPP2 | P29144 | 7174 | ENSG00000134900 | 800 |
| TRIM28 | Q13263 | 10155 | ENSG00000130726 | 801 |
| TRIP10 | Q15642 | 9322 | ENSG00000125733 | 802 |
| TRMT1 | Q9NXH9 | 55621 | ENSG00000104907 | 803 |
| TSKU | Q8WUA8 | 25987 | ENSG00000182704 | 804 |
| TTR | P02766 | 7276 | ENSG00000118271 | 805 |
| TUBB4A | P04350 | 10382 | ENSG00000104833 | 806 |
| TUFM | P49411 | 7284 | ENSG00000178952 | 807 |
| TWSG1 | Q9GZX9 | 57045 | ENSG00000128791 | 808 |
| TXN | P10599 | 7295 | ENSG00000136810 | 809 |
| TXNDC16 | Q9P2K2 | 57544 | ENSG00000087301 | 810 |
| TXNDC5 | Q8NBS9 | 81567 | ENSG00000239264 | 811 |
| U2AF2 | P26368 | 11338 | ENSG00000063244 | 812 |
| UBE20 | Q9C0C9 | 63893 | ENSG00000175931 | 813 |
| UBR4 | Q5T4S7 | 23352 | ENSG00000127481 | 814 |
| UCHL1 | P09936 | 7345 | ENSG00000154277 | 815 |
| UCHL3 | P15374 | 7347 | ENSG00000118939 | 816 |
| UFL1 | O94874 | 23376 | ENSG00000014123 | 817 |
| UGP2 | Q16851 | 7360 | ENSG00000169764 | 818 |
| USP11 | P51784 | 8237 | ENSG00000102226 | 819 |
| USP14 | P54578 | 9097 | ENSG00000101557 | 820 |
| USP43 | Q70EL4 | 124739 | ENSG00000154914 | 821 |
| UTP4 | Q969X6 | 84916 | ENSG00000141076 | 822 |
| VARS | P26640 | 7407 | ENSG00000096171 | 823 |
| VASN | Q6EMK4 | 114990 | ENSG00000168140 | 824 |
| VCAN | P13611 | 1462 | ENSG00000038427 | 825 |
| VCP | P55072 | 7415 | ENSG00000165280 | 826 |
| VEGFA | P15692 | 7422 | ENSG00000112715 | 827 |
| VIT | Q6UXI7 | 5212 | ENSG00000205221 | 828 |
| VNN1 | O95497 | 8876 | ENSG00000112299 | 829 |
| VPS35 | Q96QK1 | 55737 | ENSG00000069329 | 830 |
| VTN | P04004 | 7448 | ENSG00000109072 | 831 |
| VWF | P04275 | 7450 | ENSG00000110799 | 832 |
| WDR3 | Q9UNX4 | 10885 | ENSG00000065183 | 833 |
| WDR36 | Q8NI36 | 134430 | ENSG00000134987 | 834 |
| WDR4 | P57081 | 10785 | ENSG00000160193 | 835 |
| WDR43 | Q15061 | 23160 | ENSG00000163811 | 836 |
| WFIKKN1 | Q96NZ8 | 117166 | ENSG00000127578 | 837 |
| WFIKKN2 | Q8TEU8 | 124857 | ENSG00000173714 | 838 |
| XRCC5 | P13010 | 7520 | ENSG00000079246 | 839 |
| XYLT1 | Q86Y38 | 64131 | ENSG00000103489 | 840 |
| XYLT2 | Q9H1B5 | 64132 | ENSG00000015532 | 841 |
| YBX1 | P67809 | 4904 | ENSG00000065978 | 842 |
| YBX3 | P16989 | 8531 | ENSG00000060138 | 843 |
| ZG16B | Q96DA0 | 124220 | ENSG00000162078 | 844 |
| ZNF207 | O43670 | 7756 | ENSG00000010244 | 845 |
| ZNF326 | Q5BKZ1 | 284695 | ENSG00000162664 | 846 |
| ZNF706 | Q9Y5V0 | 51123 | ENSG00000120963 | 847 |
| APLP1 | P51693 | 333 | ENSG00000105290 | 848 |
| APP | P05067 | 351 | ENSG00000142192 | 849 |
| NPNT | Q6UXI9 | 255743 | ENSG00000168743 | 850 |
| RPL22 | Q6UXI9 | 6146 | ENSG00000116251 | 851 |
| FGF19 | O95750 | 9965 | ENSG00000162344 | 852 |
| BTC | P35070 | 685 | ENSG00000174808 | 853 |
| IL13RA2 | Q14627 | 3598 | ENSG00000123496 | 854 |
| CD170 | O15389 | | ENSG00000105501 | 855 |
| IL15 | P40933 | 3600 | ENSG00000164136 | 856 |
| WAP | Q8TEU8 | | ENSG00000173714 | 857 |
| MFRP | Q9BY79 | 83552 | ENSG00000235718 | 858 |
| IL10Ra | Q13651 | 3587 | ENSG00000110324 | 859 |
| ChemR23 | Q99788 | 1240 | ENSG00000174600 | 860 |
| HBEGF | Q99075 | 1839 | ENSG00000113070 | 861 |
| IL16 | Q14005 | 3603 | ENSG00000172349 | 862 |
| IL7Ra | P16871 | 3575 | ENSG00000168685 | 863 |
| TNFSF10C | O14798 | 8794 | ENSG00000173535 | 864 |
| BMP6 | P22004 | 654 | ENSG00000153162 | 865 |
| IL36g | P14778 | 56300 | ENSG00000136688 | 866 |
| IL1RA | P18510 | 3557 | ENSG00000136689 | 867 |
| KREMEN2 | Q8NCW0 | 79412 | | 868 |
| TNFSF10D | Q9UBN6 | 8793 | ENSG00000173530 | 869 |
| CXCCR1 | P49238 | 1524 | ENSG00000168329 | 870 |
| CCL23 | P55773 | 6368 | ENSG00000276114 | 871 |
| Catenin | P35222 | 1499 | ENSG00000168036 | 872 |
| TNFSF10 | P50591 | 8743 | ENSG00000121858 | 873 |
| CCL14 | Q16627 | 6358 | ENSG00000276409 | 874 |
| IL2 | P60568 | 3558 | ENSG00000109471 | 875 |
| FGF1 | P05230 | 2246 | ENSG00000113578 | 876 |

1-24. (canceled)

25. A method for treating muscle, comprising the steps of:
obtaining a subject suffering from a muscle-wasting condition; and
treating the subject with a composition wherein the composition comprises a polypeptide of Table 2, and wherein the administration increases expression of MYOG, MYOD1, MYH3, CK, or ATP1B1.

26. The method of claim 25, wherein the polypeptide of Table 2 comprises an IGF2 polypeptide, a FGF17 polypeptide, a BMP7 polypeptide, a TGFb1 polypeptide, a FGF1 polypeptide, or a PDGFD polypeptide.

27. The method of claim 26, wherein the treating the subject increases a regenerative capability of a myoblast of the subject, the regenerative capability comprising a proliferation, a degree of differentiation, or a cellular survival.

28. The method of claim 27, wherein the increase in the regenerative capability of the myoblast comprises an increase in proliferation of the myoblast, resulting in an increase in new myofibers.

29. The method of claim 27, wherein the subject has an increase in a muscle regeneration.

30. The method of claim 25, wherein a grip strength of the subject is increased.

31. The method of claim 25, wherein a weight of muscle in the subject is increased.

32. The method of claim 25, wherein a forelimb force in the subject is increased.

33. The method of claim 25, wherein a lean body mass of the subject is increased.

34. The method of claim 25, wherein an appendicular skeletal muscle index of the subject is increased.

35. The method of claim 25, wherein the muscle-wasting condition is a sarcopenia.

36. The method of claim 25, wherein the muscle-wasting condition is a muscular dystrophy.

37. The method of claim 25, wherein the muscle-wasting condition is a result of an obesity, a disease progression, a metabolic disorder, a therapeutic treatment, or a combination thereof.

38. The method of claim 25, wherein the muscle-wasting condition is a cachexia.

39. The method of claim 37, wherein the metabolic disorder is a diabetes.

40. The method of claim 25, wherein the muscle-wasting condition results from a muscle injury, or a limb immobilization.

41. The method of claim 26, wherein the polypeptide has a modification to improve stability.

42. The method of claim 41, wherein the modification is a chemical medication.

43. The method of claim 41, wherein the modification is a conjugation to another protein.

44. The method of claim 43, wherein the other protein is a serum albumin or an immunoglobulin.
